SEQUENCE LISTING (864 sequences)

SEQ ID NO: 1 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSS

SEQ ID NO: 2 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIKDSGSWGQGTQVTVSS

SEQ ID NO: 3 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYKDSGSWGQGTQVTVSS

SEQ ID NO: 4 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYQDSGSWGQGTQVTVSS

SEQ ID NO: 5 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRYQDSGSWGQGTQVTVSS

SEQ ID NO: 6 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIQDSGSWGQGTQVTVSS

SEQ ID NO: 7 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWIIQDSGSWGQGTQVTVSS

SEQ ID NO: 8 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRKDSGSWGQGTQVTVSS

SEQ ID NO: 9 - protein, 129 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRKDSGSWGQGTQVTVSS

SEQ ID NO: 10 - protein, 129 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRQDSGSWGQGTQVTVSS

SEQ ID NO: 11 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRQDSGSWGQGTQVTVSS

SEQ ID NO: 12 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPRQDSGSWGQGTQVTVSS

SEQ ID NO: 13 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRLQDSGSWGQGTQVTVSS

SEQ ID NO: 14 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRLQDSGSWGQGTQVTVSS

SEQ ID NO: 15 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHKDSGSWGQGTQVTVSS

SEQ ID NO: 16 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHQDSGSWGQGTQVTVSS

SEQ ID NO: 17 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPHQDSGSWGQGTQVTVSS

SEQ ID NO: 18 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHGKDSGSWGQGTQVTVSS

SEQ ID NO: 19 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWHGKDSGSWGQGTQVTVSS

SEQ ID NO: 20 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHNKDSGSWGQGTQVTVSS

SEQ ID NO: 21 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHYKDSGSWGQGTQVTVSS

SEQ ID NO: 22 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKNKDSGSWGQGTQVTVSS

SEQ ID NO: 23 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKYKDSGSWGQGTQVTVSS

SEQ ID NO: 24 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNNKDSGSWGQGTQVTVSS

SEQ ID NO: 25 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNYKDSGSWGQGTQVTVSS

SEQ ID NO: 26 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQNKDSGSWGQGTQVTVSS

SEQ ID NO: 27 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQYKDSGSWGQGTQVTVSS

SEQ ID NO: 28 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRNKDSGSWGQGTQVTVSS

SEQ ID NO: 29 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSNKDSGSWGQGTQVTVSS

SEQ ID NO: 30 - protein, 129 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSYKDSGSWGQGTQVTVSS

SEQ ID NO: 31 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIHKDSGSWGQGTQVTVSS

SEQ ID NO: 32 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWILKDSGSWGQGTQVTVSS

SEQ ID NO: 33 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWINKDSGSWGQGTQVTVSS

SEQ ID NO: 34 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTHKDSGSWGQGTQVTVSS

SEQ ID NO: 35 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTIKDSGSWGQGTQVTVSS

SEQ ID NO: 36 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTLKDSGSWGQGTQVTVSS

SEQ ID NO: 37 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTNKDSGSWGQGTQVTVSS

SEQ ID NO: 38 - protein, 129 aa, synthetic construct
EVQLVESGGGLVQPGGSLRLSCAASGYNYVFHCMGWYRQAPGKGREFVALMSRSRGYYYADSVKGRFTISRDNSKNTVYLQMNSLRAEDTAVYYCAAVPRAGIESGAYCKWHGKDSGSWGQGTQVTVSS

SEQ ID NO: 39 - protein, 129 aa, synthetic construct
EVQLVESGGGLVQPGGSLRLSCAASGITYVSHCMGWYRQAPGKGREFVAAMSRSRGYYYADSVKGRFTISRDNSKNTVYLQMNSLRAEDTAVYYCAAVPRAGIESGAYCKWHGKDSGSWGQGTQVTVSS

SEQ ID NO: 40 - protein, 129 aa, synthetic construct
EVQLVESGGGLVQPGGSLRLSCAASGITYSSHCMGWYRQAPGKGREFVAAMQRSRGTYYADSVKGRFTISRDNSKNTVYLQMNSLRAEDTAVYYCAAVPRAGIESGAYCKWHGKDSGSWGQGTQVTVSS

SEQ ID NO: 41 - protein, 129 aa, synthetic construct
EVQLVESGGGLVQPGGSLRLSCAASGITYQSHCMGWYRQAPGKGREFVALIQRSRGTYYADSVKGRFTISRDNSKNTVYLQMNSLRAEDTAVYYCAAVPRAGIESGAYCKWHGKDSGSWGQGTQVTVSS

SEQ ID NO: 42 - protein, 129 aa, synthetic construct
EVQLVESGGGLVQPGGSLRLSCAASGLHYHSHCMGWYRQAPGKGREFVAAMSHSRGYYYADSVKGRFTISRDNSKNTVYLQMNSLRAEDTAVYYCAAVPRAGIESGAYCKWQNKDSGSWGQGTQVTVSS

SEQ ID NO: 43 - protein, 129 aa, synthetic construct
EVQLVESGGGLVQPGGSLRLSCAASGIHYSSHCMGWYRQAPGKGREFVALMSHSRGYYYADSVKGRFTISRDNSKNTVYLQMNSLRAEDTAVYYCAAVPRAGIESGAYCKWQNKDSGSWGQGTQVTVSS

SEQ ID NO: 44 - protein, 129 aa, synthetic construct
EVQLVESGGGLVQPGGSLRLSCAASGFTYQSHCMGWYRQAPGKGREFVALMQHSRGTYYADSVKGRFTISRDNSKNTVYLQMNSLRAEDTAVYYCAAVPRAGIESGAYCKWQNKDSGSWGQGTQVTVSS

SEQ ID NO: 45 - protein, 129 aa, synthetic construct
EVQLVESGGGLVQPGGSLRLSCAASGFLYSFHCMGWYRQAPGKGREFVALITHSRGYSYADSVKGRFTISRDNSKNTVYLQMNSLRAEDTAVYYCAAVPRAGIESGAYCKWQNKDSGSWGQGTQVTVSS

SEQ ID NO: 46 - protein, 129 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWRLQDSGSWGQGTQVTVSS

SEQ ID NO: 47 - protein, 129 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWRLQDSGSWGQGTQVTVSS

SEQ ID NO: 48 - protein, 129 aa, synthetic construct
EVQLVESGGGLVQPGGSLRLSCAASGSTYSSHCMGWYRQAPGKGREFVALMTRSRGYYYADSVKGRFTISRDNSKNTVYLQMNSLRAEDTAVYYCAAVPRAGIESGAYCKWHGKDSGSWGQGTQVTVSS

SEQ ID NO: 49 - protein, 129 aa, synthetic construct
EVQLVESGGGLVQPGGSLRLSCAASGSTYSSHCMGWYRQAPGKGREFVALMTRSRGTYYADSVKGRFTISRDNSKNTVYLQMNSLRAEDTAVYYCAAVPRAGIESGAYCKWHGKDSGSWGQGTQVTVSS

SEQ ID NO: 50 - protein, 129 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWHNKDSGSWGQGTQVTVSS

SEQ ID NO: 51 - protein, 129 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWHNKDSGSWGQGTQVTVSS

SEQ ID NO: 52 - protein, 129 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNNKDSGSWGQGTQVTVSS

SEQ ID NO: 53 - protein, 129 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNNKDSGSWGQGTQVTVSS

SEQ ID NO: 54 - protein, 129 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNYKDSGSWGQGTQVTVSS

SEQ ID NO: 55 - protein, 129 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNYKDSGSWGQGTQVTVSS

SEQ ID NO: 56 - protein, 129 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWQNKDSGSWGQGTQVTVSS

SEQ ID NO: 57 - protein, 129 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWQNKDSGSWGQGTQVTVSS

SEQ ID NO: 58 - protein, 129 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWSNKDSGSWGQGTQVTVSS

SEQ ID NO: 59 - protein, 129 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWSNKDSGSWGQGTQVTVSS

SEQ ID NO: 60 - protein, 130 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIEYSGAYCKWNMKDSGSWGQGTLVTVSS

SEQ ID NO: 61 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIESGAYCKWNMKDSGSWGQGTLVTVSS

SEQ ID NO: 62 - protein, 129 aa, synthetic construct
QVQLVESGGGSVQSGGSLTLSCAASGSTYASHCMGWFRQAPGKEREGVALMTRSRGTSYADSVKGRFTISQDNTKNILYLQMNSLKPEDTAMYYCAAVPRAGIESGAYCKWNMKDSGSWGQGTLVTVSS

SEQ ID NO: 63 - protein, 129 aa, synthetic construct
QVQLVESGGGSVQSGGSLTLSCAASGSTYSSHCMGWFRQAPGKEREGVALMARSRGTSYADSVKGRFTISQDNTKNILYLQMNSLKPEDTAMYYCAAVPRAGIESGAYCKWNMKDSGSWGQGTLVTVSS

SEQ ID NO: 64 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIESGAYCKANMKDSGSWGQGTLVTVSS

SEQ ID NO: 65 - protein, 130 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEYSGAYCKWNMKDSGSWGQGTQVTVSS

SEQ ID NO: 66 - protein, 129 aa, synthetic construct
EVQLVESGGGLVQPGGSLRLSCAASGSTYASHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSS

SEQ ID NO: 67 - protein, 129 aa, synthetic construct
EVQLVESGGGLVQPGGSLRLSCAASGSTYSSHCMGWYRQAPGKGREFVALMARSRGTSYADSVKGRFTISRDNSKNTVYLQMNSLRAEDTAVYYCAAVPRAGIESGAYCKWNMKDSGSWGQGTQVTVSS

SEQ ID NO: 68 - protein, 129 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKANMKDSGSWGQGTQVTVSS

SEQ ID NO: 69 - protein, 129 aa, synthetic construct
EVQLVESGGGLVQPGGSLRLSCAASGSTYSSHCMGWYRQAPGKGREFVALMTHSRGYSYADSVKGRFTISRDNSKNTVYLQMNSLRAEDTAVYYCAAVPRAGIESGAYCKWSYKDSGSWGQGTQVTVSS

SEQ ID NO: 70 - protein, 130 aa, synthetic construct
EVQLVESGGGLVQPGGSLRLSCAASGSTYSSHCMGWYRQAPGKGREFVALMTHSRGTSYYADSVKGRFTISRDNSKNTVYLQMNSLRAEDTAVYYCAAVPRAGIESGAYCKWSYKDSGSWGQGTQVTVSS

SEQ ID NO: 71 - protein, 130 aa, synthetic construct
EVQLVESGGGLVQPGGSLRLSCAASGLTFSSHCMGWYRQAPGKGREFVAAIQGSRGSTYYADSVKGRFTISRDNSKNTVYLQMNSLRAEDTAVYYCAAVPRAGIESGAYCKWSYKDSGSWGQGTQVTVSS

SEQ ID NO: 72 - protein, 130 aa, synthetic construct
EVQLVESGGGLVQPGGSLRLSCAASGLTFSSHCMGWYRQAPGKGREFVAAIQHSRGSTYYADSVKGRFTISRDNSKNTVYLQMNSLRAEDTAVYYCAAVPRAGIESGAYCKWSYKDSGSWGQGTQVTVSS

SEQ ID NO: 73 - protein, 129 aa, synthetic construct
EVQLVESGGGLVQPGGSLRLSCAASGLTFSSHCMGWYRQAPGKGREFVAAIQHSRGYSYADSVKGRFTISRDNSKNTVYLQMNSLRAEDTAVYYCAAVPRAGIESGAYCKWSYKDSGSWGQGTQVTVSS

SEQ ID NO: 74 - protein, 129 aa, synthetic construct
EVQLVESGGGLVQPGGSLRLSCAASGLTFSSHCMGWYRQAPGKGREFVAAIQRSRGYSYADSVKGRFTISRDNSKNTVYLQMNSLRAEDTAVYYCAAVPRAGIESGAYCKWSYKDSGSWGQGTQVTVSS

SEQ ID NO: 75 - protein, 129 aa, synthetic construct
EVQLVESGGGLVQPGGSLRLSCAASGLTFSSHCMGWYRQAPGKGREFVAAISHSRGYSYADSVKGRFTISRDNSKNTVYLQMNSLRAEDTAVYYCAAVPRAGIESGAYCKWSYKDSGSWGQGTQVTVSS

SEQ ID NO: 76 - protein, 129 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSS

SEQ ID NO: 77 - protein, 129 aa, synthetic construct
EVQLVESGGGLVQPGGSLRLSCAASGLTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSS

SEQ ID NO: 78 - protein, 129 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSS

SEQ ID NO: 79 - protein, 129 aa, synthetic construct
EVQLVESGGGLVQPGGSLRLSCAASGLTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSS

SEQ ID NO: 80 - protein, 129 aa, synthetic construct
EVQLVESGGGLVQPGGSLRLSCAASGFTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSS

SEQ ID NO: 81 - protein, 129 aa, synthetic construct
EVQLVESGGGLVQPGGSLRLSCAASGFTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSS

SEQ ID NO: 82 - protein, 129 aa, synthetic construct
EVQLVESGGGLVQPGGSLRLSCAASGFTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSS

SEQ ID NO: 83 - protein, 129 aa, synthetic construct
EVQLVESGGGLVQPGGSLRLSCAASGFTFSSYCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSS

SEQ ID NO: 84 - protein, 129 aa, synthetic construct
EVQLVESGGGLVQPGGSLRLSCAASGFTFSSYCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSS

SEQ ID NO: 85 - protein, 10 aa, synthetic construct
GSTYSSHCMG

SEQ ID NO: 86 - protein, 10 aa, synthetic construct
LMTRSRGTSY

SEQ ID NO: 87 - protein, 23 aa, synthetic construct
AAVPRAGIESGAYCKWNMKDSGS

SEQ ID NO: 88 - protein, 10 aa, synthetic construct
GYNYVFHCMG

SEQ ID NO: 89 - protein, 10 aa, synthetic construct
LMTHSRGTSY

SEQ ID NO: 90 - protein, 23 aa, synthetic construct
AAVPRAGIESGAYCKWIIKDSGS

SEQ ID NO: 91 - protein, 10 aa, synthetic construct
GITYVSHCMG

SEQ ID NO: 92 - protein, 10 aa, synthetic construct
LMTRSYGTSY

SEQ ID NO: 93 - protein, 23 aa, synthetic construct
AAVPRAGIESGAYCKWRYKDSGS

SEQ ID NO: 94 - protein, 10 aa, synthetic construct
GITYSSHCMG

SEQ ID NO: 95 - protein, 10 aa, synthetic construct
LMSRSRGYYY

SEQ ID NO: 96 - protein, 23 aa, synthetic construct
AAVPRAGIESGAYCKWRYQDSGS

SEQ ID NO: 97 - protein, 10 aa, synthetic construct
GITYQSHCMG

SEQ ID NO: 98 - protein, 10 aa, synthetic construct
AMSRSRGYYY

SEQ ID NO: 99 - protein, 23 aa, synthetic construct
AAVPRAGIESGAYCKWIIQDSGS

SEQ ID NO: 100 - protein, 10 aa, synthetic construct
GLHYHSHCMG

SEQ ID NO: 101 - protein, 10 aa, synthetic construct
AMQRSRGTYY

SEQ ID NO: 102 - protein, 23 aa, synthetic construct
AAVPRAGIESGAYCKWPRKDSGS

SEQ ID NO: 103 - protein, 10 aa, synthetic construct
GIHYSSHCMG

SEQ ID NO: 104 - protein, 10 aa, synthetic construct
LIQRSRGTYY

SEQ ID NO: 105 - protein, 23 aa, synthetic construct
AAVPRAGIESGAYCKWPRQDSGS

SEQ ID NO: 106 - protein, 10 aa, synthetic construct
GFTYQSHCMG

SEQ ID NO: 107 - protein, 10 aa, synthetic construct
AMSHSRGYYY

SEQ ID NO: 108 - protein, 23 aa, synthetic construct
AAVPRAGIESGAYCKWRLQDSGS

SEQ ID NO: 109 - protein, 10 aa, synthetic construct
GFLYSFHCMG

SEQ ID NO: 110 - protein, 10 aa, synthetic construct
LMSHSRGYYY

SEQ ID NO: 111 - protein, 23 aa, synthetic construct
AAVPRAGIESGAYCKWPHKDSGS

SEQ ID NO: 112 - protein, 10 aa, synthetic construct
GSTYASHCMG

SEQ ID NO: 113 - protein, 10 aa, synthetic construct
LMQHSRGTYY

SEQ ID NO: 114 - protein, 23 aa, synthetic construct
AAVPRAGIESGAYCKWPHQDSGS

SEQ ID NO: 115 - protein, 10 aa, synthetic construct
GLTFSSHCMG

SEQ ID NO: 116 - protein, 10 aa, synthetic construct
LITHSRGYSY

SEQ ID NO: 117 - protein, 23 aa, synthetic construct
AAVPRAGIESGAYCKWHGKDSGS

SEQ ID NO: 118 - protein, 10 aa, synthetic construct
GFTFSSHCMG

SEQ ID NO: 119 - protein, 10 aa, synthetic construct
LMTHSRGYYY

SEQ ID NO: 120 - protein, 23 aa, synthetic construct
AAVPRAGIESGAYCKWHNKDSGS

SEQ ID NO: 121 - protein, 10 aa, synthetic construct
GFTFSSYCMG

SEQ ID NO: 122 - protein, 10 aa, synthetic construct
LMTHSRGTYY

SEQ ID NO: 123 - protein, 23 aa, synthetic construct
AAVPRAGIESGAYCKWHYKDSGS

SEQ ID NO: 124 - protein, 10 aa, synthetic construct
LMTRSRGYYY

SEQ ID NO: 125 - protein, 23 aa, synthetic construct
AAVPRAGIESGAYCKWKNKDSGS

SEQ ID NO: 126 - protein, 10 aa, synthetic construct
LMTRSRGTYY

SEQ ID NO: 127 - protein, 23 aa, synthetic construct
AAVPRAGIESGAYCKWKYKDSGS

SEQ ID NO: 128 - protein, 10 aa, synthetic construct
LMARSRGTSY

SEQ ID NO: 129 - protein, 23 aa, synthetic construct
AAVPRAGIESGAYCKWNNKDSGS

SEQ ID NO: 130 - protein, 10 aa, synthetic construct
LMTHSRGYSY

SEQ ID NO: 131 - protein, 23 aa, synthetic construct
AAVPRAGIESGAYCKWNYKDSGS

SEQ ID NO: 132 - protein, 11 aa, synthetic construct
LMTHSRGTSYY

SEQ ID NO: 133 - protein, 23 aa, synthetic construct
AAVPRAGIESGAYCKWQNKDSGS

SEQ ID NO: 134 - protein, 11 aa, synthetic construct
AIQGSRGSTYY

SEQ ID NO: 135 - protein, 23 aa, synthetic construct
AAVPRAGIESGAYCKWQYKDSGS

SEQ ID NO: 136 - protein, 11 aa, synthetic construct
AIQHSRGSTYY

SEQ ID NO: 137 - protein, 23 aa, synthetic construct
AAVPRAGIESGAYCKWRNKDSGS

SEQ ID NO: 138 - protein, 10 aa, synthetic construct
AIQHSRGYSY

SEQ ID NO: 139 - protein, 23 aa, synthetic construct
AAVPRAGIESGAYCKWSNKDSGS

SEQ ID NO: 140 - protein, 10 aa, synthetic construct
AIQRSRGYSY

SEQ ID NO: 141 - protein, 23 aa, synthetic construct
AAVPRAGIESGAYCKWSYKDSGS

SEQ ID NO: 142 - protein, 10 aa, synthetic construct
AISHSRGYSY

SEQ ID NO: 143 - protein, 23 aa, synthetic construct
AAVPRAGIESGAYCKWIHKDSGS

SEQ ID NO: 144 - protein, 10 aa, synthetic construct
AISGSRGYSY

SEQ ID NO: 145 - protein, 23 aa, synthetic construct
AAVPRAGIESGAYCKWILKDSGS

SEQ ID NO: 146 - protein, 10 aa, synthetic construct
AISHSRGSSY

SEQ ID NO: 147 - protein, 23 aa, synthetic construct
AAVPRAGIESGAYCKWINKDSGS

SEQ ID NO: 148 - protein, 10 aa, synthetic construct
AIQHSRGSSY

SEQ ID NO: 149 - protein, 23 aa, synthetic construct
AAVPRAGIESGAYCKWTHKDSGS

SEQ ID NO: 150 - protein, 10 aa, synthetic construct
AIQGSRGSSY

SEQ ID NO: 151 - protein, 23 aa, synthetic construct
AAVPRAGIESGAYCKWTIKDSGS

SEQ ID NO: 152 - protein, 23 aa, synthetic construct
AAVPRAGIESGAYCKWTLKDSGS

SEQ ID NO: 153 - protein, 23 aa, synthetic construct
AAVPRAGIESGAYCKWTNKDSGS

SEQ ID NO: 154 - protein, 24 aa, synthetic construct
AAVPRAGIEYSGAYCKWNMKDSGS

SEQ ID NO: 155 - protein, 23 aa, synthetic construct
AAVPRAGIESGAYCKANMKDSGS

SEQ ID NO: 156 - protein, 125 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCARTVQRRTLDGYRSSFDYWGQGTQVTVSS

SEQ ID NO: 157 - protein, 125 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCRSRKQRRTLDGYRSSFDYWGQGTQVTVSS

SEQ ID NO: 158 - protein, 125 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTIFDYWGQGTQVTVSS

SEQ ID NO: 159 - protein, 125 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTIFDYWGQGTQVTVSS

SEQ ID NO: 160 - protein, 125 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTVYDYWGQGTQVTVSS

SEQ ID NO: 161 - protein, 125 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTVYDYWGQGTQVTVSS

SEQ ID NO: 162 - protein, 125 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRTIFDYWGQGTQVTVSS

SEQ ID NO: 163 - protein, 125 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGFRTVYDYWGQGTQVTVSS

SEQ ID NO: 164 - protein, 125 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSS

SEQ ID NO: 165 - protein, 125 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTIFDYWGQGTQVTVSS

SEQ ID NO: 166 - protein, 125 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTVYDYWGQGTQVTVSS

SEQ ID NO: 167 - protein, 125 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTVYDYWGQGTQVTVSS

SEQ ID NO: 168 - protein, 125 aa, synthetic construct
EVQLVESGGGLVQPGGSLRLSCAASQQTFSSYAMGWYRQAPGKGREFVASTSGSGGSTYYADSVKGRFTISRDNSKNTVYLQMNSLRAEDTAVYYCRAMRQRRTLDGYRSSFDYWGQGTQVTVSS

SEQ ID NO: 169 - protein, 125 aa, synthetic construct
EVQLVESGGGLVQPGGSLRLSCAASQVTFSSYAMGWYRQAPGKGREFVASISHYDGSTYYADSVKGRFTISRDNSKNTVYLQMNSLRAEDTAVYYCRAGRQRRTLDGYRSSFDYWGQGTQVTVSS

SEQ ID NO: 170 - protein, 125 aa, synthetic construct
EVQLVESGGGLVQPGGSLRLSCAASGFTFPPYAMGWYRQAPGKGREFVASISGSGGSTYYADSVKGRFTISRDNSKNTVYLQMNSLRAEDTAVYYCRGMKQRRTLDGYRSSFDYWGQGTQVTVSS

SEQ ID NO: 171 - protein, 125 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRSSFDYWGQGTQVTVSS

SEQ ID NO: 172 - protein, 125 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCCAVKQRRTLDGYRSSFDYWGQGTQVTVSS

SEQ ID NO: 173 - protein, 125 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCSAVKQRRTLDGYRSSFDYWGQGTQVTVSS

SEQ ID NO: 174 - protein, 125 aa, synthetic construct
EVQLVESGGGLVQPGGSLRLSCAASGFPFSSYAMGWYRQAPGKGREFVAAISFLEGSTYYADSVKGRFTISRDNSKNTVYLQMNSLRAEDTAVYYCRAGKQRRTLDGYRSSFDYWGQGTQVTVSS

SEQ ID NO: 175 - protein, 125 aa, synthetic construct
EVQLVESGGGLVQPGGSLRLSCAASGFDISSYAMGWYRQAPGKGREFVAAISFVAGSTYYADSVKGRFTISRDNSKNTVYLQMNSLRAEDTAVYYCEARRQRRTLDGYRSSFDYWGQGTQVTVSS

SEQ ID NO: 176 - protein, 125 aa, synthetic construct
EVQLVESGGGLVQPGGSLRLSCAASGFVGSSYAMGWYRQAPGKGREFVASIGGSGGSTYYADSVKGRFTISRDNSKNTVYLQMNSLRAEDTAVYYCRVARQRRTLDGYRSSFDYWGQGTQVTVSS

SEQ ID NO: 177 - protein, 125 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRSSFDYWGQGTQVTVSS

SEQ ID NO: 178 - protein, 125 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQHRTLDGYRSSFDYWGQGTQVTVSS

SEQ ID NO: 179 - protein, 125 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRVSLQRRTLDGYRSSFDYWGQGTQVTVSS

SEQ ID NO: 180 - protein, 125 aa, synthetic construct
EVQLVESGGGLVQPGGSLRLSCAASGFPHPSYPMGWYRQAPGKGREFVAGPGGSGGSTYYADSVKGRFTISRDNSKNTVYLQMNSLRAEDTAVYYCRGARQRRTLDGYRSSFDYWGQGTQVTVSS

SEQ ID NO: 181 - protein, 125 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSS

SEQ ID NO: 182 - protein, 125 aa, synthetic construct
EVQLVESGGGLVQPGGSLRLSCAASGFTFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSS

SEQ ID NO: 183 - protein, 125 aa, synthetic construct
EVQLVESGGGLVQPGGSLRLSCAASGFTFSSYAMGWYRQAPGKGREFVASISFSGGSTYYADSVKGRFTISRDNSKNTVYLQMNSLRAEDTAVYYCRMMSQRRTLDGYRTIFDYWGQGTQVTVSS

SEQ ID NO: 184 - protein, 125 aa, synthetic construct
EVQLVESGGGLVQPGGSLRLSCAASGFTFSSYAMGWYRQAPGKGREFVASISGSGGSTQYADSVKGRFTISRDNSKNTVYLQMNSLRAEDTAVYYCRMMSQRRTLDGYRTIFDYWGQGTQVTVSS

SEQ ID NO: 185 - protein, 125 aa, synthetic construct
EVQLVESGGGLVQPGGSLRLSCAASGFTFSSYAMGWYRQAPGKGREFVAAISFSGGSTQYADSVKGRFTISRDNSKNTVYLQMNSLRAEDTAVYYCRMMSQRRTLDGYRTIFDYWGQGTQVTVSS

SEQ ID NO: 186 - protein, 125 aa, synthetic construct
EVQLVESGGGLVQPGGSLRLSCAASGFTFSSYAMGWYRQAPGKGREFVAAISFSGGSTYYADSVKGRFTISRDNSKNTVYLQMNSLRAEDTAVYYCRMMSQRRTLDGYRTIFDYWGQGTQVTVSS

SEQ ID NO: 187 - protein, 125 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSS

SEQ ID NO: 188 - protein, 125 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSS

SEQ ID NO: 189 - protein, 125 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSS

SEQ ID NO: 190 - protein, 125 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSS

SEQ ID NO: 191 - protein, 125 aa, synthetic construct
EVQLVESGGGLVQPGGSLRLSCAASGFTHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSS

SEQ ID NO: 192 - protein, 10 aa, synthetic construct
GFTFSSYAMG

SEQ ID NO: 193 - protein, 16 aa, synthetic construct
AISGSGGSTYYADSVK

SEQ ID NO: 194 - protein, 18 aa, synthetic construct
ARTVQRRTLDGYRSSFDY

SEQ ID NO: 195 - protein, 10 aa, synthetic construct
GFTFSSPHMG

SEQ ID NO: 196 - protein, 16 aa, synthetic construct
AISGSGTAGYYADSVK

SEQ ID NO: 197 - protein, 18 aa, synthetic construct
RSRKQRRTLDGYRSSFDY

SEQ ID NO: 198 - protein, 10 aa, synthetic construct
GFTHSSYAMG

SEQ ID NO: 199 - protein, 16 aa, synthetic construct
VISGSGGSTQLLDSVK

SEQ ID NO: 200 - protein, 18 aa, synthetic construct
RSVLQRRTLDGYRTIFDY

SEQ ID NO: 201 - protein, 10 aa, synthetic construct
GFHHSSYAMG

SEQ ID NO: 202 - protein, 16 aa, synthetic construct
SISGSGGSTYYADSVK

SEQ ID NO: 203 - protein, 18 aa, synthetic construct
RSVLQRRTLDGFRTIFDY

SEQ ID NO: 204 - protein, 10 aa, synthetic construct
QQTFSSYAMG

SEQ ID NO: 205 - protein, 16 aa, synthetic construct
STSGSGGSTYYADSVK

SEQ ID NO: 206 - protein, 18 aa, synthetic construct
RSVLQRRTLDGYRTVYDY

SEQ ID NO: 207 - protein, 10 aa, synthetic construct
QVTFSSYAMG

SEQ ID NO: 208 - protein, 16 aa, synthetic construct
SISHYDGSTYYADSVK

SEQ ID NO: 209 - protein, 18 aa, synthetic construct
RSVLQRRTLDGFRTVYDY

SEQ ID NO: 210 - protein, 10 aa, synthetic construct
GFTFPPYAMG

SEQ ID NO: 211 - protein, 16 aa, synthetic construct
AISFIAGSTYYADSVK

SEQ ID NO: 212 - protein, 18 aa, synthetic construct
RAERQRRTLDGYRTIFDY

SEQ ID NO: 213 - protein, 10 aa, synthetic construct
GFAISSYAMG

SEQ ID NO: 214 - protein, 16 aa, synthetic construct
AISFLEGSTYYADSVK

SEQ ID NO: 215 - protein, 18 aa, synthetic construct
RAERQRRTLDGFRTVYDY

SEQ ID NO: 216 - protein, 10 aa, synthetic construct
GFPFSSYAMG

SEQ ID NO: 217 - protein, 16 aa, synthetic construct
AISFVAGSTYYADSVK

SEQ ID NO: 218 - protein, 18 aa, synthetic construct
RMMSQRRTLDGYRTIFDY

SEQ ID NO: 219 - protein, 10 aa, synthetic construct
GFDISSYAMG

SEQ ID NO: 220 - protein, 16 aa, synthetic construct
SIGGSGGSTYYADSVK

SEQ ID NO: 221 - protein, 18 aa, synthetic construct
RMMSQRRTLDGFRTIFDY

SEQ ID NO: 222 - protein, 10 aa, synthetic construct
GFVGSSYAMG

SEQ ID NO: 223 - protein, 16 aa, synthetic construct
GPGGSGGSTYYADSVK

SEQ ID NO: 224 - protein, 18 aa, synthetic construct
RMMSQRRTLDGYRTVYDY

SEQ ID NO: 225 - protein, 10 aa, synthetic construct
GFPHPSYPMG

SEQ ID NO: 226 - protein, 16 aa, synthetic construct
SISFSGGSTYYADSVK

SEQ ID NO: 227 - protein, 18 aa, synthetic construct
RMMSQRRTLDGFRTVYDY

SEQ ID NO: 228 - protein, 10 aa, synthetic construct
GFHFSSYAMG

SEQ ID NO: 229 - protein, 16 aa, synthetic construct
SISGSGGSTQYADSVK

SEQ ID NO: 230 - protein, 18 aa, synthetic construct
RAMRQRRTLDGYRSSFDY

SEQ ID NO: 231 - protein, 16 aa, synthetic construct
AISFSGGSTQYADSVK

SEQ ID NO: 232 - protein, 18 aa, synthetic construct
RAGRQRRTLDGYRSSFDY

SEQ ID NO: 233 - protein, 16 aa, synthetic construct
AISFSGGSTYYADSVK

SEQ ID NO: 234 - protein, 18 aa, synthetic construct
RGMKQRRTLDGYRSSFDY

SEQ ID NO: 235 - protein, 18 aa, synthetic construct
RMMSQRRTLDGYRSSFDY

SEQ ID NO: 236 - protein, 18 aa, synthetic construct
CAVKQRRTLDGYRSSFDY

SEQ ID NO: 237 - protein, 18 aa, synthetic construct
SAVKQRRTLDGYRSSFDY

SEQ ID NO: 238 - protein, 18 aa, synthetic construct
RAGKQRRTLDGYRSSFDY

SEQ ID NO: 239 - protein, 18 aa, synthetic construct
EARRQRRTLDGYRSSFDY

SEQ ID NO: 240 - protein, 18 aa, synthetic construct
RVARQRRTLDGYRSSFDY

SEQ ID NO: 241 - protein, 18 aa, synthetic construct
RAERQRRTLDGYRSSFDY

SEQ ID NO: 242 - protein, 18 aa, synthetic construct
RAERQHRTLDGYRSSFDY

SEQ ID NO: 243 - protein, 18 aa, synthetic construct
RVSLQRRTLDGYRSSFDY

SEQ ID NO: 244 - protein, 18 aa, synthetic construct
RGARQRRTLDGYRSSFDY

SEQ ID NO: 245 - protein, 18 aa, synthetic construct
RSMSQRRTLDGYRTIFDY

SEQ ID NO: 246 - protein, 18 aa, synthetic construct
RMVSQRRTLDGYRTIFDY

SEQ ID NO: 247-248 - skipped (2 entries)

SEQ ID NO: 249 - protein, 24 aa, synthetic construct
  VARIANT 9: note = E, F, Y, or W
  VARIANT 10: note = Y, F, P, K, R, W, L, I, or absent
  VARIANT 11: note = S, F, Y, or W
  VARIANT 17: note = W, A, F, or Y
  VARIANT 18: note = S, H, I, K, N, P, Q, R, or T
  VARIANT 19: note = Y, G, H, I, L, M, N, or R
  VARIANT 20: note = K or Q
AAVPRAGIXXXGAYCKXXXXDSGS

SEQ ID NO: 250-251 - skipped (2 entries)

SEQ ID NO: 252 - protein, 18 aa, synthetic construct
  VARIANT 1: note = R, A, C, E, or S
  VARIANT 2: note = V, A, G, M, R, or S
  VARIANT 3: note = S, A, E, G, M, R, T, or V
  VARIANT 4: note = L, K, R, S, or V
  VARIANT 5: note = Q or G
  VARIANT 6: note = R or H
  VARIANT 8: note = T, L, or M
  VARIANT 10: note = D or E
  VARIANT 12: note = Y or F
  VARIANT 14: note = S or T
  VARIANT 15: note = S, I, V, or L
  VARIANT 16: note = F or Y
XXXXXXRXLXGXRXXXDY

SEQ ID NO: 253 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 257 - protein, 232 aa, synthetic construct
EPKSSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 232 aa, synthetic construct
EPKSSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 232 aa, synthetic construct
EPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 232 aa, synthetic construct
EPKSSDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 262 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 263 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 264 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 265 - protein, 231 aa, synthetic construct
EPKSSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 266 - protein, 231 aa, synthetic construct
EPKSSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 267 - protein, 231 aa, synthetic construct
EPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 231 aa, synthetic construct
EPKSSDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 269 - protein, 5 aa, synthetic construct
GGGGS

SEQ ID NO: 270 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRYQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWIIQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 366 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 366 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPHQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 288 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 289 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 291 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 292 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 294 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 296 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 297 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWILKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWINKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTIKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTLKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 366 aa, synthetic construct
EVQLVESGGGLVQPGGSLRLSCAASGYNYVFHCMGWYRQAPGKGREFVALMSRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 366 aa, synthetic construct
EVQLVESGGGLVQPGGSLRLSCAASGITYVSHCMGWYRQAPGKGREFVAAMSRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 366 aa, synthetic construct
EVQLVESGGGLVQPGGSLRLSCAASGITYSSHCMGWYRQAPGKGREFVAAMQRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 366 aa, synthetic construct
EVQLVESGGGLVQPGGSLRLSCAASGITYQSHCMGWYRQAPGKGREFVALIQRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 366 aa, synthetic construct
EVQLVESGGGLVQPGGSLRLSCAASGLHYHSHCMGWYRQAPGKGREFVAAMSHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 366 aa, synthetic construct
EVQLVESGGGLVQPGGSLRLSCAASGIHYSSHCMGWYRQAPGKGREFVALMSHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 366 aa, synthetic construct
EVQLVESGGGLVQPGGSLRLSCAASGFTYQSHCMGWYRQAPGKGREFVALMQHSRGTYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 366 aa, synthetic construct
EVQLVESGGGLVQPGGSLRLSCAASGFLYSFHCMGWYRQAPGKGREFVALITHSRGYSYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 366 aa, synthetic construct
EVQLVESGGGLVQPGGSLRLSCAASGSTYSSHCMGWYRQAPGKGREFVALMTRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 366 aa, synthetic construct
EVQLVESGGGLVQPGGSLRLSCAASGSTYSSHCMGWYRQAPGKGREFVALMTRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 367 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIEYSGAYCKWNMKDSGSWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 366 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIESGAYCKWNMKDSGSWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 366 aa, synthetic construct
QVQLVESGGGSVQSGGSLTLSCAASGSTYASHCMGWFRQAPGKEREGVALMTRSRGTSYADSVKGRFTISQDNTKNILYLQMNSLKPEDTAMYYCAAVPRAGIESGAYCKWNMKDSGSWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 366 aa, synthetic construct
QVQLVESGGGSVQSGGSLTLSCAASGSTYSSHCMGWFRQAPGKEREGVALMARSRGTSYADSVKGRFTISQDNTKNILYLQMNSLKPEDTAMYYCAAVPRAGIESGAYCKWNMKDSGSWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 366 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIESGAYCKANMKDSGSWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 367 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEYSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 366 aa, synthetic construct
EVQLVESGGGLVQPGGSLRLSCAASGSTYASHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 366 aa, synthetic construct
EVQLVESGGGLVQPGGSLRLSCAASGSTYSSHCMGWYRQAPGKGREFVALMARSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKANMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 366 aa, synthetic construct
EVQLVESGGGLVQPGGSLRLSCAASGSTYSSHCMGWYRQAPGKGREFVALMTHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 367 aa, synthetic construct
EVQLVESGGGLVQPGGSLRLSCAASGSTYSSHCMGWYRQAPGKGREFVALMTHSRGTSYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 340 - protein, 367 aa, synthetic construct
EVQLVESGGGLVQPGGSLRLSCAASGLTFSSHCMGWYRQAPGKGREFVAAIQGSRGSTYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 367 aa, synthetic construct
EVQLVESGGGLVQPGGSLRLSCAASGLTFSSHCMGWYRQAPGKGREFVAAIQHSRGSTYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 366 aa, synthetic construct
EVQLVESGGGLVQPGGSLRLSCAASGLTFSSHCMGWYRQAPGKGREFVAAIQHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 366 aa, synthetic construct
EVQLVESGGGLVQPGGSLRLSCAASGLTFSSHCMGWYRQAPGKGREFVAAIQRSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 366 aa, synthetic construct
EVQLVESGGGLVQPGGSLRLSCAASGLTFSSHCMGWYRQAPGKGREFVAAISHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 366 aa, synthetic construct
EVQLVESGGGLVQPGGSLRLSCAASGLTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 366 aa, synthetic construct
EVQLVESGGGLVQPGGSLRLSCAASGLTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 366 aa, synthetic construct
EVQLVESGGGLVQPGGSLRLSCAASGFTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 366 aa, synthetic construct
EVQLVESGGGLVQPGGSLRLSCAASGFTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 366 aa, synthetic construct
EVQLVESGGGLVQPGGSLRLSCAASGFTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 366 aa, synthetic construct
EVQLVESGGGLVQPGGSLRLSCAASGFTFSSYCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 366 aa, synthetic construct
EVQLVESGGGLVQPGGSLRLSCAASGFTFSSYCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 8 aa, synthetic construct
GSTYSSHC

SEQ ID NO: 355 - protein, 7 aa, synthetic construct
MTRSRGT

SEQ ID NO: 356 - protein, 8 aa, synthetic construct
GYNYVFHC

SEQ ID NO: 357 - protein, 7 aa, synthetic construct
MTHSRGT

SEQ ID NO: 358 - protein, 8 aa, synthetic construct
GITYVSHC

SEQ ID NO: 359 - protein, 7 aa, synthetic construct
MTRSYGT

SEQ ID NO: 360 - protein, 8 aa, synthetic construct
GITYSSHC

SEQ ID NO: 361 - protein, 7 aa, synthetic construct
MSRSRGY

SEQ ID NO: 362 - protein, 8 aa, synthetic construct
GITYQSHC

SEQ ID NO: 363 - protein, 7 aa, synthetic construct
MQRSRGT

SEQ ID NO: 364 - protein, 8 aa, synthetic construct
GLHYHSHC

SEQ ID NO: 365 - protein, 7 aa, synthetic construct
IQRSRGT

SEQ ID NO: 366 - protein, 8 aa, synthetic construct
GIHYSSHC

SEQ ID NO: 367 - protein, 7 aa, synthetic construct
MSHSRGY

SEQ ID NO: 368 - protein, 8 aa, synthetic construct
GFTYQSHC

SEQ ID NO: 369 - protein, 7 aa, synthetic construct
MQHSRGT

SEQ ID NO: 370 - protein, 8 aa, synthetic construct
GFLYSFHC

SEQ ID NO: 371 - protein, 7 aa, synthetic construct
ITHSRGY

SEQ ID NO: 372 - protein, 8 aa, synthetic construct
GSTYASHC

SEQ ID NO: 373 - protein, 7 aa, synthetic construct
MTHSRGY

SEQ ID NO: 374 - protein, 8 aa, synthetic construct
GLTFSSHC

SEQ ID NO: 375 - protein, 7 aa, synthetic construct
MTRSRGY

SEQ ID NO: 376 - protein, 8 aa, synthetic construct
GFTFSSHC

SEQ ID NO: 377 - protein, 7 aa, synthetic construct
MARSRGT

SEQ ID NO: 378 - protein, 8 aa, synthetic construct
GFTFSSYC

SEQ ID NO: 379 - protein, 7 aa, synthetic construct
IQGSRGS

SEQ ID NO: 380 - protein, 7 aa, synthetic construct
IQHSRGS

SEQ ID NO: 381 - protein, 7 aa, synthetic construct
IQHSRGY

SEQ ID NO: 382 - protein, 7 aa, synthetic construct
IQRSRGY

SEQ ID NO: 383 - protein, 7 aa, synthetic construct
ISHSRGY

SEQ ID NO: 384 - protein, 7 aa, synthetic construct
ISGSRGY

SEQ ID NO: 385 - protein, 7 aa, synthetic construct
ISHSRGS

SEQ ID NO: 386-390 - skipped (5 entries)

SEQ ID NO: 391 - protein, 361 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCARTVQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 392 - protein, 361 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCRSRKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 393 - protein, 361 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 394 - protein, 361 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 395 - protein, 361 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 396 - protein, 361 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 397 - protein, 361 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 398 - protein, 361 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 399 - protein, 361 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 400 - protein, 361 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 401 - protein, 361 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 402 - protein, 361 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 403 - protein, 361 aa, synthetic construct
EVQLVESGGGLVQPGGSLRLSCAASQQTFSSYAMGWYRQAPGKGREFVASTSGSGGSTYYADSVKGRFTISRDNSKNTVYLQMNSLRAEDTAVYYCRAMRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 404 - protein, 361 aa, synthetic construct
EVQLVESGGGLVQPGGSLRLSCAASQVTFSSYAMGWYRQAPGKGREFVASISHYDGSTYYADSVKGRFTISRDNSKNTVYLQMNSLRAEDTAVYYCRAGRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 405 - protein, 361 aa, synthetic construct
EVQLVESGGGLVQPGGSLRLSCAASGFTFPPYAMGWYRQAPGKGREFVASISGSGGSTYYADSVKGRFTISRDNSKNTVYLQMNSLRAEDTAVYYCRGMKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 406 - protein, 361 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 407 - protein, 361 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCCAVKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 408 - protein, 361 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCSAVKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 409 - protein, 361 aa, synthetic construct
EVQLVESGGGLVQPGGSLRLSCAASGFPFSSYAMGWYRQAPGKGREFVAAISFLEGSTYYADSVKGRFTISRDNSKNTVYLQMNSLRAEDTAVYYCRAGKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 410 - protein, 361 aa, synthetic construct
EVQLVESGGGLVQPGGSLRLSCAASGFDISSYAMGWYRQAPGKGREFVAAISFVAGSTYYADSVKGRFTISRDNSKNTVYLQMNSLRAEDTAVYYCEARRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 411 - protein, 361 aa, synthetic construct
EVQLVESGGGLVQPGGSLRLSCAASGFVGSSYAMGWYRQAPGKGREFVASIGGSGGSTYYADSVKGRFTISRDNSKNTVYLQMNSLRAEDTAVYYCRVARQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 412 - protein, 361 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 413 - protein, 361 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQHRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 414 - protein, 361 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRVSLQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 415 - protein, 361 aa, synthetic construct
EVQLVESGGGLVQPGGSLRLSCAASGFPHPSYPMGWYRQAPGKGREFVAGPGGSGGSTYYADSVKGRFTISRDNSKNTVYLQMNSLRAEDTAVYYCRGARQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 416 - protein, 361 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 417 - protein, 361 aa, synthetic construct
EVQLVESGGGLVQPGGSLRLSCAASGFTFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 418 - protein, 361 aa, synthetic construct
EVQLVESGGGLVQPGGSLRLSCAASGFTFSSYAMGWYRQAPGKGREFVASISFSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 419 - protein, 361 aa, synthetic construct
EVQLVESGGGLVQPGGSLRLSCAASGFTFSSYAMGWYRQAPGKGREFVASISGSGGSTQYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 420 - protein, 361 aa, synthetic construct
EVQLVESGGGLVQPGGSLRLSCAASGFTFSSYAMGWYRQAPGKGREFVAAISFSGGSTQYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 421 - protein, 361 aa, synthetic construct
EVQLVESGGGLVQPGGSLRLSCAASGFTFSSYAMGWYRQAPGKGREFVAAISFSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 422 - protein, 361 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 423 - protein, 361 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 424 - protein, 361 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 425 - protein, 361 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 426 - protein, 361 aa, synthetic construct
EVQLVESGGGLVQPGGSLRLSCAASGFTHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 427 - protein, 8 aa, synthetic construct
GFTFSSYA

SEQ ID NO: 428 - protein, 7 aa, synthetic construct
ISGSGGS

SEQ ID NO: 429 - protein, 8 aa, synthetic construct
GFTFSSPH

SEQ ID NO: 430 - protein, 7 aa, synthetic construct
ISGSGTA

SEQ ID NO: 431 - protein, 8 aa, synthetic construct
GFTHSSYA

SEQ ID NO: 432 - protein, 7 aa, synthetic construct
TSGSGGS

SEQ ID NO: 433 - protein, 8 aa, synthetic construct
GFHHSSYA

SEQ ID NO: 434 - protein, 7 aa, synthetic construct
ISHYDGS

SEQ ID NO: 435 - protein, 8 aa, synthetic construct
QQTFSSYA

SEQ ID NO: 436 - protein, 7 aa, synthetic construct
ISFIAGS

SEQ ID NO: 437 - protein, 8 aa, synthetic construct
QVTFSSYA

SEQ ID NO: 438 - protein, 7 aa, synthetic construct
ISFLEGS

SEQ ID NO: 439 - protein, 8 aa, synthetic construct
GFTFPPYA

SEQ ID NO: 440 - protein, 7 aa, synthetic construct
ISFVAGS

SEQ ID NO: 441 - protein, 8 aa, synthetic construct
GFAISSYA

SEQ ID NO: 442 - protein, 7 aa, synthetic construct
IGGSGGS

SEQ ID NO: 443 - protein, 8 aa, synthetic construct
GFPFSSYA

SEQ ID NO: 444 - protein, 7 aa, synthetic construct
PGGSGGS

SEQ ID NO: 445 - protein, 8 aa, synthetic construct
GFDISSYA

SEQ ID NO: 446 - protein, 7 aa, synthetic construct
ISFSGGS

SEQ ID NO: 447 - protein, 8 aa, synthetic construct
GFVGSSYA

SEQ ID NO: 448 - protein, 8 aa, synthetic construct
GFPHPSYP

SEQ ID NO: 449 - protein, 8 aa, synthetic construct
GFHFSSYA

SEQ ID NO: 450 - protein, 232 aa, synthetic construct
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 451 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSAYTQKSLSLSPGK

SEQ ID NO: 452 - protein, 232 aa, synthetic construct
EPKSSDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 453 - protein, 232 aa, synthetic construct
EPKSSDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 454 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSRDELTKNQVSLTCLVKGFYPSDIAVAWESNGQPENNYKTTPPVLDSDGSFFLYSKLTVDKSRWQQGNVFSCSVMHEALHAHYTQKSLSLSPGK

SEQ ID NO: 455 - protein, 232 aa, synthetic construct
EPKSSDKTHTCPPCPAPEFQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 456 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 457 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSAYTQKSLSLSPG

SEQ ID NO: 458 - protein, 231 aa, synthetic construct
EPKSSDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 459 - protein, 231 aa, synthetic construct
EPKSSDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 460 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSRDELTKNQVSLTCLVKGFYPSDIAVAWESNGQPENNYKTTPPVLDSDGSFFLYSKLTVDKSRWQQGNVFSCSVMHEALHAHYTQKSLSLSPG

SEQ ID NO: 461 - protein, 231 aa, synthetic construct
EPKSSDKTHTCPPCPAPEFQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 462 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 463 - protein, 231 aa, synthetic construct
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 464 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 465 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 466 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 467 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 468 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRYQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 469 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 470 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWIIQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 471 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 472 - protein, 366 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 473 - protein, 366 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 474 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 475 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 476 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 477 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 478 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 479 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 480 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPHQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 481 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 482 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 483 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 484 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 485 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 486 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 487 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 488 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 489 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 490 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 491 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 492 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 493 - protein, 366 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 494 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 495 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWILKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 496 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWINKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 497 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 498 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTIKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 499 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTLKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 500 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 501 - protein, 366 aa, synthetic construct
EVQLVESGGGLVQPGGSLRLSCAASGYNYVFHCMGWYRQAPGKGREFVALMSRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 502 - protein, 366 aa, synthetic construct
EVQLVESGGGLVQPGGSLRLSCAASGITYVSHCMGWYRQAPGKGREFVAAMSRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 503 - protein, 366 aa, synthetic construct
EVQLVESGGGLVQPGGSLRLSCAASGITYSSHCMGWYRQAPGKGREFVAAMQRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 504 - protein, 366 aa, synthetic construct
EVQLVESGGGLVQPGGSLRLSCAASGITYQSHCMGWYRQAPGKGREFVALIQRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 505 - protein, 366 aa, synthetic construct
EVQLVESGGGLVQPGGSLRLSCAASGLHYHSHCMGWYRQAPGKGREFVAAMSHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 506 - protein, 366 aa, synthetic construct
EVQLVESGGGLVQPGGSLRLSCAASGIHYSSHCMGWYRQAPGKGREFVALMSHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 507 - protein, 366 aa, synthetic construct
EVQLVESGGGLVQPGGSLRLSCAASGFTYQSHCMGWYRQAPGKGREFVALMQHSRGTYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 508 - protein, 366 aa, synthetic construct
EVQLVESGGGLVQPGGSLRLSCAASGFLYSFHCMGWYRQAPGKGREFVALITHSRGYSYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 509 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 510 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 511 - protein, 366 aa, synthetic construct
EVQLVESGGGLVQPGGSLRLSCAASGSTYSSHCMGWYRQAPGKGREFVALMTRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 512 - protein, 366 aa, synthetic construct
EVQLVESGGGLVQPGGSLRLSCAASGSTYSSHCMGWYRQAPGKGREFVALMTRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 513 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 514 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 515 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 516 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 517 - protein, 367 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMLHEALHSHYTQKSLSLSPGK

SEQ ID NO: 518 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 519 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 520 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 521 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 522 - protein, 366 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 523-527 - skipped (5 entries)

SEQ ID NO: 528 - protein, 367 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEYSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 529 - protein, 366 aa, synthetic construct
EVQLVESGGGLVQPGGSLRLSCAASGSTYASHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 530 - protein, 366 aa, synthetic construct
EVQLVESGGGLVQPGGSLRLSCAASGSTYSSHCMGWYRQAPGKGREFVALMARSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 531 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKANMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 532 - protein, 366 aa, synthetic construct
EVQLVESGGGLVQPGGSLRLSCAASGSTYSSHCMGWYRQAPGKGREFVALMTHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 533 - protein, 367 aa, synthetic construct
EVQLVESGGGLVQPGGSLRLSCAASGSTYSSHCMGWYRQAPGKGREFVALMTHSRGTSYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 534 - protein, 367 aa, synthetic construct
EVQLVESGGGLVQPGGSLRLSCAASGLTFSSHCMGWYRQAPGKGREFVAAIQGSRGSTYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 535 - protein, 367 aa, synthetic construct
EVQLVESGGGLVQPGGSLRLSCAASGLTFSSHCMGWYRQAPGKGREFVAAIQHSRGSTYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 536 - protein, 366 aa, synthetic construct
EVQLVESGGGLVQPGGSLRLSCAASGLTFSSHCMGWYRQAPGKGREFVAAIQHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 537 - protein, 366 aa, synthetic construct
EVQLVESGGGLVQPGGSLRLSCAASGLTFSSHCMGWYRQAPGKGREFVAAIQRSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 538 - protein, 366 aa, synthetic construct
EVQLVESGGGLVQPGGSLRLSCAASGLTFSSHCMGWYRQAPGKGREFVAAISHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 539 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 540 - protein, 366 aa, synthetic construct
EVQLVESGGGLVQPGGSLRLSCAASGLTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 541 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 542 - protein, 366 aa, synthetic construct
EVQLVESGGGLVQPGGSLRLSCAASGLTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 543 - protein, 366 aa, synthetic construct
EVQLVESGGGLVQPGGSLRLSCAASGFTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 544 - protein, 366 aa, synthetic construct
EVQLVESGGGLVQPGGSLRLSCAASGFTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 545 - protein, 366 aa, synthetic construct
EVQLVESGGGLVQPGGSLRLSCAASGFTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 546 - protein, 366 aa, synthetic construct
EVQLVESGGGLVQPGGSLRLSCAASGFTFSSYCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 547 - protein, 366 aa, synthetic construct
EVQLVESGGGLVQPGGSLRLSCAASGFTFSSYCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 548 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 549 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 550 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 551 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRYQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 552 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRYQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 553 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIIQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 554 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWIIQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 555 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 556 - protein, 365 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 557 - protein, 365 aa, synthetic construct
EVQLVESGGGLVQPGGSLRLSCAASGSTYSSHCMGWYRQAPGKGREFVALMTRSYGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 558 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 559 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPRQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 560 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 561 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 562 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 563 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWPHQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 564 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWPHQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 565 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 566 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 567 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 568 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWHYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 569 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 570 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWKYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 571 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 572 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 573 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 574 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWQYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 575 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWRNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 576 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 577 - protein, 365 aa, synthetic construct
EVQLVESGGGLVQPGGSLRLSCAASGSTYSSHCMGWYRQAPGKGREFVALMTHSRGTSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 578 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWIHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 579 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWILKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 580 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWINKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 581 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTHKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 582 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTIKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 583 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTLKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 584 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKWTNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 585 - protein, 365 aa, synthetic construct
EVQLVESGGGLVQPGGSLRLSCAASGYNYVFHCMGWYRQAPGKGREFVALMSRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 586 - protein, 365 aa, synthetic construct
EVQLVESGGGLVQPGGSLRLSCAASGITYVSHCMGWYRQAPGKGREFVAAMSRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 587 - protein, 365 aa, synthetic construct
EVQLVESGGGLVQPGGSLRLSCAASGITYSSHCMGWYRQAPGKGREFVAAMQRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 588 - protein, 365 aa, synthetic construct
EVQLVESGGGLVQPGGSLRLSCAASGITYQSHCMGWYRQAPGKGREFVALIQRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 589 - protein, 365 aa, synthetic construct
EVQLVESGGGLVQPGGSLRLSCAASGLHYHSHCMGWYRQAPGKGREFVAAMSHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 590 - protein, 365 aa, synthetic construct
EVQLVESGGGLVQPGGSLRLSCAASGIHYSSHCMGWYRQAPGKGREFVALMSHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 591 - protein, 365 aa, synthetic construct
EVQLVESGGGLVQPGGSLRLSCAASGFTYQSHCMGWYRQAPGKGREFVALMQHSRGTYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 592 - protein, 365 aa, synthetic construct
EVQLVESGGGLVQPGGSLRLSCAASGFLYSFHCMGWYRQAPGKGREFVALITHSRGYSYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 593 - protein, 365 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 594 - protein, 365 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWRLQDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 595 - protein, 365 aa, synthetic construct
EVQLVESGGGLVQPGGSLRLSCAASGSTYSSHCMGWYRQAPGKGREFVALMTRSRGYYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 596 - protein, 365 aa, synthetic construct
EVQLVESGGGLVQPGGSLRLSCAASGSTYSSHCMGWYRQAPGKGREFVALMTRSRGTYYADSVKGRFTISRDNSKNTVYLQMNSLRAEDTAVYYCAAVPRAGIESGAYCKWHGKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 597 - protein, 365 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 598 - protein, 365 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWHNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 599 - protein, 365 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 600 - protein, 365 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 601 - protein, 366 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMLHEALHSHYTQKSLSLSPG

SEQ ID NO: 602 - protein, 365 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 603 - protein, 365 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 604 - protein, 365 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWQNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 605 - protein, 365 aa, synthetic construct
EVQLVESGGGLVQPGGSLRLSCAASGSTYSSHCMGWYRQAPGKGREFVALMTHSRGYYYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 606 - protein, 365 aa, synthetic construct
EVQLVESGGGLVQPGGSLRLSCAASGSTYSSHCMGWYRQAPGKGREFVALMTHSRGTYYADSVKGRFTISRDNSKNTVYLQMNSLRAEDTAVYYCAAVPRAGIESGAYCKWSNKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 607-611 - skipped (5 entries)

SEQ ID NO: 612 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEYSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 613 - protein, 365 aa, synthetic construct
EVQLVESGGGLVQPGGSLRLSCAASGSTYASHCMGWYRQAPGKGREFVALMTRSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 614 - protein, 365 aa, synthetic construct
EVQLVESGGGLVQPGGSLRLSCAASGSTYSSHCMGWYRQAPGKGREFVALMARSRGTSYADSVKGRFTISRDNSKNTVYLQMNSLRAEDTAVYYCAAVPRAGIESGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 615 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKANMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 616 - protein, 365 aa, synthetic construct
EVQLVESGGGLVQPGGSLRLSCAASGSTYSSHCMGWYRQAPGKGREFVALMTHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 617 - protein, 366 aa, synthetic construct
EVQLVESGGGLVQPGGSLRLSCAASGSTYSSHCMGWYRQAPGKGREFVALMTHSRGTSYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 618 - protein, 366 aa, synthetic construct
EVQLVESGGGLVQPGGSLRLSCAASGLTFSSHCMGWYRQAPGKGREFVAAIQGSRGSTYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 619 - protein, 366 aa, synthetic construct
EVQLVESGGGLVQPGGSLRLSCAASGLTFSSHCMGWYRQAPGKGREFVAAIQHSRGSTYYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 620 - protein, 365 aa, synthetic construct
EVQLVESGGGLVQPGGSLRLSCAASGLTFSSHCMGWYRQAPGKGREFVAAIQHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 621 - protein, 365 aa, synthetic construct
EVQLVESGGGLVQPGGSLRLSCAASGLTFSSHCMGWYRQAPGKGREFVAAIQRSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 622 - protein, 365 aa, synthetic construct
EVQLVESGGGLVQPGGSLRLSCAASGLTFSSHCMGWYRQAPGKGREFVAAISHSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 623 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 624 - protein, 365 aa, synthetic construct
EVQLVESGGGLVQPGGSLRLSCAASGLTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 625 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 626 - protein, 365 aa, synthetic construct
EVQLVESGGGLVQPGGSLRLSCAASGLTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 627 - protein, 365 aa, synthetic construct
EVQLVESGGGLVQPGGSLRLSCAASGFTFSSHCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 628 - protein, 365 aa, synthetic construct
EVQLVESGGGLVQPGGSLRLSCAASGFTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 629 - protein, 365 aa, synthetic construct
EVQLVESGGGLVQPGGSLRLSCAASGFTFSSHCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 630 - protein, 365 aa, synthetic construct
EVQLVESGGGLVQPGGSLRLSCAASGFTFSSYCMGWYRQAPGKGREFVAAISHSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 631 - protein, 365 aa, synthetic construct
EVQLVESGGGLVQPGGSLRLSCAASGFTFSSYCMGWYRQAPGKGREFVAAIQGSRGSSYADSVKGRFTISRDNSKNTVYLQMNSLRAEDTAVYYCAAVPRAGIESGAYCKWNYKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 632 - protein, 362 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCARTVQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 633 - protein, 362 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCRSRKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 634 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 635 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 636 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 637 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 638 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 639 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 641 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 643 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 362 aa, synthetic construct
EVQLVESGGGLVQPGGSLRLSCAASQQTFSSYAMGWYRQAPGKGREFVASTSGSGGSTYYADSVKGRFTISRDNSKNTVYLQMNSLRAEDTAVYYCRAMRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 645 - protein, 362 aa, synthetic construct
EVQLVESGGGLVQPGGSLRLSCAASQVTFSSYAMGWYRQAPGKGREFVASISHYDGSTYYADSVKGRFTISRDNSKNTVYLQMNSLRAEDTAVYYCRAGRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 646 - protein, 362 aa, synthetic construct
EVQLVESGGGLVQPGGSLRLSCAASGFTFPPYAMGWYRQAPGKGREFVASISGSGGSTYYADSVKGRFTISRDNSKNTVYLQMNSLRAEDTAVYYCRGMKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 647 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 648 - protein, 362 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCCAVKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 649 - protein, 362 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCSAVKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 650 - protein, 362 aa, synthetic construct
EVQLVESGGGLVQPGGSLRLSCAASGFPFSSYAMGWYRQAPGKGREFVAAISFLEGSTYYADSVKGRFTISRDNSKNTVYLQMNSLRAEDTAVYYCRAGKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 651 - protein, 362 aa, synthetic construct
EVQLVESGGGLVQPGGSLRLSCAASGFDISSYAMGWYRQAPGKGREFVAAISFVAGSTYYADSVKGRFTISRDNSKNTVYLQMNSLRAEDTAVYYCEARRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 652 - protein, 362 aa, synthetic construct
EVQLVESGGGLVQPGGSLRLSCAASGFVGSSYAMGWYRQAPGKGREFVASIGGSGGSTYYADSVKGRFTISRDNSKNTVYLQMNSLRAEDTAVYYCRVARQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 653 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 654 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQHRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 655 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRVSLQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 656 - protein, 362 aa, synthetic construct
EVQLVESGGGLVQPGGSLRLSCAASGFPHPSYPMGWYRQAPGKGREFVAGPGGSGGSTYYADSVKGRFTISRDNSKNTVYLQMNSLRAEDTAVYYCRGARQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 657 - protein, 362 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 658 - protein, 362 aa, synthetic construct
EVQLVESGGGLVQPGGSLRLSCAASGFTFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 659 - protein, 362 aa, synthetic construct
EVQLVESGGGLVQPGGSLRLSCAASGFTFSSYAMGWYRQAPGKGREFVASISFSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 660 - protein, 362 aa, synthetic construct
EVQLVESGGGLVQPGGSLRLSCAASGFTFSSYAMGWYRQAPGKGREFVASISGSGGSTQYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 661 - protein, 362 aa, synthetic construct
EVQLVESGGGLVQPGGSLRLSCAASGFTFSSYAMGWYRQAPGKGREFVAAISFSGGSTQYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 662 - protein, 362 aa, synthetic construct
EVQLVESGGGLVQPGGSLRLSCAASGFTFSSYAMGWYRQAPGKGREFVAAISFSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 663 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 664 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 665 - protein, 362 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 362 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 667 - protein, 362 aa, synthetic construct
EVQLVESGGGLVQPGGSLRLSCAASGFTHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 668 - protein, 362 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCARTVQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 669 - protein, 362 aa, synthetic construct
EVQLVESGGGLVQPGGSLRLSCAASGFTFSSYAMGWYRQAPGKGREFVAAISGSGGSTYYADSVKGRFTISRDNSKNTVYLQMNSLRAEDTAVYYCRSRKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 670 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 671 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 672 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGYRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 673 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRSVLQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 674 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 675 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 676 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 677 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 678 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 679 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGFRTVYDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 680 - protein, 362 aa, synthetic construct
EVQLVESGGGLVQPGGSLRLSCAASQQTFSSYAMGWYRQAPGKGREFVASTSGSGGSTYYADSVKGRFTISRDNSKNTVYLQMNSLRAEDTAVYYCRAMRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 681 - protein, 362 aa, synthetic construct
EVQLVESGGGLVQPGGSLRLSCAASQVTFSSYAMGWYRQAPGKGREFVASISHYDGSTYYADSVKGRFTISRDNSKNTVYLQMNSLRAEDTAVYYCRAGRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 682 - protein, 362 aa, synthetic construct
EVQLVESGGGLVQPGGSLRLSCAASGFTFPPYAMGWYRQAPGKGREFVASISGSGGSTYYADSVKGRFTISRDNSKNTVYLQMNSLRAEDTAVYYCRGMKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 683 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMMSQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 684 - protein, 362 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCCAVKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 685 - protein, 362 aa, synthetic construct
EVQLVESGGGLVQPGGSLRLSCAASGFAISSYAMGWYRQAPGKGREFVAAISFIAGSTYYADSVKGRFTISRDNSKNTVYLQMNSLRAEDTAVYYCSAVKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 686 - protein, 362 aa, synthetic construct
EVQLVESGGGLVQPGGSLRLSCAASGFPFSSYAMGWYRQAPGKGREFVAAISFLEGSTYYADSVKGRFTISRDNSKNTVYLQMNSLRAEDTAVYYCRAGKQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 687 - protein, 362 aa, synthetic construct
EVQLVESGGGLVQPGGSLRLSCAASGFDISSYAMGWYRQAPGKGREFVAAISFVAGSTYYADSVKGRFTISRDNSKNTVYLQMNSLRAEDTAVYYCEARRQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 688 - protein, 362 aa, synthetic construct
EVQLVESGGGLVQPGGSLRLSCAASGFVGSSYAMGWYRQAPGKGREFVASIGGSGGSTYYADSVKGRFTISRDNSKNTVYLQMNSLRAEDTAVYYCRVARQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 689 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 690 - protein, 362 aa, synthetic construct
EVQLVESGGGLVQPGGSLRLSCAASGFTHSSYAMGWYRQAPGKGREFVAVISGSGGSTQLLDSVKGRFTISRDNSKNTVYLQMNSLRAEDTAVYYCRAERQHRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 691 - protein, 362 aa, synthetic construct
EVQLVESGGGLVQPGGSLRLSCAASGFTFSSPHMGWYRQAPGKGREFVAAISGSGTAGYYADSVKGRFTISRDNSKNTVYLQMNSLRAEDTAVYYCRVSLQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 692 - protein, 362 aa, synthetic construct
EVQLVESGGGLVQPGGSLRLSCAASGFPHPSYPMGWYRQAPGKGREFVAGPGGSGGSTYYADSVKGRFTISRDNSKNTVYLQMNSLRAEDTAVYYCRGARQRRTLDGYRSSFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 693 - protein, 362 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 694 - protein, 362 aa, synthetic construct
EVQLVESGGGLVQPGGSLRLSCAASGFTFSSYAMGWYRQAPGKGREFVASISGSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 695 - protein, 362 aa, synthetic construct
EVQLVESGGGLVQPGGSLRLSCAASGFTFSSYAMGWYRQAPGKGREFVASISFSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 696 - protein, 362 aa, synthetic construct
EVQLVESGGGLVQPGGSLRLSCAASGFTFSSYAMGWYRQAPGKGREFVASISGSGGSTQYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 697 - protein, 362 aa, synthetic construct
EVQLVESGGGLVQPGGSLRLSCAASGFTFSSYAMGWYRQAPGKGREFVAAISFSGGSTQYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 698 - protein, 362 aa, synthetic construct
EVQLVESGGGLVQPGGSLRLSCAASGFTFSSYAMGWYRQAPGKGREFVAAISFSGGSTYYADSVKGRFTISRDNSKNTVYLQMNSLRAEDTAVYYCRMMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 699 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 700 - protein, 362 aa, synthetic construct
EVQLVESGGGLVQPGGSLRLSCAASGFHHSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 701 - protein, 362 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 702 - protein, 362 aa, synthetic construct
EVQLVESGGGLVQPGGSLRLSCAASGFHFSSYAMGWYRQAPGKGREFVASISGSGGSTYYADSVKGRFTISRDNSKNTVYLQMNSLRAEDTAVYYCRMVSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 703 - protein, 362 aa, synthetic construct
EVQLVESGGGLVQPGGSLRLSCAASGFTHSSYAMGWYRQAPGKGREFVASISGSGGSTYYADSVKGRFTISRDNSKNTVYLQMNSLRAEDTAVYYCRSMSQRRTLDGYRTIFDYWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 704 - protein, 6 aa, synthetic construct
TCPPCP

SEQ ID NO: 705 - protein, 11 aa, synthetic construct
EPKSSTCPPCP

SEQ ID NO: 706 - protein, 8 aa, synthetic construct
THTCPPCP

SEQ ID NO: 707 - protein, 11 aa, synthetic construct
GGGGSTCPPCP

SEQ ID NO: 708 - protein, 15 aa, synthetic construct
GGGGSGGGGTCPPCP

SEQ ID NO: 709 - protein, 15 aa, synthetic construct
AHHPEEPSSQCPKCP

SEQ ID NO: 710 - protein, 15 aa, synthetic construct
AQQPEEPSSQCPKCP

SEQ ID NO: 711 - protein, 12 aa, synthetic construct
GTNEVCKCPKCP

SEQ ID NO: 712 - protein, 14 aa, synthetic construct
EPKSSKTHTCPPCP

SEQ ID NO: 713 - protein, 13 aa, synthetic construct
EPKSKTHTCPPCP

SEQ ID NO: 714 - protein, 15 aa, synthetic construct
EPKSSQKTHTCPPCP

SEQ ID NO: 715 - protein, 15 aa, synthetic construct
EPKSAQKTHTCPPCP

SEQ ID NO: 716 - protein, 11 aa, synthetic construct
EPKSATCPPCP

SEQ ID NO: 717 - protein, 15 aa, synthetic construct
GGGGSGGGGQCPPCP

SEQ ID NO: 718 - protein, 15 aa, synthetic construct
GGGGSGGGGACPPCP

SEQ ID NO: 719 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 720 - protein, 228 aa, synthetic construct
EPKSSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 721 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 722 - protein, 225 aa, synthetic construct
THTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 723 - protein, 228 aa, synthetic construct
GGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 724 - protein, 232 aa, synthetic construct
GGGGSGGGGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 725 - protein, 232 aa, synthetic construct
AHHPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 726 - protein, 232 aa, synthetic construct
AQQPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 727 - protein, 229 aa, synthetic construct
GTNEVCKCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 728 - protein, 231 aa, synthetic construct
EPKSSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 729 - protein, 230 aa, synthetic construct
EPKSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 730 - protein, 232 aa, synthetic construct
EPKSSQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 731 - protein, 232 aa, synthetic construct
EPKSAQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 732 - protein, 228 aa, synthetic construct
EPKSATCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 733 - protein, 232 aa, synthetic construct
GGGGSGGGGQCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 734 - protein, 232 aa, synthetic construct
GGGGSGGGGACPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 735 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 736 - protein, 227 aa, synthetic construct
EPKSSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 737 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 738 - protein, 224 aa, synthetic construct
THTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 739 - protein, 227 aa, synthetic construct
GGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 740 - protein, 231 aa, synthetic construct
GGGGSGGGGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 741 - protein, 231 aa, synthetic construct
AHHPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 742 - protein, 231 aa, synthetic construct
AQQPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 743 - protein, 228 aa, synthetic construct
GTNEVCKCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 744 - protein, 230 aa, synthetic construct
EPKSSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 745 - protein, 229 aa, synthetic construct
EPKSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 746 - protein, 231 aa, synthetic construct
EPKSSQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 747 - protein, 231 aa, synthetic construct
EPKSAQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 748 - protein, 227 aa, synthetic construct
EPKSATCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 749 - protein, 231 aa, synthetic construct
GGGGSGGGGQCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 750 - protein, 231 aa, synthetic construct
GGGGSGGGGACPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 751 - protein, 366 aa, synthetic construct; identical to SEQ ID NO: 539

SEQ ID NO: 752 - protein, 366 aa, synthetic construct; identical to SEQ ID NO: 541

SEQ ID NO: 753 - protein, 357 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 754 - protein, 362 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 755 - protein, 351 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 756 - protein, 359 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 757 - protein, 357 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 758 - protein, 362 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 759 - protein, 351 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 760 - protein, 359 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 761 - protein, 362 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 762 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSGGGGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 763 - protein, 362 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 764 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSGGGGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 765 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSAHHPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 766 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSAQQPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 767 - protein, 363 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGTNEVCKCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 768 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSAHHPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 769 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSAQQPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 770 - protein, 363 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGTNEVCKCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 771 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 772 - protein, 364 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 773 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 774 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSAQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 775 - protein, 362 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSATCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 776 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSGGGGQCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 777 - protein, 366 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSGGGGACPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 778 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 779 - protein, 364 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 780 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 781 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSAQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 782 - protein, 362 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSATCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 783 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSGGGGQCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 784 - protein, 366 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSGGGGACPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 785 - protein, 365 aa, synthetic construct; identical to SEQ ID NO: 623

SEQ ID NO: 786 - protein, 365 aa, synthetic construct; identical to SEQ ID NO: 625

SEQ ID NO: 787 - protein, 356 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 788 - protein, 361 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 789 - protein, 350 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 790 - protein, 358 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 791 - protein, 356 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 792 - protein, 361 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 793 - protein, 350 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 794 - protein, 358 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 795 - protein, 361 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 796 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSGGGGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 797 - protein, 361 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 798 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSGGGGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 799 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSAHHPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 800 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSAQQPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 801 - protein, 362 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGTNEVCKCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 802 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSAHHPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 803 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSAQQPEEPSSQCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 804 - protein, 362 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGTNEVCKCPKCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 805 - protein, 364 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 806 - protein, 363 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 807 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSSQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 808 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSAQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 809 - protein, 361 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSEPKSATCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 810 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSGGGGQCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 811 - protein, 365 aa, synthetic construct
EVQLVESGGGLVQPGGSLRLSCAASGLTFSSHCMGWYRQAPGKGREFVAAISGSRGYSYADSVKGRFTISRDNSKNTVYLQMNSLRAEDTAVYYCAAVPRAGIESGAYCKWSYKDSGSWGQGTQVTVSSGGGGSGGGGSGGGGACPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 812 - protein, 364 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 813 - protein, 363 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 814 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSSQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 815 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSAQKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 816 - protein, 361 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSEPKSATCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 817 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSGGGGQCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 818 - protein, 365 aa, synthetic construct
EVQLVESGGGLVQPGGSLRLSCAASGLTFSSHCMGWYRQAPGKGREFVAAIQHSRGSSYADSVKGRFTISRDNSKNTVYLQMNSLRAEDTAVYYCAAVPRAGIESGAYCKWNYKDSGSWGQGTQVTVSSGGGGSGGGGSGGGGACPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 819 - protein, 25 aa, synthetic construct [sequence omitted]

SEQ ID NO: 820 - protein, 14 aa, synthetic construct
WYRQAPGKGREFVA

SEQ ID NO: 821 - protein, 31 aa, synthetic construct
GRFTISRDNSKNTVYLQMNSLRAEDTAVYYC

SEQ ID NO: 822 - protein, 11 aa, synthetic construct
WGQGTQVTVSS

SEQ ID NO: 823 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIFSGAYCKWNMKDSGSWGQGTLVTVSS

SEQ ID NO: 824 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIWSGAYCKWNMKDSGSWGQGTLVTVSS

SEQ ID NO: 825 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIYSGAYCKWNMKDSGSWGQGTLVTVSS

SEQ ID NO: 826 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIEFGAYCKWNMKDSGSWGQGTLVTVSS

SEQ ID NO: 827 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIEWGAYCKWNMKDSGSWGQGTLVTVSS

SEQ ID NO: 828 - protein, 129 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIEYGAYCKWNMKDSGSWGQGTLVTVSS

SEQ ID NO: 829 - protein, 130 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIEFSGAYCKWNMKDSGSWGQGTLVTVSS

SEQ ID NO: 830 - protein, 130 aa, synthetic construct
QVQLVESGGGSVQSGGSLTLSCAASGSTYSSHCMGWFRQAPGKEREGVALMTRSRGTSYADSVKGRFTISQDNTKNILYLQMNSLKPEDTAMYYCAAVPRAGIEWSGAYCKWNMKDSGSWGQGTLVTVSS

SEQ ID NO: 831 - protein, 15 aa, Homo sapiens
EPKSSDKTHTCPPCP

SEQ ID NO: 832 - skipped

SEQ ID NO: 833 - protein, 11 aa, synthetic construct
AISGSGGSTYY

SEQ ID NO: 834 - protein, 11 aa, synthetic construct
AISGSGTAGYY

SEQ ID NO: 835 - protein, 11 aa, synthetic construct
VISGSGGSTQL

SEQ ID NO: 836 - protein, 11 aa, synthetic construct
SISGSGGSTYY

SEQ ID NO: 837 - protein, 11 aa, synthetic construct
STSGSGGSTYY

SEQ ID NO: 838 - protein, 11 aa, synthetic construct
SISHYDGSTYY

SEQ ID NO: 839 - protein, 11 aa, synthetic construct
AISFIAGSTYY

SEQ ID NO: 840 - protein, 11 aa, synthetic construct
AISFLEGSTYY

SEQ ID NO: 841 - protein, 11 aa, synthetic construct
AISFVAGSTYY

SEQ ID NO: 842 - protein, 11 aa, synthetic construct
SIGGSGGSTYY

SEQ ID NO: 843 - protein, 11 aa, synthetic construct
GPGGSGGSTYY

SEQ ID NO: 844 - protein, 11 aa, synthetic construct
SISFSGGSTYY

SEQ ID NO: 845 - protein, 11 aa, synthetic construct
SISGSGGSTQY

SEQ ID NO: 846 - protein, 11 aa, synthetic construct
AISFSGGSTQY

SEQ ID NO: 847 - protein, 11 aa, synthetic construct
AISFSGGSTYY

SEQ ID NO: 848 - protein, 5 aa, synthetic construct
CPPCP

SEQ ID NO: 849 - protein, 36 aa, synthetic construct
ADSVKGRFTISRDNSKNTVYLQMNSLRAEDTAVYYC

SEQ ID NO: 850 - protein, 8 aa, synthetic construct
DYKDDDDK

SEQ ID NO: 851 - protein, 10 aa, synthetic construct
HHHHHHHHHH

SEQ ID NO: 852 - protein, 380 aa, Homo sapiens
MEEGGDFDNYYGADNQSECEYTDWKSSGALIPAIYMLVFLLGTTGNGLVLWTVFRSSREKRRSADIFIASLAVADLTFVVTLPLWATYTYRDYDWPFGTFFCKLSSYLIFVNMYASVFCLTGLSFDRYLAIVRPVANARLRLRVSGAVATAVLWVLAALLAMPVMVLRTTGDLENTTKVQCYMDYSMVATVSSEWAWEVGLGVSSTTVGFVVPFTIMLTCYFFIAQTIAGHFRKERIEGLRKRRRLLSIIVVLVVTFALCWMPYHLVKTLYMLGSLLHWPCDFDLFLMNIFPYCTCISYVNSCLNPFLYAFFDPRFRQACTSMLCCGQSRCAGTSHSSSGEKSASYSSGHSQGPGPNMGKGGEQMHEKSIPYSQETLVVD

SEQ ID NO: 853 - protein, 367 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEPSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 854 - protein, 367 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIERSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 855 - protein, 367 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEWSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 856 - protein, 367 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEKSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 857 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKFNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 858 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKYNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 859 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEPSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 860 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIERSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 861 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEWSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 862 - protein, 366 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIEKSGAYCKWNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 863 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKFNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 864 - protein, 365 aa, synthetic construct
EVQLVESGGGLVQPGGSLRLSCAASGSTYSSHCMGWYRQAPGKGREFVALMTRSRGTSYADSVKGRFTISRDNSKNTVYLQMNSLRAEDTAVYYCAAVPRAGIESGAYCKYNMKDSGSWGQGTQVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0085125 A1
(43) Pub. Date: Mar. 26, 2026

(54) APELIN RECEPTOR BINDING POLYPEPTIDES AND METHODS

(71) Applicant: TECTONIC OPERATING COMPANY, INC., Watertown, MA (US)

(72) Inventors: Peter James MAREK, Watertown, MA (US); Yao FAN, Watertown, MA (US); John L. DIENER, Watertown, MA (US); Peter Jeremiah MCNAMARA, Watertown, MA (US); Ryan KNIHTILA, Watertown, MA (US); Jaime Rene DARCE, Watertown, MA (US); Christian CORTIS, Watertown, MA (US); Ximena BARROS ALVAREZ, Watertown, MA (US)

(21) Appl. No.: 19/401,078

(22) Filed: Nov. 25, 2025

Related U.S. Application Data
(63) Continuation of application No. PCT/US2025/043193, filed on Aug. 22, 2025.
(60) Provisional application No. 63/686,558, filed on Aug. 23, 2024.

(51) Int. Cl. C07K 16/28; A61K 39/00; A61P 7/04

(52) U.S. Cl. CPC C07K 16/2869; A61P 7/04; A61K 2039/505; C07K 2317/33; C07K 2317/34; C07K 2317/52; C07K 2317/565; C07K 2317/76

(57) ABSTRACT

The present disclosure provides antibodies and polypeptides that specifically bind to apelin receptor (APJ). Also provided are compositions comprising these antibodies, nucleic acids encoding these antibodies, expression vectors and host cells for making these antibodies, and methods of treating a subject using these antibodies.

### RELATED APPLICATIONS

This application is a U.S. bypass continuation application of PCT/US2025/043193, filed Aug. 22, 2025, which claims priority to U.S. Provisional Patent Application Ser. No. 63/686,558, filed Aug. 23, 2024, the entire disclosures of which are hereby incorporated by reference herein.

### REFERENCE TO SEQUENCE LISTING

This application contains a sequence listing which has been submitted electronically in XML format and is hereby incorporated by reference in its entirety (said XML copy, created Sep. 26, 2025, is named “TETH-005_02US_337527-2003_SeqList_ST26.xml” and is 927,738 bytes in size).

### BACKGROUND

The apelin receptor (APJ, also known as APLNR) is a G protein-coupled receptor that recognizes two endogenous ligands, apelin and elabela. Signaling through APJ plays a role in important early development processes, including gastrulation, blood vessel formation, and heart morphogenesis. APJ signaling also regulates blood vessel formation in adults, and it has been reported to play a role in other processes including regulation of blood pressure, heart contractility, and heart failure. APJ dysfunction has been linked to the etiology of various diseases or disorders, including pulmonary arterial hypertension, obesity, heart failure, diabetes, and cancer.

Thus, there is a need for therapies targeting APJ.

### SUMMARY

The present disclosure provides antibodies and polypeptides that specifically bind to human apelin receptor (APJ). In certain embodiments, the anti-APJ antibodies are APJ antagonists. In certain embodiments, the anti-APJ antibodies are APJ agonists. Also provided are pharmaceutical compositions comprising these antibodies, nucleic acids encoding these antibodies, expression vectors and host cells for making these antibodies, and methods of treating a subject using these antibodies. The antibodies provided herein specifically bind APJ and modulate APJ activity and therefore have utility in the treatment of APJ-associated diseases or disorders (e.g., hereditary hemorrhagic telangiectasia (HHT), pulmonary arterial hypertension, obesity, cancer, etc.) in a subject. In certain embodiments, antibodies disclosed herein appear to be particularly advantageous relative to prior art antibodies evaluated in the Examples herein.

Accordingly, in one aspect, provided herein is an antibody that specifically binds human apelin receptor (APJ), the antibody comprising a heavy chain variable domain comprising complementarity determining regions CDRH1, CDRH2, and CDRH3, wherein: the CDRH1 comprises the amino acid sequence of GX₁X₂X₃X₄X₅X₆CX₇X₈ (SEQ ID NO: 247), wherein: X₁ is L, F, I, S, Y, A, H, V, or Q; X₂ is T, H, L, N, Q, or S; X₃ is F, Y, I, L, or V; X₄ is S, A, H, Q, V, I, or T; X₅ is S, F, H, or Y; X₆ is H or Y; X₇ is M or absent; and X₈ is G, S, L, Y, or absent; the CDRH2 comprises the amino acid sequence of X₉X₁₀X₁₁X₁₂SX₁₃GX₁₄X₁₅X₁₆X₁₇ (SEQ ID NO: 248), wherein: X₉ is A, L, or absent; X₁₀ is I or M; X₁₁ is S, A, Q, or T; X₁₂ is G, H, or R; X₁₃ is R or Y; X₁₄ is Y, S, T, F, or H; X₁₅ is S, T, Y, Q, or absent; X₁₆ is Y or absent; and X₁₇ is absent or Y; and the CDRH3 comprises the amino acid sequence of AAVPRAGIX₁₈X₁₉X₂₀GAYCKX₂₁X₂₂X₂₃X₂₄DSGS (SEQ ID NO: 249), wherein: X₁₈ is E, F, Y, or W; X₁₉ is absent, Y, F, P, K, R, W, L, or I; X₂₀ is S, F, Y, or W; X₂₁ is W, A, F or Y; X₂₂ is S, H, I, K, N, P, Q, R, or T; X₂₃ is Y, G, H, I, L, M, N, or R; and X₂₄ is K or Q, wherein the VH does not comprise the amino acid sequence set forth in SEQ ID NO: 60-64 or 823-830.

In certain embodiments, X₁ is L, F, I, S, or Y; X₂ is T, H, L, or N; X₃ is F or Y; X₄ is S, A, H, Q, or V; X₅ is S or F; X₆ is H or Y; X₇ is M or absent; X₈ is G or absent; X₉ is A, L, or absent; X₁₀ is I or M; X₁₁ is S, A, Q, or T; X₁₂ is G, H, or R; X₁₃ is R or Y; X₁₄ is Y, S, or T; X₁₅ is S, T, Y, or absent; X₁₆ is Y or absent; X₁₇ is absent or Y; X₁₈ is E; X₁₉ is absent or Y; X₂₀ is S; X₂₁ is W or A; X₂₂ is S, H, I, K, N, P, Q, R, or T; X₂₃ is Y, G, H, I, L, M, N, or R; and X₂₄ is K or Q.

In certain embodiments, X₄ is S, H, Q, or V; X₁₁ is S, Q, or T; and X₂₁ is W.

In certain embodiments, X₁₉ is absent. In certain embodiments, X₁₉ is Y or F.

In certain embodiments, X₇, X₈, X₉, X₁₅, X₁₆, and X₁₇ are absent.

In certain embodiments, X₁ is L; X₂ is T; X₃ is F; X₄ is S; X₅ is S; X₆ is H; X₇ is absent; X₈ is absent; X₉ is absent; X₁₀ is I; X₁₁ is S or Q; X₁₂ is G or H; X₁₃ is R; X₁₄ is Y or S; X₁₈ is absent; X₁₆ is absent; X₁₇ is absent; X₁₈ is E; X₁₉ is absent; X₂₀ is S; X₂₁ is W; X₂₂ is S or N; X₂₃ is Y; and X₂₄ is K.

In certain embodiments, X₁ is L; X₂ is T; X₃ is F; X₄ is S; X₅ is S; X₆ is H; X₇ is M; X₈ is G; X₉ is A; X₁₀ is I; X₁₁ is S or Q; X₁₂ is G or H; X₁₃ is R; X₁₄ is Y or S; X₁₅ is S; X₁₆ is Y; X₁₇ is absent; X₁₈ is E; X₁₉ is absent; X₂₀ is S; X₂₁ is W; X₂₂ is S or N; X₂₃ is Y; and X₂₄ is K.

In certain embodiments, X₁ is L; X₃ is F; X₉ is A; X₁₄ is Y; X₂₂ is S; and/or X₂₃ is Y.

In certain embodiments, X₁ is L; X₃ is F; X₉ is A; X₁₄ is Y; X₂₂ is S; and X₂₃ is Y.

In another aspect, provided herein is an antibody that specifically binds human APJ, the antibody comprising a heavy chain variable domain (VH) comprising the CDRH1, CDRH2, and CDRH3 amino acid sequences of any one of the VH amino acid sequences set forth in SEQ ID NOs: 1-84, wherein the VH does not comprise the amino acid sequence set forth in SEQ ID NO: 60-64 or 823-830.

In certain embodiments, the VH comprises the CDRH1, CDRH2, and CDRH3 amino acid sequences, respectively, set forth in SEQ ID NOs: 374, 384, and 141; 354, 355, and 87; 354, 355, and 90; 354, 357, and 93; 354, 357, and 96; 354, 355, and 96; 354, 355, and 99; 354, 357, and 99; 354, 355, and 102; 354, 359, and 102; 354, 359, and 105; 354, 355, and 105; 354, 357, and 105; 354, 355, and 108; 354, 357, and 108; 354, 357, and 111; 354, 357, and 114; 354, 355, and 114; 354, 357, and 117; 354, 355, and 117; 354, 357, and 120; 354, 357, and 123; 354, 357, and 125; 354, 357, and 127; 354, 357, and 129; 354, 357, and 131; 354, 357, and 133; 354, 357, and 135; 354, 357, and 137; 354, 357, and 139; 354, 357, and 141; 354, 355, and 143; 354, 355, and 145; 354, 355, and 147; 354, 355, and 149; 354, 355, and 151; 354, 355, and 152; 354, 355, and 153; 356, 361, and 117; 358, 361, and 117; 360, 363, and 117; 362, 365, and 117; 364, 367, and 133; 366, 367, and 133; 368, 369, and 133; 370, 371, and 133; 354, 373, and 108; 354, 375, and 117; 354, 373, and 120; 354, 373, and 129; 354, 373, and 131; 354, 373, and 133; 354, 373, and 139; 354, 355, and 154; 372, 355, and 87; 354, 377, and 87; 354, 355, and 155; 354, 373, and 141; 374, 379, and 141; 374, 380, and 141; 374, 381, and 141; 374, 382, and 141; 374, 383, and 141; 374, 385, and 131; 374, 380, and 131; 374, 379, and 131; 376, 385, and 131; 376, 380, and 131; 376, 379, and 131; 378, 385, and 131; or 378, 379, and 131.

In certain embodiments, the VH comprises the CDRH1, CDRH2, and CDRH3 amino acid sequences, respectively, set forth in SEQ ID NOs: 115, 144, and 141; 85, 86, and 87; 85, 86, and 90; 85, 89, and 93; 85, 89, and 96; 85, 86, and 96; 85, 86, and 99; 85, 89, and 99; 85, 86, and 102; 85, 92, and 102; 85, 92, and 105; 85, 86, and 105; 85, 89, and 105; 85, 86, and 108; 85, 89, and 108; 85, 89, and 111; 85, 89, and 114; 85, 86, and 114; 85, 89, and 117; 85, 86, and 117; 85, 89, and 120; 85, 89, and 123; 85, 89, and 125; 85, 89, and 127; 85, 89, and 129; 85, 89, and 131; 85, 89, and 133; 85, 89, and 135; 85, 89, and 137; 85, 89, and 139; 85, 89, and 141; 85, 86, and 143; 85, 86, and 145; 85, 86, and 147; 85, 86, and 149; 85, 86, and 151; 85, 86, and 152; 85, 86, and 153; 88, 95, and 117; 91, 98, and 117; 94, 101, and 117; 97, 104, and 117; 100, 107, and 133; 103, 110, and 133; 106, 113, and 133; 109, 116, and 133; 85, 119, and 108; 85, 122, and 108; 85, 124, and 117; 85, 126, and 117; 85, 119, and 120; 85, 122, and 120; 85, 119, and 129; 85, 122, and 129; 85, 119, and 131; 85, 122, and 131; 85, 119, and 133; 85, 122, and 133; 85, 119, and 139; 85, 122, and 139; 85, 86, and 154; 112, 86, and 87; 85, 128, and 87; 85, 86, and 155; 85, 130, and 141; 85, 132, and 141; 115, 134, and 141; 115, 136, and 141; 115, 138, and 141; 115, 140, and 141; 115, 142, and 141; 115, 146, and 131; 115, 148, and 131; 115, 150, and 131; 118, 146, and 131; 118, 148, and 131; 118, 150, and 131; 121, 146, and 131; or 121, 150, and 131.

In certain embodiments, the VH comprises an amino acid sequence having at least 85%, 86%, 87%, 88%, 89%, 90%, 91%, 92%, 93%, 94%, 95%, 96%, 97%, 98%, 99%, or 100% sequence identity to any one of SEQ ID NOs: 1-59 and 65-84. In certain embodiments, the VH comprises the amino acid sequence of any one of SEQ ID NOs: 1-59 and 65-84. In certain embodiments, the amino acid sequence of the VH consists of the amino acid sequence of any one of SEQ ID NOs: 1-59 and 65-84.

In another aspect, provided herein is an antibody that specifically binds human APJ, the antibody comprising a heavy chain variable domain (VH) comprising complementarity determining regions CDRH1, CDRH2, and CDRH3, wherein: the CDRH1 comprises the amino acid sequence of X₂₅X₂₆X₂₇X₂₈X₂₉X₃₀X₃₁X₃₂X₃₃X₃₄ (SEQ ID NO: 250), wherein: X₂₅ is G or Q; X₂₆ is F, Q, or V; X₂₇ is T, A, D, H, P, V, R, K, or E; X₂₈ is F, G, H, I, or V; X₂₉ is S, P, R, or K; X₃₀ is S or P; X₃₁ is P or Y; X₃₂ is H, A, P, R, or K; X₃₃ is M or absent; and X₃₄ is G, R, K, H, or absent; the CDRH2 comprises the amino acid sequence of X₃₅X₃₆X₃₇X₃₈X₃₉X₄₀X₄₁X₄₂X₄₃X₄₄X₄₅X₄₆X₄₇X₄₈X₄₉X₅₀ (SEQ ID NO: 251), wherein: X₃₅ is A, G, S, V, R, K, H, or absent; X₃₆ is I, P, or T; X₃₇ is S or G; X₃₈ is G, F, or H; X₃₉ is S, I, L, V, or Y; X₄₀ is G, A, D, or E; X₄₁ is T, G, R, K, or H; X₄₂ is A or S; X₄₃ is G, T, or absent; X₄₄ is Y, Q, R, K, H, or absent; X₄₅ is Y, L, E, D, or absent; X₄₆ is A, L, or absent; X₄₇ is D, H, P, or absent; X₄₈ is S or absent; X₄₉ is V or absent; and X₅₀ is K, Q, or absent; and the CDRH3 comprises the amino acid sequence of X₅₁X₅₂X₅₃X₅₄X₅₅X₅₆RX₅₇LX₅₈GX₅₉RX₆₀X₆₁X₆₂DY (SEQ ID NO: 252), wherein: X₅₁ is R, A, C, E, or S; X₅₂ is V, A, G, M, R, or S; X₅₃ is S, A, E, G, M, R, T, or V; X₅₄ is L, K, R, S, or V; X₅₅ is Q or G; X₅₆ is R or H; X₅₇ is T, L, or M; X₅₈ is D or E; X₅₉ is Y or F; X₆₀ is S or T; X₆₁ is S, I, V, or L; and X₆₂ is F or Y.

In certain embodiments, X₂₅ is G or Q; X₂₆ is F, Q, or V; X₂₇ is T, A, D, H, P, or V; X₂₈ is F, G, H, or I; X₂₉ is S or P; X₃₀ is S or P; X₃₁ is P or Y; X₃₂ is H, A, or P; X₃₃ is M or absent; X₃₄ is G or absent; X₃₅ is A, G, S, V, or absent; X₃₆ is I, P, or T; X₃₇ is S or G; X₃₈ is G, F, or H; X₃₉ is S, I, L, V, or Y; X₄₀ is G, A, D, or E; X₄₁ is T or G; X₄₂ is A or S; X₄₃ is G, T, or absent; X₄₄ is Y, Q, or absent; X₄₅ is Y, L, or absent; X₄₆ is A, L, or absent; X₄₇ is D or absent; X₄₈ is S or absent; X₄₉ is V or absent; X₅₀ is K or absent; X₅₁ is R, A, C, E, or S; X₅₂ is V, A, G, M, R, or S; X₅₃ is S, A, E, G, M, R, T, or V; X₅₄ is L, K, R, S, or V; X₅₅ is Q; X₅₆ is R or H; X₅₇ is T; X₅₈ is D; X₅₉ is Y or F; X₆₀ is S or T; X₆₁ is S, I, or V; and X₆₂ is F or Y.

In certain embodiments, X₃₃, X₃₄, X₃₅, X₄₃, X₄₄, X₄₅, X₄₆, X₄₇, X₄₈, X₄₉, and X₅₀ are absent.

In certain embodiments, X₃₃ is M; X₃₄ is G; X₃₅ is A, G, S, or V; X₄₃ is G or T; X₄₄ is Y or Q; X₄₅ is Y or L; and X₄₆, X₄₇, X₄₈, X₄₉, and X₅₀ are absent.

In certain embodiments, X₃₃ is M; X₃₄ is G; X₃₅ is A, G, S, or V; X₄₃ is G or T; X₄₄ is Y or Q; X₄₅ is Y or L; X₄₆ is A or L; X₄₇ is D; X₄₈ is S; X₄₉ is V; and X₅₀ is K.

In another aspect, provided herein is an antibody that specifically binds human APJ, the antibody comprising a VH comprising the CDRH1, CDRH2, and CDRH3 amino acid sequences of any one of the VH amino acid sequences set forth in SEQ ID NOs: 156-191.

In certain embodiments, the VH comprises the CDRH1, CDRH2, and CDRH3 amino acid sequences, respectively, set forth in SEQ ID NOs: 429, 430, and 243; 427, 428, and 194; 427, 428, and 197; 429, 430, and 200; 429, 430, and 203; 429, 430, and 206; 429, 430, and 209; 431, 428, and 212; 431, 428, and 215; 433, 428, and 218; 433, 428, and 221; 433, 428, and 224; 433, 428, and 227; 435, 432, and 230; 437, 434, and 232; 439, 428, and 234; 433, 428, and 235; 441, 436, and 236; 441, 436, and 237; 443, 438, and 238; 445, 440, and 239; 447, 442, and 240; 431, 428, and 241; 431, 428, and 242; 448, 444, and 244; 449, 428, and 218; 427, 428, and 218; 427, 446, and 218; 433, 428, and 245; 433, 428, and 246; 449, 428, and 245; 449, 428, and 246; or 431, 428, and 245.

In certain embodiments, the VH comprises the CDRH1, CDRH2, and CDRH3 amino acid sequences, respectively, set forth in SEQ ID NOs: 195, 834, and 243; 192, 833, and 194; 192, 833, and 197; 195, 834, and 200; 195, 834, and 203; 195, 834, and 206; 195, 834, and 209; 198, 835, and 212; 198, 835, and 215; 201, 836, and 218; 201, 836, and 221; 201, 836, and 224; 201, 836, and 227; 204, 837, and 230; 207, 838, and 232; 210, 836, and 234; 201, 836, and 235; 213, 839, and 236; 213, 839, and 237; 216, 840, and 238; 219, 841, and 239; 222, 842, and 240; 198, 835, and 241; 198, 835, and 242; 225, 843, and 244; 228, 836, and 218; 192, 836, and 218; 192, 844, and 218; 192, 845, and 218; 192, 846, and 218; 192, 847, and 218; 201, 836, and 245; 201, 836, and 246; 228, 836, and 245; 228, 836, and 246; or 198, 836, and 245.

In certain embodiments, the VH comprises the CDRH1, CDRH2, and CDRH3 amino acid sequences, respectively, set forth in SEQ ID NOs: 195, 196, and 243; 192, 193, and 194; 192, 193, and 197; 195, 196, and 200; 195, 196, and 203; 195, 196, and 206; 195, 196, and 209; 198, 199, and 212; 198, 199, and 215; 201, 202, and 218; 201, 202, and 221; 201, 202, and 224; 201, 202, and 227; 204, 205, and 230; 207, 208, and 232; 202, 210, and 234; 201, 202, and 235; 211, 213, and 236; 211, 213, and 237; 214, 216, and 238; 217, 219, and 239; 220, 222, and 240; 198, 199, and 241; 198, 199, and 242; 223, 225, and 244; 202, 218, and 228; 192, 202, and 218; 192, 218, and 226; 192, 218, and 229; 192, 218, and 231; 192, 218, and 233; 201, 202, and 245; 201, 202, and 246; 202, 228, and 245; 202, 228, and 246; or 198, 202, and 245.

In certain embodiments, the VH comprises an amino acid sequence having at least 85%, 86%, 87%, 88%, 89%, 90%, 91%, 92%, 93%, 94%, 95%, 96%, 97%, 98%, 99%, or 100% sequence identity to any one of SEQ ID NOs: 156-191. In certain embodiments, the VH comprises the amino acid sequence of any one of SEQ ID NOs: 156-191. In certain embodiments, the amino acid sequence of the VH consists of the amino acid sequence of any one of SEQ ID NOs: 156-191.

In certain embodiments, any of the antibodies described above further comprise an IgG Fc. In certain embodiments, the IgG Fc comprises alanine at each of EU positions 234 and 235. In certain embodiments, the IgG Fc comprises alanine at EU position 329. In certain embodiments, the IgG Fc comprises alanine at each of EU positions 234, 235, and 329. In certain embodiments, the IgG Fc comprises glycine at EU position 329. In certain embodiments, the IgG Fc comprises alanine, alanine, and glycine at EU positions 234, 235, and 329, respectively. In certain embodiments, the IgG Fc comprises leucine and serine at EU positions 428 and 434, respectively. In certain embodiments, the IgG Fc comprises alanine, alanine, alanine, leucine, and serine at EU positions 234, 235, 329, 428, and 434, respectively. In certain embodiments, the IgG Fc comprises alanine at EU position 435. In certain embodiments, the IgG Fc comprises alanine, alanine, alanine, and alanine at EU positions 234, 235, 329, and 435, respectively. In certain embodiments, the IgG Fc comprises tyrosine, threonine, and glutamate at EU positions 252, 254, and 256, respectively. In certain embodiments, the IgG Fc comprises phenylalanine, glutamate, and serine at EU positions 234, 235, and 331, respectively. In certain embodiments, IgG Fc comprises phenylalanine, glutamate, tyrosine, threonine, glutamate, and serine at EU positions 234, 235, 252, 254, 256, and 331 respectively. In certain embodiments, the IgG Fc comprises leucine and serine at EU positions 428 and 434, respectively. In certain embodiments, the IgG Fc comprises tyrosine, threonine, glutamate, leucine, and serine at EU positions 252, 254, 256, 428, and 434, respectively. In certain embodiments, the IgG Fc comprises alanine at each of EU positions 265 and 329. In certain embodiments, the IgG Fc comprises alanine at each of EU positions 265, 297, and 329. In certain embodiments, the IgG Fc comprises alanine at each of EU positions 253, 310, and 435. In certain embodiments, the IgG Fc comprises glutamine and leucine at EU positions 250 and 428, respectively. In certain embodiments, the IgG Fc comprises alanine at each of EU positions 307, 380, and 434. In certain embodiments, the IgG Fc comprises phenylalanine, glutamine, and glutamine at EU positions 234, 235, and 322, respectively.

In certain embodiments, the IgG Fc comprises an amino acid sequence having at least 85%, 86%, 87%, 88%, 89%, 90%, 91%, 92%, 93%, 94%, 95%, 96%, 97%, 98%, 99%, or 100% sequence identity to the amino acid sequence of a human IgG1 Fc. In certain embodiments, the IgG Fc comprises the amino acid sequence of a human IgG1 Fc. In certain embodiments, the IgG Fc comprises an amino acid sequence having at least 95%, 96%, 97%, 98%, 99%, or 100% sequence identity to an amino acid sequence selected from the group consisting of SEQ ID NOs: 253-268, and 450-463. In certain embodiments, the amino acid sequence of the IgG Fc consists of the amino acid sequence selected from the group consisting of SEQ ID NOs: 253-268, and 450-463.

In certain embodiments, the N-terminus of the IgG Fc is linked to the C-terminus of the VH, optionally via a linker. In certain embodiments, the C-terminus of the IgG Fc is linked to the N-terminus of the VH, optionally via a linker. In certain embodiments, the linker comprises five amino acids. In certain embodiments, the linker comprises or consists of the amino acid sequence GGGGS (SEQ ID NO: 269).

In certain embodiments, the IgG Fc comprises a hinge region comprising SEQ ID NO: 831. In certain embodiments, the IgG Fc comprises a modified hinge region. In certain embodiments, the modified hinge region comprises one or more mutations (e.g., amino acid substitutions, insertions or deletions) relative to SEQ ID NO: 831. In certain embodiments, the modified hinge region comprises an amino acid substitution, insertion, and/or deletion. In certain embodiments, the substitution, insertion, and/or deletion is in the region spanning EU positions 216 to 230. In certain embodiments, the modified hinge region comprises a deletion of one or more amino acids at EU positions 216 to 230. In certain embodiments, the modified hinge region comprises 0, 1, 2, 3, 4, 5, 6, 7, 8, 9, 10, 11, 12, 13, 14 or 15 amino acids in the region spanning EU positions 216 to 230. In certain embodiments, the modified hinge region comprises the sequence CPPCP (SEQ ID NO: 848) in the region spanning EU positions 216 to 230. In certain embodiments, the modified hinge region comprises 0, 1, 2, 3, 4, 5, 6, 7, 8, 9, or 10 amino acids N-terminal to the sequence CPPCP (SEQ ID NO: 848) in the region spanning EU positions 216 to 230. In certain embodiments, the modified hinge region comprises any one of the amino acid sequences set forth in SEQ ID NOs: 704-718. In certain embodiments, the IgG Fc comprises any one of the amino acid sequences set forth in SEQ ID NOs: 719-750.

In certain embodiments of the antibodies provided herein, the antibody comprises an amino acid sequence having at least 85%, 86%, 87%, 88%, 89%, 90%, 91%, 92%, 93%, 94%, 95%, 96%, 97%, 98%, 99%, or 100% sequence identity to any one of SEQ ID NOs: 270-353, 464-522, 528-606, 612-631, 751-818, and 853-864. In certain embodiments, the amino acid sequence of the antibody consists of the amino acid sequence selected from the group consisting of SEQ ID NOs: 270-353, 464-522, 528-606, 612-631, 751-818, and 853-864. In certain embodiments, the antibody comprises an amino acid sequence having at least 85%, 86%, 87%, 88%, 89%, 90%, 91%, 92%, 93%, 94%, 95%, 96%, 97%, 98%, 99%, or 100% sequence identity to any one of SEQ ID NOs: 391-426 and 632-703. In certain embodiments, the amino acid sequence of the antibody consists of the amino acid sequence selected from the group consisting of SEQ ID NOs: 391-426 and 632-703.

In another aspect, provided herein is an antibody that specifically binds human apelin receptor (APJ), the APJ comprising the amino acid sequence of SEQ ID NO: 852, wherein: (a) the antibody specifically interacts with the aspartate residue at position 172 of SEQ ID NO: 852; and/or (b) the antibody does not specifically interact with the cysteine residue at position 281 of SEQ ID NO: 852.

In certain embodiments, the antibody specifically interacts with the aspartate residue at position 172 of SEQ ID NO: 852; and the antibody does not specifically interact with the cysteine residue at position 281 of SEQ ID NO: 852.

In another aspect, provided herein is an antibody that specifically binds human apelin receptor (APJ), the APJ comprising the amino acid sequence of SEQ ID NO: 852, wherein the antibody comprises a heavy chain variable domain (VH) comprising complementarity determining regions CDRH1, CDRH2, and CDRH3, and wherein: (a) the antibody comprises a tyrosine residue in the CDRH2 that specifically interacts with the tyrosine residue at position 21 of SEQ ID NO: 852; (b) the antibody comprises a serine residue in the CDRH3 that specifically interacts with the aspartate residue at position 172 of SEQ ID NO: 852; and/or (c) the antibody comprises a tyrosine residue in the CDRH3 that specifically interacts with the aspartate residue at position 184 of SEQ ID NO: 852.

In certain embodiments, the antibody comprises a tyrosine residue in the CDRH2 that specifically interacts with the tyrosine residue at position 21 of SEQ ID NO: 852; the antibody comprises a serine residue in CDRH3 that specifically interacts with the aspartate residue at position 172 of SEQ ID NO: 852; and the antibody comprises a tyrosine residue in CDRH3 that specifically interacts with the aspartate residue at position 184 of SEQ ID NO: 852.

In certain embodiments, the backbone N-H group of the tyrosine residue in the CDRH2 of the antibody specifically interacts with the hydroxyl group of the side chain of the tyrosine residue at position 21 of SEQ ID NO: 852.

In certain embodiments, the hydroxyl group of the side chain of the serine residue in CDRH3 of the antibody specifically interacts with the backbone carbonyl of the aspartate residue at position 172 of SEQ ID NO: 852.

In certain embodiments, the hydroxyl group of the side chain of the tyrosine residue in CDRH3 of the antibody specifically interacts with the carboxylate of the side chain of the aspartate residue at position 184 of SEQ ID NO: 852.

In certain embodiments, one or more of the specific interactions comprises a hydrogen bond.

In certain embodiments, the tyrosine residue in the CDRH2 is at a position corresponding to X₁₄ in SEQ ID NO: 248; the serine residue in CDRH3 is at a position corresponding to X₂₂ in SEQ ID NO: 249; and/or the tyrosine residue in CDRH3 is at a position corresponding to X₂₃ in SEQ ID NO: 249.

In certain embodiments, the tyrosine residue in the CDRH2 is at a position corresponding to X₁₄ in SEQ ID NO: 248; the serine residue in CDRH3 is at a position corresponding to X₂₂ in SEQ ID NO: 249; and the tyrosine residue in CDRH3 is at a position corresponding to X₂₃ in SEQ ID NO: 249.

In certain embodiments, the antibody is conjugated to a cytotoxic agent, cytostatic agent, toxin, radionuclide, or detectable label. In certain embodiments, the antibody is homodimeric.

In another aspect, provided herein is a polynucleotide encoding an antibody provided herein. In another aspect, provided herein is a vector comprising a polynucleotide provided herein. In another aspect, provided herein is a recombinant host cell comprising a polynucleotide or vector provided herein. In another aspect, provided herein is a composition comprising an antibody, polynucleotide, vector, or host cell provided herein and a pharmaceutically acceptable carrier or excipient.

In another aspect, provided herein is a method of producing an antibody, the method comprising culturing a recombinant host cell comprising a polynucleotide provided herein under suitable conditions such that the polynucleotide is expressed, and the antibody is produced.

In another aspect, provided herein is a method of treating an APJ-associated disease or disorder in a subject, the method comprising administering to the subject an effective amount of (a) an antibody that specifically binds human apelin receptor (APJ), (b) a polynucleotide encoding the antibody, (c) a vector comprising the polynucleotide, (d) a recombinant host cell comprising the polynucleotide or the vector, or (e) a composition comprising any of (a)-(d) and a pharmaceutically acceptable carrier or excipient, wherein the APJ-associated disease or disorder is selected from the group consisting of hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD).

In certain embodiments, the APJ-associated disease or disorder is HHT (e.g., HHT1, HHT2, HHT3, HHT4, HHT5, or JP-HHT). In certain embodiments, the APJ-associated disease or disorder is selected from the group consisting of angiodysplasia, arteriovenous malformation (AVM), brain AVM, bleeding, and telangiectasia, and wherein the subject has been diagnosed with HHT (e.g., HHT1, HHT2, HHT3, HHT4, HHT5, or JP-HHT). In certain embodiments, the APJ-associated disease or disorder is telangiectasia, and wherein the subject has been diagnosed with pulmonary hypertension. In certain embodiments, the subject has been treated with and/or is being treated with sotatercept. In certain embodiments, the APJ-associated disease or disorder is selected from the group consisting of heart failure, acute decompensated heart failure, and congestive heart failure, and wherein the subject has been treated with and/or is being treated with a left ventricular assist device (LVAD), optionally wherein the LVAD is a continuous-flow LVAD.

In certain embodiments, the APJ-associated disease or disorder is a vascular eye disease or disorder selected from the group consisting of diabetic retinopathy, proliferative diabetic retinopathy, diabetic macular edema, macular degeneration, age-related macular degeneration, wet age-related macular degeneration, geographic atrophy, retinal neovascularization, central retinal vein occlusion, branched retinal vein occlusion, polypoidal choroidal vasculopathy, choroidal neovascularization (CNV), degenerative myopia (myopic CNV), neovascular glaucoma, and retinopathy of prematurity.

In certain embodiments, the APJ-associated disease or disorder is stroke, and administration of the antibody, polynucleotide, vector, host cell, or composition prevents or delays the stroke.

In certain embodiments, the APJ-associated disease or disorder is idiopathic PAH, heritable PAH, toxin- or drug-induced PAH, or PAH associated with one or more of congenital heart disease, a connective tissue disorder, portal hypertension, a BMPR2 mutation, and Schistosomiasis. In certain embodiments, the connective tissue disorder is selected from the group consisting of scleroderma, systemic lupus erythematosus, systemic sclerosis, Hashimoto's thyroiditis, Sjögren's Syndrome, and antiphospholipid antibody syndrome. In certain embodiments, the APJ-associated disease or disorder is fibrosis associated with an organ or tissue selected from the group consisting of lung, liver, heart, mediastinum, bone marrow, retroperitoneum, skin, intestine, joint, a reproductive organ, and a combination thereof. In certain embodiments, the APJ-associated disease or disorder is a connective tissue disorder selected from the group consisting of scleroderma, systemic lupus erythematosus, systemic sclerosis, Hashimoto's thyroiditis, Sjögren's Syndrome, and antiphospholipid antibody syndrome.

In certain embodiments, the antibody is an APJ antagonist antibody. In certain embodiments, the antibody is an APJ agonist antibody.

In another aspect, provided herein is a method of treating an APJ-associated disease or disorder in a subject, the method comprising administering to the subject an effective amount of (a) an antibody that specifically binds human apelin receptor (APJ), (b) a polynucleotide encoding the antibody, (c) a vector comprising the polynucleotide, (d) a recombinant host cell comprising the polynucleotide or the vector, or (e) a composition comprising any of (a)-(d) and a pharmaceutically acceptable carrier or excipient, wherein the antibody is an APJ antagonist antibody and wherein the APJ-associated disease or disorder is selected from the group consisting of hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD).

In another aspect, provided herein is a method of treating an APJ-associated disease or disorder in a subject, the method comprising administering to the subject an effective amount of (a) an antibody that specifically binds human apelin receptor (APJ), (b) a polynucleotide encoding the antibody, (c) a vector comprising the polynucleotide, (d) a recombinant host cell comprising the polynucleotide or the vector, or (e) a composition comprising any of (a)-(d) and a pharmaceutically acceptable carrier or excipient, wherein the antibody is an APJ agonist antibody and wherein the APJ-associated disease or disorder is selected from the group consisting of obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD).

In certain embodiments of the methods of treating an APJ-associated disease or disorder in a subject provided herein, the methods comprise administering to the subject an effective amount of an antibody, polynucleotide, vector, host cell, or composition provided herein. In certain embodiments, the APJ-associated disease or disorder is HHT (e.g., HHT1, HHT2, HHT3, HHT4, HHT5, or JP-HHT).

In another aspect, provided herein is a polypeptide comprising any one of the amino acid sequences set forth in SEQ ID NOs: 704-750. In certain embodiments, the polypeptide comprises an IgG Fc comprising a modified hinge region comprising any one of the amino acid sequences set forth in SEQ ID NOs: 704-718. In certain embodiments, the IgG Fc comprises any one of the amino acid sequences set forth in SEQ ID NOs: 253-268 and 450-463 comprising the modified hinge region.

In another aspect, provided herein is a use of an antibody that specifically binds to human APJ, a polynucleotide encoding the antibody, a vector comprising the polynucleotide, a recombinant host cell comprising the polynucleotide or the vector, and/or a composition comprising the antibody, polynucleotide, vector, and/or recombinant host cell and a pharmaceutically acceptable carrier or excipient in the manufacture of a medicament for the treatment of an APJ-associated disease or disorder in a subject in need thereof. In certain aspects, provided herein is a use of an anti-APJ antibody, polynucleotide, vector, host cell, or composition described herein in the manufacture of a medicament for the treatment of an APJ-associated disease or disorder in a subject in need thereof. In certain embodiments, the APJ-associated disease or disorder is hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD).

In another aspect, provided herein is an antibody that specifically binds to human APJ, a polynucleotide encoding the antibody, a vector comprising the polynucleotide, a recombinant host cell comprising the polynucleotide or the vector, and/or a composition comprising the antibody, polynucleotide, vector, and/or recombinant host cell and a pharmaceutically acceptable carrier or excipient for use in a method of treatment of an APJ-associated disease or disorder in a subject in need thereof. In certain aspects, provided herein is an anti-APJ antibody, polynucleotide, vector, host cell, or composition described herein for use in a method of treatment of an APJ-associated disease or disorder in a subject in need thereof. In certain embodiments, the APJ-associated disease or disorder is hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD).

### BRIEF DESCRIPTION OF THE DRAWINGS

FIGS. 1A-1J are graphs of APJ antagonist in vitro activity measured via β-arrestin recruitment in the presence of apelin and increasing amounts of anti-APJ antibodies described in Example 4. Decreases in percent activity represent blocking of ligand-mediated β-arrestin recruitment.

FIGS. 2A-2J are graphs of APJ agonist activity measured via β-arrestin recruitment in the presence of increasing amounts of anti-APJ antibodies described in Example 4. Increases in percent activity represent β-arrestin recruitment stimulated by the anti-APJ antibodies.

FIG. 3 is a Cryo-EM map and ribbon diagram model of Ab076 VHH bound to human APJ modified as described in Example 1, according to certain aspects of the present disclosure. The left panel is a Cryo-EM map of the Ab076 VHH/APJ complex at 3.9 Å overall resolution, with APJ in white and Ab076 VHH in grey. The right panel is a ribbon diagram model depicting APJ in white and Ab076 VHH in grey.

FIG. 4 is a pair of ribbon diagram models showing particular residues in the structures of JN241 (white/light grey, with ECL2 of APJ shown in black) (top panel) and Ab076 VHH (darker grey, with ECL2 of APJ shown in white) (bottom panel) bound to APJ.

FIG. 5 is a pair of ribbon diagram models showing particular residues in the interface between APJ (black) and JN241 (white/light grey) (top panel) or the interface between APJ (white/light grey) and Ab076 VHH (darker grey) (bottom panel).

FIG. 6A is a ribbon diagram model showing an overall superposition of bound APJ models depicting the conformation of APJ N-terminus (left) or other regions of APJ (right) when bound to JN241 (black) or to Ab076 VHH (white). The movement of APJ N-terminal region residue Y21 between models is illustrated by a dotted arrow. FIG. 6B is a ribbon diagram model showing particular residues in the interface between APJ (black) and JN241 (white/light grey).

FIG. 6C is a ribbon diagram model showing particular residues in the interface between APJ (white) and Ab076 VHH (darker grey).

FIG. 7 are two ribbon diagram models showing particular residues in the interface between APJ (black) and JN241 (white/light grey) (top panel) or APJ (white/light grey) and Ab076 VHH (darker grey) (bottom panel).

FIG. 8 is a graph of the fold change in APLN mRNA expression level in P6 neonatal mice treated with anti-BMP9/10 antibodies (“anti-BMP9/10”) relative to mice treated with vehicle control (“PBS”). Data represent mean±SEM (n=14 and 15 pups for PBS and anti-BMP9/10 groups, respectively). ****: p<0.0001, unpaired student t-test.

FIGS. 9A and 9B are plots of retinal plexus area vascular density (FIG. 9A) and retinal arteriovenous malformation (“AVM”) number (FIG. 9B) in P6 neonatal mice treated with IgG2a/2b antibodies (“isotype control”) and PBS as a negative control or with anti-BMP9/10 antibodies (“+anti-BMP9/10”) and negative control AbNC, test molecule Ab108, or G6.31 (anti-VEGFA neutralizing antibody positive control). Data represent mean±SEM (n=7 pups per group, vascular densities of 4 different fields per retina were measured). ****: p<0.0001; ***: p<0.001; ns: not significant, one-way ANOVA with Tukey's multiple comparison post hoc test.

FIG. 10 is a plot of serum Fc concentration in P9 neonatal mice treated with anti-BMP 9/10 and either test molecule Ab108 or AbNC. Data represent mean±SEM (n=14 and 18 pups for anti-BMP9/10 and Ab108 and anti-BMP9/10 and Ab108 groups, respectively).

FIG. 11 is a plot of retinal AVM numbers in P9 neonatal mice treated with isotype control and PBS or with anti-BMP9/10 and either AbNC or test molecule Ab108. Data represent mean±SEM (n=9, 15, and 9 pups for isotype control and PBS, anti-BMP9/10 and AbNC, and anti-BMP9/10 and Ab108 groups, respectively). *: p<0.05, ***: p<0.001, ****: p<0.0001, one-way ANOVA with Tukey's multiple comparison post hoc test.

FIG. 12 is a plot of percent retinal bleeding area in P9 neonatal mice treated with isotype control and PBS or with anti-BMP9/10 and either AbNC or test molecule Ab108. Data represent mean±SEM (n=9, 18, and 14 pups for isotype control and PBS, anti-BMP9/10 and AbNC, and anti-BMP9/10 and Ab108 groups, respectively). **: p<0.01, ****: p<0.0001, ns: not significant, one-way ANOVA with Tukey's multiple comparison post hoc test.

FIG. 13 is a plot of retinal vasculature radial length in P9 neonatal mice treated with isotype control and PBS or with anti-BMP9/10 and either AbNC or test molecule Ab108. Data represent mean±SEM (n=9, 18, and 14 pups for isotype control and PBS, anti-BMP9/10 and AbNC, and anti-BMP9/10 and Ab108 groups, respectively). ***: p<0.001, ****: p<0.0001, one-way ANOVA with Tukey's multiple comparison post hoc test.

FIG. 14 is a plot of hemoglobin levels in P9 neonatal mice treated with isotype control and PBS or with anti-BMP9/10 and either AbNC or test molecule Ab108. Data represent mean±SEM (n=9, 18, and 14 pups for isotype control and PBS, anti-BMP9/10 and AbNC, and anti-BMP9/10 and Ab108 groups, respectively, average of 2-3 measurements per animal). ****: P<0.0001, ns: no significance, one-way ANOVA with Tukey's multiple comparison post hoc test.

FIGS. 15A and 15B are plots of retinal vasculature radial length (FIG. 15A) and percent retinal vascularized area (FIG. 15B) in P6 neonatal mice treated with AbNC, test molecule Ab108, or sirolimus positive control. Data represent mean±SEM (n=8, 6, and 3 pups (FIG. 15A) or 7, 6, and 3 pups (FIG. 15B) for AbNC, Ab108, and sirolimus groups, respectively). ****: p<0.0001, one-way ANOVA with Tukey's multiple comparison post hoc test.

FIG. 16A is a dose-response curve showing in vitro potency of Ab108. cAMP levels were measured using a highly sensitive HTRF-based competitive immunoassay in the presence of apelin and increasing amounts of Ab108. FIG. 16B is a plot of retinal vasculature radial length in P6 neonatal mice treated with varying concentrations of Ab108. Data are shown as a percentage change in radial length vs. baseline as a function of Ab108 plasma concentration (determined via anti-human Fc ELISA). Data represent mean±s.e.m. (n=7-13 pups for each group).

FIGS. 17A and 17B are plots showing hemoglobin (Hb) levels in no cre control mice treated with PBS and ALK1 iKO mice treated with isotype control AbNC, Ab108, and Anti-VEGFA G6.31. FIG. 17A shows hemoglobin level change of each group at Day 0, 7, 9, 11, and 12. FIG. 17B shows hemoglobin level at Day 12. Each data point represents one mouse. The black dotted line indicates a hemoglobin level of 10 g/dL or lower, which is generally considered indicative of anemia. Data represent mean±s.e.m. (n=15-17); 2-3 measurements were averaged for one animal. * p<0.05, ** p<0.01, **** p<0.0001, ns, no significance, one-way ANOVA with Tukey's multiple comparison.

FIG. 18 is a plot showing the GI index of no cre control mice treated with PBS and ALK1 iKO mice treated with isotype control AbNC, Ab108, or Anti-VEGFA G6.31. Data represent mean±s.e.m. (n=15-17). **** p<0.0001, ns, no significance, one-way ANOVA with Tukey's multiple comparison.

FIGS. 19A-19D are representative photographic images showing latex blue perfused blood vessels in small intestine near Peyer's patch from no Cre control mice treated with PBS (FIG. 19A) and ALK1 iKO mice treated with isotype control AbNC (FIG. 19B), Ab108 (FIG. 19C), or Anti-VEGFA G6.31 (FIG. 19D). Arteries (a) and veins (v) are indicated. GI hemorrhage areas are shown in dashed line boxes. Scale bar: 0.5 mm.

FIGS. 20A and 20B are plots showing vascular analysis results in ALK1 iKO mice treated with isotype control AbNC, Ab108, and Anti-VEGFA G6.31. FIG. 20A shows vascular density. n=15-17 mice. FIG. 20B shows vein diameter. n=15-17 mice, 2-3 measurements per mouse. For FIGS. 20A and 20B, data represent mean±s.e.m., ** p<0.01, **** p<0.0001, ns, no significance, one-way ANOVA with Tukey's multiple comparison.

### DETAILED DESCRIPTION

The instant disclosure provides anti-APJ antibodies and polypeptides. Also provided are pharmaceutical compositions comprising these antibodies, nucleic acids encoding these antibodies, expression vectors and host cells for making these antibodies, and methods of treating a subject using these antibodies. The antibodies provided herein appear to be particularly advantageous because they modulate APJ activity with more potency, and display lower polyreactivity and higher stability, relative to prior art antibodies tested.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The section headings used herein are for organizational purposes only and are not to be construed as limiting the subject matter described.

As used herein, the term “APJ” refers to the apelin receptor, also known as APLNR. As used herein, the terms “human APJ” and “hAPJ” are used interchangeably and refer to a protein encoded by a wild-type human APJ gene (e.g., the human APJ gene set forth in RefSeq NM_005161.6). The amino acid sequence of an exemplary human APJ protein is set forth in RefSeq NP_005152.1 and below:

(SEQ ID NO: 852)
MEEGGDFDNYYGADNQSECEYTDWKSSGALIPAIYMLVFLLGTTGNGL
VLWTVFRSSREKRRSADIFIASLAVADLTFVVTLPLWATYTYRDYDWP
FGTFFCKLSSYLIFVNMYASVFCLTGLSFDRYLAIVRPVANARLRLRV
SGAVATAVLWVLAALLAMPVMVLRTTGDLENTTKVQCYMDYSMVATVS
SEWAWEVGLGVSSTTVGFVVPFTIMLTCYFFIAQTIAGHFRKERIEGL
RKRRRLLSIIVVLVVTFALCWMPYHLVKTLYMLGSLLHWPCDFDLFLM
NIFPYCTCISYVNSCLNPFLYAFFDPRFRQACTSMLCCGQSRCAGTSH
SSSGEKSASYSSGHSQGPGPNMGKGGEQMHEKSIPYSQETLVVD.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, bispecific T cell engagers (BiTEs), chimeric antigen receptors, single domain antibodies, monovalent antibodies, single-chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, antibodies described herein are IgG antibodies, or a class (e.g., human IgG₁ or IgG₄) or subclass thereof.

As used herein, the term “EU numbering system” refers to the EU numbering convention for the constant regions of an antibody, as described in Edelman, G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. Descriptions herein of an amino acid residue at a particular “EU position” of an antibody heavy chain constant region are with reference to the EU numbering system.

“Multispecific antibodies” are antibodies (e.g., bispecific antibodies) that specifically bind to two or more different antigens or two or more different regions of the same antigen. Multispecific antibodies include bispecific antibodies that contain two different antigen-binding sites (exclusive of the Fc region). Multispecific antibodies can include, for example, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, heteroconjugate antibodies, linked single-chain antibodies or linked-single-chain Fvs (scFv), camelized antibodies, affybodies, linked Fab fragments, F(ab′)₂ fragments, chemically-linked Fvs, and disulfide-linked Fvs (sdFv). Multispecific antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG2a or IgG2b) of immunoglobulin molecule. In certain embodiments, multispecific antibodies described herein are IgG antibodies, or a class (e.g., human IgG₁, IgG₂, or IgG₄) or subclass thereof.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest. (1991), by Chothia et al., J. Mol. Biol. 196: 901-917 (1987), and by MacCallum et al., J. Mol. Biol. 262: 732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human APJ). CDRH1, CDRH2, and CDRH3 denote the heavy chain CDRs, and CDRL1, CDRL2, and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FRs). Without wishing to be bound by any particular mechanism or theory, it is believed that the CDRs of the light and heavy chains are primarily responsible for the interaction and specificity of the antibody with antigen. Exemplary antibody variable regions are described in Kabat et al., (1991) Sequences of Proteins of Immunological Interest (NIH Publication No. 91-3242, Bethesda), which is herein incorporated by reference in its entirety. Further exemplary antibody variable regions include: a variable heavy domain of heavy chain (VHH); and engineered antibody variable regions that comprise one or more camelid CDRs (or engineered variants thereof) grafted into non-camelid framework regions (e.g., human framework regions, or engineered variants thereof). As described herein, the CDR of an antibody can be defined using a variety of numbering systems. Accordingly, as one of skill in the art will appreciate, an amino acid residue specified as “absent” at the N or C terminus of a CDR sequence disclosed herein may be present in the adjacent framework region of the variable region containing that CDR sequence.

As used herein, the term “VH” refers to an antibody heavy chain variable region and includes, without limitation, a variable heavy domain of heavy chain (VHH) and engineered antibody variable regions that comprise one or more camelid CDRs (or engineered variants thereof) grafted into non-camelid framework regions (e.g., human framework regions, or engineered variants thereof).

As used herein, the term “VL” refers to an antibody light chain variable region.

As used herein, the term “constant region” is common in the art. The constant region is an antibody portion, e.g., a carboxyl terminal portion of a light and/or heavy chain, which is not directly involved in binding of an antibody to antigen but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In specific embodiments, the light chain is a human light chain.

As used herein, the term “Ig Fc” refers to the portion of an immunoglobulin heavy chain polypeptide that can dimerize to form an Fc region. As used herein, the term “IgG Fc” refers to an Ig Fc of an IgG subclass (e.g., human IgG). As used herein, the term “IgG1 Fc” refers to an Ig Fc of an IgG1 subclass (e.g., human IgG1). Ig Fcs can comprise wild-type immunoglobulin heavy chain polypeptide sequences or engineered variants of wild-type immunoglobulin heavy chain polypeptide sequences.

As used herein, the term “specifically binds” refers to the specificity of a binding molecule (e.g., an antibody) for an antigen, as is understood by one skilled in the art. Binding molecules that specifically bind to an antigen typically bind to the antigen with an equilibrium dissociation constant (KD) of less than 1×10⁻⁶ M, as measured by, e.g., ELISA assay, surface plasmon resonance, or other suitable assays known in the art. The skilled worker will appreciate that, in certain embodiments, a binding molecule can specifically bind to different antigens, e.g., different antigens that share a common epitope that is recognized by the binding molecule.

As used herein, the term “specifically interacts” refers to the formation of one or more non-covalent interactions (including, but not limited to, hydrogen bonds, ionic interactions, van der Waals forces, hydrophobic interactions, π-πstacking, π-cation stacking, and π-anion stacking) between one or more atoms or molecular groups of an antibody (e.g., an antibody disclosed herein) and a target antigen (e.g., human APJ), which contribute to specific and reversible binding between the antibody and the target antigen.

As used herein, the term “linked to” refers to covalent or noncovalent binding between two molecules or moieties. The skilled worker will appreciate that when a first molecule or moiety is linked to a second molecule or moiety, the linkage need not be direct, but instead, can be via an intervening molecule or moiety.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

As used herein, the terms “treat,” “treating,” and “treatment” refer to therapeutic or preventative measures described herein. In certain embodiments, the methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “pharmaceutically active substance” refers to a molecule or moiety that is used to achieve a beneficial outcome in a subject. Beneficial outcomes include, but are not limited to, diagnosis, prognosis, treatment, cure, and prevention (prophylaxis) of diseases and/or symptoms and/or health problems.

As used herein, the term “effective amount” in the context of the administration of a therapy to a subject refers to the amount of a therapy that achieves a desired prophylactic or therapeutic effect.

As used herein, the term “subject” includes any human or non-human animal. In one embodiment, the subject is a human or non-human mammal. In one embodiment, the subject is a human.

The term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “protein” and “polypeptide” are used interchangeably herein and refer to a polymer of amino acids connected by one or more peptide bonds. As used herein, “amino acid sequence” refers to the information describing the relative order and identity of amino acid residues which make up a polypeptide.

As used herein, the term “an amino acid sequence that has 0, 1, 2, 3, 4, or 5 amino acid modifications” with reference to an amino acid sequence, refers to an amino acid sequence that comprises up to 5 amino acid substitutions, alterations, inversions, additions, or deletions compared to a reference amino acid sequence.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87: 2264-2268, modified as in Karlin S & Altschul S F, (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein with respect to an antibody, polypeptide, or polynucleotide, the term “isolated” refers to an antibody, polypeptide, or polynucleotide that is separated from one or more contaminants (e.g., polypeptides, polynucleotides, lipids, or carbohydrates, etc.) which are present in a natural source (e.g., in a mouse or a human) of the antibody, polypeptide, or polynucleotide. Moreover, an “isolated” antibody, polypeptide, or polynucleotide can be substantially free of other cellular material, or culture medium when produced by recombinant techniques, or substantially free of chemical precursors or other chemicals when chemically synthesized. For example, the language “substantially free” includes preparations of antibody, polypeptide, or polynucleotide having less than about 15%, 10%, 5%, 2%, 1%, 0.5%, or 0.1% (in particular less than about 10%) of other material, e.g., cellular material, culture medium, other nucleic acid molecules, chemical precursors, and/or other chemicals. All instances of “isolated antibodies” described herein are additionally contemplated as antibodies that may be, but need not be, isolated. All instances of “isolated polypeptides” described herein are additionally contemplated as polypeptides that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polypeptides” described herein are additionally contemplated as polypeptides that may be, but need not be, isolated. All instances of “polynucleotides” described herein are additionally contemplated as polynucleotides that may be, but need not be, isolated.

### Anti-APJ Antibodies

In one aspect, the instant disclosure provides antibodies that specifically bind to APJ (e.g., human APJ). The amino acid sequences of exemplary antibodies provided herein are set forth in Table 1.

TABLE 1
Amino acid sequences of exemplary anti-APJ antibodies.

| | | SEQ ID |
|---|---|---|
| Ab | Amino acid sequence | NO |
| Ab001 | EVQLVESGGGLVQPGGSLRLSCAASGSTYSSHCMGWYRQAPGKGREFVALMTRSRG | 1 |
| | TSYADSVKGRFTISRDNSKNTVYLQMNSLRAEDTAVYYCAAVPRAGIESGAYCKWN | |
| | MKDSGSWGQGTQVTVSS | |
| Ab002 | EVQLVESGGGLVQPGGSLRLSCAASGSTYSSHCMGWYRQAPGKGREFVALMTRSRG | 2 |
| | TSYADSVKGRFTISRDNSKNTVYLQMNSLRAEDTAVYYCAAVPRAGIESGAYCKWI | |
| | IKDSGSWGQGTQVTVSS | |
| Ab003 | EVQLVESGGGLVQPGGSLRLSCAASGSTYSSHCMGWYRQAPGKGREFVALMTHSRG | 3 |
| | TSYADSVKGRFTISRDNSKNTVYLQMNSLRAEDTAVYYCAAVPRAGIESGAYCKWR | |
| | YKDSGSWGQGTQVTVSS | |
| Ab004 | EVQLVESGGGLVQPGGSLRLSCAASGSTYSSHCMGWYRQAPGKGREFVALMTHSRG | 4 |
| | TSYADSVKGRFTISRDNSKNTVYLQMNSLRAEDTAVYYCAAVPRAGIESGAYCKWR | |
| | YQDSGSWGQGTQVTVSS | |
| Ab005 | EVQLVESGGGLVQPGGSLRLSCAASGSTYSSHCMGWYRQAPGKGREFVALMTRSRG | 5 |
| | TSYADSVKGRFTISRDNSKNTVYLQMNSLRAEDTAVYYCAAVPRAGIESGAYCKWR | |
| | YQDSGSWGQGTQVTVSS | |
| Ab006 | EVQLVESGGGLVQPGGSLRLSCAASGSTYSSHCMGWYRQAPGKGREFVALMTRSRG | 6 |
| | TSYADSVKGRFTISRDNSKNTVYLQMNSLRAEDTAVYYCAAVPRAGIESGAYCKWI | |
| | IQDSGSWGQGTQVTVSS | |
| Ab007 | EVQLVESGGGLVQPGGSLRLSCAASGSTYSSHCMGWYRQAPGKGREFVALMTHSRG | 7 |
| | TSYADSVKGRFTISRDNSKNTVYLQMNSLRAEDTAVYYCAAVPRAGIESGAYCKWI | |
| | IQDSGSWGQGTQVTVSS | |
| Ab008 | EVQLVESGGGLVQPGGSLRLSCAASGSTYSSHCMGWYRQAPGKGREFVALMTRSRG | 8 |
| | TSYADSVKGRFTISRDNSKNTVYLQMNSLRAEDTAVYYCAAVPRAGIESGAYCKWP | |
| | RKDSGSWGQGTQVTVSS | |
| Ab009 | EVQLVESGGGLVQPGGSLRLSCAASGSTYSSHCMGWYRQAPGKGREFVALMTRSYG | 9 |
| | TSYADSVKGRFTISRDNSKNTVYLQMNSLRAEDTAVYYCAAVPRAGIESGAYCKWP | |
| | RKDSGSWGQGTQVTVSS | |
| Ab010 | EVQLVESGGGLVQPGGSLRLSCAASGSTYSSHCMGWYRQAPGKGREFVALMTRSYG | 10 |
| | TSYADSVKGRFTISRDNSKNTVYLQMNSLRAEDTAVYYCAAVPRAGIESGAYCKWP | |
| | RQDSGSWGQGTQVTVSS | |
| Ab011 | EVQLVESGGGLVQPGGSLRLSCAASGSTYSSHCMGWYRQAPGKGREFVALMTRSRG | 11 |
| | TSYADSVKGRFTISRDNSKNTVYLQMNSLRAEDTAVYYCAAVPRAGIESGAYCKWP | |
| | RQDSGSWGQGTQVTVSS | |
| Ab012 | EVQLVESGGGLVQPGGSLRLSCAASGSTYSSHCMGWYRQAPGKGREFVALMTHSRG | 12 |
| | TSYADSVKGRFTISRDNSKNTVYLQMNSLRAEDTAVYYCAAVPRAGIESGAYCKWP | |
| | RQDSGSWGQGTQVTVSS | |
| Ab013 | EVQLVESGGGLVQPGGSLRLSCAASGSTYSSHCMGWYRQAPGKGREFVALMTRSRG | 13 |
| | TSYADSVKGRFTISRDNSKNTVYLQMNSLRAEDTAVYYCAAVPRAGIESGAYCKWR | |
| | LQDSGSWGQGTQVTVSS | |
| Ab014 | EVQLVESGGGLVQPGGSLRLSCAASGSTYSSHCMGWYRQAPGKGREFVALMTHSRG | 14 |
| | TSYADSVKGRFTISRDNSKNTVYLQMNSLRAEDTAVYYCAAVPRAGIESGAYCKWR | |
| | LQDSGSWGQGTQVTVSS | |
| Ab015 | EVQLVESGGGLVQPGGSLRLSCAASGSTYSSHCMGWYRQAPGKGREFVALMTHSRG | 15 |
| | TSYADSVKGRFTISRDNSKNTVYLQMNSLRAEDTAVYYCAAVPRAGIESGAYCKWP | |
| | HKDSGSWGQGTQVTVSS | |
| Ab016 | EVQLVESGGGLVQPGGSLRLSCAASGSTYSSHCMGWYRQAPGKGREFVALMTHSRG | 16 |
| | TSYADSVKGRFTISRDNSKNTVYLQMNSLRAEDTAVYYCAAVPRAGIESGAYCKWP | |
| | HQDSGSWGQGTQVTVSS | |
| Ab017 | EVQLVESGGGLVQPGGSLRLSCAASGSTYSSHCMGWYRQAPGKGREFVALMTRSRG | 17 |
| | TSYADSVKGRFTISRDNSKNTVYLQMNSLRAEDTAVYYCAAVPRAGIESGAYCKWP | |
| | HQDSGSWGQGTQVTVSS | |
| Ab018 | EVQLVESGGGLVQPGGSLRLSCAASGSTYSSHCMGWYRQAPGKGREFVALMTHSRG | 18 |
| | TSYADSVKGRFTISRDNSKNTVYLQMNSLRAEDTAVYYCAAVPRAGIESGAYCKWH | |
| | GKDSGSWGQGTQVTVSS | |
| Ab019 | EVQLVESGGGLVQPGGSLRLSCAASGSTYSSHCMGWYRQAPGKGREFVALMTRSRG | 19 |
| | TSYADSVKGRFTISRDNSKNTVYLQMNSLRAEDTAVYYCAAVPRAGIESGAYCKWH | |
| | GKDSGSWGQGTQVTVSS | |
| Ab020 | EVQLVESGGGLVQPGGSLRLSCAASGSTYSSHCMGWYRQAPGKGREFVALMTHSRG | 20 |
| | TSYADSVKGRFTISRDNSKNTVYLQMNSLRAEDTAVYYCAAVPRAGIESGAYCKWH | |
| | NKDSGSWGQGTQVTVSS | |
| Ab021 | EVQLVESGGGLVQPGGSLRLSCAASGSTYSSHCMGWYRQAPGKGREFVALMTHSRG | 21 |
| | TSYADSVKGRFTISRDNSKNTVYLQMNSLRAEDTAVYYCAAVPRAGIESGAYCKWH | |
| | YKDSGSWGQGTQVTVSS | |
| Ab022 | EVQLVESGGGLVQPGGSLRLSCAASGSTYSSHCMGWYRQAPGKGREFVALMTHSRG | 22 |
| | TSYADSVKGRFTISRDNSKNTVYLQMNSLRAEDTAVYYCAAVPRAGIESGAYCKWK | |
| | NKDSGSWGQGTQVTVSS | |
| Ab023 | EVQLVESGGGLVQPGGSLRLSCAASGSTYSSHCMGWYRQAPGKGREFVALMTHSRG | 23 |
| | TSYADSVKGRFTISRDNSKNTVYLQMNSLRAEDTAVYYCAAVPRAGIESGAYCKWK | |
| | YKDSGSWGQGTQVTVSS | |
| Ab024 | EVQLVESGGGLVQPGGSLRLSCAASGSTYSSHCMGWYRQAPGKGREFVALMTHSRG | 24 |
| | TSYADSVKGRFTISRDNSKNTVYLQMNSLRAEDTAVYYCAAVPRAGIESGAYCKWN | |
| | NKDSGSWGQGTQVTVSS | |
| Ab025 | EVQLVESGGGLVQPGGSLRLSCAASGSTYSSHCMGWYRQAPGKGREFVALMTHSRG | 25 |
| | TSYADSVKGRFTISRDNSKNTVYLQMNSLRAEDTAVYYCAAVPRAGIESGAYCKWN | |
| | YKDSGSWGQGTQVTVSS | |
| Ab026 | EVQLVESGGGLVQPGGSLRLSCAASGSTYSSHCMGWYRQAPGKGREFVALMTHSRG | 26 |
| | TSYADSVKGRFTISRDNSKNTVYLQMNSLRAEDTAVYYCAAVPRAGIESGAYCKWQ | |
| | NKDSGSWGQGTQVTVSS | |
| Ab027 | EVQLVESGGGLVQPGGSLRLSCAASGSTYSSHCMGWYRQAPGKGREFVALMTHSRG | 27 |
| | TSYADSVKGRFTISRDNSKNTVYLQMNSLRAEDTAVYYCAAVPRAGIESGAYCKWQ | |
| | YKDSGSWGQGTQVTVSS | |
| Ab028 | EVQLVESGGGLVQPGGSLRLSCAASGSTYSSHCMGWYRQAPGKGREFVALMTHSRG | 28 |
| | TSYADSVKGRFTISRDNSKNTVYLQMNSLRAEDTAVYYCAAVPRAGIESGAYCKWR | |
| | NKDSGSWGQGTQVTVSS | |
| Ab029 | EVQLVESGGGLVQPGGSLRLSCAASGSTYSSHCMGWYRQAPGKGREFVALMTHSRG | 29 |
| | TSYADSVKGRFTISRDNSKNTVYLQMNSLRAEDTAVYYCAAVPRAGIESGAYCKWS | |
| | NKDSGSWGQGTQVTVSS | |
| Ab030 | EVQLVESGGGLVQPGGSLRLSCAASGSTYSSHCMGWYRQAPGKGREFVALMTHSRG | 30 |
| | TSYADSVKGRFTISRDNSKNTVYLQMNSLRAEDTAVYYCAAVPRAGIESGAYCKWS | |
| | YKDSGSWGQGTQVTVSS | |
| Ab031 | EVQLVESGGGLVQPGGSLRLSCAASGSTYSSHCMGWYRQAPGKGREFVALMTRSRG | 31 |
| | TSYADSVKGRFTISRDNSKNTVYLQMNSLRAEDTAVYYCAAVPRAGIESGAYCKWI | |
| | HKDSGSWGQGTQVTVSS | |
| Ab032 | EVQLVESGGGLVQPGGSLRLSCAASGSTYSSHCMGWYRQAPGKGREFVALMTRSRG | 32 |
| | TSYADSVKGRFTISRDNSKNTVYLQMNSLRAEDTAVYYCAAVPRAGIESGAYCKWI | |
| | LKDSGSWGQGTQVTVSS | |
| Ab033 | EVQLVESGGGLVQPGGSLRLSCAASGSTYSSHCMGWYRQAPGKGREFVALMTRSRG | 33 |
| | TSYADSVKGRFTISRDNSKNTVYLQMNSLRAEDTAVYYCAAVPRAGIESGAYCKWI | |
| | NKDSGSWGQGTQVTVSS | |
| Ab034 | EVQLVESGGGLVQPGGSLRLSCAASGSTYSSHCMGWYRQAPGKGREFVALMTRSRG | 34 |
| | TSYADSVKGRFTISRDNSKNTVYLQMNSLRAEDTAVYYCAAVPRAGIESGAYCKWT | |
| | HKDSGSWGQGTQVTVSS | |
| Ab035 | EVQLVESGGGLVQPGGSLRLSCAASGSTYSSHCMGWYRQAPGKGREFVALMTRSRG | 35 |
| | TSYADSVKGRFTISRDNSKNTVYLQMNSLRAEDTAVYYCAAVPRAGIESGAYCKWT | |
| | IKDSGSWGQGTQVTVSS | |
| Ab036 | EVQLVESGGGLVQPGGSLRLSCAASGSTYSSHCMGWYRQAPGKGREFVALMTRSRG | 36 |
| | TSYADSVKGRFTISRDNSKNTVYLQMNSLRAEDTAVYYCAAVPRAGIESGAYCKWT | |
| | LKDSGSWGQGTQVTVSS | |
| Ab037 | EVQLVESGGGLVQPGGSLRLSCAASGSTYSSHCMGWYRQAPGKGREFVALMLRSRG | 37 |
| | TSYADSVKGRFTISRDNSKNTVYLQMNSLRAEDTAVYYCAAVPRAGIESGAYCKWT | |
| | NKDSGSWGQGTQVTVSS | |
| Ab038 | EVQLVESGGGLVQPGGSLRLSCAASGYNYVFHCMGWYRQAPGKGREFVALMSRSRG | 38 |
| | YYYADSVKGRFTISRDNSKNTVYLQMNSLRAEDTAVYYCAAVPRAGIESGAYCKWH | |
| | GKDSGSWGQGTQVTVSS | |
| Ab039 | EVQLVESGGGLVQPGGSLRLSCAASGITYVSHCMGWYRQAPGKGREFVAAMSRSRG | 39 |
| | YYYADSVKGRFTISRDNSKNTVYLQMNSLRAEDTAVYYCAAVPRAGIESGAYCKWH | |
| | GKDSGSWGQGTQVTVSS | |
| Ab040 | EVQLVESGGGLVQPGGSLRLSCAASGITYSSHCMGWYRQAPGKGREFVAAMQRSRG | 40 |
| | TYYADSVKGRFTISRDNSKNTVYLQMNSLRAEDTAVYYCAAVPRAGIESGAYCKWH | |
| | GKDSGSWGQGTQVTVSS | |
| Ab041 | EVQLVESGGGLVQPGGSLRLSCAASGITYQSHCMGWYRQAPGKGREFVALIQRSRG | 41 |
| | TYYADSVKGRFTISRDNSKNTVYLQMNSLRAEDTAVYYCAAVPRAGIESGAYCKWH | |
| | GKDSGSWGQGTQVTVSS | |
| Ab042 | EVQLVESGGGLVQPGGSLRLSCAASGLHYHSHCMGWYRQAPGKGREFVAAMSHSRG | 42 |
| | YYYADSVKGRFTISRDNSKNTVYLQMNSLRAEDTAVYYCAAVPRAGIESGAYCKWQ | |
| | NKDSGSWGQGTQVTVSS | |
| Ab043 | EVQLVESGGGLVQPGGSLRLSCAASGIHYSSHCMGWYRQAPGKGREFVALMSHSRG | 43 |
| | YYYADSVKGRFTISRDNSKNTVYLQMNSLRAEDTAVYYCAAVPRAGIESGAYCKWQ | |
| | NKDSGSWGQGTQVTVSS | |
| Ab044 | EVQLVESGGGLVQPGGSLRLSCAASGFTYQSHCMGWYRQAPGKGREFVALMQHSRG | 44 |
| | TYYADSVKGRFTISRDNSKNTVYLQMNSLRAEDTAVYYCAAVPRAGIESGAYCKWQ | |
| | NKDSGSWGQGTQVTVSS | |
| Ab045 | EVQLVESGGGLVQPGGSLRLSCAASGFLYSFHCMGWYRQAPGKGREFVALITHSRG | 45 |
| | YSYADSVKGRFTISRDNSKNTVYLQMNSLRAEDTAVYYCAAVPRAGIESGAYCKWQ | |
| | NKDSGSWGQGTQVTVSS | |
| Ab046 | EVQLVESGGGLVQPGGSLRLSCAASGSTYSSHCMGWYRQAPGKGREFVALMTHSRG | 46 |
| | YYYADSVKGRFTISRDNSKNTVYLQMNSLRAEDTAVYYCAAVPRAGIESGAYCKWR | |
| | LQDSGSWGQGTQVTVSS | |
| Ab047 | EVQLVESGGGLVQPGGSLRLSCAASGSTYSSHCMGWYRQAPGKGREFVALMTHSRG | 47 |
| | TYYADSVKGRFTISRDNSKNTVYLQMNSLRAEDTAVYYCAAVPRAGIESGAYCKWR | |
| | LQDSGSWGQGTQVTVSS | |
| Ab048 | EVQLVESGGGLVQPGGSLRLSCAASGSTYSSHCMGWYRQAPGKGREFVALMTRSRG | 48 |
| | YYYADSVKGRFTISRDNSKNTVYLQMNSLRAEDTAVYYCAAVPRAGIESGAYCKWH | |
| | GKDSGSWGQGTQVTVSS | |
| Ab049 | EVQLVESGGGLVQPGGSLRLSCAASGSTYSSHCMGWYRQAPGKGREFVALMTRSRG | 49 |
| | TYYADSVKGRFTISRDNSKNTVYLQMNSLRAEDTAVYYCAAVPRAGIESGAYCKWH | |
| | GKDSGSWGQGTQVTVSS | |
| Ab050 | EVQLVESGGGLVQPGGSLRLSCAASGSTYSSHCMGWYRQAPGKGREFVALMTHSRG | 50 |
| | YYYADSVKGRFTISRDNSKNTVYLQMNSLRAEDTAVYYCAAVPRAGIESGAYCKWH | |
| | NKDSGSWGQGTQVTVSS | |
| Ab051 | EVQLVESGGGLVQPGGSLRLSCAASGSTYSSHCMGWYRQAPGKGREFVALMTHSRG | 51 |
| | TYYADSVKGRFTISRDNSKNTVYLQMNSLRAEDTAVYYCAAVPRAGIESGAYCKWH | |
| | NKDSGSWGQGTQVTVSS | |
| Ab052 | EVQLVESGGGLVQPGGSLRLSCAASGSTYSSHCMGWYRQAPGKGREFVALMTHSRG | 52 |
| | YYYADSVKGRFTISRDNSKNTVYLQMNSLRAEDTAVYYCAAVPRAGIESGAYCKWN | |
| | NKDSGSWGQGTQVTVSS | |
| Ab053 | EVQLVESGGGLVQPGGSLRLSCAASGSTYSSHCMGWYRQAPGKGREFVALMTHSRG | 53 |
| | TYYADSVKGRFTISRDNSKNTVYLQMNSLRAEDTAVYYCAAVPRAGIESGAYCKWN | |
| | NKDSGSWGQGTQVTVSS | |
| Ab054 | EVQLVESGGGLVQPGGSLRLSCAASGSTYSSHCMGWYRQAPGKGREFVALMTHSRG | 54 |
| | YYYADSVKGRFTISRDNSKNTVYLQMNSLRAEDTAVYYCAAVPRAGIESGAYCKWN | |
| | YKDSGSWGQGTQVTVSS | |
| Ab055 | EVQLVESGGGLVQPGGSLRLSCAASGSTYSSHCMGWYRQAPGKGREFVALMTHSRG | 55 |
| | TYYADSVKGRFTISRDNSKNTVYLQMNSLRAEDTAVYYCAAVPRAGIESGAYCKWN | |
| | YKDSGSWGQGTQVTVSS | |
| Ab056 | EVQLVESGGGLVQPGGSLRLSCAASGSTYSSHCMGWYRQAPGKGREFVALMTHSRG | 56 |
| | YYYADSVKGRFTISRDNSKNTVYLQMNSLRAEDTAVYYCAAVPRAGIESGAYCKWQ | |
| | NKDSGSWGQGTQVTVSS | |
| Ab057 | EVQLVESGGGLVQPGGSLRLSCAASGSTYSSHCMGWYRQAPGKGREFVALMTHSRG | 57 |
| | TYYADSVKGRFTISRDNSKNTVYLQMNSLRAEDTAVYYCAAVPRAGIESGAYCKWQ | |
| | NKDSGSWGQGTQVTVSS | |
| Ab058 | EVQLVESGGGLVQPGGSLRLSCAASGSTYSSHCMGWYRQAPGKGREFVALMTHSRG | 58 |
| | YYYADSVKGRFTISRDNSKNTVYLQMNSLRAEDTAVYYCAAVPRAGIESGAYCKWS | |
| | NKDSGSWGQGTQVTVSS | |
| Ab059 | EVQLVESGGGLVQPGGSLRLSCAASGSTYSSHCMGWYRQAPGKGREFVALMTHSRG | 59 |
| | TYYADSVKGRFTISRDNSKNTVYLQMNSLRAEDTAVYYCAAVPRAGIESGAYCKWS | |
| | NKDSGSWGQGTQVTVSS | |
| Ab060 | QVQLVESGGGSVQSGGSLTLSCAASGSTYSSHCMGWFRQAPGKEREGVALMIRSRG | 60 |
| | TSYADSVKGRFTISQDNTKNILYLQMNSLKPEDTAMYYCAAVPRAGIEYSGAYCKW | |
| | NMKDSGSWGQGTLVTVSS | |
| Ab061 | QVQLVESGGGSVQSGGSLTLSCAASGSTYSSHCMGWFRQAPGKEREGVALMIRSRG | 61 |
| | TSYADSVKGRFTISQDNTKNILYLQMNSLKPEDTAMYYCAAVPRAGIESGAYCKWN | |
| | MKDSGSWGQGTLVTVSS | |
| Ab062 | QVQLVESGGGSVQSGGSLTLSCAASGSTYASHCMGWERQAPGKEREGVALMTRSRG | 62 |
| | TSYADSVKGRFTISQDNTKNILYLQMNSLKPEDTAMYYCAAVPRAGIESGAYCKWN | |
| | MKDSGSWGQGTLVTVSS | |
| Ab063 | QVQLVESGGGSVQSGGSLTLSCAASGSTYSSHCMGWFRQAPGKEREGVALMARSRG | 63 |
| | TSYADSVKGRFTISQDNTKNILYLQMNSLKPEDTAMYYCAAVPRAGIESGAYCKWN | |
| | MKDSGSWGQGTLVTVSS | |
| Ab064 | QVQLVESGGGSVQSGGSLTLSCAASGSTYSSHCMGWFRQAPGKEREGVALMTRSRG | 64 |
| | TSYADSVKGRFTISQDNTKNILYLQMNSLKPEDTAMYYCAAVPRAGIESGAYCKAN | |
| | MKDSGSWGQGTLVTVSS | |
| Ab065 | EVQLVESGGGLVQPGGSLRLSCAASGSTYSSHCMGWYRQAPGKGREFVALMTRSRG | 65 |
| | TSYADSVKGRFTISRDNSKNTVYLQMNSLRAEDTAVYYCAAVPRAGIEYSGAYCKW | |
| | NMKDSGSWGQGTQVTVSS | |
| Ab066 | EVQLVESGGGLVQPGGSLRLSCAASGSTYASHCMGWYRQAPGKGREFVALMTRSRG | 66 |
| | TSYADSVKGRFTISRDNSKNTVYLQMNSLRAEDTAVYYCAAVPRAGIESGAYCKWN | |
| | MKDSGSWGQGTQVTVSS | |
| Ab067 | EVQLVESGGGLVQPGGSLRLSCAASGSTYSSHCMGWYRQAPGKGREFVALMARSRG | 67 |
| | TSYADSVKGRFTISRDNSKNTVYLQMNSLRAEDTAVYYCAAVPRAGIESGAYCKWN | |
| | MKDSGSWGQGTQVTVSS | |
| Ab068 | EVQLVESGGGLVQPGGSLRLSCAASGSTYSSHCMGWYRQAPGKGREFVALMTRSRG | 68 |
| | TSYADSVKGRFTISRDNSKNTVYLQMNSLRAEDTAVYYCAAVPRAGIESGAYCKAN | |
| | MKDSGSWGQGTQVTVSS | |
| Ab069 | EVQLVESGGGLVQPGGSLRLSCAASGSTYSSHCMGWYRQAPGKGREFVALMTHSRG | 69 |
| | YSYADSVKGRFTISRDNSKNTVYLQMNSLRAEDTAVYYCAAVPRAGIESGAYCKWS | |
| | YKDSGSWGQGTQVTVSS | |
| Ab070 | EVQLVESGGGLVQPGGSLRLSCAASGSTYSSHCMGWYRQAPGKGREFVALMTHSRG | 70 |
| | TSYYADSVKGRFTISRDNSKNTVYLQMNSLRAEDTAVYYCAAVPRAGIESGAYCKW | |
| | SYKDSGSWGQGTQVTVSS | |
| Ab071 | EVQLVESGGGLVQPGGSLRLSCAASGLTFSSHCMGWYRQAPGKGREFVAAIQGSRG | 71 |
| | STYYADSVKGRFTISRDNSKNTVYLQMNSLRAEDTAVYYCAAVPRAGIESGAYCKW | |
| | SYKDSGSWGQGTQVTVSS | |
| Ab072 | EVQLVESGGGLVQPGGSLRLSCAASGLTFSSHCMGWYRQAPGKGREFVAAIQHSRG | 72 |
| | STYYADSVKGRFTISRDNSKNTVYLQMNSLRAEDTAVYYCAAVPRAGIESGAYCKW | |
| | SYKDSGSWGQGTQVTVSS | |
| Ab073 | EVQLVESGGGLVQPGGSLRLSCAASGLTFSSHCMGWYRQAPGKGREFVAAIQHSRG | 73 |
| | YSYADSVKGRFTISRDNSKNTVYLQMNSLRAEDTAVYYCAAVPRAGIESGAYCKWS | |
| | YKDSGSWGQGTQVTVSS | |
| Ab074 | EVQLVESGGGLVQPGGSLRLSCAASGLTFSSHCMGWYRQAPGKGREFVAAIQRSRG | 74 |
| | YSYADSVKGRFTISRDNSKNTVYLQMNSLRAEDTAVYYCAAVPRAGIESGAYCKWS | |
| | YKDSGSWGQGTQVTVSS | |
| Ab075 | EVQLVESGGGLVQPGGSLRLSCAASGLTFSSHCMGWYRQAPGKGREFVAAISHSRG | 75 |
| | YSYADSVKGRFTISRDNSKNTVYLQMNSLRAEDTAVYYCAAVPRAGIESGAYCKWS | |
| | YKDSGSWGQGTQVTVSS | |
| Ab076 | EVQLVESGGGLVQPGGSLRLSCAASGLTFSSHCMGWYRQAPGKGREFVAAISGSRG | 76 |
| | YSYADSVKGRFTISRDNSKNTVYLQMNSLRAEDTAVYYCAAVPRAGIESGAYCKWS | |
| | YKDSGSWGQGTQVTVSS | |
| Ab077 | EVQLVESGGGLVQPGGSLRLSCAASGLTFSSHCMGWYRQAPGKGREFVAAISHSRG | 77 |
| | SSYADSVKGRFTISRDNSKNTVYLQMNSLRAEDTAVYYCAAVPRAGIESGAYCKWN | |
| | YKDSGSWGQGTQVTVSS | |
| Ab078 | EVQLVESGGGLVQPGGSLRLSCAASGLTFSSHCMGWYRQAPGKGREFVAAIQHSRG | 78 |
| | SSYADSVKGRFTISRDNSKNTVYLQMNSLRAEDTAVYYCAAVPRAGIESGAYCKWN | |
| | YKDSGSWGQGTQVTVSS | |
| Ab079 | EVQLVESGGGLVQPGGSLRLSCAASGLTFSSHCMGWYRQAPGKGREFVAAIQGSRG | 79 |
| | SSYADSVKGRFTISRDNSKNTVYLQMNSLRAEDTAVYYCAAVPRAGIESGAYCKWN | |
| | YKDSGSWGQGTQVTVSS | |
| Ab080 | EVQLVESGGGLVQPGGSLRLSCAASGFTFSSHCMGWYRQAPGKGREFVAAISHSRG | 80 |
| | SSYADSVKGRFTISRDNSKNTVYLQMNSLRAEDTAVYYCAAVPRAGIESGAYCKWN | |
| | YKDSGSWGQGTQVTVSS | |
| Ab081 | EVQLVESGGGLVQPGGSLRLSCAASGFTFSSHCMGWYRQAPGKGREFVAAIQHSRG | 81 |
| | SSYADSVKGRFTISRDNSKNTVYLQMNSLRAEDTAVYYCAAVPRAGIESGAYCKWN | |
| | YKDSGSWGQGTQVTVSS | |
| Ab082 | EVQLVESGGGLVQPGGSLRLSCAASGFTFSSHCMGWYRQAPGKGREFVAAIQGSRG | 82 |
| | SSYADSVKGRFTISRDNSKNTVYLQMNSLRAEDTAVYYCAAVPRAGIESGAYCKWN | |
| | YKDSGSWGQGTQVTVSS | |
| Ab083 | EVQLVESGGGLVQPGGSLRLSCAASGFTFSSYCMGWYRQAPGKGREFVAAISHSRG | 83 |
| | SSYADSVKGRFTISRDNSKNTVYLQMNSLRAEDTAVYYCAAVPRAGIESGAYCKWN | |
| | YKDSGSWGQGTQVTVSS | |
| Ab084 | EVQLVESGGGLVQPGGSLRLSCAASGFTFSSYCMGWYRQAPGKGREFVAAIQGSRG | 84 |
| | SSYADSVKGRFTISRDNSKNTVYLQMNSLRAEDTAVYYCAAVPRAGIESGAYCKWN | |
| | YKDSGSWGQGTQVTVSS | |
| Ab085 | EVQLVESGGGLVQPGGSLRLSCAASGFTFSSYAMGWYRQAPGKGREFVAAISGSGG | 156 |
| | STYYADSVKGRFTISRDNSKNTVYLQMNSLRAEDTAVYYCARTVQRRTLDGYRSSF | |
| | DYWGQGTQVTVSS | |
| Ab086 | EVQLVESGGGLVQPGGSLRLSCAASGFTFSSYAMGWYRQAPGKGREFVAAISGSGG | 157 |
| | STYYADSVKGRFTISRDNSKNTVYLQMNSLRAEDTAVYYCRSRKQRRTLDGYRSSF | |
| | DYWGQGTQVTVSS | |
| Ab087 | EVQLVESGGGLVQPGGSLRLSCAASGFTFSSPHMGWYRQAPGKGREFVAAISGSGT | 158 |
| | AGYYADSVKGRFTISRDNSKNTVYLQMNSLRAEDTAVYYCRSVLQRRTLDGYRTIF | |
| | DYWGQGTQVTVSS | |
| Ab088 | EVQLVESGGGLVQPGGSLRLSCAASGFTFSSPHMGWYRQAPGKGREFVAAISGSGT | 159 |
| | AGYYADSVKGRFTISRDNSKNTVYLQMNSLRAEDTAVYYCRSVLQRRTLDGFRTIF | |
| | DYWGQGTQVTVSS | |
| Ab089 | EVQLVESGGGLVQPGGSLRLSCAASGFTFSSPHMGWYRQAPGKGREFVAAISGSGT | 160 |
| | AGYYADSVKGRFTISRDNSKNTVYLQMNSLRAEDTAVYYCRSVLQRRTLDGYRTVY | |
| | DYWGQGTQVTVSS | |
| Ab090 | EVQLVESGGGLVQPGGSLRLSCAASGFTFSSPHMGWYRQAPGKGREFVAAISGSGT | 161 |
| | AGYYADSVKGRFTISRDNSKNTVYLQMNSLRAEDTAVYYCRSVLQRRTLDGFRTVY | |
| | DYWGQGTQVTVSS | |
| Ab091 | EVQLVESGGGLVQPGGSLRLSCAASGFTHSSYAMGWYRQAPGKGREFVAVISGSGG | 162 |
| | STQLLDSVKGRFTISRDNSKNTVYLQMNSLRAEDTAVYYCRAERQRRTLDGYRTIF | |
| | DYWGQGTQVTVSS | |
| Ab092 | EVQLVESGGGLVQPGGSLRLSCAASGFTHSSYAMGWYRQAPGKGREFVAVISGSGG | 163 |
| | STQLLDSVKGRFTISRDNSKNTVYLQMNSLRAEDTAVYYCRAERQRRTLDGFRTVY | |
| | DYWGQGTQVTVSS | |
| Ab093 | EVQLVESGGGLVQPGGSLRLSCAASGFHHSSYAMGWYRQAPGKGREFVASISGSGG | 164 |
| | STYYADSVKGRFTISRDNSKNTVYLQMNSLRAEDTAVYYCRMMSQRRTLDGYRTIF | |
| | DYWGQGTQVTVSS | |
| Ab094 | EVQLVESGGGLVQPGGSLRLSCAASGFHHSSYAMGWYRQAPGKGREFVASISGSGG | 165 |
| | STYYADSVKGRFTISRDNSKNTVYLQMNSLRAEDTAVYYCRMMSQRRTLDGERTIF | |
| | DYWGQGTQVTVSS | |
| Ab095 | EVQLVESGGGLVQPGGSLRLSCAASGFHHSSYAMGWYRQAPGKGREFVASISGSGG | 166 |
| | STYYADSVKGRFTISRDNSKNTVYLQMNSLRAEDTAVYYCRMMSQRRTLDGYRTVY | |
| | DYWGQGTQVTVSS | |
| Ab096 | EVQLVESGGGLVQPGGSLRLSCAASGFHHSSYAMGWYRQAPGKGREFVASISGSGG | 167 |
| | STYYADSVKGRFTISRDNSKNTVYLQMNSLRAEDTAVYYCRMMSQRRTLDGERTVY | |
| | DYWGQGTQVTVSS | |
| Ab097 | EVQLVESGGGLVQPGGSLRLSCAASQQTFSSYAMGWYRQAPGKGREFVASTSGSGG | 168 |
| | STYYADSVKGRFTISRDNSKNTVYLQMNSLRAEDTAVYYCRAMRQRRTLDGYRSSF | |
| | DYWGQGTQVTVSS | |
| Ab098 | EVQLVESGGGLVQPGGSLRLSCAASQVTFSSYAMGWYRQAPGKGREFVASISHYDG | 169 |
| | STYYADSVKGRFTISRDNSKNTVYLQMNSLRAEDTAVYYCRAGRQRRTLDGYRSSF | |
| | DYWGQGTQVTVSS | |
| Ab099 | EVQLVESGGGLVQPGGSLRLSCAASGFTFPPYAMGWYRQAPGKGREFVASISGSGG | 170 |
| | STYYADSVKGRFTISRDNSKNTVYLQMNSLRAEDTAVYYCRGMKQRRTLDGYRSSF | |
| | DYWGQGTQVTVSS | |
| Ab100 | EVQLVESGGGLVQPGGSLRLSCAASGFHHSSYAMGWYRQAPGKGREFVASISGSGG | 171 |
| | STYYADSVKGRFTISRDNSKNTVYLQMNSLRAEDTAVYYCRMMSQRRTLDGYRSSF | |
| | DYWGQGTQVTVSS | |
| Ab101 | EVQLVESGGGLVQPGGSLRLSCAASGFAISSYAMGWYRQAPGKGREFVAAISFIAG | 172 |
| | STYYADSVKGRFTISRDNSKNTVYLQMNSLRAEDTAVYYCCAVKQRRTLDGYRSSF | |
| | DYWGQGTQVTVSS | |
| Ab102 | EVQLVESGGGLVQPGGSLRLSCAASGFAISSYAMGWYRQAPGKGREFVAAISFIAG | 173 |
| | STYYADSVKGRFTISRDNSKNTVYLQMNSLRAEDTAVYYCSAVKQRRTLDGYRSSF | |
| | DYWGQGTQVTVSS | |
| Ab103 | EVQLVESGGGLVQPGGSLRLSCAASGFPFSSYAMGWYRQAPGKGREFVAAISFLEG | 174 |
| | STYYADSVKGRFTISRDNSKNTVYLQMNSLRAEDTAVYYCRAGKQRRTLDGYRSSF | |
| | DYWGQGTQVTVSS | |
| Ab104 | EVQLVESGGGLVQPGGSLRLSCAASGFDISSYAMGWYRQAPGKGREFVAAISFVAG | 175 |
| | STYYADSVKGRFTISRDNSKNTVYLQMNSLRAEDTAVYYCEARRQRRTLDGYRSSF | |
| | DYWGQGTQVTVSS | |
| Ab105 | EVQLVESGGGLVQPGGSLRLSCAASGFVGSSYAMGWYRQAPGKGREFVASIGGSGG | 176 |
| | STYYADSVKGRFTISRDNSKNTVYLQMNSLRAEDTAVYYCRVARQRRTLDGYRSSF | |
| | DYWGQGTQVTVSS | |
| Ab106 | EVQLVESGGGLVQPGGSLRLSCAASGFTHSSYAMGWYRQAPGKGREFVAVISGSGG | 177 |
| | STQLLDSVKGRFTISRDNSKNTVYLQMNSLRAEDTAVYYCRAERQRRTLDGYRSSF | |
| | DYWGQGTQVTVSS | |
| Ab107 | EVQLVESGGGLVQPGGSLRLSCAASGFTHSSYAMGWYRQAPGKGREFVAVISGSGG | 178 |
| | STQLLDSVKGRFTISRDNSKNTVYLQMNSLRAEDTAVYYCRAERQHRTLDGYRSSF | |
| | DYWGQGTQVTVSS | |
| Ab108 | EVQLVESGGGLVQPGGSLRLSCAASGFTFSSPHMGWYRQAPGKGREFVAAISGSGT | 179 |
| | AGYYADSVKGRFTISRDNSKNTVYLQMNSLRAEDTAVYYCRVSLQRRTLDGYRSSF | |
| | DYWGQGTQVTVSS | |
| Ab109 | EVQLVESGGGLVQPGGSLRLSCAASGFPHPSYPMGWYRQAPGKGREFVAGPGGSGG | 180 |
| | STYYADSVKGRFTISRDNSKNTVYLQMNSLRAEDTAVYYCRGARQRRTLDGYRSSF | |
| | DYWGQGTQVTVSS | |
| Ab110 | EVQLVESGGGLVQPGGSLRLSCAASGFHFSSYAMGWYRQAPGKGREFVASISGSGG | 181 |
| | STYYADSVKGRFTISRDNSKNTVYLQMNSLRAEDTAVYYCRMMSQRRTLDGYRTIF | |
| | DYWGQGTQVTVSS | |
| Ab111 | EVQLVESGGGLVQPGGSLRLSCAASGFTFSSYAMGWYRQAPGKGREFVASISGSGG | 182 |
| | STYYADSVKGRFTISRDNSKNTVYLQMNSLRAEDTAVYYCRMMSQRRTLDGYRTIF | |
| | DYWGQGTQVTVSS | |
| Ab112 | EVQLVESGGGLVQPGGSLRLSCAASGFTFSSYAMGWYRQAPGKGREFVASISFSGG | 183 |
| | STYYADSVKGRFTISRDNSKNTVYLQMNSLRAEDTAVYYCRMMSQRRTLDGYRTIF | |
| | DYWGQGTQVTVSS | |
| Ab113 | EVQLVESGGGLVQPGGSLRLSCAASGFTFSSYAMGWYRQAPGKGREFVASISGSGG | 184 |
| | STQYADSVKGRFTISRDNSKNTVYLQMNSLRAEDTAVYYCRMMSQRRTLDGYRTIF | |
| | DYWGQGTQVTVSS | |
| Ab114 | EVQLVESGGGLVQPGGSLRLSCAASGFTFSSYAMGWYRQAPGKGREFVAAISFSGG | 185 |
| | STQYADSVKGRFTISRDNSKNTVYLQMNSLRAEDTAVYYCRMMSQRRTLDGYRTIF | |
| | DYWGQGTQVTVSS | |
| Ab115 | EVQLVESGGGLVQPGGSLRLSCAASGFTFSSYAMGWYRQAPGKGREFVAAISFSGG | 186 |
| | STYYADSVKGRFTISRDNSKNTVYLQMNSIRAEDTAVYYCRMMSQRRTLDGYRTIF | |
| | DYWGQGTQVTVSS | |
| Ab116 | EVQLVESGGGLVQPGGSLRLSCAASGFHHSSYAMGWYRQAPGKGREFVASISGSGG | 187 |
| | STYYADSVKGRFTISRDNSKNTVYLQMNSLRAEDTAVYYCRSMSQRRTLDGYRTIF | |
| | DYWGQGTQVTVSS | |
| Ab117 | EVQLVESGGGLVQPGGSLRLSCAASGFHHSSYAMGWYRQAPGKGREFVASISGSGG | 188 |
| | STYYADSVKGRFTISRDNSKNTVYLQMNSLRAEDTAVYYCRMVSQRRTLDGYRTIF | |
| | DYWGQGTQVTVSS | |
| Ab118 | EVQLVESGGGLVQPGGSLRLSCAASGFHFSSYAMGWYRQAPGKGREFVASISGSGG | 189 |
| | STYYADSVKGRFTISRDNSKNTVYLQMNSLRAEDTAVYYCRSMSQRRTLDGYRTIF | |
| | DYWGQGTQVTVSS | |
| Ab119 | EVQLVESGGGLVQPGGSLRLSCAASGFHFSSYAMGWYRQAPGKGREFVASISGSGG | 190 |
| | STYYADSVKGRFTISRDNSKNTVYLQMNSLRAEDTAVYYCRMVSQRRTLDGYRTIF | |
| | DYWGQGTQVTVSS | |
| Ab120 | EVQLVESGGGLVQPGGSLRLSCAASGFTHSSYAMGWYRQAPGKGREFVASISGSGG | 191 |
| | STYYADSVKGRFTISRDNSKNTVYLQMNSLRAEDTAVYYCRSMSQRRTLDGYRTIF | |
| | DYWGQGTQVTVSS | |
| JN241-1 | QVQLVESGGGSVQSGGSLTLSCAASGSTYSSHCMGWFRQAPGKEREGVALMTRSRG | 823 |
| | TSYADSVKGRFTISQDNTKNILYLQMNSLKPEDTAMYYCAAVPRAGIFSGAYCKWN | |
| | MKDSGSWGQGTLVTVSS | |
| JN241-2 | QVQLVESGGGSVQSGGSLTLSCAASGSTYSSHCMGWFRQAPGKEREGVALMTRSRG | 824 |
| | TSYADSVKGRFTISQDNTKNILYLQMNSLKPEDTAMYYCAAVPRAGIWSGAYCKWN | |
| | MKDSGSWGQGTLVTVSS | |
| JN241-3 | QVQLVESGGGSVQSGGSLTLSCAASGSTYSSHCMGWFRQAPGKEREGVALMIRSRG | 825 |
| | TSYADSVKGRFTISQDNTKNILYLQMNSLKPEDTAMYYCAAVPRAGIYSGAYCKWN | |
| | MKDSGSWGQGTLVTVSS | |
| JN241-4 | QVQLVESGGGSVQSGGSLTLSCAASGSTYSSHCMGWFRQAPGKEREGVALMTRSRG | 826 |
| | TSYADSVKGRFTISQDNTKNILYLQMNSLKPEDTAMYYCAAVPRAGIEFGAYCKWN | |
| | MKDSGSWGQGTLVTVSS | |
| JN241-5 | QVQLVESGGGSVQSGGSLTLSCAASGSTYSSHCMGWFRQAPGKEREGVALMIRSRG | 827 |
| | TSYADSVKGRFTISQDNTKNILYLQMNSLKPEDTAMYYCAAVPRAGIEWGAYCKWN | |
| | MKDSGSWGQGTLVTVSS | |
| JN241-6 | QVQLVESGGGSVQSGGSLTLSCAASGSTYSSHCMGWFRQAPGKEREGVALMTRSRG | 828 |
| | TSYADSVKGRFTISQDNTKNILYLQMNSLKPEDTAMYYCAAVPRAGIEYGAYCKWN | |
| | MKDSGSWGQGTLVTVSS | |
| JN241-7 | QVQLVESGGGSVQSGGSLTLSCAASGSTYSSHCMGWFRQAPGKEREGVALMTRSRG | 829 |
| | TSYADSVKGRFTISQDNTKNILYLQMNSLKPEDTAMYYCAAVPRAGIEFSGAYCKW | |
| | NMKDSGSWGQGTLVTVSS | |
| JN241-8 | QVQLVESGGGSVQSGGSLTLSCAASGSTYSSHCMGWFRQAPGKEREGVALMIRSRG | 830 |
| | TSYADSVKGRFTISQDNTKNILYLQMNSLKPEDTAMYYCAAVPRAGIEWSGAYCKW | |
| | NMKDSGSWGQGTLVTVSS | |

The individual CDRs of an antibody disclosed herein can be determined according to any CDR numbering scheme known in the art.

In certain embodiments, one or more of the CDRs of an antibody disclosed herein can be determined according to Kabat et al., J. Biol. Chem. 252, 6609-6616 (1977) and Kabat et al., Sequences of protein of immunological interest (1991), each of which is herein incorporated by reference in its entirety.

In certain embodiments, an antibody provided herein comprises the CDRH1, CDRH2, and/or CDRH3 of a VH amino acid sequence set forth in any of SEQ ID NOs: 1-84 or 156-191 as determined by the Kabat numbering scheme. In certain embodiments, an antibody provided herein comprises the CDRH1, CDRH2, and/or CDRH3 of a VH amino acid sequence set forth in any of SEQ ID NOs: 1-84 as determined by the Kabat numbering scheme. In certain embodiments, an antibody provided herein comprises the CDRH1, CDRH2, and/or CDRH3 of a VH amino acid sequence set forth in any of SEQ ID NOs: 156-191 as determined by the Kabat numbering scheme.

In certain embodiments, one or more of the CDRs of an antibody disclosed herein can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226, all of which are herein incorporated by reference in their entireties).

In certain embodiments, an antibody provided herein comprises the CDRH1, CDRH2, and/or CDRH3 of a VH amino acid sequence set forth in any of SEQ ID NOs: 1-84 or 156-191 as determined by the Chothia numbering system. In certain embodiments, an antibody provided herein comprises the CDRH1, CDRH2, and/or CDRH3 of a VH amino acid sequence set forth in any of SEQ ID NOs: 1-84 as determined by the Chothia numbering system. In certain embodiments, an antibody provided herein comprises the CDRH1, CDRH2, and/or CDRH3 of a VH amino acid sequence set forth in any of SEQ ID NOs: 156-191 as determined by the Chothia numbering system.

In certain embodiments, one or more of the CDRs of an antibody disclosed herein can be determined according to MacCallum R M et al., (1996) J Mol Biol 262: 732-745, herein incorporated by reference in its entirety. See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001), herein incorporated by reference in its entirety.

In certain embodiments, an antibody provided herein comprises the CDRH1, CDRH2, and/or CDRH3 of a VH amino acid sequence set forth in any of SEQ ID NOs: 1-84 or 156-191 as determined by the MacCallum numbering system. In certain embodiments, an antibody provided herein comprises the CDRH1, CDRH2, and/or CDRH3 of a VH amino acid sequence set forth in any of SEQ ID NOs: 1-84 as determined by the MacCallum numbering system. In certain embodiments, an antibody provided herein comprises the CDRH1, CDRH2, and/or CDRH3 of a VH amino acid sequence set forth in any of SEQ ID NOs: 156-191 as determined by the MacCallum numbering system.

In certain embodiments, the CDRs of an antibody disclosed herein can be determined according to the IMGT numbering system as described in: Lefranc M-P, (1999) The Immunologist 7: 132-136; Lefranc M-P et al., (1999) Nucleic Acids Res 27: 209-212, each of which is herein incorporated by reference in its entirety; and Lefranc M-P et al., (2009) Nucleic Acids Res 37: D1006-D1012.

In certain embodiments, an antibody provided herein comprises the CDRH1, CDRH2, and/or CDRH3 of a VH amino acid sequence set forth in any of SEQ ID NOs: 1-84 or 156-191 as determined by the IMGT numbering system. In certain embodiments, an antibody provided herein comprises the CDRH1, CDRH2, and/or CDRH3 of a VH amino acid sequence set forth in any of SEQ ID NOs: 1-84 as determined by the IMGT numbering system. In certain embodiments, an antibody provided herein comprises the CDRH1, CDRH2, and/or CDRH3 of a VH amino acid sequence set forth in any of SEQ ID NOs: 156-191 as determined by the IMGT numbering system.

In certain embodiments, the CDRs of an antibody disclosed herein can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

In certain embodiments, an antibody provided herein comprises the CDRH1, CDRH2, and/or CDRH3 of a VH amino acid sequence set forth in any of SEQ ID NOs: 1-84 or 156-191 as determined by the AbM numbering scheme. In certain embodiments, an antibody provided herein comprises the CDRH1, CDRH2, and/or CDRH3 of a VH amino acid sequence set forth in any of SEQ ID NOs: 1-84 as determined by the AbM numbering scheme. In certain embodiments, an antibody provided herein comprises the CDRH1, CDRH2, and/or CDRH3 of a VH amino acid sequence set forth in any of SEQ ID NOs: 156-191 as determined by the AbM numbering scheme.

In certain embodiments, the CDRs of an antibody disclosed herein can be determined according to the AHo numbering system, as described in Honegger and Pluckthun, A., J. Mol. Biol. 309:657-670 (2001), herein incorporated by reference in its entirety.

In certain embodiments, an antibody provided herein comprises the CDRH1, CDRH2, and/or CDRH3 of a VH amino acid sequence set forth in any of SEQ ID NOs: 1-84 or 156-191 as determined by the AHo numbering system. In certain embodiments, an antibody provided herein comprises the CDRH1, CDRH2, and/or CDRH3 of a VH amino acid sequence set forth in any of SEQ ID NOs: 1-84 as determined by the AHo numbering system. In certain embodiments, an antibody provided herein comprises the CDRH1, CDRH2, and/or CDRH3 of a VH amino acid sequence set forth in any of SEQ ID NOs: 156-191 as determined by the AHo numbering system.

In certain embodiments, the CDRs of an antibody disclosed herein can be determined utilizing the CDR boundaries described in Ma et al., 2020, Sci. Adv. 6:eaax7379, herein incorporated by reference in its entirety.

In certain embodiments, an antibody provided herein comprises the CDRH1, CDRH2, and/or CDRH3 of a VH amino acid sequence set forth in any of SEQ ID NOs: 1-84 or 156-191 as determined utilizing the CDR boundaries described in Ma et al., 2020, supra. In certain embodiments, an antibody provided herein comprises the CDRH1, CDRH2, and/or CDRH3 of a VH amino acid sequence set forth in any of SEQ ID NOs: 1-84 as determined utilizing the CDR boundaries described in Ma et al., 2020, supra. In certain embodiments, an antibody provided herein comprises the CDRH1, CDRH2, and/or CDRH3 of a VH amino acid sequence set forth in any of SEQ ID NOs: 156-191 as determined utilizing the CDR boundaries described in Ma et al., 2020, supra.

In certain embodiments, the individual CDRs of an antibody disclosed herein are each independently determined according to one of the Kabat, Chothia, MacCallum, IMGT, AHo, or AbM numbering schemes, utilizing the CDR boundaries described in Ma et al., 2020, supra, or by structural analysis of the antibody, wherein the structural analysis identifies residues in the variable region(s) predicted to make contact with an epitope region of APJ.

In certain embodiments, the instant disclosure provides an antibody that specifically binds APJ (e.g., human APJ) comprising a VH comprising the CDRH1, CDRH2, and CDRH3 amino acid sequences of a VH amino acid sequence set forth in any one of SEQ ID NOs: 1-84 or 156-191, wherein each CDR is independently determined according to one of the Kabat, Chothia, MacCallum, IMGT, AHo, or AbM numbering schemes, utilizing the CDR boundaries described in Ma et al., 2020, supra, or by structural analysis of the antibody, wherein the structural analysis identifies residues in the variable region(s) predicted to make contact with an epitope region of APJ (e.g., human APJ). In certain embodiments, the instant disclosure provides an antibody that specifically binds APJ (e.g., human APJ) comprising a VH comprising the CDRH1, CDRH2, and CDRH3 amino acid sequences of a VH amino acid sequence set forth in any one of SEQ ID NOs: 1-84, wherein each CDR is independently determined according to one of the Kabat, Chothia, MacCallum, IMGT, AHo, or AbM numbering schemes, utilizing the CDR boundaries described in Ma et al., 2020, supra, or by structural analysis of the antibody, wherein the structural analysis identifies residues in the variable region(s) predicted to make contact with an epitope region of APJ (e.g., human APJ). In certain embodiments, the instant disclosure provides an antibody that specifically binds APJ (e.g., human APJ) comprising a VH comprising the CDRH1, CDRH2, and CDRH3 amino acid sequences of a VH amino acid sequence set forth in any one of SEQ ID NOs: 156-191, wherein each CDR is independently determined according to one of the Kabat, Chothia, MacCallum, IMGT, AHo, or AbM numbering schemes, utilizing the CDR boundaries described in Ma et al., 2020, supra, or by structural analysis of the antibody, wherein the structural analysis identifies residues in the variable region(s) predicted to make contact with an epitope region of APJ (e.g., human APJ).

In certain embodiments, the instant disclosure provides an antibody that specifically binds to APJ (e.g., human APJ), the antibody comprising CDRH1, CDRH2, and CDRH3 consensus amino acid sequences set forth in Table 2.

TABLE 2
CDRH consensus sequences of exemplary anti-APJ antibodies.

| | | SEQ |
|---|---|---|
| | | ID |
| CDRH | Amino acid sequence | NO |
| CDRH1 | GX₁X₂X₃X₄X₅X₆CX₇X₈, wherein: | 247 |
| cons. | X₁ is L, F, I, S, Y, A, H, V, or Q; | |
| seq. 1 | X₂ is T, H, L, N, Q, or S; | |
| | X₃ is F, Y, I, L, or V; | |
| | X₄ is S, A, H, Q, V, I, or T; | |
| | X₅ is S, F, H, or Y; | |
| | X₆ is H or Y; | |
| | X₇ is M or absent; and | |
| | X₈ is G, S, L, Y, or absent | |
| CDRH2 | X₉X₁₀X₁₁X₁₂SX₁₃GX₁₄X₁₅X₁₆X₁₇, wherein: | 248 |
| cons. | X₉ is A, L, or absent; | |
| seq. 1 | X₁₀ is I or M; | |
| | X₁₁ is S, A, Q, or T; | |
| | X₁₂ is G, H, or R; | |
| | X₁₃ is R or Y; | |
| | X₁₄ is Y, S, T, F, or H; | |
| | X₁₅ is S, T, Y, Q, or absent; | |
| | X₁₆ is Y or absent; and | |
| | X₁₇ is absent or Y | |
| CDRH3 | AAVPRAGIX₁₈X₁₉X₂₀GAYCKX₂₁X₂₂X₂₃X₂₄DSGS, wherein: | 249 |
| cons. | X₁₈ is E, F, Y, or W; | |
| seq. 1 | X₁₉ is absent, Y, F, P, K, R, W, L, or I; | |
| | X₂₀ is S, F, Y, or W; | |
| | X₂₁ is W, A, F or Y; | |
| | X₂₂ is S, H, I, K, N, P, Q, R, or T; | |
| | X₂₃ is Y, G, H, I, L, M, N, or R; and | |
| | X₂₄ is K or Q | |
| CDRH1 | X₂₅X₂₆X₂₇X₂₈X₂₉X₃₀X₃₁X₃₂X₃₃X₃₄, wherein: | 250 |
| cons. | X₂₅ is G or Q; | |
| seq. 2 | X₂₆ is F, Q, or V; | |
| | X₂₇ is T, A, D, H, P, V, R, K, or E; | |
| | X₂₈ is F, G, H, I, or V; | |
| | X₂₉ is S, P, R, or K; | |
| | X₃₀ is S or P; | |
| | X₃₁ is P or Y; | |
| | X₃₂ is H, A, P, R, or K; | |
| | X₃₃ is M or absent; and | |
| | X₃₄ is G, R, K, H, or absent | |
| CDRH2 | X₃₅X₃₆X₃₇X₃₈X₃₉X₄₀X₄₁X₄₂X₄₃X₄₄X₄₅X₄₆X₄₇X₄₈X₄₉X₅₀, wherein: | 251 |
| cons. | X₃₅ is A, G, S, V, R, K, H, or absent; | |
| seq. 2 | X₃₆ is I, P, or T; | |
| | X₃₇ is S or G; | |
| | X₃₈ is G, F, or H; | |
| | X₃₉ is S, I, L, V, or Y; | |
| | X₄₀ is G, A, D, or E; | |
| | X₄₁ is T, G, R, K, or H; | |
| | X₄₂ is A or S; | |
| | X₄₃ is G, T, or absent; | |
| | X₄₄ is Y, Q, R, K, H, or absent; | |
| | X₄₅ is Y, L, E, D, or absent; | |
| | X₄₆ is A, L, or absent; | |
| | X₄₇ is D, H, P, or absent; | |
| | X₄₈ is S or absent; | |
| | X₄₉ is V or absent; and | |
| | X₅₀ is K, Q, or absent | |
| CDRH3 | X₅₁X₅₂X₅₃X₅₄X₅₅X₅₆RX₅₇LX₅₈GX₅₉RX₆₀X₆₁X₆₂DY, wherein: | 252 |
| cons. | X₅₁ is R, A, C, E, or S; | |
| seq. 2 | X₅₂ is V, A, G, M, R, or S; | |
| | X₅₃ is S, A, E, G, M, R, T, or V; | |
| | X₅₄ is L, K, R, S, or V; | |
| | X₅₅ is Q or G; | |
| | X₅₆ is R or H; | |
| | X₅₇ is T, L, or M; | |
| | X₅₈ is D or E; | |
| | X₅₉ is Y or F; | |
| | X₆₀ is S or T; | |
| | X₆₁ is S, I, V, or L; and | |
| | X₆₂ is F or Y | |

In certain embodiments, the instant disclosure provides an antibody that specifically binds to APJ (e.g., human APJ), the antibody comprising a) a CDRH1 comprising the amino acid sequence of GX₁X₂X₃X₄X₅X₆CX₇X₈ (SEQ ID NO: 247), wherein X₁ is leucine (L), phenylalanine (F), isoleucine (I), serine (S), tyrosine (Y), alanine (A), histidine (H), valine (V), or glutamine (Q); X₂ is threonine (T), histidine (H), leucine (L), asparagine (N), glutamine (Q), or serine (S); X₃ is phenylalanine (F), tyrosine (Y), isoleucine (I), leucine (L), or valine (V); X₄ is serine (S), alanine (A), histidine (H), glutamine (Q), valine (V), isoleucine (I), or threonine (T); X₅ is serine (S), phenylalanine (F), histidine (H), or tyrosine (Y); X₆ is histidine (H) or tyrosine (Y); X₇ is methionine (M) or absent; and X₈ is G, serine (S), leucine (L), tyrosine (Y), or absent, b) a CDRH2 comprising the amino acid sequence of X₉X₁₀X₁₁X₁₂SX₁₃GX₁₄X₁₅X₁₆X₁₇ (SEQ ID NO: 248), wherein X₉ is alanine (A), leucine (L), or absent; X₁₀ is isoleucine (I) or methionine (M); X₁₁ is serine (S), alanine (A), glutamine (Q), or threonine (T); X₁₂ is glycine (G), histidine (H), or arginine (R); X₁₃ is arginine (R) or tyrosine (Y); X₁₄ is tyrosine (Y), serine (S), threonine (T), phenylalanine (F), or histidine (H); X₁₅ is serine (S), threonine (T), tyrosine (Y), glutamine (Q), or absent; X₁₆ is tyrosine (Y) or absent; and X₁₇ is absent or tyrosine (Y), and/or c) a CDRH3 comprising the amino acid sequence of AAVPRAGIX₁₈X₁₉X₂₀GAYCKX₂₁X₂₂X₂₃X₂₄DSGS (SEQ ID NO: 249), wherein X₁₈ is glutamic acid (E), phenylalanine (F), tyrosine (Y), or tryptophan (W); X₁₉ is absent, tyrosine (Y), phenylalanine (F), proline (P), lysine (K), arginine (R), tryptophan (W), leucine (L), or isoleucine (I); X₂₀ is serine (S), phenylalanine (F), tyrosine (Y), or tryptophan (W); X₂₁ is tryptophan (W), alanine (A), phenylalanine (F) or tyrosine (Y); X₂₂ is serine (S), histidine (H), isoleucine (I), lysine (K), asparagine (N), proline (P), glutamine (Q), arginine (R), or threonine (T); X₂₃ is tyrosine (Y), glycine (G), histidine (H), isoleucine (I), leucine (L), methionine (M), asparagine (N), or arginine (R); and X₂₄ is lysine (K) or glutamine (Q).

In certain embodiments, the instant disclosure provides an antibody that specifically binds to APJ (e.g., human APJ), the antibody comprising a) a CDRH1 comprising the amino acid sequence of GX₁X₂X₃X₄X₅X₆CX₇X₈ (SEQ ID NO: 247), wherein X₁ is leucine (L), phenylalanine (F), isoleucine (I), serine (S), tyrosine (Y), alanine (A), histidine (H), valine (V), or glutamine (Q); X₂ is threonine (T), histidine (H), leucine (L), asparagine (N), glutamine (Q), or serine (S); X₃ is phenylalanine (F), tyrosine (Y), isoleucine (I), leucine (L), or valine (V); X₄ is serine (S), alanine (A), histidine (H), glutamine (Q), valine (V), isoleucine (I), or threonine (T); X₅ is serine (S), phenylalanine (F), histidine (H), or tyrosine (Y); X₆ is histidine (H) or tyrosine (Y); X₇ is methionine (M) or absent; and X₈ is G, serine (S), leucine (L), tyrosine (Y), or absent, b) a CDRH2 comprising the amino acid sequence of X₉X₁₀X₁₁X₁₂SX₁₃GX₁₄X₁₅X₁₆X₁₇ (SEQ ID NO: 248), wherein X₉ is alanine (A), leucine (L), or absent; X₁₀ is isoleucine (I) or methionine (M); X₁₁ is serine (S), alanine (A), glutamine (Q), or threonine (T); X₁₂ is glycine (G), histidine (H), or arginine (R); X₁₃ is arginine (R) or tyrosine (Y); X₁₄ is tyrosine (Y), serine (S), threonine (T), phenylalanine (F), or histidine (H); X₁₅ is serine (S), threonine (T), tyrosine (Y), glutamine (Q), or absent; X₁₆ is tyrosine (Y) or absent; and X₁₇ is absent or tyrosine (Y), and c) a CDRH3 comprising the amino acid sequence of AAVPRAGIX₁₈X₁₉X₂₀GAYCKX₂₁X₂₂X₂₃X₂₄DSGS (SEQ ID NO: 249), wherein X₁₈ is glutamic acid (E), phenylalanine (F), tyrosine (Y), or tryptophan (W); X₁₉ is absent, tyrosine (Y), phenylalanine (F), proline (P), lysine (K), arginine (R), tryptophan (W), leucine (L), or isoleucine (I); X₂₀ is serine (S), phenylalanine (F), tyrosine (Y), or tryptophan (W); X₂₁ is tryptophan (W), alanine (A), phenylalanine (F) or tyrosine (Y); X₂₂ is serine (S), histidine (H), isoleucine (I), lysine (K), asparagine (N), proline (P), glutamine (Q), arginine (R), or threonine (T); X₂₃ is tyrosine (Y), glycine (G), histidine (H), isoleucine (I), leucine (L), methionine (M), asparagine (N), or arginine (R); and X₂₄ is lysine (K) or glutamine (Q).

In certain embodiments, X₁ is leucine (L), phenylalanine (F), isoleucine (I), serine (S), or tyrosine (Y); X₂ is threonine (T), histidine (H), leucine (L), or asparagine (N); X₃ is phenylalanine (F) or tyrosine (Y); X₄ is serine (S), alanine (A), histidine (H), glutamine (Q), or valine (V); X₅ is serine (S) or phenylalanine (F); X₆ is histidine (H) or tyrosine (Y); X₇ is methionine (M) or absent; X₈ is glycine (G) or absent; X₉ is alanine (A), leucine (L), or absent; X₁₀ is isoleucine (I) or methionine (M); X₁₁ is serine (S), alanine (A), glutamine (Q), or threonine (T); X₁₂ is glycine (G), histidine (H), or arginine (R); X₁₃ is arginine (R) or tyrosine (Y); X₁₄ is tyrosine (Y), serine (S), or threonine (T); X₁₈ is serine (S), threonine (T), tyrosine (Y), or absent; X₁₆ is tyrosine (Y) or absent; X₁₇ is absent or tyrosine (Y); X₁₈ is glutamic acid (E); X₁₉ is absent or tyrosine (Y); X₂₀ is serine (S); X₂₁ is tryptophan (W) or A; X₂₂ is serine (S), histidine (H), isoleucine (I), lysine (K), asparagine (N), proline (P), glutamine (Q), arginine (R), or threonine (T); X₂₃ is tyrosine (Y), glycine (G), histidine (H), isoleucine (I), leucine (L), methionine (M), asparagine (N), or arginine (R); and X₂₄ is lysine (K) or glutamine (Q).

In certain embodiments, X₄ is serine (S), histidine (H), glutamine (Q), or valine (V); X₁₁ is serine (S), glutamine (Q), or threonine (T); and X₂₁ is tryptophan (W).

In certain embodiments, X₁₉ is absent.

In certain embodiments, X₁₉ is tyrosine (Y) or phenylalanine (F).

In certain embodiments, X₇, X₈, X₉, X₁₅, X₁₆, and X₁₇ are absent.

In certain embodiments, X₁ is leucine (L); X₂ is threonine (T); X₃ is phenylalanine (F); X₄ is serine (S); X₅ is serine (S); X₆ is histidine (H); X₇ is absent; X₈ is absent; X₉ is absent; X₁₀ is isoleucine (I); X₁₁ is serine (S) or glutamine (Q); X₁₂ is glycine (G) or histidine (H); X₁₃ is arginine (R); X₁₄ is tyrosine (Y) or serine (S); X₁₈ is absent; X₁₆ is absent; X₁₇ is absent; X₁₈ is glutamic acid (E); X₁₉ is absent; X₂₀ is serine (S); X₂₁ is tryptophan (W); X₂₂ is serine (S) or asparagine (N); X₂₃ is tyrosine (Y); and X₂₄ is lysine (K).

In certain embodiments, X₁ is leucine (L); X₂ is threonine (T); X₃ is phenylalanine (F); X₄ is serine (S); X₅ is serine (S); X₆ is histidine (H); X₇ is methionine (M); X₈ is glycine (G); X₉ is A; X₁₀ is isoleucine (I); X₁₁ is serine (S) or glutamine (Q); X₁₂ is glycine (G) or histidine (H); X₁₃ is arginine (R); X₁₄ is tyrosine (Y) or serine (S); X₁₅ is serine (S); X₁₆ is tyrosine (Y); X₁₇ is absent; X₁₈ is glutamic acid (E); X₁₉ is absent; X₂₀ is serine (S); X₂₁ is tryptophan (W); X₂₂ is serine (S) or asparagine (N); X₂₃ is tyrosine (Y); and X₂₄ is lysine (K).

In certain embodiments, the instant disclosure provides an antibody that specifically binds to APJ (e.g., human APJ), the antibody comprising a) a CDRH1 comprising the amino acid sequence of X₂₅X₂₆X₂₇X₂₈X₂₉X₃₀X₃₁X₃₂X₃₃X₃₄ (SEQ ID NO: 250), wherein X₂₅ is glycine (G) or glutamine (Q); X₂₆ is phenylalanine (F), glutamine (Q), or valine (V); X₂₇ is threonine (T), alanine (A), aspartic acid (D), histidine (H), proline (P), valine (V), arginine (R), lysine (K), or glutamic acid (E); X₂₈ is phenylalanine (F), glycine (G), histidine (H), isoleucine (I), or valine (V); X₂₉ is serine (S), proline (P), arginine (R), or lysine (K); X₃₀ is serine (S) or proline (P); X₃₁ is proline (P) or tyrosine (Y); X₃₂ is histidine (H), alanine (A), proline (P), arginine (R), or lysine (K); X₃₃ is methionine (M) or absent; and X₃₄ is glycine (G), arginine (R), lysine (K), histidine (H), or absent, b) a CDRH2 comprising the amino acid sequence of X₃₅X₃₆X₃₇X₃₈X₃₉X₄₀X₄₁X₄₂X₄₃X₄₄X₄₅X₄₆X₄₇X₄₈X₄₉X₅₀ (SEQ ID NO: 251), wherein X₃₅ is alanine (A), glycine (G), serine (S), valine (V), arginine (R), lysine (K), histidine (H), or absent; X₃₆ is isoleucine (I), proline (P), or threonine (T); X₃₇ is serine (S) or glycine (G); X₃₈ is glycine (G), phenylalanine (F), or histidine (H); X₃₉ is serine (S), isoleucine (I), leucine (L), valine (V), or tyrosine (Y); X₄₀ is glycine (G), alanine (A), aspartic acid (D), or glutamic acid (E); X₄₁ is threonine (T), glycine (G), arginine (R), lysine (K), or histidine (H); X₄₂ is alanine (A) or serine (S); X₄₃ is glycine (G), threonine (T), or absent; X₄₄ is tyrosine (Y), glutamine (Q), arginine (R), lysine (K), histidine (H), or absent; X₄₅ is tyrosine (Y), leucine (L), glutamic acid (E), aspartic acid (D), or absent; X₄₆ is alanine (A), leucine (L), or absent; X₄₇ is aspartic acid (D), histidine (H), proline (P), or absent; X₄₈ is serine (S) or absent; X₄₉ is valine (V) or absent; X₅₀ is lysine (K) or absent, and/or c) a CDRH3 comprising the amino acid sequence of X₅₁X₅₂X₅₃X₅₄X₅₅X₅₆RX₅₇LX₅₈GX₅₉RX₆₀X₆₁X₆₂DY (SEQ ID NO: 252), wherein X₅₁ is arginine (R), alanine (A), cysteine (C), glutamic acid (E), or serine (S); X₅₂ is valine (V), alanine (A), glycine (G), methionine (M), arginine (R), or serine (S); X₅₃ is serine (S), alanine (A), glutamic acid (E), glycine (G), methionine (M), arginine (R), threonine (T), or valine (V); X₅₄ is leucine (L), lysine (K), arginine (R), serine (S), or valine (V); X₅₅ glutamine (Q) or glycine (G); X₅₆ is arginine (R) or histidine (H); X₅₇ is threonine (T), leucine (L), or methionine (M); X₅₈ is aspartic acid (D) or glutamic acid (E); X₅₉ is tyrosine (Y) or phenylalanine (F); X₆₀ is serine (S) or threonine (T); X₆₁ is serine (S), isoleucine (I), valine (V), or leucine (L); and X₆₂ is phenylalanine (F) or tyrosine (Y).

In certain embodiments, the instant disclosure provides an antibody that specifically binds to APJ (e.g., human APJ), the antibody comprising a) a CDRH1 comprising the amino acid sequence of X₂₅X₂₆X₂₇X₂₈X₂₉X₃₀X₃₁X₃₂X₃₃X₃₄ (SEQ ID NO: 250), wherein X₂₅ is glycine (G) or glutamine (Q); X₂₆ is phenylalanine (F), glutamine (Q), or valine (V); X₂₇ is threonine (T), alanine (A), aspartic acid (D), histidine (H), proline (P), valine (V), arginine (R), lysine (K), or glutamic acid (E); X₂₈ is phenylalanine (F), glycine (G), histidine (H), isoleucine (I), or valine (V); X₂₉ is serine (S), proline (P), arginine (R), or lysine (K); X₃₀ is serine (S) or proline (P); X₃₁ is proline (P) or tyrosine (Y); X₃₂ is histidine (H), alanine (A), proline (P), arginine (R), or lysine (K); X₃₃ is methionine (M) or absent; and X₃₄ is glycine (G), arginine (R), lysine (K), histidine (H), or absent, b) a CDRH2 comprising the amino acid sequence of X₃₅X₃₆X₃₇X₃₈X₃₉X₄₀X₄₁X₄₂X₄₃X₄₄X₄₅X₄₆X₄₇X₄₈X₄₉X₅₀ (SEQ ID NO: 251), wherein X₃₅ is alanine (A), glycine (G), serine (S), valine (V), arginine (R), lysine (K), histidine (H), or absent; X₃₆ is isoleucine (I), proline (P), or threonine (T); X₃₇ is serine (S) or glycine (G); X₃₈ is glycine (G), phenylalanine (F), or histidine (H); X₃₉ is serine (S), isoleucine (I), leucine (L), valine (V), or tyrosine (Y); X₄₀ is glycine (G), alanine (A), aspartic acid (D), or glutamic acid (E); X₄₁ is threonine (T), glycine (G), arginine (R), lysine (K), or histidine (H); X₄₂ is alanine (A) or serine (S); X₄₃ is glycine (G), threonine (T), or absent; X₄₄ is tyrosine (Y), glutamine (Q), arginine (R), lysine (K), histidine (H), or absent; X₄₅ is tyrosine (Y), leucine (L), glutamic acid (E), aspartic acid (D), or absent; X₄₆ is alanine (A), leucine (L), or absent; X₄₇ is aspartic acid (D), histidine (H), proline (P), or absent; X₄₈ is serine (S) or absent; X₄₉ is valine (V) or absent; X₅₀ is lysine (K) or absent, and c) a CDRH3 comprising the amino acid sequence of X₅₁X₅₂X₅₃X₅₄X₅₅X₅₆RX₅₇LX₅₈GX₅₉RX₆₀X₆₁X₆₂DY (SEQ ID NO: 252), wherein X₅₁ is arginine (R), alanine (A), cysteine (C), glutamic acid (E), or serine (S); X₅₂ is valine (V), alanine (A), glycine (G), methionine (M), arginine (R), or serine (S); X₅₃ is serine (S), alanine (A), glutamic acid (E), glycine (G), methionine (M), arginine (R), threonine (T), or valine (V); X₅₄ is leucine (L), lysine (K), arginine (R), serine (S), or valine (V); X₅₅ glutamine (Q) or glycine (G); X₅₆ is arginine (R) or histidine (H); X₅₇ is threonine (T), leucine (L), or methionine (M); X₅₈ is aspartic acid (D) or glutamic acid (E); X₅₉ is tyrosine (Y) or phenylalanine (F); X₆₀ is serine (S) or threonine (T); X₆₁ is serine (S), isoleucine (I), valine (V), or leucine (L); and X₆₂ is phenylalanine (F) or tyrosine (Y).

In certain embodiments, X₂₅ is glycine (G) or glutamine (Q); X₂₆ is phenylalanine (F), glutamine (Q), or valine (V); X₂₇ is threonine (T), alanine (A), aspartic acid (D), histidine (H), proline (P), or valine (V); X₂₈ is phenylalanine (F), glycine (G), histidine (H), or isoleucine (I); X₂₉ is serine (S) or proline (P); X₃₀ is serine (S) or proline (P); X₃₁ is proline (P) or tyrosine (Y); X₃₂ is histidine (H), alanine (A), or proline (P); X₃₃ is methionine (M) or absent; X₃₄ is glycine (G) or absent; X₃₅ is alanine (A), glycine (G), serine (S), valine (V), or absent; X₃₆ is isoleucine (I), proline (P), or threonine (T); X₃₇ is serine (S) or glycine (G); X₃₈ is glycine (G), phenylalanine (F), or histidine (H); X₃₉ is serine (S), isoleucine (I), leucine (L), valine (V), or tyrosine (Y); X₄₀ is glycine (G), alanine (A), aspartic acid (D), or glutamic acid (E); X₄₁ is threonine (T) or glycine (G); X₄₂ is alanine (A) or serine (S); X₄₃ is glycine (G), threonine (T), or absent; X₄₄ is tyrosine (Y), glutamine (Q), or absent; X₄₅ is tyrosine (Y), leucine (L), or absent; X₄₆ is alanine (A), leucine (L), or absent; X₄₇ is aspartic acid (D) or absent; X₄₈ is serine (S) or absent; X₄₉ is valine (V) or absent; X₅₀ is lysine (K) or absent; X₅₁ is arginine (R), alanine (A), cysteine (C), glutamic acid (E), or serine (S); X₅₂ is valine (V), alanine (A), glycine (G), methionine (M), arginine (R), or serine (S); X₅₃ is serine (S), alanine (A), glutamic acid (E), glycine (G), methionine (M), arginine (R), threonine (T), or valine (V); X₅₄ is leucine (L), lysine (K), arginine (R), serine (S), or valine (V); X₅₅ is glutamine (Q); X₅₆ is arginine (R) or histidine (H); X₅₇ is threonine (T); X₅₈ is aspartic acid (D); X₅₉ is tyrosine (Y) or phenylalanine (F); X₆₀ is serine (S) or threonine (T); X₆₁ is serine (S), isoleucine (I), or valine (V); and X₆₂ is phenylalanine (F) or tyrosine (Y).

In certain embodiments, X₃₃, X₃₄, X₃₅, X₄₃, X₄₄, X₄₅, X₄₆, X₄₇, X₄₈, X₄₉, and X₅₀ are absent.

In certain embodiments, X₃₃ is methionine (M); X₃₄ is glycine (G); X₃₅ is alanine (A), glycine (G), serine (S), or valine (V); X₄₃ is glycine (G) or threonine (T); X₄₄ is tyrosine (Y) or glutamine (Q); X₄₅ is tyrosine (Y) or leucine (L); and X₄₆, X₄₇, X₄₈, X₄₉, and X₅₀ are absent.

In certain embodiments, X₃₃ is methionine (M); X₃₄ is glycine (G); X₃₅ is alanine (A), glycine (G), serine (S), or valine (V); X₄₃ is glycine (G) or threonine (T); X₄₄ is tyrosine (Y) or glutamine (Q); X₄₅ is tyrosine (Y) or leucine (L); X₄₆ is alanine (A) or leucine (L); X₄₇ is aspartic acid (D); X₄₈ is serine (S); X₄₉ is valine (V); and X₅₀ is lysine (K).

In certain embodiments, the VH of an anti-APJ antibody provided herein does not comprise the amino acid sequence set forth in any one of SEQ ID NOs: 60-64 and 823-830.

The CDRH amino acid sequences of exemplary anti-APJ antibodies provided herein are set forth in Tables 3-7.

TABLE 3
CDRH amino acid sequences of exemplary anti-APJ antibodies.

| | | SEQ | | SEQ | | SEQ |
|---|---|---|---|---|---|---|
| | | ID | | ID | | ID |
| Ab | CDRH1 | NO | CDRH2 | NO | CDRH3 | NO |
| Ab001 | GSTYSSHC | 354 | MTRSRGT | 355 | AAVPRAGIESGAYCKWNMKDSGS | 87 |
| Ab002 | GSTYSSHC | 354 | MTRSRGT | 355 | AAVPRAGIESGAYCKWIIKDSGS | 90 |
| Ab003 | GSTYSSHC | 354 | MTHSRGT | 357 | AAVPRAGIESGAYCKWRYKDSGS | 93 |
| Ab004 | GSTYSSHC | 354 | MTHSRGT | 357 | AAVPRAGIESGAYCKWRYQDSGS | 96 |
| Ab005 | GSTYSSHC | 354 | MTRSRGT | 355 | AAVPRAGIESGAYCKWRYQDSGS | 96 |
| Ab006 | GSTYSSHC | 354 | MTRSRGT | 355 | AAVPRAGIESGAYCKWIIQDSGS | 99 |
| Ab007 | GSTYSSHC | 354 | MTHSRGT | 357 | AAVPRAGIESGAYCKWIIQDSGS | 99 |
| Ab008 | GSTYSSHC | 354 | MTRSRGT | 355 | AAVPRAGIESGAYCKWPRKDSGS | 102 |
| Ab009 | GSTYSSHC | 354 | MTRSYGT | 359 | AAVPRAGIESGAYCKWPRKDSGS | 102 |
| Ab010 | GSTYSSHC | 354 | MTRSYGT | 359 | AAVPRAGIESGAYCKWPRQDSGS | 105 |
| Ab011 | GSTYSSHC | 354 | MTRSRGT | 355 | AAVPRAGIESGAYCKWPRQDSGS | 105 |
| Ab012 | GSTYSSHC | 354 | MTHSRGT | 357 | AAVPRAGIESGAYCKWPRQDSGS | 105 |
| Ab013 | GSTYSSHC | 354 | MTRSRGT | 355 | AAVPRAGIESGAYCKWRLQDSGS | 108 |
| Ab014 | GSTYSSHC | 354 | MTHSRGT | 357 | AAVPRAGIESGAYCKWRLQDSGS | 108 |
| Ab015 | GSTYSSHC | 354 | MTHSRGT | 357 | AAVPRAGIESGAYCKWPHKDSGS | 111 |
| Ab016 | GSTYSSHC | 354 | MTHSRGT | 357 | AAVPRAGIESGAYCKWPHQDSGS | 114 |
| Ab017 | GSTYSSHC | 354 | MTRSRGT | 355 | AAVPRAGIESGAYCKWPHQDSGS | 114 |
| Ab018 | GSTYSSHC | 354 | MTHSRGT | 357 | AAVPRAGIESGAYCKWHGKDSGS | 117 |
| Ab019 | GSTYSSHC | 354 | MTRSRGT | 355 | AAVPRAGIESGAYCKWHGKDSGS | 117 |
| Ab020 | GSTYSSHC | 354 | MTHSRGT | 357 | AAVPRAGIESGAYCKWHNKDSGS | 120 |
| Ab021 | GSTYSSHC | 354 | MTHSRGT | 357 | AAVPRAGIESGAYCKWHYKDSGS | 123 |
| Ab022 | GSTYSSHC | 354 | MTHSRGT | 357 | AAVPRAGIESGAYCKWKNKDSGS | 125 |
| Ab023 | GSTYSSHC | 354 | MTHSRGT | 357 | AAVPRAGIESGAYCKWKYKDSGS | 127 |
| Ab024 | GSTYSSHC | 354 | MTHSRGT | 357 | AAVPRAGIESGAYCKWNNKDSGS | 129 |
| Ab025 | GSTYSSHC | 354 | MTHSRGT | 357 | AAVPRAGIESGAYCKWNYKDSGS | 131 |
| Ab026 | GSTYSSHC | 354 | MTHSRGT | 357 | AAVPRAGIESGAYCKWQNKDSGS | 133 |
| Ab027 | GSTYSSHC | 354 | MTHSRGT | 357 | AAVPRAGIESGAYCKWQYKDSGS | 135 |
| Ab028 | GSTYSSHC | 354 | MTHSRGT | 357 | AAVPRAGIESGAYCKWRNKDSGS | 137 |
| Ab029 | GSTYSSHC | 354 | MTHSRGT | 357 | AAVPRAGIESGAYCKWSNKDSGS | 139 |
| Ab030 | GSTYSSHC | 354 | MTHSRGT | 357 | AAVPRAGIESGAYCKWSYKDSGS | 141 |
| Ab031 | GSTYSSHC | 354 | MTRSRGT | 355 | AAVPRAGIESGAYCKWIHKDSGS | 143 |
| Ab032 | GSTYSSHC | 354 | MTRSRGT | 355 | AAVPRAGIESGAYCKWILKDSGS | 145 |
| Ab033 | GSTYSSHC | 354 | MTRSRGT | 355 | AAVPRAGIESGAYCKWINKDSGS | 147 |
| Ab034 | GSTYSSHC | 354 | MTRSRGT | 355 | AAVPRAGIESGAYCKWTHKDSGS | 149 |
| Ab035 | GSTYSSHC | 354 | MTRSRGT | 355 | AAVPRAGIESGAYCKWTIKDSGS | 151 |
| Ab036 | GSTYSSHC | 354 | MTRSRGT | 355 | AAVPRAGIESGAYCKWTLKDSGS | 152 |
| Ab037 | GSTYSSHC | 354 | MTRSRGT | 355 | AAVPRAGIESGAYCKWTNKDSGS | 153 |
| Ab038 | GYNYVFHC | 356 | MSRSRGY | 361 | AAVPRAGIESGAYCKWHGKDSGS | 117 |
| Ab039 | GITYVSHC | 358 | MSRSRGY | 361 | AAVPRAGIESGAYCKWHGKDSGS | 117 |
| Ab040 | GITYSSHC | 360 | MQRSRGT | 363 | AAVPRAGIESGAYCKWHGKDSGS | 117 |
| Ab041 | GITYQSHC | 362 | IQRSRGT | 365 | AAVPRAGIESGAYCKWHGKDSGS | 117 |
| Ab042 | GLHYHSHC | 364 | MSHSRGY | 367 | AAVPRAGIESGAYCKWQNKDSGS | 133 |
| Ab043 | GIHYSSHC | 366 | MSHSRGY | 367 | AAVPRAGIESGAYCKWQNKDSGS | 133 |
| Ab044 | GFTYQSHC | 368 | MQHSRGT | 369 | AAVPRAGIESGAYCKWQNKDSGS | 133 |
| Ab045 | GFLYSFHC | 370 | ITHSRGY | 371 | AAVPRAGIESGAYCKWQNKDSGS | 133 |
| Ab046 | GSTYSSHC | 354 | MTHSRGY | 373 | AAVPRAGIESGAYCKWRLQDSGS | 108 |
| Ab047 | GSTYSSHC | 354 | MTHSRGT | 357 | AAVPRAGIESGAYCKWRLQDSGS | 108 |
| Ab048 | GSTYSSHC | 354 | MTRSRGY | 375 | AAVPRAGIESGAYCKWHGKDSGS | 117 |
| Ab049 | GSTYSSHC | 354 | MTRSRGT | 355 | AAVPRAGIESGAYCKWHGKDSGS | 117 |
| Ab050 | GSTYSSHC | 354 | MTHSRGY | 373 | AAVPRAGIESGAYCKWHNKDSGS | 120 |
| Ab051 | GSTYSSHC | 354 | MTHSRGT | 357 | AAVPRAGIESGAYCKWHNKDSGS | 120 |
| Ab052 | GSTYSSHC | 354 | MTHSRGY | 373 | AAVPRAGIESGAYCKWNNKDSGS | 129 |
| Ab053 | GSTYSSHC | 354 | MTHSRGT | 357 | AAVPRAGIESGAYCKWNNKDSGS | 129 |
| Ab054 | GSTYSSHC | 354 | MTHSRGY | 373 | AAVPRAGIESGAYCKWNYKDSGS | 131 |
| Ab055 | GSTYSSHC | 354 | MTHSRGT | 357 | AAVPRAGIESGAYCKWNYKDSGS | 131 |
| Ab056 | GSTYSSHC | 354 | MTHSRGY | 373 | AAVPRAGIESGAYCKWQNKDSGS | 133 |
| Ab057 | GSTYSSHC | 354 | MTHSRGT | 357 | AAVPRAGIESGAYCKWQNKDSGS | 133 |
| Ab058 | GSTYSSHC | 354 | MTHSRGY | 373 | AAVPRAGIESGAYCKWSNKDSGS | 139 |
| Ab059 | GSTYSSHC | 354 | MTHSRGT | 357 | AAVPRAGIESGAYCKWSNKDSGS | 139 |
| Ab060 | GSTYSSHC | 354 | MTRSRGT | 355 | AAVPRAGIEYSGAYCKWNMKDSGS | 154 |
| Ab061 | GSTYSSHC | 354 | MTRSRGT | 355 | AAVPRAGIESGAYCKWNMKDSGS | 87 |
| Ab062 | GSTYASHC | 372 | MTRSRGT | 355 | AAVPRAGIESGAYCKWNMKDSGS | 87 |
| Ab063 | GSTYSSHC | 354 | MARSRGT | 377 | AAVPRAGIESGAYCKWNMKDSGS | 87 |
| Ab064 | GSTYSSHC | 354 | MTRSRGT | 355 | AAVPRAGIESGAYCKANMKDSGS | 155 |
| Ab065 | GSTYSSHC | 354 | MTRSRGT | 355 | AAVPRAGIEYSGAYCKWNMKDSGS | 154 |
| Ab066 | GSTYASHC | 372 | MTRSRGT | 355 | AAVPRAGIESGAYCKWNMKDSGS | 87 |
| Ab067 | GSTYSSHC | 354 | MARSRGT | 377 | AAVPRAGIESGAYCKWNMKDSGS | 87 |
| Ab068 | GSTYSSHC | 354 | MTRSRGT | 355 | AAVPRAGIESGAYCKANMKDSGS | 155 |
| Ab069 | GSTYSSHC | 354 | MTHSRGY | 373 | AAVPRAGIESGAYCKWSYKDSGS | 141 |
| Ab070 | GSTYSSHC | 354 | MTHSRGT | 357 | AAVPRAGIESGAYCKWSYKDSGS | 141 |
| Ab071 | GLTFSSHC | 374 | IQGSRGS | 379 | AAVPRAGIESGAYCKWSYKDSGS | 141 |
| Ab072 | GLTFSSHC | 374 | IQHSRGS | 380 | AAVPRAGIESGAYCKWSYKDSGS | 141 |
| Ab073 | GLTFSSHC | 374 | IQHSRGY | 381 | AAVPRAGIESGAYCKWSYKDSGS | 141 |
| Ab074 | GLTFSSHC | 374 | IQRSRGY | 382 | AAVPRAGIESGAYCKWSYKDSGS | 141 |
| Ab075 | GLTFSSHC | 374 | ISHSRGY | 383 | AAVPRAGIESGAYCKWSYKDSGS | 141 |
| Ab076 | GLTFSSHC | 374 | ISGSRGY | 384 | AAVPRAGIESGAYCKWSYKDSGS | 141 |
| Ab077 | GLTFSSHC | 374 | ISHSRGS | 385 | AAVPRAGIESGAYCKWNYKDSGS | 131 |
| Ab078 | GLTFSSHC | 374 | IQHSRGS | 380 | AAVPRAGIESGAYCKWNYKDSGS | 131 |
| Ab079 | GLTFSSHC | 374 | IQGSRGS | 379 | AAVPRAGIESGAYCKWNYKDSGS | 131 |
| Ab080 | GFTFSSHC | 376 | ISHSRGS | 385 | AAVPRAGIESGAYCKWNYKDSGS | 131 |
| Ab081 | GFTFSSHC | 376 | IQHSRGS | 380 | AAVPRAGIESGAYCKWNYKDSGS | 131 |
| Ab082 | GFTFSSHC | 376 | IQGSRGS | 379 | AAVPRAGIESGAYCKWNYKDSGS | 131 |
| Ab083 | GFTFSSYC | 378 | ISHSRGS | 385 | AAVPRAGIESGAYCKWNYKDSGS | 131 |
| Ab084 | GFTFSSYC | 378 | IQGSRGS | 379 | AAVPRAGIESGAYCKWNYKDSGS | 131 |

TABLE 4
CDRH amino acid sequences of exemplary anti-APJ antibodies.

| | | SEQ | | SEQ | | SEQ |
|---|---|---|---|---|---|---|
| | | ID | | ID | | ID |
| Ab | CDRH1 | NO | CDRH2 | NO | CDRH3 | NO |
| Ab001 | GSTYSSHCMG | 85 | LMTRSRGTSY | 86 | AAVPRAGIESGAYCKWN | 87 |
| | | | | | MKDSGS | |
| Ab002 | GSTYSSHCMG | 85 | LMTRSRGTSY | 86 | AAVPRAGIESGAYCKWI | 90 |
| | | | | | IKDSGS | |
| Ab003 | GSTYSSHCMG | 85 | LMTHSRGTSY | 89 | AAVPRAGIESGAYCKWR | 93 |
| | | | | | YKDSGS | |
| Ab004 | GSTYSSHCMG | 85 | LMTHSRGTSY | 89 | AAVPRAGIESGAYCKWR | 96 |
| | | | | | YQDSGS | |
| Ab005 | GSTYSSHCMG | 85 | LMTRSRGTSY | 86 | AAVPRAGIESGAYCKWR | 96 |
| | | | | | YQDSGS | |
| Ab006 | GSTYSSHCMG | 85 | LMTRSRGTSY | 86 | AAVPRAGIESGAYCKWI | 99 |
| | | | | | IQDSGS | |
| Ab007 | GSTYSSHCMG | 85 | LMTHSRGTSY | 89 | AAVPRAGIESGAYCKWI | 99 |
| | | | | | IQDSGS | |
| Ab008 | GSTYSSHCMG | 85 | LMTRSRGTSY | 86 | AAVPRAGIESGAYCKWP | 102 |
| | | | | | RKDSGS | |
| Ab009 | GSTYSSHCMG | 85 | LMTRSYGTSY | 92 | AAVPRAGIESGAYCKWP | 102 |
| | | | | | RKDSGS | |
| Ab010 | GSTYSSHCMG | 85 | LMTRSYGTSY | 92 | AAVPRAGIESGAYCKWP | 105 |
| | | | | | RQDSGS | |
| Ab011 | GSTYSSHCMG | 85 | LMTRSRGTSY | 86 | AAVPRAGIESGAYCKWP | 105 |
| | | | | | RQDSGS | |
| Ab012 | GSTYSSHCMG | 85 | LMTHSRGTSY | 89 | AAVPRAGIESGAYCKWP | 105 |
| | | | | | RQDSGS | |
| Ab013 | GSTYSSHCMG | 85 | LMTRSRGTSY | 86 | AAVPRAGIESGAYCKWR | 108 |
| | | | | | LQDSGS | |
| Ab014 | GSTYSSHCMG | 85 | LMTHSRGTSY | 89 | AAVPRAGIESGAYCKWR | 108 |
| | | | | | LQDSGS | |
| Ab015 | GSTYSSHCMG | 85 | LMTHSRGTSY | 89 | AAVPRAGIESGAYCKWP | 111 |
| | | | | | HKDSGS | |
| Ab016 | GSTYSSHCMG | 85 | LMTHSRGTSY | 89 | AAVPRAGIESGAYCKWP | 114 |
| | | | | | HQDSGS | |
| Ab017 | GSTYSSHCMG | 85 | LMTRSRGTSY | 86 | AAVPRAGIESGAYCKWP | 114 |
| | | | | | HQDSGS | |
| Ab018 | GSTYSSHCMG | 85 | LMTHSRGTSY | 89 | AAVPRAGIESGAYCKWH | 117 |
| | | | | | GKDSGS | |
| Ab019 | GSTYSSHCMG | 85 | LMTRSRGTSY | 86 | AAVPRAGIESGAYCKWH | 117 |
| | | | | | GKDSGS | |
| Ab020 | GSTYSSHCMG | 85 | LMTHSRGTSY | 89 | AAVPRAGIESGAYCKWH | 120 |
| | | | | | NKDSGS | |
| Ab021 | GSTYSSHCMG | 85 | LMTHSRGTSY | 89 | AAVPRAGIESGAYCKWH | 123 |
| | | | | | YKDSGS | |
| Ab022 | GSTYSSHCMG | 85 | LMTHSRGTSY | 89 | AAVPRAGIESGAYCKWK | 125 |
| | | | | | NKDSGS | |
| Ab023 | GSTYSSHCMG | 85 | LMTHSRGTSY | 89 | AAVPRAGIESGAYCKWK | 127 |
| | | | | | YKDSGS | |
| Ab024 | GSTYSSHCMG | 85 | LMTHSRGTSY | 89 | AAVPRAGIESGAYCKWN | 129 |
| | | | | | NKDSGS | |
| Ab025 | GSTYSSHCMG | 85 | LMTHSRGTSY | 89 | AAVPRAGIESGAYCKWN | 131 |
| | | | | | YKDSGS | |
| Ab026 | GSTYSSHCMG | 85 | LMTHSRGTSY | 89 | AAVPRAGIESGAYCKWQ | 133 |
| | | | | | NKDSGS | |
| Ab027 | GSTYSSHCMG | 85 | LMTHSRGTSY | 89 | AAVPRAGIESGAYCKWQ | 135 |
| | | | | | YKDSGS | |
| Ab028 | GSTYSSHCMG | 85 | LMTHSRGTSY | 89 | AAVPRAGIESGAYCKWR | 137 |
| | | | | | NKDSGS | |
| Ab029 | GSTYSSHCMG | 85 | LMTHSRGTSY | 89 | AAVPRAGIESGAYCKWS | 139 |
| | | | | | NKDSGS | |
| Ab030 | GSTYSSHCMG | 85 | LMTHSRGTSY | 89 | AAVPRAGIESGAYCKWS | 141 |
| | | | | | YKDSGS | |
| Ab031 | GSTYSSHCMG | 85 | LMTRSRGTSY | 86 | AAVPRAGIESGAYCKWI | 143 |
| | | | | | HKDSGS | |
| Ab032 | GSTYSSHCMG | 85 | LMTRSRGTSY | 86 | AAVPRAGIESGAYCKWI | 145 |
| | | | | | LKDSGS | |
| Ab033 | GSTYSSHCMG | 85 | LMTRSRGTSY | 86 | AAVPRAGIESGAYCKWI | 147 |
| | | | | | NKDSGS | |
| Ab034 | GSTYSSHCMG | 85 | LMTRSRGTSY | 86 | AAVPRAGIESGAYCKWT | 149 |
| | | | | | HKDSGS | |
| Ab035 | GSTYSSHCMG | 85 | LMTRSRGTSY | 86 | AAVPRAGIESGAYCKWT | 151 |
| | | | | | IKDSGS | |
| Ab036 | GSTYSSHCMG | 85 | LMTRSRGTSY | 86 | AAVPRAGIESGAYCKWT | 152 |
| | | | | | LKDSGS | |
| Ab037 | GSTYSSHCMG | 85 | LMTRSRGTSY | 86 | AAVPRAGIESGAYCKWT | 153 |
| | | | | | NKDSGS | |
| Ab038 | GYNYVFHCMG | 88 | LMSRSRGYYY | 95 | AAVPRAGIESGAYCKWH | 117 |
| | | | | | GKDSGS | |
| Ab039 | GITYVSHCMG | 91 | AMSRSRGYYY | 98 | AAVPRAGIESGAYCKWH | 117 |
| | | | | | GKDSGS | |
| Ab040 | GITYSSHCMG | 94 | AMQRSRGTYY | 101 | AAVPRAGIESGAYCKWH | 117 |
| | | | | | GKDSGS | |
| Ab041 | GITYQSHCMG | 97 | LIQRSRGTYY | 104 | AAVPRAGIESGAYCKWH | 117 |
| | | | | | GKDSGS | |
| Ab042 | GLHYHSHCMG | 100 | AMSHSRGYYY | 107 | AAVPRAGIESGAYCKWQ | 133 |
| | | | | | NKDSGS | |
| Ab043 | GIHYSSHCMG | 103 | LMSHSRGYYY | 110 | AAVPRAGIESGAYCKWQ | 133 |
| | | | | | NKDSGS | |
| Ab044 | GFTYQSHCMG | 106 | LMQHSRGTYY | 113 | AAVPRAGIESGAYCKWQ | 133 |
| | | | | | NKDSGS | |
| Ab045 | GFLYSFHCMG | 109 | LITHSRGYSY | 116 | AAVPRAGIESGAYCKWQ | 133 |
| | | | | | NKDSGS | |
| Ab046 | GSTYSSHCMG | 85 | LMTHSRGYYY | 119 | AAVPRAGIESGAYCKWR | 108 |
| | | | | | LQDSGS | |
| Ab047 | GSTYSSHCMG | 85 | LMTHSRGTYY | 122 | AAVPRAGIESGAYCKWR | 108 |
| | | | | | LQDSGS | |
| Ab048 | GSTYSSHCMG | 85 | LMTRSRGYYY | 124 | AAVPRAGIESGAYCKWH | 117 |
| | | | | | GKDSGS | |
| Ab049 | GSTYSSHCMG | 85 | LMTRSRGTYY | 126 | AAVPRAGIESGAYCKWH | 117 |
| | | | | | GKDSGS | |
| Ab050 | GSTYSSHCMG | 85 | LMTHSRGYYY | 119 | AAVPRAGIESGAYCKWH | 120 |
| | | | | | NKDSGS | |
| Ab051 | GSTYSSHCMG | 85 | LMTHSRGTYY | 122 | AAVPRAGIESGAYCKWH | 120 |
| | | | | | NKDSGS | |
| Ab052 | GSTYSSHCMG | 85 | LMTHSRGYYY | 119 | AAVPRAGIESGAYCKWN | 129 |
| | | | | | NKDSGS | |
| Ab053 | GSTYSSHCMG | 85 | LMTHSRGTYY | 122 | AAVPRAGIESGAYCKWN | 129 |
| | | | | | NKDSGS | |
| Ab054 | GSTYSSHCMG | 85 | LMTHSRGYYY | 119 | AAVPRAGIESGAYCKWN | 131 |
| | | | | | YKDSGS | |
| Ab055 | GSTYSSHCMG | 85 | LMTHSRGTYY | 122 | AAVPRAGIESGAYCKWN | 131 |
| | | | | | YKDSGS | |
| Ab056 | GSTYSSHCMG | 85 | LMTHSRGYYY | 119 | AAVPRAGIESGAYCKWQ | 133 |
| | | | | | NKDSGS | |
| Ab057 | GSTYSSHCMG | 85 | LMTHSRGTYY | 122 | AAVPRAGIESGAYCKWQ | 133 |
| | | | | | NKDSGS | |
| Ab058 | GSTYSSHCMG | 85 | LMTHSRGYYY | 119 | AAVPRAGIESGAYCKWS | 139 |
| | | | | | NKDSGS | |
| Ab059 | GSTYSSHCMG | 85 | LMTHSRGTYY | 122 | AAVPRAGIESGAYCKWS | 139 |
| | | | | | NKDSGS | |
| Ab060 | GSTYSSHCMG | 85 | LMTRSRGTSY | 86 | AAVPRAGIEYSGAYCKW | 154 |
| | | | | | NMKDSGS | |
| Ab061 | GSTYSSHCMG | 85 | LMTRSRGTSY | 86 | AAVPRAGIESGAYCKWN | 87 |
| | | | | | MKDSGS | |
| Ab062 | GSTYASHCMG | 112 | LMTRSRGTSY | 86 | AAVPRAGIESGAYCKWN | 87 |
| | | | | | MKDSGS | |
| Ab063 | GSTYSSHCMG | 85 | LMARSRGTSY | 128 | AAVPRAGIESGAYCKWN | 87 |
| | | | | | MKDSGS | |
| Ab064 | GSTYSSHCMG | 85 | LMTRSRGTSY | 86 | AAVPRAGIESGAYCKAN | 155 |
| | | | | | MKDSGS | |
| Ab065 | GSTYSSHCMG | 85 | LMTRSRGTSY | 86 | AAVPRAGIEYSGAYCKW | 154 |
| | | | | | NMKDSGS | |
| Ab066 | GSTYASHCMG | 112 | LMTRSRGTSY | 86 | AAVPRAGIESGAYCKWN | 87 |
| | | | | | MKDSGS | |
| Ab067 | GSTYSSHCMG | 85 | LMARSRGTSY | 128 | AAVPRAGIESGAYCKWN | 87 |
| | | | | | MKDSGS | |
| Ab068 | GSTYSSHCMG | 85 | LMTRSRGTSY | 86 | AAVPRAGIESGAYCKAN | 155 |
| | | | | | MKDSGS | |
| Ab069 | GSTYSSHCMG | 85 | LMTHSRGYSY | 130 | AAVPRAGIESGAYCKWS | 141 |
| | | | | | YKDSGS | |
| Ab070 | GSTYSSHCMG | 85 | LMTHSRGTSYY | 132 | AAVPRAGIESGAYCKWS | 141 |
| | | | | | YKDSGS | |
| Ab071 | GLTFSSHCMG | 115 | AIQGSRGSTYY | 134 | AAVPRAGIESGAYCKWS | 141 |
| | | | | | YKDSGS | |
| Ab072 | GLTFSSHCMG | 115 | AIQHSRGSTYY | 136 | AAVPRAGIESGAYCKWS | 141 |
| | | | | | YKDSGS | |
| Ab073 | GLIFSSHCMG | 115 | AIQHSRGYSY | 138 | AAVPRAGIESGAYCKWS | 141 |
| | | | | | YKDSGS | |
| Ab074 | GLTFSSHCMG | 115 | AIQRSRGYSY | 140 | AAVPRAGIESGAYCKWS | 141 |
| | | | | | YKDSGS | |
| Ab075 | GLTFSSHCMG | 115 | AISHSRGYSY | 142 | AAVPRAGIESGAYCKWS | 141 |
| | | | | | YKDSGS | |
| Ab076 | GLTFSSHCMG | 115 | AISGSRGYSY | 144 | AAVPRAGIESGAYCKWS | 141 |
| | | | | | YKDSGS | |
| Ab077 | GLTFSSHCMG | 115 | AISHSRGSSY | 146 | AAVPRAGIESGAYCKWN | 131 |
| | | | | | YKDSGS | |
| Ab078 | GLTFSSHCMG | 115 | AIQHSRGSSY | 148 | AAVPRAGIESGAYCKWN | 131 |
| | | | | | YKDSGS | |
| Ab079 | GLIFSSHCMG | 115 | AIQGSRGSSY | 150 | AAVPRAGIESGAYCKWN | 131 |
| | | | | | YKDSGS | |
| Ab080 | GFTFSSHCMG | 118 | AISHSRGSSY | 146 | AAVPRAGIESGAYCKWN | 131 |
| | | | | | YKDSGS | |
| Ab081 | GFTFSSHCMG | 118 | AIQHSRGSSY | 148 | AAVPRAGIESGAYCKWN | 131 |
| | | | | | YKDSGS | |
| Ab082 | GFTFSSHCMG | 118 | AIQGSRGSSY | 150 | AAVPRAGIESGAYCKWN | 131 |
| | | | | | YKDSGS | |
| Ab083 | GFTFSSYCMG | 121 | AISHSRGSSY | 146 | AAVPRAGIESGAYCKWN | 131 |
| | | | | | YKDSGS | |
| Ab084 | GFTFSSYCMG | 121 | AIQGSRGSSY | 150 | AAVPRAGIESGAYCKWN | 131 |
| | | | | | YKDSGS | |

TABLE 5
CDRH amino acid sequences of exemplary anti-APJ antibodies.

| | | SEQ | | SEQ | | SEQ |
|---|---|---|---|---|---|---|
| Ab | CDRH1 | ID NO | CDRH2 | ID NO | CDRH3 | ID NO |
| Ab085 | GFTFSSYA | 427 | ISGSGGS | 428 | ARTVQRRTLDGYRSSFDY | 194 |
| Ab086 | GFTFSSYA | 427 | ISGSGGS | 428 | RSRKQRRTLDGYRSSFDY | 197 |
| Ab087 | GFTFSSPH | 429 | ISGSGTA | 430 | RSVLQRRTLDGYRTIFDY | 200 |
| Ab088 | GFTFSSPH | 429 | ISGSGTA | 430 | RSVLQRRTLDGFRTIFDY | 203 |
| Ab089 | GFTFSSPH | 429 | ISGSGTA | 430 | RSVLQRRTLDGYRTVYDY | 206 |
| Ab090 | GFTFSSPH | 429 | ISGSGTA | 430 | RSVLQRRTLDGFRTVYDY | 209 |
| Ab091 | GFTHSSYA | 431 | ISGSGGS | 428 | RAERQRRTLDGYRTIFDY | 212 |
| Ab092 | GFTHSSYA | 431 | ISGSGGS | 428 | RAERQRRTLDGFRTVYDY | 215 |
| Ab093 | GFHHSSYA | 433 | ISGSGGS | 428 | RMMSQRRTLDGYRTIFDY | 218 |
| Ab094 | GFHHSSYA | 433 | ISGSGGS | 428 | RMMSQRRTLDGFRTIFDY | 221 |
| Ab095 | GFHHSSYA | 433 | ISGSGGS | 428 | RMMSQRRTLDGYRTVYDY | 224 |
| Ab096 | GFHHSSYA | 433 | ISGSGGS | 428 | RMMSQRRTLDGERTVYDY | 227 |
| Ab097 | QQTFSSYA | 435 | TSGSGGS | 432 | RAMRQRRTLDGYRSSEDY | 230 |
| Ab098 | QVTFSSYA | 437 | ISHYDGS | 434 | RAGRORRTLDGYRSSFDY | 232 |
| Ab099 | GFTFPPYA | 439 | ISGSGGS | 428 | RGMKQRRTLDGYRSSFDY | 234 |
| Ab100 | GFHHSSYA | 433 | ISGSGGS | 428 | RMMSQRRTLDGYRSSFDY | 235 |
| Ab101 | GFAISSYA | 441 | ISFIAGS | 436 | CAVKQRRTLDGYRSSFDY | 236 |
| Ab102 | GFAISSYA | 441 | ISFIAGS | 436 | SAVKQRRTLDGYRSSFDY | 237 |
| Ab103 | GFPFSSYA | 443 | ISFLEGS | 438 | RAGKQRRTLDGYRSSFDY | 238 |
| Ab104 | GFDISSYA | 445 | ISFVAGS | 440 | EARRQRRTLDGYRSSFDY | 239 |
| Ab105 | GFVGSSYA | 447 | IGGSGGS | 442 | RVARQRRTLDGYRSSFDY | 240 |
| Ab106 | GFTHSSYA | 431 | ISGSGGS | 428 | RAERQRRTLDGYRSSFDY | 241 |
| Ab107 | GFTHSSYA | 431 | ISGSGGS | 428 | RAERQHRTLDGYRSSFDY | 242 |
| Ab108 | GFTFSSPH | 429 | ISGSGTA | 430 | RVSLQRRTLDGYRSSFDY | 243 |
| Ab109 | GFPHPSYP | 448 | PGGSGGS | 444 | RGARQRRTLDGYRSSFDY | 244 |
| Ab110 | GFHFSSYA | 449 | ISGSGGS | 428 | RMMSQRRTLDGYRTIFDY | 218 |
| Ab111 | GFTFSSYA | 427 | ISGSGGS | 428 | RMMSQRRTLDGYRTIFDY | 218 |
| Ab112 | GFTFSSYA | 427 | ISFSGGS | 446 | RMMSQRRTLDGYRTIFDY | 218 |
| Ab113 | GFTFSSYA | 427 | ISGSGGS | 428 | RMMSQRRTLDGYRTIFDY | 218 |
| Ab114 | GFTFSSYA | 427 | ISFSGGS | 446 | RMMSQRRTLDGYRTIFDY | 218 |
| Ab115 | GETFSSYA | 427 | ISFSGGS | 446 | RMMSQRRTLDGYRTIFDY | 218 |
| Ab116 | GFHHSSYA | 433 | ISGSGGS | 428 | RSMSQRRTLDGYRTIFDY | 245 |
| Ab117 | GFHHSSYA | 433 | ISGSGGS | 428 | RMVSQRRTLDGYRTIFDY | 246 |
| Ab118 | GFHFSSYA | 449 | ISGSGGS | 428 | RSMSQRRTLDGYRTIFDY | 245 |
| Ab119 | GFHFSSYA | 449 | ISGSGGS | 428 | RMVSQRRTLDGYRTIFDY | 246 |
| Ab120 | GFTHSSYA | 431 | ISGSGGS | 428 | RSMSQRRTLDGYRTIFDY | 245 |

TABLE 6
CDRH amino acid sequences of exemplary anti-APJ antibodies.

| | | SEQ | | SEQ | | |
|---|---|---|---|---|---|---|
| | | ID | | ID | | SEQ |
| Ab | CDRH1 | NO | CDRH2 | NO | CDRH3 | ID NO |
| Ab085 | GFTFSSYAM | 192 | AISGSGGSTYY | 833 | ARTVQRRTLDGYRSSFD | 194 |
| | G | | | | Y | |
| Ab086 | GFTFSSYAM | 192 | AISGSGGSTYY | 833 | RSRKQRRTLDGYRSSFD | 197 |
| | G | | | | Y | |
| Ab087 | GFTFSSPHM | 195 | AISGSGTAGYY | 834 | RSVLQRRTLDGYRTIFD | 200 |
| | G | | | | Y | |
| Ab088 | GFTFSSPHM | 195 | AISGSGTAGYY | 834 | RSVLQRRTLDGFRIIFD | 203 |
| | G | | | | Y | |
| Ab089 | GFTFSSPHM | 195 | AISGSGTAGYY | 834 | RSVLQRRTLDGYRTVYD | 206 |
| | G | | | | Y | |
| Ab090 | GFTFSSPHM | 195 | AISGSGTAGYY | 834 | RSVLQRRTLDGFRTVYD | 209 |
| | G | | | | Y | |
| Ab091 | GFTHSSYAM | 198 | VISGSGGSTQL | 835 | RAERQRRTLDGYRTIFD | 212 |
| | G | | | | Y | |
| Ab092 | GFTHSSYAM | 198 | VISGSGGSTQL | 835 | RAERQRRTLDGFRTVYD | 215 |
| | G | | | | Y | |
| Ab093 | GFHHSSYAM | 201 | SISGSGGSTYY | 836 | RMMSQRRTLDGYRTIFD | 218 |
| | G | | | | Y | |
| Ab094 | GFHHSSYAM | 201 | SISGSGGSTYY | 836 | RMMSQRRTLDGERTIFD | 221 |
| | G | | | | Y | |
| Ab095 | GFHHSSYAM | 201 | SISGSGGSTYY | 836 | RMMSQRRTLDGYRTVYD | 224 |
| | G | | | | Y | |
| Ab096 | GFHHSSYAM | 201 | SISGSGGSTYY | 836 | RMMSQRRTLDGFRTVYD | 227 |
| | G | | | | Y | |
| Ab097 | QQTFSSYAM | 204 | STSGSGGSTYY | 837 | RAMRQRRTLDGYRSSFD | 230 |
| | G | | | | Y | |
| Ab098 | QVTFSSYAM | 207 | SISHYDGSTYY | 838 | RAGRQRRTLDGYRSSFD | 232 |
| | G | | | | Y | |
| Ab099 | GFTFPPYAM | 210 | SISGSGGSTYY | 836 | RGMKQRRTLDGYRSSFD | 234 |
| | G | | | | Y | |
| Ab100 | GFHHSSYAM | 201 | SISGSGGSTYY | 836 | RMMSQRRTLDGYRSSFD | 235 |
| | G | | | | Y | |
| Ab101 | GFAISSYAM | 213 | AISFIAGSTYY | 839 | CAVKQRRTLDGYRSSED | 236 |
| | G | | | | Y | |
| Ab102 | GFAISSYAM | 213 | AISFIAGSTYY | 839 | SAVKQRRTLDGYRSSFD | 237 |
| | G | | | | Y | |
| Ab103 | GFPFSSYAM | 216 | AISFLEGSTYY | 840 | RAGKQRRTLDGYRSSFD | 238 |
| | G | | | | Y | |
| Ab104 | GFDISSYAM | 219 | AISFVAGSTYY | 841 | EARRQRRTLDGYRSSFD | 239 |
| | G | | | | Y | |
| Ab105 | GFVGSSYAM | 222 | SIGGSGGSTYY | 842 | RVARQRRTLDGYRSSFD | 240 |
| | G | | | | Y | |
| Ab106 | GFTHSSYAM | 198 | VISGSGGSTQL | 835 | RAERQRRTLDGYRSSFD | 241 |
| | G | | | | Y | |
| Ab107 | GFTHSSYAM | 198 | VISGSGGSTQL | 835 | RAERQHRTLDGYRSSED | 242 |
| | G | | | | Y | |
| Ab108 | GFTFSSPHM | 195 | AISGSGTAGYY | 834 | RVSLQRRTLDGYRSSFD | 243 |
| | G | | | | Y | |
| Ab109 | GFPHPSYPM | 225 | GPGGSGGSTYY | 843 | RGARQRRTLDGYRSSFD | 244 |
| | G | | | | Y | |
| Ab110 | GFHFSSYAM | 228 | SISGSGGSTYY | 836 | RMMSQRRTLDGYRTIFD | 218 |
| | G | | | | Y | |
| Ab111 | GFTESSYAM | 192 | SISGSGGSTYY | 836 | RMMSQRRTLDGYRTIFD | 218 |
| | G | | | | Y | |
| Ab112 | GFTFSSYAM | 192 | SISFSGGSTYY | 844 | RMMSQRRTLDGYRTIFD | 218 |
| | G | | | | Y | |
| Ab113 | GFTFSSYAM | 192 | SISGSGGSTQY | 845 | RMMSQRRTLDGYRTIFD | 218 |
| | G | | | | Y | |
| Ab114 | GFTFSSYAM | 192 | AISFSGGSTQY | 846 | RMMSQRRTLDGYRTIFD | 218 |
| | G | | | | Y | |
| Ab115 | GFTFSSYAM | 192 | AISFSGGSTYY | 847 | RMMSQRRTLDGYRTIFD | 218 |
| | G | | | | Y | |
| Ab116 | GFHHSSYAM | 201 | SISGSGGSTYY | 836 | RSMSQRRTLDGYRTIFD | 245 |
| | G | | | | Y | |
| Ab117 | GFHHSSYAM | 201 | SISGSGGSTYY | 836 | RMVSQRRTLDGYRTIFD | 246 |
| | G | | | | Y | |
| Ab118 | GFHFSSYAM | 228 | SISGSGGSTYY | 836 | RSMSQRRTLDGYRTIFD | 245 |
| | G | | | | Y | |
| Ab119 | GFHFSSYAM | 228 | SISGSGGSTYY | 836 | RMVSQRRTLDGYRTIFD | 246 |
| | G | | | | Y | |
| Ab120 | GFTHSSYAM | 198 | SISGSGGSTYY | 836 | RSMSQRRTLDGYRTIFD | 245 |
| | G | | | | Y | |

TABLE 7
CDRH amino acid sequences of exemplary anti-APJ antibodies.

| | | SEQ | | SEQ | | SEQ |
|---|---|---|---|---|---|---|
| | | ID | | ID | | ID |
| Ab | CDRH1 | NO | CDRH2 | NO | CDRH3 | NO |
| Ab085 | GFTFSSYAMG | 192 | AISGSGGSTYYADSVK | 193 | ARTVQRRTLDG | 194 |
| | | | | | YRSSFDY | |
| Ab086 | GFTFSSYAMG | 192 | AISGSGGSTYYADSVK | 193 | RSRKQRRTLDG | 197 |
| | | | | | YRSSEDY | |
| Ab087 | GFTFSSPHMG | 195 | AISGSGTAGYYADSVK | 196 | RSVLQRRTLDG | 200 |
| | | | | | YRTIFDY | |
| Ab088 | GFTFSSPHMG | 195 | AISGSGTAGYYADSVK | 196 | RSVLQRRTLDG | 203 |
| | | | | | FRTIFDY | |
| Ab089 | GFTFSSPHMG | 195 | AISGSGTAGYYADSVK | 196 | RSVLQRRTLDG | 206 |
| | | | | | YRTVYDY | |
| Ab090 | GFTFSSPHMG | 195 | AISGSGTAGYYADSVK | 196 | RSVLQRRTLDG | 209 |
| | | | | | FRTVYDY | |
| Ab091 | GFTHSSYAMG | 198 | VISGSGGSTQLLDSVK | 199 | RAERQRRTLDG | 212 |
| | | | | | YRTIFDY | |
| Ab092 | GFTHSSYAMG | 198 | VISGSGGSTQLLDSVK | 199 | RAERQRRTLDG | 215 |
| | | | | | FRTVYDY | |
| Ab093 | GFHHSSYAMG | 201 | SISGSGGSTYYADSVK | 202 | RMMSQRRTLDG | 218 |
| | | | | | YRTIFDY | |
| Ab094 | GFHHSSYAMG | 201 | SISGSGGSTYYADSVK | 202 | RMMSQRRTLDG | 221 |
| | | | | | FRTIFDY | |
| Ab095 | GFHHSSYAMG | 201 | SISGSGGSTYYADSVK | 202 | RMMSQRRTLDG | 224 |
| | | | | | YRTVYDY | |
| Ab096 | GFHHSSYAMG | 201 | SISGSGGSTYYADSVK | 202 | RMMSQRRTLDG | 227 |
| | | | | | FRTVYDY | |
| Ab097 | QQTFSSYAMG | 204 | STSGSGGSTYYADSVK | 205 | RAMRQRRTLDG | 230 |
| | | | | | YRSSFDY | |
| Ab098 | QVTESSYAMG | 207 | SISHYDGSTYYADSVK | 208 | RAGRORRTLDG | 232 |
| | | | | | YRSSFDY | |
| Ab099 | GFTFPPYAMG | 210 | SISGSGGSTYYADSVK | 202 | RGMKQRRTLDG | 234 |
| | | | | | YRSSFDY | |
| Ab100 | GFHHSSYAMG | 201 | SISGSGGSTYYADSVK | 202 | RMMSQRRTLDG | 235 |
| | | | | | YRSSFDY | |
| Ab101 | GFAISSYAMG | 213 | AISFIAGSTYYADSVK | 211 | CAVKQRRTLDG | 236 |
| | | | | | YRSSFDY | |
| Ab102 | GFAISSYAMG | 213 | AISFIAGSTYYADSVK | 211 | SAVKQRRTLDG | 237 |
| | | | | | YRSSFDY | |
| Ab103 | GFPFSSYAMG | 216 | AISFLEGSTYYADSVK | 214 | RAGKQRRTLDG | 238 |
| | | | | | YRSSFDY | |
| Ab104 | GFDISSYAMG | 219 | AISFVAGSTYYADSVK | 217 | EARRQRRTLDG | 239 |
| | | | | | YRSSFDY | |
| Ab105 | GFVGSSYAMG | 222 | SIGGSGGSTYYADSVK | 220 | RVARQRRTLDG | 240 |
| | | | | | YRSSFDY | |
| Ab106 | GFTHSSYAMG | 198 | VISGSGGSTQLLDSVK | 199 | RAERQRRTLDG | 241 |
| | | | | | YRSSFDY | |
| Ab107 | GFTHSSYAMG | 198 | VISGSGGSTQLLDSVK | 199 | RAERQHRTLDG | 242 |
| | | | | | YRSSFDY | |
| Ab108 | GFTFSSPHMG | 195 | AISGSGTAGYYADSVK | 196 | RVSLQRRTLDG | 243 |
| | | | | | YRSSFDY | |
| Ab109 | GFPHPSYPMG | 225 | GPGGSGGSTYYADSVK | 223 | RGARQRRTLDG | 244 |
| | | | | | YRSSFDY | |
| Ab110 | GFHFSSYAMG | 228 | SISGSGGSTYYADSVK | 202 | RMMSQRRTLDG | 218 |
| | | | | | YRTIFDY | |
| Ab111 | GFTFSSYAMG | 192 | SISGSGGSTYYADSVK | 202 | RMMSQRRTLDG | 218 |
| | | | | | YRTIFDY | |
| Ab112 | GFTFSSYAMG | 192 | SISFSGGSTYYADSVK | 226 | RMMSQRRTLDG | 218 |
| | | | | | YRTIFDY | |
| Ab113 | GFTFSSYAMG | 192 | SISGSGGSTQYADSVK | 229 | RMMSQRRTLDG | 218 |
| | | | | | YRTIFDY | |
| Ab114 | GFTESSYAMG | 192 | AISFSGGSTQYADSVK | 231 | RMMSQRRTLDG | 218 |
| | | | | | YRTIFDY | |
| Ab115 | GFTFSSYAMG | 192 | AISFSGGSTYYADSVK | 233 | RMMSQRRTLDG | 218 |
| | | | | | YRTIFDY | |
| Ab116 | GFHHSSYAMG | 201 | SISGSGGSTYYADSVK | 202 | RSMSQRRTLDG | 245 |
| | | | | | YRTIFDY | |
| Ab117 | GFHHSSYAMG | 201 | SISGSGGSTYYADSVK | 202 | RMVSQRRTLDG | 246 |
| | | | | | YRTIFDY | |
| Ab118 | GFHFSSYAMG | 228 | SISGSGGSTYYADSVK | 202 | RSMSQRRTLDG | 245 |
| | | | | | YRTIFDY | |
| Ab119 | GFHESSYAMG | 228 | SISGSGGSTYYADSVK | 202 | RMVSQRRTLDG | 246 |
| | | | | | YRTIFDY | |
| Ab120 | GFTHSSYAMG | 198 | SISGSGGSTYYADSVK | 202 | RSMSQRRTLDG | 245 |
| | | | | | YRTIFDY | |

In certain embodiments, the instant disclosure provides an antibody that specifically binds to APJ (e.g., human APJ), wherein the antibody comprises a VH comprising a CDRH1, CDRH2, and/or CDRH3 amino acid sequence set forth in Tables 3-7. In certain embodiments, the instant disclosure provides an antibody that specifically binds to APJ (e.g., human APJ), wherein the antibody comprises a VH comprising the CDRH1, CDRH2, and CDRH3 amino acid sequences of any of the antibodies in Tables 3-7.

In certain embodiments, the instant disclosure provides an antibody that specifically binds to APJ (e.g., human APJ), wherein the antibody comprises a VH comprising the CDRH1, CDRH2, and CDRH3 amino acid sequences of any of the antibodies in Table 3. In certain embodiments, the antibody comprises a VH comprising the CDRH1, CDRH2, and CDRH3 amino acid sequences set forth in SEQ ID NOs: 374, 384, and 141; 354, 355, and 87; 354, 355, and 90; 354, 357, and 93; 354, 357, and 96; 354, 355, and 96; 354, 355, and 99; 354, 357, and 99; 354, 355, and 102; 354, 359, and 102; 354, 359, and 105; 354, 355, and 105; 354, 357, and 105; 354, 355, and 108; 354, 357, and 108; 354, 357, and 111; 354, 357, and 114; 354, 355, and 114; 354, 357, and 117; 354, 355, and 117; 354, 357, and 120; 354, 357, and 123; 354, 357, and 125; 354, 357, and 127; 354, 357, and 129; 354, 357, and 131; 354, 357, and 133; 354, 357, and 135; 354, 357, and 137; 354, 357, and 139; 354, 357, and 141; 354, 355, and 143; 354, 355, and 145; 354, 355, and 147; 354, 355, and 149; 354, 355, and 151; 354, 355, and 152; 354, 355, and 153; 356, 361, and 117; 358, 361, and 117; 360, 363, and 117; 362, 365, and 117; 364, 367, and 133; 366, 367, and 133; 368, 369, and 133; 370, 371, and 133; 354, 373, and 108; 354, 375, and 117; 354, 373, and 120; 354, 373, and 129; 354, 373, and 131; 354, 373, and 133; 354, 373, and 139; 354, 355, and 154; 372, 355, and 87; 354, 377, and 87; 354, 355, and 155; 354, 373, and 141; 374, 379, and 141; 374, 380, and 141; 374, 381, and 141; 374, 382, and 141; 374, 383, and 141; 374, 385, and 131; 374, 380, and 131; 374, 379, and 131; 376, 385, and 131; 376, 380, and 131; 376, 379, and 131; 378, 385, and 131; or 378, 379, and 131, respectively. In certain embodiments, the antibody comprises a VH comprising the CDRH1, CDRH2, and CDRH3 amino acid sequences set forth in SEQ ID NOs: 374, 384, and 141, respectively. In certain embodiments, the antibody comprises a VH comprising the CDRH1, CDRH2, and CDRH3 amino acid sequences set forth in SEQ ID NOs: 374, 380, and 131, respectively.

In certain embodiments, the instant disclosure provides an antibody that specifically binds to APJ (e.g., human APJ), wherein the antibody comprises a VH comprising the CDRH1, CDRH2, and CDRH3 amino acid sequences of any of the antibodies in Table 4. In certain embodiments, the antibody comprises a VH comprising the CDRH1, CDRH2, and CDRH3 amino acid sequences set forth in SEQ ID NOs: 115, 144, and 141; 85, 86, and 87; 85, 86, and 90; 85, 89, and 93; 85, 89, and 96; 85, 86, and 96; 85, 86, and 99; 85, 89, and 99; 85, 86, and 102; 85, 92, and 102; 85, 92, and 105; 85, 86, and 105; 85, 89, and 105; 85, 86, and 108; 85, 89, and 108; 85, 89, and 111; 85, 89, and 114; 85, 86, and 114; 85, 89, and 117; 85, 86, and 117; 85, 89, and 120; 85, 89, and 123; 85, 89, and 125; 85, 89, and 127; 85, 89, and 129; 85, 89, and 131; 85, 89, and 133; 85, 89, and 135; 85, 89, and 137; 85, 89, and 139; 85, 89, and 141; 85, 86, and 143; 85, 86, and 145; 85, 86, and 147; 85, 86, and 149; 85, 86, and 151; 85, 86, and 152; 85, 86, and 153; 88, 95, and 117; 91, 98, and 117; 94, 101, and 117; 97, 104, and 117; 100, 107, and 133; 103, 110, and 133; 106, 113, and 133; 109, 116, and 133; 85, 119, and 108; 85, 122, and 108; 85, 124, and 117; 85, 126, and 117; 85, 119, and 120; 85, 122, and 120; 85, 119, and 129; 85, 122, and 129; 85, 119, and 131; 85, 122, and 131; 85, 119, and 133; 85, 122, and 133; 85, 119, and 139; 85, 122, and 139; 85, 86, and 154; 112, 86, and 87; 85, 128, and 87; 85, 86, and 155; 85, 130, and 141; 85, 132, and 141; 115, 134, and 141; 115, 136, and 141; 115, 138, and 141; 115, 140, and 141; 115, 142, and 141; 115, 146, and 131; 115, 148, and 131; 115, 150, and 131; 118, 146, and 131; 118, 148, and 131; 118, 150, and 131; 121, 146, and 131; or 121, 150, and 131, respectively. In certain embodiments, the antibody comprises a VH comprising the CDRH1, CDRH2, and CDRH3 amino acid sequences set forth in SEQ ID NOs: 115, 144, and 141, respectively. In certain embodiments, the antibody comprises a VH comprising the CDRH1, CDRH2, and CDRH3 amino acid sequences set forth in SEQ ID NOs: 115, 148, and 131, respectively.

In certain embodiments, the instant disclosure provides an antibody that specifically binds to APJ (e.g., human APJ), wherein the antibody comprises a VH comprising the CDRH1, CDRH2, and CDRH3 amino acid sequences of any of the antibodies in Table 5. In certain embodiments, the antibody comprises a VH comprising the CDRH1, CDRH2, and CDRH3 amino acid sequences set forth in SEQ ID NOs: 429, 430, and 243; 427, 428, and 194; 427, 428, and 197; 429, 430, and 200; 429, 430, and 203; 429, 430, and 206; 429, 430, and 209; 431, 428, and 212; 431, 428, and 215; 433, 428, and 218; 433, 428, and 221; 433, 428, and 224; 433, 428, and 227; 435, 432, and 230; 437, 434, and 232; 439, 428, and 234; 433, 428, and 235; 441, 436, and 236; 441, 436, and 237; 443, 438, and 238; 445, 440, and 239; 447, 442, and 240; 431, 428, and 241; 431, 428, and 242; 448, 444, and 244; 449, 428, and 218; 427, 428, and 218; 427, 446, and 218; 433, 428, and 245; 433, 428, and 246; 449, 428, and 245; 449, 428, and 246; or 431, 428, and 245, respectively. In certain embodiments, the antibody comprises a VH comprising the CDRH1, CDRH2, and CDRH3 amino acid sequences set forth in SEQ ID NOs: 429, 430, and 243, respectively.

In certain embodiments, the instant disclosure provides an antibody that specifically binds to APJ (e.g., human APJ), wherein the antibody comprises a VH comprising the CDRH1, CDRH2, and CDRH3 amino acid sequences of any of the antibodies in Table 6. In certain embodiments, the antibody comprises a VH comprising the CDRH1, CDRH2, and CDRH3 amino acid sequences set forth in SEQ ID NOs: 195, 834, and 243; 192, 833, and 194; 192, 833, and 197; 195, 834, and 200; 195, 834, and 203; 195, 834, and 206; 195, 834, and 209; 198, 835, and 212; 198, 835, and 215; 201, 836, and 218; 201, 836, and 221; 201, 836, and 224; 201, 836, and 227; 204, 837, and 230; 207, 838, and 232; 210, 836, and 234; 201, 836, and 235; 213, 839, and 236; 213, 839, and 237; 216, 840, and 238; 219, 841, and 239; 222, 842, and 240; 198, 835, and 241; 198, 835, and 242; 225, 843, and 244; 228, 836, and 218; 192, 836, and 218; 192, 844, and 218; 192, 845, and 218; 192, 846, and 218; 192, 847, and 218; 201, 836, and 245; 201, 836, and 246; 228, 836, and 245; 228, 836, and 246; or 198, 836, and 245, respectively. In certain embodiments, the antibody comprises a VH comprising the CDRH1, CDRH2, and CDRH3 amino acid sequences set forth in SEQ ID NOs: 195, 834, and 243, respectively.

In certain embodiments, the instant disclosure provides an antibody that specifically binds to APJ (e.g., human APJ), wherein the antibody comprises a VH comprising the CDRH1, CDRH2, and CDRH3 amino acid sequences of any of the antibodies in Table 7. In certain embodiments, the antibody comprises a VH comprising the CDRH1, CDRH2, and CDRH3 amino acid sequences set forth in SEQ ID NOs: 195, 196, and 243; 192, 193, and 194; 192, 193, and 197; 195, 196, and 200; 195, 196, and 203; 195, 196, and 206; 195, 196, and 209; 198, 199, and 212; 198, 199, and 215; 201, 202, and 218; 201, 202, and 221; 201, 202, and 224; 201, 202, and 227; 204, 205, and 230; 207, 208, and 232; 202, 210, and 234; 201, 202, and 235; 211, 213, and 236; 211, 213, and 237; 214, 216, and 238; 217, 219, and 239; 220, 222, and 240; 198, 199, and 241; 198, 199, and 242; 223, 225, and 244; 202, 218, and 228; 192, 202, and 218; 192, 218, and 226; 192, 218, and 229; 192, 218, and 231; 192, 218, and 233; 201, 202, and 245; 201, 202, and 246; 202, 228, and 245; 202, 228, and 246; or 198, 202, and 245, respectively. In certain embodiments, the antibody comprises a VH comprising the CDRH1, CDRH2, and CDRH3 amino acid sequences set forth in SEQ ID NOs: 195, 196, and 243, respectively.

In certain embodiments, the instant disclosure provides an antibody that specifically binds to APJ (e.g., human APJ) comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in any one of SEQ ID NOs: 1-59, 65-84, or 156-191. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 1. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 2. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 3. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 4. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 5. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 6. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 7. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 8. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 9. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 10. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 11. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 12. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 13. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 14. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 15. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 16. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 17. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 18. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 19. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 20. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 21. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 22. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 23. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 24. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 25. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 26. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 27. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 28. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 29. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 30. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 31. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 32. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 33. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 34. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 35. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 36. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 37. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 38. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 39. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 40. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 41. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 42. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 43. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 44. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 45. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 46. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 47. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 48. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 49. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 50. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 51. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 52. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 53. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 54. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 55. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 56. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 57. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 58. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 59. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 65. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 66. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 67. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 68. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 69. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 70. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 71. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 72. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 73. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 74. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 75. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 76. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 77. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 78. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 79. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 80. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 81. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 82. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 83. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 84. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 156. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 157. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 158. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 159. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 160. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 161. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 162. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 163. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 164. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 165. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 166. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 167. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 168. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 169. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 170. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 171. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 172. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 173. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 174. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 175. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 176. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 177. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 178. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 179. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 180. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 181. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 182. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 183. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 184. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 185. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 186. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 187. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 188. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 189. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 190. In certain embodiments, the VH comprises an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 191.

In certain embodiments, the instant disclosure provides an antibody that specifically binds to APJ (e.g., human APJ), comprising a VH comprising an amino acid sequence set forth in any one of SEQ ID NOs: 1-59, 65-84, or 156-191. In certain embodiments, the amino acid sequence of the VH consists of the amino acid sequence set forth in any one of SEQ ID NOs: 1-59, 65-84, or 156-191.

In certain embodiments, the antibodies provided herein specifically bind to APJ (e.g., human APJ) and act as APJ antagonists. In certain embodiments, APJ antagonist antibodies reduce or inhibit a function of APJ by at least 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art, relative to the APJ function without any antibody or with an unrelated antibody (e.g., an antibody that does not specifically bind to APJ). In certain embodiments, the APJ antagonist antibodies provided herein specifically bind to APJ (e.g., human APJ) and reduce or inhibit a function of APJ by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein and/or known to one of skill in the art, relative to the APJ function without any antibody or with an unrelated antibody (e.g., an antibody that does not specifically bind to APJ). Non-limiting examples of APJ functions include APJ signaling, APJ binding to ligands (e.g., apelin, elabela), cyclic AMP production, and β-arrestin production. In certain embodiments, reduction or inhibition of a function of APJ is assessed as described in the Examples herein.

In certain embodiments, an APJ antagonist antibody provided herein comprises a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in any one of SEQ ID NOs: 1-59, 66-84, or 156-191. In certain embodiments, an APJ antagonist antibody provided herein comprises a VH comprising an amino acid sequence set forth in any one of SEQ ID NOs: 1-59, 66-84, or 156-191. In certain embodiments, the amino acid sequence of the APJ antagonist antibody VH consists of the amino acid sequence set forth in any one of SEQ ID NOs: 1-59, 66-84, or 156-191.

In certain embodiments, the antibodies provided herein specifically bind to APJ (e.g., human APJ) and act as APJ agonists. In certain embodiments, APJ antagonist antibodies increase a function of APJ by at least 5%, 10%, 15%, 20%, 25%, 30%, 35%, 40%, 45%, 50%, 55%, 60%, 65%, 70%, 75%, 80%, 85%, 90%, 95%, 98%, 99%, 100%, or more as assessed by methods described herein and/or known to one of skill in the art, relative to the APJ function without any antibody or with an unrelated antibody (e.g., an antibody that does not specifically bind to APJ). In certain embodiments, the APJ agonist antibodies provided herein specifically bind to APJ (e.g., human APJ) and increase a function of APJ by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or more as assessed by methods described herein and/or known to one of skill in the art, relative to the APJ function without any antibody or with an unrelated antibody (e.g., an antibody that does not specifically bind to APJ). In certain embodiments, increase of a function of APJ is assessed as described in the Examples herein.

In certain embodiments, an APJ agonist antibody provided herein comprises a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 65. In certain embodiments, an APJ agonist antibody provided herein comprises a VH comprising an amino acid sequence set forth SEQ ID NO: 65. In certain embodiments, the amino acid sequence of the APJ agonist antibody VH consists of the amino acid sequence set forth in SEQ ID NO: 65.

In certain embodiments, the instant disclosure provides an antibody that cross-competes for binding to APJ (e.g., human APJ) with any of the antibodies described above. In certain embodiments, the instant disclosure provides an antibody that binds to the same or an overlapping epitope of APJ (e.g., an epitope of human APJ) as an antibody described above.

In certain embodiments, the epitope of an antibody can be determined by, e.g., NMR spectroscopy, surface plasmon resonance (BIAcore®), X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303, all of which are herein incorporated by reference in their entireties). Antibody:antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds. Wyckoff H W et al.; U.S. Patent Application No. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323, all of which are herein incorporated by reference in their entireties). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In a specific embodiment, the epitope of an antibody is determined using alanine scanning mutagenesis studies. In addition, or antibodies that recognize and bind to the same or overlapping epitopes of APJ (e.g., human APJ) can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as APJ (e.g., human APJ). Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9: 242-253); solid phase direct biotin-avidin EIA (see Kirkland T N et al., (1986) J Immunol 137: 3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25(1): 7-15); solid phase direct biotin-avidin EIA (see Cheung R C et al., (1990) Virology 176: 546-52); and direct labeled RIA (see Moldenhauer G et al., (1990) Scand J Immunol 32: 77-82), all of which are herein incorporated by reference in their entireties. Typically, such an assay involves the use of purified antigen (e.g., APJ, such as human APJ) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually, the test immunoglobulin is present in excess. Usually, when a competing antibody is present in excess, it will inhibit specific binding of a reference or antibody to a common antigen by at least 50-55%, 55-60%, 60-65%, 65-70%, 70-75%, or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, e.g., Wagener C et al., (1983) J Immunol 130: 2308-2315; Wagener C et al., (1984) J Immunol Methods 68: 269-274; Kuroki M et al., (1990) Cancer Res 50: 4872-4879; Kuroki M et al., (1992) Immunol Invest 21: 523-538; Kuroki M et al., (1992) Hybridoma 11: 391-407 and Antibodies: A Laboratory Manual, Ed Harlow E & Lane D editors supra, pp. 386-389, all of which are herein incorporated by reference in their entireties.

In certain embodiments, the antibodies provided herein comprise an engineered human heavy chain variable domain (VH) framework. In certain embodiments, the engineered human VH framework is suitable for formation of antibodies comprising only a single variable domain (e.g., a VH). In certain embodiments, the engineered human VH framework comprises an amino acid sequence having at least 90%, 91%, 92%, 93%, 94%, 95%, 96%, 97%, 98%, 99%, or 100% sequence identity to an amino acid sequence provided below in Table 8. In certain embodiments, the engineered human VH framework comprises an amino acid sequence having at least 90%, 91%, 92%, 93%, 94%, 95%, 96%, 97%, 98%, 99%, or 100% sequence identity to any one of SEQ ID NOs: 819-821, 849, and 822. In certain embodiments, the engineered human VH framework comprises a framework region (FR) 1 sequence comprising an amino acid sequence having at least 90%, 91%, 92%, 93%, 94%, 95%, 96%, 97%, 98%, 99%, or 100% sequence identity to SEQ ID NO: 819. In certain embodiments, the engineered human VH framework comprises an FR 2 sequence comprising an amino acid sequence having at least 90%, 91%, 92%, 93%, 94%, 95%, 96%, 97%, 98%, 99%, or 100% sequence identity to SEQ ID NO: 820. In certain embodiments, the engineered human VH framework comprises an FR 3 sequence comprising an amino acid sequence having at least 90%, 91%, 92%, 93%, 94%, 95%, 96%, 97%, 98%, 99%, or 100% sequence identity to SEQ ID NO: 821. In certain embodiments, the engineered human VH framework comprises an FR 3 sequence comprising an amino acid sequence having at least 90%, 91%, 92%, 93%, 94%, 95%, 96%, 97%, 98%, 99%, or 100% sequence identity to SEQ ID NO: 849. In certain embodiments, the engineered human VH framework comprises an FR 4 sequence comprising an amino acid sequence having at least 90%, 91%, 92%, 93%, 94%, 95%, 96%, 97%, 98%, 99%, or 100% sequence identity to SEQ ID NO: 822. In certain embodiments, the engineered human VH framework comprises an FR 1 sequence comprising or consisting of the amino acid sequence set forth in SEQ ID NO: 819. In certain embodiments, the engineered human VH framework comprises an FR 2 sequence comprising or consisting of the amino acid sequence set forth in SEQ ID NO: 820. In certain embodiments, the engineered human VH framework comprises an FR 3 sequence comprising or consisting of the amino acid sequence set forth in SEQ ID NO: 821. In certain embodiments, the engineered human VH framework comprises an FR 3 sequence comprising or consisting of the amino acid sequence set forth in SEQ ID NO: 849. In certain embodiments, the engineered human VH framework comprises an FR 4 sequence comprising or consisting of the amino acid sequence set forth in SEQ ID NO: 822. In certain embodiments, the engineered human VH framework comprises an FR 1 sequence comprising or consisting of the amino acid sequence set forth in SEQ ID NO: 819; an FR 2 sequence comprising or consisting of the amino acid sequence set forth in SEQ ID NO: 820; an FR 3 sequence comprising or consisting of the amino acid sequence set forth in SEQ ID NO: 821; and/or an FR 4 sequence comprising or consisting of the amino acid sequence set forth in SEQ ID NO: 822. In certain embodiments, the engineered human VH framework comprises an FR 1 sequence comprising or consisting of the amino acid sequence set forth in SEQ ID NO: 819; an FR 2 sequence comprising or consisting of the amino acid sequence set forth in SEQ ID NO: 820; an FR 3 sequence comprising or consisting of the amino acid sequence set forth in SEQ ID NO: 849; and/or an FR 4 sequence comprising or consisting of the amino acid sequence set forth in SEQ ID NO: 822.

TABLE 8
Engineered human VH framework amino acid
sequences.

| | | SEQ |
|---|---|---|
| | | ID |
| Description | Sequence | NO |
| Framework | EVQLVESGGGLVQPGGSLRLSCAAS | 819 |
| region 1 | | |
| Framework | WYRQAPGKGREFVA | 820 |
| region 2 | | |
| Framework | GRFTISRDNSKNTVYLQMNSLRAEDTAVYYC | 821 |
| region 3 | | |
| (sequence 1) | | |
| Framework | ADSVKGRFTISRDNSKNTVYLQMNSLRAEDT | 849 |
| region 3 | AVYYC | |
| (sequence 2) | | |
| Framework | WGQGTQVTVSS | 822 |
| region 4 | | |

In certain embodiments, the antibodies provided herein comprise an immunoglobulin (Ig) constant region or a portion thereof (e.g., an Ig Fc). Any immunoglobulin (Ig) constant region can be used in the antibodies disclosed herein. In certain embodiments, the Ig region is a human IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂a and IgG₂b) of immunoglobulin molecule.

In certain embodiments, the instant disclosure provides an antibody that specifically binds to APJ (e.g., human APJ), the antibody comprising a heavy chain constant region, optionally selected from the group consisting of human IgG₁, IgG₂, IgG₃, IgG₄, IgA₁, and IgA₂.

In certain embodiments, the instant disclosure provides an antibody that specifically binds to APJ (e.g., human APJ), the antibody comprising a light chain constant region, optionally selected from the group consisting of a human IgG kappa light chain constant region and a human IgG lambda light chain constant region.

In certain embodiments, the antibodies provided herein further comprise an Ig Fc. In certain embodiments, the Ig Fc is an IgG Fc. The Ig Fc can be a wild-type Ig Fc (native Ig Fc) or a variant Ig Fc. In certain embodiments, the antibodies provided herein form dimers (e.g., homodimers via interaction between Ig Fcs). In certain embodiments, two antibodies are linked into a dimer (e.g., a homodimer) via 2 hinge region interchain disulfide bonds between the Ig Fc of each antibody (e.g., at the N-terminus). In certain embodiments, the Ig Fc comprises one intrachain disulfide bond in the CH2 domain and one intrachain disulfide bond in the CH3 domain. In certain embodiments, the antibodies provided herein are homodimeric.

The Ig Fc of the antibodies provided herein can be derived from any native immunoglobulin. In certain embodiments, the Ig Fc is formed from an IgA, IgD, IgE, or IgG heavy chain constant region. In certain embodiments, the Ig Fc is formed from an IgG heavy chain constant region. In certain embodiments, the IgG heavy chain is an IgG1, IgG2, IgG3 or IgG4 heavy chain constant region. In certain embodiments, the Ig Fc is formed from an IgG1 heavy chain constant region. In certain embodiments, the IgG1 heavy chain constant region comprises a G1m1(a), G1m2(x), G1m3(f), or G1m17(z) allotype. See, e.g., Jefferis and Lefranc (2009) mAbs 1(4): 332-338, and de Taeye et al. (2020) Front Immunol. 11:740, incorporated herein by reference in their entirety. The N-terminus of the IgG Fc or the C-terminus of the IgG Fc can be linked to the N-terminus of the heavy chain variable domain (VH) or the C-terminus of the VH. The IgG Fc can be linked directly to the N-terminal peptide or the C-terminal peptide, or the IgG Fc can be linked to the N-terminal peptide or the C-terminal peptide through a linker.

In certain embodiments, the N-terminus of the IgG Fc is linked to the C-terminus of the VH. In certain embodiments, the N-terminus of the IgG Fc is linked to the C-terminus of the VH via a linker. In certain embodiments, the C-terminus of the IgG Fc is linked to the N-terminus of the VH. In certain embodiments, the C-terminus of the IgG Fc is linked to the N-terminus of the VH via a linker. In certain embodiments, the linker comprises or consists of 1, 2, 3, 4, 5, 6, 7, 8, 9, or 10 amino acids. In certain embodiments, the linker comprises or consists of 1, 2, 3, 4, or 5 amino acids. In certain embodiments, the linker comprises or consists of 5 amino acids. In certain embodiments, the linker comprises or consists of the amino acid sequence GGGGS (SEQ ID NO: 269).

In certain embodiments, the IgG Fc comprises a C-terminal lysine (K). It is known in the art that the C-terminal lysine (K) in many monoclonal antibodies is flexible and is often clipped off during expression and purification with no known impairment in activity. In certain embodiments, the IgG Fc does not comprise a C-terminal lysine (K). In certain embodiments, the C-terminal lysine (K) is replaced with a C-terminal glutamic acid (E). As such, in certain embodiments, the IgG Fc comprises a C-terminal glutamic acid (E).

In certain embodiments, one, two, or more mutations (e.g., amino acid substitutions, insertions or deletions) are introduced into the Ig Fc of an antibody described herein (e.g., CH2 domain (residues 231-340 of human IgG1) and/or CH3 domain (residues 341-447 of human IgG1)) and/or the hinge region, numbered according to the EU numbering system, to alter one or more functional properties of the antibody, such as serum half-life, complement fixation, Fc receptor binding, and/or antigen-dependent cellular cytotoxicity.

In a specific embodiment, one, two, or more amino acid mutations (e.g., substitutions, insertions or deletions) are introduced into an IgG constant domain, or FcRn-binding fragment thereof (preferably an Ig Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375, and 6,165,745, all of which are herein incorporated by reference in their entireties, for examples of mutations that will alter (e.g., decrease or increase) the half-life of an antibody in vivo. In certain embodiments, one, two, or more amino acid mutations (e.g., substitutions, insertions, or deletions) are introduced into an IgG constant domain, or FcRn-binding fragment thereof (preferably an Ig Fc or hinge-Fc domain fragment) to decrease the half-life of the antibody in vivo. In other embodiments, one, two, or more amino acid mutations (e.g., substitutions, insertions, or deletions) are introduced into an IgG constant domain, or FcRn-binding fragment thereof (preferably an Ig Fc or hinge-Fc domain fragment) to increase the half-life of the antibody in vivo. In a specific embodiment, the antibodies may have one or more amino acid mutations (e.g., substitutions) in the second constant (CH2) domain (residues 231-340 of human IgG1) and/or the third constant (CH3) domain (residues 341-447 of human IgG1), numbered according to the EU numbering system. In a specific embodiment, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU numbering system. See, U.S. Pat. No. 7,658,921, which is herein incorporated by reference in its entirety. This type of mutant IgG, referred to as “YTE mutant” has been shown to display fourfold increased half-life as compared to wild-type versions of the same antibody (see, Dall'Acqua W F et al., (2006) J Biol Chem 281: 23514-24, which is herein incorporated by reference in its entirety). In certain embodiments, the constant region of the IgG1 of an antibody described herein comprises a threonine (T) to glutamine (Q) substation in position 250 and a methionine (M) to leucine (L) substitution in position 428, numbered according to the EU numbering system. See, Hinton et al., (2004) J Biol Chem 279(8):6213-6, which is herein incorporated by reference in its entirety. In certain embodiments, the constant region of the IgG1 of an antibody described herein comprises a threonine (T) to alanine (A) substitution in position 307, a glutamate (E) to alanine (A) substitution in position 380, and an asparagine (N) to alanine (A) substitution in position 434, numbered according to the EU numbering system. See, Petkova et al., (2006) Int Immunol 18(12):1759-69, which is herein incorporated by reference in its entirety. In certain embodiments, an antibody comprises an IgG constant domain comprising one, two, three or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into the Ig Fc of an antibody described herein (e.g., CH2 domain (residues 231-340 of human IgG1) and/or CH3 domain (residues 341-447 of human IgG1)) and/or the hinge region, numbered according to the EU numbering system, to increase or decrease the affinity of the antibody for an Fc receptor (e.g., an activated Fc receptor) on the surface of an effector cell. Mutations in the Ig Fc of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, all of which are herein incorporated by reference in their entireties.

In certain embodiments, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG1, a variant human IgG2, or a variant human IgG4 heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In certain embodiments, the FcγRIIB is expressed on a cell selected from the group consisting of macrophages, monocytes, B cells, dendritic cells, endothelial cells, and activated T cells.

In a further embodiment, one, two, or more amino acid substitutions are introduced into an IgG Fc to alter the effector function(s) of the antibody. For example, one or more amino acids selected from amino acid residues 234, 235, 236, 237, 239, 243, 253, 267, 292, 297, 300, 310, 318, 320, 322, 328, 329, 330, 331, 332, 396, and 435, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant domain and thereby increase tumor localization. In certain embodiments, one or more amino acid substitutions may be introduced into the Ig Fc of an antibody described herein to remove potential glycosylation sites on the Ig Fc, which may reduce Fc receptor binding (see, e.g., Shields R L et al., (2001) J Biol Chem 276: 6591-604, which is herein incorporated by reference in its entirety). In various embodiments, one or more of the following mutations in the constant region of an antibody described herein may be made: an N297A substitution; an N297Q substitution; an E233P substitution; an L234A substitution; an L234F substitution; an L234V substitution; an L235A substitution; an L235E substitution; an L235Q substitution; an L235F substitution; an L235V substitution; an L237A substitution; an S239D substitution; a C236 deletion; a P238A substitution; an S239D substitution; an F243L substitution; an I253A substitution; a D265A substitution; an S267E substitution; an L328F substitution; an R292P substitution; a Y300L substitution; an H310A substitution; a K322Q substitution; an A327Q substitution; a P329A substitution (PA); a P329G substitution; a P331S substitution; an A332L substitution; an 1332E substitution; a P396L substitution; or an H435A substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, 1332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In a specific embodiment, an antibody described herein comprises the constant domain of an IgG1 with an N297Q or N297A amino acid substitution, numbered according to the EU numbering system. In one embodiment, an antibody described herein comprises the constant domain of an IgG1 with a mutation selected from the group consisting of D265A, P329A, and a combination thereof, numbered according to the EU numbering system. In another embodiment, an antibody described herein comprises the constant domain of an IgG1 with a mutation selected from the group consisting of L234A, L235A (LALA), and a combination thereof, numbered according to the EU numbering system. In another embodiment, an antibody described herein comprises the constant domain of an IgG1 with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG1 heavy chain, numbered according to the EU numbering system, are not L, L, and D, respectively. This approach is described in detail in International Publication No. WO 14/108483, which is herein incorporated by reference in its entirety. In a particular embodiment, the amino acids corresponding to positions L234, L235, and D265 in a human IgG1 heavy chain are F, E, and A; or A, A, and A, respectively, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In certain embodiments, the Ig Fc of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In certain embodiments, any of the constant region mutations or modifications described herein can be introduced into one or both heavy chain constant regions of an antibody described herein having two heavy chain constant regions.

In certain embodiments, the IgG Fc is an IgG1 Fc, or a derivative thereof. In certain embodiments, the IgG Fc comprises an amino acid sequence having at least 85%, 86%, 87%, 88%, 89%, 90%, 91%, 92%, 93%, 94%, 95%, 96%, 97%, 98%, 99%, or 100% sequence identity to the amino acid sequence of a human IgG1 Fc. In certain embodiments, the IgG Fc comprises the amino acid sequence of a human IgG1 Fc. In certain embodiments, the IgG Fc comprises or consists of an amino acid sequence having at least 85%, 86%, 87%, 88%, 89%, 90%, 91%, 92%, 93%, 94%, 95%, 96%, 97%, 98%, 99%, or 100% sequence identity to an amino acid sequence provided below in Table 9. In certain embodiments, the IgG Fc comprises or consists of an amino acid sequence having at least 85%, 86%, 87%, 88%, 89%, 90%, 91%, 92%, 93%, 94%, 95%, 96%, 97%, 98%, 99%, or 100% sequence identity to any one of SEQ ID NOs: 253-268 and 450-463. In certain embodiments, the IgG Fc comprises or consists of an amino acid sequence having at least 95%, 96%, 97%, 98%, 99%, or 100% sequence identity to any one of SEQ ID NOs: 253-268 and 450-463. In certain embodiments, the IgG Fc comprises or consists of an amino acid sequence having at least 85%, 86%, 87%, 88%, 89%, 90%, 91%, 92%, 93%, 94%, 95%, 96%, 97%, 98%, 99%, or 100% sequence identity to an amino acid sequence selected from the group consisting of SEQ ID NOs: 253-268 and 450-463. In certain embodiments, the IgG Fc comprises or consists of an amino acid sequence having at least 95%, 96%, 97%, 98%, 99%, or 100% sequence identity to an amino acid sequence selected from the group consisting of SEQ ID NOs: 253-268 and 450-463.

TABLE 9
IgG Fc amino acid sequences.

| SEQ | | |
|---|---|---|
| ID | Description | Sequence |
| 253 | IgG1 Fc | EPKSSDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 254 | IgG1 Fc LALA | EPKSSDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRD |
| | | ELTKNQVSLTCLVKGFYPSDIAVEWESNGQPENNYKITPPVLDSDGS |
| | | FFLYSKLTVDKSRWQQGNVFSCSVMHEALHNHYTQKSLSLSPGK |
| 255 | IgG1 Fc LALAPA | EPKSSDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSRD |
| | | ELTKNQVSLTCLVKGFYPSDIAVEWESNGQPENNYKITPPVLDSDGS |
| | | FFLYSKLTVDKSRWQQGNVFSCSVMHEALHNHYTOKSLSLSPGK |
| 256 | IgG1 Fc | EPKSSDKTHTCPPCPAPEAAGGPSVFLFPPKPKDTLMISRTPEVTCV |
| | LALAPALS | VVDVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVLHEALHSHYTQKSLSLSPGK |
| 257 | IgG1 Fc DAPA | EPKSSDKTHTCPPCPAPELLGGPSVFLFPPKPKDTLMISRTPEVTCV |
| | (D265A/P329A) | VVAVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 258 | IgG1 Fc | EPKSSDKTHTCPPCPAPELLGGPSVFLFPPKPKDTLMISRTPEVTCV |
| | DANAPA | VVAVSHEDPEVKFNWYVDGVEVHNAKTKPREEQYASTYRVVSVLTVL |
| | (D265A/N297A/ | HQDWLNGKEYKCKVSNKALAAPIEKTISKAKGQPREPQVYTLPPSRD |
| | P329A) | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 259 | Y IgG1 Fc TE | EPKSSDKTHTCPPCPAPELLGGPSVFLFPPKPKDTLYITREPEVTCV |
| | (M252Y/S254T/ | VVDVSHEDPEVKFNWYVDGVEVHNAKTKPREEQYNSTYRVVSVLTVL |
| | T256E) | HQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 260 | IgG1 Fc FES-YTE | EPKSSDKTHTCPPCPAPEFEGGPSVFLFPPKPKDTLYITREPEVTCV |
| | (L234F/L235E/ | VVDVSHEDPEVKFNWYVDGVEVHNAKTKPREEQYNSTYRVVSVLTVL |
| | P331S/M252Y/ | HQDWLNGKEYKCKVSNKALPASIEKTISKAKGQPREPQVYTLPPSRD |
| | S254T/ T256E) | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 450 | IgG1 Fc FES-LS | EPKSSDKTHTCPPCPAPEFEGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALPASIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVLHEALHSHYTQKSLSLSPGK |
| 451 | IgG1 Fc | EPKSSDKTHTCPPCPAPEAAGGPSVFLFPPKPKDTLMISRTPEVTCV |
| | LALAPA-H435A | VVDVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVLHEALHSAYTQKSLSLSPGK |
| 452 | IgG1 Fc IHH | EPKSSDKTHTCPPCPAPELLGGPSVFLFPPKPKDTLMASRTPEVTCV |
| | (I253A, H310A, | VVDVSHEDPEVKFNWYVDGVEVHNAKTKPREEQYNSTYRVVSVLTVL |
| | and H435A) | AQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNAYTQKSLSLSPGK |
| 453 | IgG1 Fc QL | EPKSSDKTHTCPPCPAPELLGGPSVFLFPPKPKDQLMISRTPEVTCV |
| | (T250Q/ M428L) | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTIPPSRD |
| | | ELTKNQVSLTCLVKGFYPSDIAVEWESNGQPENNYKTTPPVLDSDGS |
| | | FFLYSKLTVDKSRWQQGNVFSCSVLHEALHNHYTQKSLSLSPGK |
| 454 | IgG1 Fc | EPKSSDKTHTCPPCPAPELLGGPSVFLFPPKPKDTLMISRTPEVTCV |
| | T307A/E380A/ | VVDVSHEDPEVKFNWYVDGVEVHNAKTKPREEQYNSTYRVVSVLAVL |
| | N434A | HQDWLNGKEYKCKVSNKALPAPIEKTISKAKGQPREPQVYTLPPSRD |
| | | ELTKNQVSLTCLVKGFYPSDIAVAWESNGQPENNYKTTPPVLDSDGS |
| | | FFLYSKLTVDKSRWQQGNVFSCSVMHEALHAHYTQKSLSLSPGK |
| 455 | IgG1 Fc FQQ | EPKSSDKTHTCPPCPAPEFQGGPSVFLFPPKPKDTLMISRTPEVTCV |
| | L234F/L235Q/ | VVDVSHEDPEVKFNWYVDGVEVHNAKTKPREEQYNSTYRVVSVLTVL |
| | K322Q | HQDWLNGKEYKCQVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 456 | IgG1 Fc LALAPG | EPKSSDKTHTCPPCPAPEAAGGPSVFLFPPKPKDTLMISRTPEVTCV |
| | L234A/L235A/ | VVDVSHEDPEVKFNWYVDGVEVHNAKTKPREEQYNSTYRVVSVLTVL |
| | P329 | HQDWLNGKEYKCKVSNKALGAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVLHEALHSHYTQKSLSLSPGK |
| 261 | IgG1 Fc without | EPKSSDKTHTCPPCPAPELLGGPSVFLFPPKPKDTLMISRTPEVTCV |
| | C-term. K | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 262 | IgG1 Fc LALA | EPKSSDKTHTCPPCPAPEAAGGPSVFLFPPKPKDTLMISRTPEVTCV |
| | without C-term. K | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 263 | IgG1 Fc LALAPA | EPKSSDKTHTCPPCPAPEAAGGPSVFLFPPKPKDTLMISRTPEVTCV |
| | without C-term. K | VVDVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 264 | IgG1 Fc | EPKSSDKTHTCPPCPAPEAAGGPSVFLFPPKPKDTLMISRTPEVTCV |
| | LALAPALS | VVDVSHEDPEVKFNWYVDGVEVHNAKTKPREEQYNSTYRVVSVLTVL |
| | without C-term. K | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVLHEALHSHYTQKSLSLSPG |
| 265 | IgG1 Fc DAPA | EPKSSDKTHTCPPCPAPELLGGPSVFLFPPKPKDTLMISRTPEVTCV |
| | without C-term. K | VVAVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 266 | IgG1 Fc DNAPA | EPKSSDKTHTCPPCPAPELLGGPSVFLFPPKPKDTLMISRTPEVTCV |
| | without C-term. K | VVAVSHEDPEVKFNWYVDGVEVHNAKTKPREEQYASTYRVVSVLTVL |
| | | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 267 | IgG1 Fc YTE | EPKSSDKTHTCPPCPAPELLGGPSVFLFPPKPKDTLYITREPEVTCV |
| | without C-term. K | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 268 | IgG1 Fc YTE-FES | EPKSSDKTHTCPPCPAPEFEGGPSVFLFPPKPKDTLYITREPEVTCV |
| | without C-term. K | VVDVSHEDPEVKFNWYVDGVEVHNAKTKPREEQYNSTYRVVSVLTVL |
| | | HQDWLNGKEYKCKVSNKALPASIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 457 | IgG1 Fc | EPKSSDKTHTCPPCPAPEAAGGPSVFLFPPKPKDTLMISRTPEVTCV |
| | LALAPA-H435A | VVDVSHEDPEVKFNWYVDGVEVHNAKTKPREEQYNSTYRVVSVLTVL |
| | without C-term. K | HQDWLNGKEYKCKVSNKALAAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVLHEALHSAYTQKSLSLSPG |
| 458 | IgG1 Fc IHH | EPKSSDKTHTCPPCPAPELLGGPSVFLFPPKPKDTLMASRTPEVTCV |
| | (I253A, H310A, | VVDVSHEDPEVKFNWYVDGVEVHNAKTKPREEQYNSTYRVVSVLTVL |
| | and H435A) | AQDWLNGKEYKCKVSNKALPAPIEKTISKAKGQPREPQVYTLPPSRD |
| | without C-term. K | ELTKNQVSLTCLVKGFYPSDIAVEWESNGQPENNYKTTPPVLDSDGS |
| | | FFLYSKLTVDKSRWQQGNVFSCSVMHEALHNAYTOKSLSLSPG |
| 459 | IgG1 Fc QL | EPKSSDKTHTCPPCPAPELLGGPSVFLFPPKPKDQLMISRTPEVTCV |
| | (T250Q/ M428L) | VVDVSHEDPEVKFNWYVDGVEVHNAKTKPREEQYNSTYRVVSVLTVL |
| | without C-term. K | HQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGS |
| | | FFLYSKLTVDKSRWQQGNVFSCSVLHEALHNHYTQKSLSLSPG |
| 460 | IgG1 Fc | EPKSSDKTHTCPPCPAPELLGGPSVFLFPPKPKDTLMISRTPEVTCV |
| | T307A/E380A/ | VVDVSHEDPEVKFNWYVDGVEVHNAKTKPREEQYNSTYRVVSVLAVL |
| | N434A without C- | HQDWLNGKEYKCKVSNKALPAPIEKTISKAKGQPREPQVYTLPPSRD |
| | term. K | ELTKNQVSLTCLVKGFYPSDIAVAWESNGQPENNYKTTPPVLDSDGS |
| | | FFLYSKLTVDKSRWQQGNVFSCSVMHEALHAHYTQKSLSLSPG |
| 461 | IgG1 Fc FQQ | EPKSSDKTHTCPPCPAPEFQGGPSVFLFPPKPKDTLMISRTPEVTCV |
| | L234F/L235Q/ | VVDVSHEDPEVKFNWYVDGVEVHNAKTKPREEQYNSTYRVVSVLTVL |
| | K322Q without C- | HQDWLNGKEYKCQVSNKALPAPIEKTISKAKGQPREPQVYTLPPSRD |
| | term. K | ELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 462 | IgG1 Fc LALAPG | EPKSSDKTHTCPPCPAPEAAGGPSVFLFPPKPKDTLMISRTPEVTCV |
| | L234A/L235A/ | VVDVSHEDPEVKFNWYVDGVEVHNAKTKPREEQYNSTYRVVSVLTVL |
| | P329 without C- | HQDWLNGKEYKCKVSNKALGAPIEKTISKAKGQPREPQVYTLPPSRD |
| | term. K | ELTKNQVSLTCLVKGFYPSDIAVEWESNGQPENNYKTTPPVLDSDGS |
| | | FFLYSKLTVDKSRWQQGNVFSCSVLHEALHSHYTQKSLSLSPG |
| 463 | IgG1 Fc FES-LS | EPKSSDKTHTCPPCPAPEFEGGPSVFLFPPKPKDTLMISRTPEVTCV |
| | without C-term. K | VVDVSHEDPEVKFNWYVDGVEVHNAKTKPREEQYNSTYRVVSVLTVL |
| | | HQDWLNGKEYKCKVSNKALPASIEKTISKAKGQPREPQVYTLPPSRD |
| | | ELTKNQVSLTCLVKGFYPSDIAVEWESNGQPENNYKTTPPVLDSDGS |
| | | FFLYSKLIVDKSRWQQGNVFSCSVLHEALHSHYTQKSLSLSPG |

In certain embodiments, an IgG Fe, or a derivative thereof, is linked to the N-terminus or C-terminus of any of the variable heavy chain domains described above with or without a linker. In certain embodiments, human IgG1 Fc, or a derivative thereof, is linked to the N-terminus or C-terminus of any of the variable heavy chain domains described above with or without a linker. In certain embodiments, the amino acid sequence of the human IgG1 Fc comprises or consists of the amino acid sequence of SEQ ID NO: 253 or 261. In certain embodiments, the derivative of human IgG1 Fc comprises or consists of an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to the amino acid sequence of SEQ ID NO: 253 or 261.

In certain embodiments, a human IgG1 Fc comprising a LALA mutation, or a derivative thereof, is linked to the N-terminus or C-terminus of any of the variable heavy chain domains described above with or without a linker. In certain embodiments, the amino acid sequence of the human IgG1 Fc comprising a LALA mutation comprises or consists of the amino acid sequence of SEQ ID NO: 254 or 262. In certain embodiments, the derivative of human IgG1 Fc comprising a LALA mutation comprises or consists of an amino acid sequence at least 85, 90, 95, 96, 97, 98, or 99% identical to the amino acid sequence of SEQ ID NO: 254 or 262.

In certain embodiments, a human IgG1 Fc comprising a LALAPA mutation, or a derivative thereof, is linked to the N-terminus or C-terminus of any of the variable heavy chain domains described above with or without a linker. In certain embodiments, the amino acid sequence of the human IgG1 Fc comprising a LALAPA mutation comprises or consists of the amino acid sequence of SEQ ID NO: 255 or 263. In certain embodiments, the derivative of human IgG1 Fc comprising a LALAPA mutation comprises or consists of an amino acid sequence at least 85, 90, 95, 96, 97, 98, or 99% identical to the amino acid sequence of SEQ ID NO: 255 or 263.

In certain embodiments, a human IgG1 Fc comprising a LALAPALS mutation, or a derivative thereof, is linked to the N-terminus or C-terminus of any of the variable heavy chain domains described above with or without a linker. In certain embodiments, the amino acid sequence of the human IgG1 Fc comprising a LALAPALS mutation comprises or consists of the amino acid sequence of SEQ ID NO: 256 or 264. In certain embodiments, the derivative of human IgG1 Fc comprising a LALAPALS mutation comprises or consists of an amino acid sequence at least 85, 90, 95, 96, 97, 98, or 99% identical to the amino acid sequence of SEQ ID NO: 256 or 264.

In certain embodiments, a human IgG1 Fc comprising a DAPA mutation (D265A/P239A), or a derivative thereof, is linked to the N-terminus or C-terminus of any of the variable heavy chain domains described above with or without a linker. In certain embodiments, the amino acid sequence of the human IgG1 Fc comprising a DAPA mutation comprises or consists of the amino acid sequence of SEQ ID NO: 257 or 265. In certain embodiments, the derivative of human IgG1 Fc comprising a DAPA mutation comprises or consists of an amino acid sequence at least 85, 90, 95, 96, 97, 98, or 99% identical to the amino acid sequence of SEQ ID NO: 257 or 265.

In certain embodiments, a human IgG1 Fc comprising a DANAPA mutation (D265A/N297A/P239A), or a derivative thereof, is linked to the N-terminus or C-terminus of any of the variable heavy chain domains described above with or without a linker. In certain embodiments, the amino acid sequence of the human IgG1 Fc comprising a DANAPA mutation comprises or consists of the amino acid sequence of SEQ ID NO: 258 or 266. In certain embodiments, the derivative of human IgG1 Fc comprising a DANAPA mutation comprises or consists of an amino acid sequence at least 85, 90, 95, 96, 97, 98, or 99% identical to the amino acid sequence of SEQ ID NO: 258 or 266.

In certain embodiments, a human IgG1 Fc comprising a YTE mutation, or a derivative thereof, is linked to the N-terminus or C-terminus of any of the variable heavy chain domains described above with or without a linker. In certain embodiments, the amino acid sequence of the human IgG1 Fc comprising a YTE mutation comprises or consists of the amino acid sequence of SEQ ID NO: 259 or 267. In certain embodiments, the derivative of human IgG1 Fc comprising a YTE mutation comprises or consists of an amino acid sequence at least 85, 90, 95, 96, 97, 98, or 99% identical to the amino acid sequence of SEQ ID NO: 259 or 267.

In certain embodiments, a human IgG1 Fc comprising an FES-YTE mutation, or a derivative thereof, is linked to the N-terminus or C-terminus of any of the variable heavy chain domains described above with or without a linker. In certain embodiments, the amino acid sequence of the human IgG1 Fc comprising an FES-YTE mutation comprises or consists of the amino acid sequence of SEQ ID NO: 260 or 268. In certain embodiments, the derivative of human IgG1 Fc comprising an FES-YTE mutation comprises or consists of an amino acid sequence at least 85, 90, 95, 96, 97, 98, or 99% identical to the amino acid sequence of SEQ ID NO: 260 or 268.

In certain embodiments, a human IgG1 Fc comprising an FES-LS mutation, or a derivative thereof, is linked to the N-terminus or C-terminus of any of the variable heavy chain domains described above with or without a linker. In certain embodiments, the amino acid sequence of the human IgG1 Fc comprising an FES-LS mutation comprises or consists of the amino acid sequence of SEQ ID NO: 450 or 463. In certain embodiments, the derivative of human IgG1 Fc comprising an FES-LS mutation comprises or consists of an amino acid sequence at least 85, 90, 95, 96, 97, 98, or 99% identical to the amino acid sequence of SEQ ID NO: 450 or 463.

In certain embodiments, a human IgG1 Fc comprising a LALAPA-H435A mutation, or a derivative thereof, is linked to the N-terminus or C-terminus of any of the variable heavy chain domains described above with or without a linker. In certain embodiments, the amino acid sequence of the human IgG1 Fc comprising a LALAPA-H435A mutation comprises or consists of the amino acid sequence of SEQ ID NO: 451 or 457. In certain embodiments, the derivative of human IgG1 Fc comprising a LALAPA-H435A mutation comprises or consists of an amino acid sequence at least 85, 90, 95, 96, 97, 98, or 99% identical to the amino acid sequence of SEQ ID NO: 451 or 457.

In certain embodiments, a human IgG1 Fc comprising an IHH mutation (I253A, H310A, and H435A), or a derivative thereof, is linked to the N-terminus or C-terminus of any of the variable heavy chain domains described above with or without a linker. In certain embodiments, the amino acid sequence of the human IgG1 Fc comprising an IHH mutation comprises or consists of the amino acid sequence of SEQ ID NO: 452 or 458. In certain embodiments, the derivative of human IgG1 Fc comprising an IHH mutation comprises or consists of an amino acid sequence at least 85, 90, 95, 96, 97, 98, or 99% identical to the amino acid sequence of SEQ ID NO: 452 or 458.

In certain embodiments, a human IgG1 Fc comprising a QL mutation (T250Q and M428L), or a derivative thereof, is linked to the N-terminus or C-terminus of any of the variable heavy chain domains described above with or without a linker. In certain embodiments, the amino acid sequence of the human IgG1 Fc comprising a QL mutation comprises or consists of the amino acid sequence of SEQ ID NO: 453 or 459. In certain embodiments, the derivative of human IgG1 Fc comprising a QL mutation comprises or consists of an amino acid sequence at least 85, 90, 95, 96, 97, 98, or 99% identical to the amino acid sequence of SEQ ID NO: 453 or 459.

In certain embodiments, a human IgG1 Fc comprising a T307A/E380A/N434A mutation, or a derivative thereof, is linked to the N-terminus or C-terminus of any of the variable heavy chain domains described above with or without a linker. In certain embodiments, the amino acid sequence of the human IgG1 Fc comprising a T307A/E380A/N434A mutation comprises or consists of the amino acid sequence of SEQ ID NO: 454 or 460. In certain embodiments, the derivative of human IgG1 Fc comprising a T307A/E380A/N434A mutation comprises or consists of an amino acid sequence at least 85, 90, 95, 96, 97, 98, or 99% identical to the amino acid sequence of SEQ ID NO: 454 or 460.

In certain embodiments, a human IgG1 Fc comprising an FQQ mutation (L234F, L235Q, and K322Q), or a derivative thereof, is linked to the N-terminus or C-terminus of any of the variable heavy chain domains described above with or without a linker. In certain embodiments, the amino acid sequence of the human IgG1 Fc comprising an FQQ mutation comprises or consists of the amino acid sequence of SEQ ID NO: 455 or 461. In certain embodiments, the derivative of human IgG1 Fc comprising an FQQ mutation comprises or consists of an amino acid sequence at least 85, 90, 95, 96, 97, 98, or 99% identical to the amino acid sequence of SEQ ID NO: 455 or 461.

In certain embodiments, a human IgG1 Fc comprising a LALAPG mutation, or a derivative thereof, is linked to the N-terminus or C-terminus of any of the variable heavy chain domains described above with or without a linker. In certain embodiments, the amino acid sequence of the human IgG1 Fc comprising a LALAPG mutation comprises or consists of the amino acid sequence of SEQ ID NO: 456 or 462. In certain embodiments, the derivative of human IgG1 Fc comprising a LALAPG mutation comprises or consists of an amino acid sequence at least 85, 90, 95, 96, 97, 98, or 99% identical to the amino acid sequence of SEQ ID NO: 456 or 462.

In certain embodiments, the antibodies provided herein comprise an Ig Fc comprising a modified hinge region. As used herein, the term “modified hinge region” refers to an Ig Fc hinge region with an amino acid sequence that has one or more mutations (e.g., amino acid substitutions, insertions, or deletions) relative to the sequence of a wild type Ig Fc hinge region (e.g., an IgG1 Fc hinge region). In certain embodiments, the wild type Ig Fc hinge region comprises SEQ ID NO: 831. In certain embodiments, the modified hinge region comprises one or more mutations relative to the sequence of SEQ ID NO: 831. In certain embodiments, the modified hinge region comprises an amino acid substitution, insertion, and/or deletion. In certain embodiments, the amino acid substitution, insertion, and/or deletion is in the region spanning EU positions 216 to 230. For example, a modified hinge region can comprise a substitution of the amino acid residue at one or more of EU positions 216, 217, 218, 219, 220, 221, 222, 223, 224, 225, 226, 227, 228, 229, and 230, a deletion of the amino acid residue at one or more of EU positions 216, 217, 218, 219, 220, 221, 222, 223, 224, 225, 226, 227, 228, 229, and 230, and/or an insertion following the amino acid residue at one or more of EU positions 216, 217, 218, 219, 220, 221, 222, 223, 224, 225, 226, 227, 228, and 229 (i.e., between positions 216 and 217, 217 and 218, etc.).

In certain embodiments, the modified hinge region comprises a deletion of one or more amino acids at EU positions 216 to 230. In certain embodiments, the modified hinge region comprises 0, 1, 2, 3, 4, 5, 6, 7, 8, 9, 10, 11, 12, 13, 14, or 15 amino acids in the region spanning EU positions 216 to 230. In certain embodiments, the modified hinge region comprises at least 1 (e.g., at least 2, at least 3, at least 4, at least 5, at least 6, at least 7, at least 8, at least 9, at least 10, at least 11, at least 12, at least 13, at least 14, or at least 15) amino acids in the region spanning EU positions 216 to 230. In certain embodiments, the modified hinge region comprises at least 5 amino acids in the region spanning EU positions 216 to 230. In certain embodiments, the modified hinge region comprises the sequence CPPCP (SEQ ID NO: 848) in the region spanning EU positions 216 to 230. In certain embodiments, the modified hinge region comprises 0, 1, 2, 3, 4, 5, 6, 7, 8, 9, or 10 amino acids N-terminal to the sequence CPPCP (SEQ ID NO: 848) in the region spanning EU positions 216 to 230.

In certain embodiments, the modified hinge region is a modified IgG1 Fc hinge region. In certain embodiments, the modified IgG1 Fc hinge region has one or more mutations (e.g., amino acid substitutions, insertions, or deletions) relative to the sequence of a wild type IgG1 Fc hinge region (e.g., the amino acid sequence set forth in SEQ ID NO: 831). In certain embodiments, the modified IgG1 Fc hinge region comprises an amino acid sequence at least 85, 90, 95, 96, 97, 98, or 99% identical to the amino acid sequence of the IgG1 Fc hinge region. In certain embodiments, the modified hinge region comprises or consists of an amino acid sequence at least 85, 90, 95, 96, 97, 98, 99, or 100% identical to an amino acid sequence provided below in Table 10. In certain embodiments, the modified hinge region comprises or consists of an amino acid sequence at least 85, 90, 95, 96, 97, 98, 99, or 100% identical to an amino acid sequence set forth in any one of SEQ ID NOs: 704-718. Table 10 also shows the sequence of an unmodified IgG1 Fc hinge region, according to certain embodiments. Dashes are shown in the modified hinge region amino acid sequences to indicate deletions relative to the unmodified hinge region.

TABLE 10
Modified hinge region amino acid sequences.

| SEQ | | |
|---|---|---|
| ID | Description | Sequence |
| 831 | Unmodified | EPKSSDKTHTCPPCP |
| 704 | Del1 | ---------TCPPCP |
| 705 | Del2 | EPKSS----TCPPCP |
| — | Del3 | --------------- |
| 706 | Del4 | -------THTCPPCP |
| 707 | Del5 | GGGGS----TCPPCP |
| 708 | Del6 | GGGGSGGGGTCPPCP |
| 709 | Del7 | AHHPEEPSSQCPKCP |
| 710 | Del8 | AQQPEEPSSQCPKCP |
| 711 | Del9 | ---GTNEVCKCPKCP |
| 712 | Del10 | EPKSS-KTHTCPPCP |
| 713 | Del11 | EPKS--KTHTCPPCP |
| 714 | Del12 | EPKSSQKTHTCPPCP |
| 715 | Del13 | EPKSAQKTHTCPPCP |
| 716 | Del14 | EPKSA----TCPPCP |
| 717 | Del15 | GGGGSGGGGQCPPCP |
| 718 | Del16 | GGGGSGGGGACPPCP |

In certain embodiments, one, two, or more mutations (e.g., amino acid substitutions, insertions, or deletions) are introduced into the hinge region of the Ig Fc such that the number of cysteine residues in the hinge region are altered (e.g., increased or decreased) as described in, e.g., U.S. Pat. No. 5,677,425, herein incorporated by reference in its entirety. The number of cysteine residues in the hinge region of the CH1 domain may be altered to, e.g., facilitate assembly of the light and heavy chains, or to alter (e.g., increase or decrease) the stability of the antibody.

In certain embodiments, the antibodies provided herein comprise an IgG Fc comprising or consisting of an amino acid sequence at least 85, 90, 95, 96, 97, 98, 99, or 100% identical to an amino acid sequence provided below in Table 11.

TABLE 11
Amino acid sequences of IgG Fcs with modified hinge regions.

| SEQ | | |
|---|---|---|
| ID | Description | Sequence |
| 719 | IgG Fc | TCPPCPAPEAAGGPSVFLFPPKPKDTLMISRTPEVTCVVVDVSHEDPEVKFNWY |
| | (LALAPALS; | VDGVEVHNAKTKPREEQYNSTYRVVSVLTVLHQDWLNGKEYKCKVSNKALAAPI |
| | Del1) + c | EKTISKAKGQPREPQVYTLPPSRDELTKNQVSLTCLVKGFYPSDIAVEWESNGQ |
| | term K | PENNYKTTPPVLDSDGSFFLYSKLTVDKSRWQQGNVFSCSVLHEALHSHYTQKS |
| | | LSLSPGK |
| 720 | IgG Fc | EPKSSTCPPCPAPEAAGGPSVFLFPPKPKDTLMISRTPEVTCVVVDVSHEDPEV |
| | (LALAPALS; | KFNWYVDGVEVHNAKTKPREEQYNSTYRVVSVLTVLHQDWLNGKEYKCKVSNKA |
| | Del2) + c | LAAPIEKTISKAKGQPREPQVYTLPPSRDELTKNQVSLTCLVKGFYPSDIAVEW |
| | term K | ESNGQPENNYKTTPPVLDSDGSFFLYSKLTVDKSRWQQGNVFSCSVLHEALHSH |
| | | YTQKSLSLSPGK |
| 721 | IgG Fc | APEAAGGPSVFLFPPKPKDTLMISRTPEVTCVVVDVSHEDPEVKFNWYVDGVEV |
| | (LALAPALS; | HNAKTKPREEQYNSTYRVVSVLTVLHQDWLNGKEYKCKVSNKALAAPIEKTISK |
| | Del3) + c | AKGQPREPQVYTLPPSRDELTKNQVSLTCLVKGFYPSDIAVEWESNGQPENNYK |
| | term K | TTPPVLDSDGSFFLYSKLTVDKSRWQQGNVFSCSVLHEALHSHYTQKSLSLSPG |
| | | K |
| 722 | IgG Fc | THTCPPCPAPEAAGGPSVFLFPPKPKDTLMISRTPEVTCVVVDVSHEDPEVKFN |
| | (LALAPALS; | WYVDGVEVHNAKTKPREEQYNSTYRVVSVLTVLHQDWLNGKEYKCKVSNKALAA |
| | Del4) + c | PIEKTISKAKGQPREPQVYTLPPSRDELTKNQVSLTCLVKGFYPSDIAVEWESN |
| | term K | GQPENNYKTTPPVLDSDGSFFLYSKLTVDKSRWQQGNVFSCSVLHEALHSHYTQ |
| | | KSLSLSPGK |
| 723 | IgG Fc | GGGGSTCPPCPAPEAAGGPSVFLFPPKPKDTLMISRTPEVTCVVVDVSHEDPEV |
| | (LALAPALS; | KFNWYVDGVEVHNAKTKPREEQYNSTYRVVSVLTVLHQDWLNGKEYKCKVSNKA |
| | Del5) + c | LAAPIEKTISKAKGQPREPQVYTLPPSRDELTKNQVSLTCLVKGFYPSDIAVEW |
| | term K | ESNGQPENNYKTTPPVLDSDGSFFLYSKLTVDKSRWQQGNVFSCSVLHEALHSH |
| | | YTQKSLSLSPGK |
| 724 | IgG Fc | GGGGSGGGGTCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del6) + c | SNKALAAPIEKTISKAKGQPREPQVYTLPPSRDELTKNQVSLTCLVKGFYPSDI |
| | term K | AVEWESNGQPENNYKTTPPVLDSDGSFFLYSKLTVDKSRWQQGNVFSCSVLHEA |
| | | LHSHYTQKSLSLSPGK |
| 725 | IgG Fc | AHHPEEPSSQCPKCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del7) + c | SNKALAAPIEKTISKAKGQPREPQVYTLPPSRDELTKNQVSLTCLVKGFYPSDI |
| | term K | AVEWESNGQPENNYKTTPPVLDSDGSFFLYSKLTVDKSRWQQGNVFSCSVLHEA |
| | | LHSHYTQKSLSLSPGK |
| 726 | IgG Fc | AQQPEEPSSQCPKCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del8) + c | SNKALAAPIEKTISKAKGQPREPQVYTLPPSRDELTKNQVSLTCLVKGFYPSDI |
| | term K | AVEWESNGQPENNYKTTPPVLDSDGSFFLYSKLTVDKSRWQQGNVFSCSVLHEA |
| | | LHSHYTQKSLSLSPGK |
| 727 | IgG Fc | GTNEVCKCPKCPAPEAAGGPSVFLFPPKPKDTLMISRTPEVTCVVVDVSHEDPE |
| | (LALAPALS; | VKFNWYVDGVEVHNAKTKPREEQYNSTYRVVSVLTVLHQDWLNGKEYKCKVSNK |
| | Del9) + c | ALAAPIEKTISKAKGQPREPQVYTLPPSRDELTKNQVSLTCLVKGFYPSDIAVE |
| | term K | WESNGQPENNYKTTPPVLDSDGSFFLYSKLTVDKSRWQQGNVFSCSVLHEALHS |
| | | HYTQKSLSLSPGK |
| 728 | IgG Fc | EPKSSKTHTCPPCPAPEAAGGPSVFLFPPKPKDTLMISRTPEVTCVVVDVSHED |
| | (LALAPALS; | PEVKFNWYVDGVEVHNAKTKPREEQYNSTYRVVSVLTVLHQDWLNGKEYKCKVS |
| | Del10) + | NKALAAPIEKTISKAKGQPREPQVYTLPPSRDELTKNQVSLTCLVKGFYPSDIA |
| | c term K | VEWESNGQPENNYKTTPPVLDSDGSFFLYSKLTVDKSRWQQGNVFSCSVLHEAL |
| | | HSHYTQKSLSLSPGK |
| 729 | IgG Fc | EPKSKTHTCPPCPAPEAAGGPSVFLFPPKPKDTLMISRTPEVTCVVVDVSHEDP |
| | (LALAPALS; | EVKFNWYVDGVEVHNAKTKPREEQYNSTYRVVSVLTVLHQDWLNGKEYKCKVSN |
| | Del11) + | KALAAPIEKTISKAKGQPREPQVYTLPPSRDELTKNQVSLTCLVKGFYPSDIAV |
| | c term K | EWESNGQPENNYKTTPPVLDSDGSFFLYSKLTVDKSRWQQGNVFSCSVLHEALH |
| | | SHYTQKSLSLSPGK |
| 730 | IgG Fc | EPKSSQKTHTCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del12) + | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPGK |
| 731 | IgG Fc | EPKSAQKTHTCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del13) + | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPGK |
| 732 | IgG Fc | EPKSATCPPCPAPEAAGGPSVFLFPPKPKDTLMISRTPEVTCVVVDVSHEDPEV |
| | (LALAPALS; | KFNWYVDGVEVHNAKTKPREEQYNSTYRVVSVLTVLHQDWLNGKEYKCKVSNKA |
| | Del14) + | LAAPIEKTISKAKGQPREPQVYTLPPSRDELTKNQVSLTCLVKGFYPSDIAVEW |
| | c term K | ESNGQPENNYKTTPPVLDSDGSFFLYSKLTVDKSRWQQGNVFSCSVLHEALHSH |
| | | YTQKSLSLSPGK |
| 733 | IgG Fc | GGGGSGGGGQCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del15) + | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPGK |
| 734 | IgG Fc | GGGGSGGGGACPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del16) + | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPGK |
| 735 | IgG Fc | TCPPCPAPEAAGGPSVFLFPPKPKDTLMISRTPEVTCVVVDVSHEDPEVKFNWY |
| | (LALAPALS; | VDGVEVHNAKTKPREEQYNSTYRVVSVLTVLHQDWLNGKEYKCKVSNKALAAPI |
| | Del1) no | EKTISKAKGQPREPQVYTLPPSRDELTKNQVSLTCLVKGFYPSDIAVEWESNGQ |
| | c term K | PENNYKTTPPVLDSDGSFFLYSKLTVDKSRWQQGNVFSCSVLHEALHSHYTQKS |
| | | LSLSPG |
| 736 | IgG Fc | EPKSSTCPPCPAPEAAGGPSVFLFPPKPKDTLMISRTPEVTCVVVDVSHEDPEV |
| | (LALAPALS; | KFNWYVDGVEVHNAKTKPREEQYNSTYRVVSVLTVLHQDWLNGKEYKCKVSNKA |
| | Del2) no | LAAPIEKTISKAKGQPREPQVYTLPPSRDELTKNQVSLTCLVKGFYPSDIAVEW |
| | c term K | ESNGQPENNYKTTPPVLDSDGSFFLYSKLTVDKSRWQQGNVFSCSVLHEALHSH |
| | | YTQKSLSLSPG |
| 737 | IgG Fc | APEAAGGPSVFLFPPKPKDTLMISRTPEVTCVVVDVSHEDPEVKFNWYVDGVEV |
| | (LALAPALS; | HNAKTKPREEQYNSTYRVVSVLTVLHQDWLNGKEYKCKVSNKALAAPIEKTISK |
| | Del3) no | AKGQPREPQVYTLPPSRDELTKNQVSLTCLVKGFYPSDIAVEWESNGQPENNYK |
| | c term K | TTPPVLDSDGSFFLYSKLTVDKSRWQQGNVFSCSVLHEALHSHYTQKSLSLSPG |
| 738 | IgG Fc | THTCPPCPAPEAAGGPSVFLFPPKPKDTLMISRTPEVTCVVVDVSHEDPEVKFN |
| | (LALAPALS; | WYVDGVEVHNAKTKPREEQYNSTYRVVSVLTVLHQDWLNGKEYKCKVSNKALAA |
| | Del4) no | PIEKTISKAKGQPREPQVYTLPPSRDELTKNQVSLTCLVKGFYPSDIAVEWESN |
| | c term K | GQPENNYKTTPPVLDSDGSFFLYSKLTVDKSRWQQGNVFSCSVLHEALHSHYTQ |
| | | KSLSLSPG |
| 739 | IgG Fc | GGGGSTCPPCPAPEAAGGPSVFLFPPKPKDTLMISRTPEVTCVVVDVSHEDPEV |
| | (LALAPALS; | KFNWYVDGVEVHNAKTKPREEQYNSTYRVVSVLTVLHQDWLNGKEYKCKVSNKA |
| | Del5) no | LAAPIEKTISKAKGQPREPQVYTLPPSRDELTKNQVSLTCLVKGFYPSDIAVEW |
| | c term K | ESNGQPENNYKTTPPVLDSDGSFFLYSKLTVDKSRWQQGNVFSCSVLHEALHSH |
| | | YTQKSLSLSPG |
| 740 | IgG Fc | GGGGSGGGGTCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del6) no | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPG |
| 741 | IgG Fc | AHHPEEPSSQCPKCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del7) no | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPG |
| 742 | IgG Fc | AQQPEEPSSQCPKCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del8) no | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPG |
| 743 | IgG Fc | GTNEVCKCPKCPAPEAAGGPSVFLFPPKPKDTLMISRTPEVTCVVVDVSHEDPE |
| | (LALAPALS; | VKFNWYVDGVEVHNAKTKPREEQYNSTYRVVSVLTVLHQDWLNGKEYKCKVSNK |
| | Del9) no | ALAAPIEKTISKAKGQPREPQVYTLPPSRDELTKNQVSLTCLVKGFYPSDIAVE |
| | c term K | WESNGQPENNYKTTPPVLDSDGSFFLYSKLTVDKSRWQQGNVFSCSVLHEALHS |
| | | HYTQKSLSLSPG |
| 744 | IgG Fc | EPKSSKTHTCPPCPAPEAAGGPSVFLFPPKPKDTLMISRTPEVTCVVVDVSHED |
| | (LALAPALS; | PEVKFNWYVDGVEVHNAKTKPREEQYNSTYRVVSVLTVLHQDWLNGKEYKCKVS |
| | Del10) no | NKALAAPIEKTISKAKGQPREPQVYTLPPSRDELTKNQVSLTCLVKGFYPSDIA |
| | c term K | VEWESNGQPENNYKTTPPVLDSDGSFFLYSKLTVDKSRWQQGNVFSCSVLHEAL |
| | | HSHYTQKSLSLSPG |
| 745 | IgG Fc | EPKSKTHTCPPCPAPEAAGGPSVFLFPPKPKDTLMISRTPEVTCVVVDVSHEDP |
| | (LALAPALS; | EVKFNWYVDGVEVHNAKTKPREEQYNSTYRVVSVLTVLHQDWLNGKEYKCKVSN |
| | Del11) no | KALAAPIEKTISKAKGQPREPQVYTLPPSRDELTKNQVSLTCLVKGFYPSDIAV |
| | c term K | EWESNGQPENNYKTTPPVLDSDGSFFLYSKLTVDKSRWQQGNVFSCSVLHEALH |
| | | SHYTQKSLSLSPG |
| 746 | IgG Fc | EPKSSQKTHTCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del12) no | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPG |
| 747 | IgG Fc | EPKSAQKTHTCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del13) no | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPG |
| 748 | IgG Fc | EPKSATCPPCPAPEAAGGPSVFLFPPKPKDTLMISRTPEVTCVVVDVSHEDPEV |
| | (LALAPALS; | KFNWYVDGVEVHNAKTKPREEQYNSTYRVVSVLTVLHQDWLNGKEYKCKVSNKA |
| | Del14) no | LAAPIEKTISKAKGQPREPQVYTLPPSRDELTKNQVSLTCLVKGFYPSDIAVEW |
| | c term K | ESNGQPENNYKTTPPVLDSDGSFFLYSKLTVDKSRWQQGNVFSCSVLHEALHSH |
| | | YTQKSLSLSPG |
| 749 | IgG Fc | GGGGSGGGGQCPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del15) no | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPG |
| 750 | IgG Fc | GGGGSGGGGACPPCPAPEAAGGPSVFLFPPKPKDTLMISRTPEVTCVVVDVSHE |
| | (LALAPALS; | DPEVKFNWYVDGVEVHNAKTKPREEQYNSTYRVVSVLTVLHQDWLNGKEYKCKV |
| | Del16) no | SNKALAAPIEKTISKAKGQPREPQVYTLPPSRDELTKNQVSLTCLVKGFYPSDI |
| | c term K | AVEWESNGQPENNYKTTPPVLDSDGSFFLYSKLTVDKSRWQQGNVFSCSVLHEA |
| | | LHSHYTQKSLSLSPG |

### Other Half-Life Extending Moieties

As used herein, the term “half-life extending moiety” includes non-proteinaceous, half-life extending moieties, such as PEG or HES, and proteinaceous half-life extending moieties. In certain embodiments, non-proteinaceous half-life extending moieties are linked to the antibodies described herein. In certain embodiments, the non-proteinaceous half-life extending moieties are linked to the antibodies instead of an Ig Fc. In certain embodiments, the non-proteinaceous half-life extending moieties are linked to the antibodies in addition to an Ig Fc.

Examples of suitable polymer molecules that act as non-proteinaceous half-life extending moieties include polymer molecules selected from the group consisting of polyalkylene oxide (PAO), including polyalkylene glycol (PAG), such as polyethylene glycol (PEG) and polypropylene glycol (PPG), branched PEGs, hydroxyalkyl starch (HAS), such as hydroxyethyl starch (HES), polysialic acid (PSA), poly-vinyl alcohol (PVA), poly-carboxylate, poly-(vinylpyrrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG has the advantage of having only few reactive groups capable of cross-linking compared to, e.g., polysaccharides such as dextran. In particular, monofunctional PEG, e.g., methoxypolyethylene glycol (mPEG), is of interest since its coupling chemistry is relatively simple (only one reactive group is available for conjugating with attachment groups on the polypeptide). Consequently, as the risk of cross-linking is eliminated, the resulting conjugated antibodies described herein are more homogeneous, and the reaction of the polymer molecules with the variant polypeptide is easier to control.

To effect covalent attachment of the polymer molecule(s) to the antibodies described herein, the hydroxyl end groups of the polymer molecule must be provided in activated form, i.e., with reactive functional groups (examples of which include primary amino groups, hydrazide (HZ), thiol, succinate (SUC), succinimidyl succinate (SS), succinimidyl succinamide (SSA), succinimidyl propionate (SPA), succinimidyl butyrate (SBA), succinimidyl carboxymethylate (SCM), benzotriazole carbonate (BTC), N-hydroxysuccinimide (NHS), aldehyde, nitrophenylcarbonate (NPC), and tresylate (TRES)). Suitable activated polymer molecules are commercially available, e.g., from Shearwater Polymers, Inc., Huntsville, Ala., USA, or from PolyMASC Pharmaceuticals plc, UK.

Alternatively, the polymer molecules can be activated by conventional methods known in the art, e.g., as disclosed in WO 90/13540. Specific examples of activated linear or branched polymer molecules for use herein are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG, BTC-PEG, EPOXPEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. Nos. 5,932,462 and 5,643,575, both of which are incorporated herein by reference. Furthermore, the following publications disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503, and EP 154 316.

Specific examples of activated PEG polymers particularly preferred for coupling to cysteine residues, include the following linear PEGs: vinylsulfone-PEG (VS-PEG), preferably vinylsulfone-mPEG (VS-mPEG); maleimide-PEG (MAL-PEG), preferably maleimide-mPEG (MAL-mPEG) and orthopyridyl-disulfide-PEG (OPSS-PEG), preferably orthopyridyl-disulfide-mPEG (OPSS-mPEG). Typically, such PEG or mPEG polymers will have a size of about 5 kDa, about 10 kDa, about 12 kDa or about 20 kDa.

The conjugation of the antibodies described herein and the activated polymer molecules is conducted by use of any conventional method, e.g., as described in the following references (which also describe suitable methods for activation of polymer molecules): Harris and Zalipsky, eds., Poly(ethylene glycol) Chemistry and Biological Applications, AZC Washington; R. F. Taylor, (1991), “Protein immobilisation. Fundamental and applications,” Marcel Dekker, N.Y.; S. S. Wong, (1992), “Chemistry of Protein Conjugation and Crosslinking,” CRC Press, Boca Raton; G. T. Hermanson et al., (1993), “Immobilized Affinity Ligand Techniques”, Academic Press, N.Y.

The skilled person will be aware that the activation method and/or conjugation chemistry to be used depends on the attachment group(s) of the antibody (examples of which are given further above), as well as the functional groups of the polymer (e.g., being amine, hydroxyl, carboxyl, aldehyde, sulfhydryl, succinimidyl, maleimide, vinylsulfone or haloacetate). The PEGylation may be directed towards conjugation to all available attachment groups on the antibody (i.e., such attachment groups that are exposed at the surface of the polypeptide) or may be directed towards one or more specific attachment groups, e.g., the N-terminal amino group as described in U.S. Pat. No. 5,985,265 or to cysteine residues. Furthermore, the conjugation may be achieved in one step or in a stepwise manner (e.g., as described in WO 99/55377).

For PEGylation to cysteine residues (see above) the antibody is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to 16 hours.

It will be understood that the PEGylation is designed so as to produce the optimal molecule with respect to the number of PEG molecules attached, the size and form of such molecules (e.g., whether they are linear or branched), and the attachment site(s) in the antibody. The molecular weight of the polymer to be used may e.g., be chosen on the basis of the desired effect to be achieved.

In connection with conjugation to only a single attachment group on the antibody (e.g., the N-terminal amino group), it may be advantageous that the polymer molecule, which may be linear or branched, has a high molecular weight, preferably about 10-25 kDa, such as about 15-25 kDa, e.g., about 20 kDa.

Normally, the polymer conjugation is performed under conditions aimed at reacting as many of the available polymer attachment groups with polymer molecules. This is achieved by means of a suitable molar excess of the polymer relative to the polypeptide. Typically, the molar ratios of activated polymer molecules to polypeptide are up to about 1000-1, such as up to about 200-1, or up to about 100-1. In some cases, the ratio may be somewhat lower, however, such as up to about 50-1, 10-1, 5-1, 2-1 or 1-1 in order to obtain optimal reaction.

It is also contemplated to couple the polymer molecules to the antibody through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation, residual activated polymer molecules are blocked according to methods known in the art, e.g., by addition of primary amine to the reaction mixture, and the resulting inactivated polymer molecules are removed by a suitable method.

It will be understood that depending on the circumstances, e.g., the amino acid sequence of the antibody, the nature of the activated PEG compound being used and the specific PEGylation conditions, including the molar ratio of PEG to polypeptide, varying degrees of PEGylation may be obtained, with a higher degree of PEGylation generally being obtained with a higher ratio of PEG to antibody. The PEGylated antibodies resulting from any given PEGylation process will, however, normally comprise a stochastic distribution of conjugated antibody having slightly different degrees of PEGylation.

For improvement of the biological half-life of the antibodies described herein, chemical modification such as PEGylation, or HESylation are applicable.

HAS and HES non-proteinaceous polymers, as well as methods of producing HAS or HES conjugates are disclosed for example in WO 02/080979, WO 03/070772, WO 057092391 and WO 057092390.

Polysialytion is another technology, which uses the natural polymer polysialic acid (PSA) to prolong the half-life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the antibody in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

### Antibody Conjugates

The anti-APJ antibodies of the present disclosure can be linked to or co-expressed with another functional molecule, e.g., another peptide or protein. For example, an antibody or fragment thereof can be functionally linked (e.g., by chemical coupling, genetic fusion, noncovalent association, or otherwise) to one or more other molecular entities, such as another antibody or antibody fragment to produce a bispecific or a multispecific antibody (e.g., a bispecific T cell engager [BiTE] or a dual-affinity re-targeting antibody [DART]) with a second or additional binding specificity. In certain embodiments, the bispecific or multispecific antibody has binding specificity for a molecule on an effector cell (e.g., CD3, CD16, CD137). In certain embodiments, an antibody provided herein is a multispecific antibody.

In certain embodiments, an antibody disclosed herein is conjugated to a pharmaceutically active substance. Pharmaceutically active substances include, but are not limited to, cytotoxic agents, cytostatic agents, toxins, radionuclides (e.g., radioisotopes), polypeptides, polynucleotides, detectable labels, and combinations thereof. In certain embodiments, the antibody is conjugated to a cytotoxic agent, a cytostatic agent, a toxin, a radionuclide, a detectable label, or a combination thereof. In certain embodiments, the pharmaceutically active substance is a cytotoxic agent. In certain embodiments, the cytotoxic agent is able to induce death or destruction of a cell in contact therewith. In certain embodiments, the pharmaceutically active substance is a cytostatic agent. In certain embodiments, the cytostatic agent is able to prevent or substantially reduce proliferation and/or inhibits the activity or function of a cell in contact therewith. In certain embodiments, the cytotoxic agent or cytostatic agent is a chemotherapeutic agent.

In certain embodiments, the pharmaceutically active substance is a radionuclide. Suitable radionuclides include, but are not limited to, beta emitters, auger emitters, converted electron emitters, alpha emitters, and low photon energy emitters. In certain embodiments, the radionuclide is selected from ³H, ¹⁴C, ³²P, ³⁵S, ³⁶Cl, ⁴⁵Ca, ⁵¹Cr, ⁵⁷Co, ⁵⁸Co, ⁵⁹Fe, ⁶⁷Cu, ⁶⁷Ga, ⁷⁶As, ⁷⁷As, ⁸⁹Sr, ⁹⁰Y ⁹⁹Tc, ^{99m}Tc ¹⁰⁵Rh, ¹¹¹In, ^{114m}In, ¹¹⁷Lu, ¹²¹I, ¹³¹I, ¹²⁴I, ¹²⁵I, ¹³¹I, ¹⁴⁹Tb, ¹⁵³Sm, ¹⁶¹Tb, ¹⁶⁶Ho, ¹⁷⁷Lu, ¹⁹⁸Au, ²⁰¹Tl, ²¹¹At, ²¹²Pb, ²¹³Bi, ²²⁵Ac, ¹⁸⁶Re, ¹⁸⁸Re, ²¹²Bi, ²¹³Bi, ²²¹At, ²²³Ac, ²²³Ra, ²²⁵Ac, ²⁵⁵Fm, and combinations thereof.

In certain embodiments, the pharmaceutically active substance is a detectable label. In certain embodiments, the detectable label comprises a fluorescent moiety, a click chemistry handle, or a combination thereof.

In certain embodiments, the pharmaceutically active substance is a drug. Suitable drugs include, but are not limited to, anti-cancer agents, anti-inflammatory agents, and anti-infective (e.g., anti-fungal, antibacterial, anti-parasitic, antiviral) agents. Suitable anti-cancer agents include, but are not limited to, alkylating agents, antimetabolites, spindle poison plant alkaloids, cytotoxic/antitumor antibiotics, topoisomerase inhibitors, photosensitizers, kinase inhibitors, anti-hormonal agents, aromatase inhibitors, anti-androgens, protein kinase inhibitors, lipid kinase inhibitors, antisense oligonucleotides (e.g., those which inhibit expression of genes in signaling pathways implicated in aberrant cell proliferation), ribozymes, (e.g., VEGF expression inhibitors and HER2 expression inhibitors), vaccines (e.g., gene therapy vaccines), topoisomerase 1 inhibitors, anti-angiogenic agents, pharmaceutically acceptable salts, acids, solvates and derivatives of any of the above, and any combination thereof.

In certain embodiments, the pharmaceutically active substance is a toxin. Suitable toxins include, but are not limited to, proteinaceous toxins (e.g., bacterial-derived toxins, and plant-derived toxins), toxins targeting tubulin filaments, toxins targeting DNA, toxins targeting RNA. Examples of proteinaceous toxins include saporin, dianthin, ricin, modeccin, abrin, volkensin, viscumin, shiga toxin, shiga-like toxin, pseudomonas exotoxin (PE, also known as exotoxin A), diphtheria toxin (DT), and cholera toxin. Examples of toxins targeting tubulin filaments include maytansinoids (e.g., DM1 and DM4), auristatins (e.g., Monomethyl auristatin E (MMAE) and Monomethyl auristatin F (MMAF)), toxoids, tubulysins, cryptophycins, rhizoxin. Examples of DNA-targeting toxins include calicheamicins: N-Acetyl-y-calicheamicin, CC-1065 analogs, duocarmycins, doxorubicin, methotrexate, benzodiazepines, camptothecin analogues, and anthracyclines. Examples of RNA-targeting toxins are amanitins, spliceostatins, and thailanstatins.

In certain embodiments, the pharmaceutically active substance is a polypeptide. Suitable polypeptides include, but are not limited to, Cas9; toxins (e.g., saporin, dianthin, gelonin, (de)bouganin, agrostin, ricin (toxin A chain); pokeweed antiviral protein, apoptin, diphtheria toxin, pseudomonas exotoxin); metabolic enzymes (e.g., argininosuccinate lyase, argininosuccinate synthetase); enzymes of the coagulation cascade; repairing enzymes; enzymes for cell signaling; cell cycle regulation factors; gene regulating factors (e.g., ranscription factors such as NF-KB or gene repressors such as methionine repressor).

In certain embodiments, the pharmaceutically active substance is a polynucleotide. In certain embodiments, the polynucleotide comprises coding information. In certain embodiments, the polynucleotide is a gene or an open reading frame encoding a protein. In certain embodiments, the polynucleotide comprises regulatory information. In certain embodiments, the polynucleotide is a promoter, a regulatory element binding region, or a sequence encoding a micro RNA. Suitable polynucleotides include natural and artificial nucleic acids. Artificial nucleic acids include, but are not limited to, peptide nucleic acids (PNA), Morpholinos and locked nucleic acids (LNA), glycol nucleic acids (GNA), and threose nucleic acids (TNA). Each of these is distinguished from naturally occurring DNA or RNA by changes to the backbone of the molecule. Suitable polynucleotides include, but are not limited to, a vector; a gene (e.g., a cell suicide-inducing transgene); single stranded DNA; linear double stranded DNA; circular double stranded DNA (e.g., a plasmid); mini-circle DNA; a DNA aptamer; single stranded RNA; linear double stranded RNA; mRNA; tRNA; rRNA; short interfering RNA (siRNA); microRNA (miRNA); antisense RNA; anti-sense oligonucleotides; peptide nucleic acid (PNA); phosphoramidate morpholino oligomer (PMO); locked nucleic acid (LNA); bridged nucleic acid (BNA); 2′-deoxy-2′-fluoroarabino nucleic acid (FANA); 2′-O-methoxyethyl-RNA (MOE); 2′-0,4′-aminoethylene bridged nucleic acid; 3′-fluoro hexitol nucleic acid (FHNA); an RNA aptamer; and combinations thereof.

### Exemplary Anti-APJ Antibody Sequences

In certain embodiments, the instant disclosure provides an antibody that specifically binds to APJ (e.g., human APJ), the antibody comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to any one of the amino acid sequences shown in Table 12. In certain embodiments, the antibody comprises or consists of any one of the amino acid sequences shown in Table 12.

TABLE 12
Anti-APJ antibody amino acid sequences.

| | SEQ | | |
|---|---|---|---|
| | ID | | |
| Ab | NO | Description | Sequence |
| Ab001 | 270 | FL VH- | EVQLVESGGGLVQPGGSLRLSCAASGSTYSSHCMGWYRQAPGKGREFVALMT |
| | | Ig F | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab002 | 271 | FL VH- | EVQLVESGGGLVQPGGSLRLSCAASGSTYSSHCMGWYRQAPGKGREFVALMT |
| | | Ig Ig Fc | RSRGTSYADSVKGRFTISRDNSKNTVYLQMNSLRAEDTAVYYCAAVPRAGIE |
| | | (LALA | SGAYCKWIIKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab003 | 272 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab004 | 273 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRYQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab005 | 274 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWRYQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab006 | 275 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIIQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab007 | 276 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWIIQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab008 | 277 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPRKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab009 | 278 | FL VH- | EVQLVESGGGLVQPGGSLRLSCAASGSTYSSHCMGWYRQAPGKGREFVALMT |
| | | Ig Fc | RSYGTSYADSVKGRFTISRDNSKNTVYLQMNSLRAEDTAVYYCAAVPRAGIE |
| | | (LALA | SGAYCKWPRKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab010 | 279 | FL VH- | EVQLVESGGGLVQPGGSLRLSCAASGSTYSSHCMGWYRQAPGKGREFVALMT |
| | | Ig Fc | RSYGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab011 | 280 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab012 | 281 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab013 | 282 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab014 | 283 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab015 | 284 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPHKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab016 | 285 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPHQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab017 | 286 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPHQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab018 | 287 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab019 | 288 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab020 | 289 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab021 | 290 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab022 | 291 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWKNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab023 | 292 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWKYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab024 | 293 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab025 | 294 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab026 | 295 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab027 | 296 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWQYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab028 | 297 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab029 | 298 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab030 | 299 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab031 | 300 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIHKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab032 | 301 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWILKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab033 | 302 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWINKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab034 | 303 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTHKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab035 | 304 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTIKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab036 | 305 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTLKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab037 | 306 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab038 | 307 | FL VH- | EVQLVESGGGLVQPGGSLRLSCAASGYNYVFHCMGWYRQAPGKGREFVALMS |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab039 | 308 | FL VH- | EVQLVESGGGLVQPGGSLRLSCAASGITYVSHCMGWYRQAPGKGREFVAAMS |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab040 | 309 | FL VH- | EVQLVESGGGLVQPGGSLRLSCAASGITYSSHCMGWYRQAPGKGREFVAAMQ |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab041 | 310 | FL VH- | EVQLVESGGGLVQPGGSLRLSCAASGITYQSHCMGWYRQAPGKGREFVALIQ |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab042 | 311 | FL VH- | EVQLVESGGGLVQPGGSLRLSCAASGLHYHSHCMGWYRQAPGKGREFVAAMS |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab043 | 312 | FL VH- | EVQLVESGGGLVQPGGSLRLSCAASGIHYSSHCMGWYRQAPGKGREFVALMS |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab044 | 313 | FL VH- | EVQLVESGGGLVQPGGSLRLSCAASGFTYQSHCMGWYRQAPGKGREFVALMQ |
| | | Ig Fc | HSRGTYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab045 | 314 | FL VH- | EVQLVESGGGLVQPGGSLRLSCAASGFLYSFHCMGWYRQAPGKGREFVALIT |
| | | Ig Fc | HSRGYSYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab046 | 315 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab047 | 316 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab048 | 317 | FL VH- | EVQLVESGGGLVQPGGSLRLSCAASGSTYSSHCMGWYRQAPGKGREFVALMT |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab049 | 318 | FL VH- | EVQLVESGGGLVQPGGSLRLSCAASGSTYSSHCMGWYRQAPGKGREFVALMT |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab050 | 319 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab051 | 320 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab052 | 321 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab053 | 322 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab054 | 323 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab055 | 324 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab056 | 325 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab057 | 326 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab058 | 327 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab059 | 328 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab060 | 329 | FL VH- | QVQLVESGGGSVQSGGSLTLSCAASGSTYSSHCMGWFRQAPGKEREGVALMT |
| | | Ig Fc | RSRGTSYADSVKGRFTISQDNTKNILYLQMNSLKPEDTAMYYCAAVPRAGIE |
| | | (LALA | YSGAYCKWNMKDSGSWGQGTLVTVSSGGGGSEPKSSDKTHTCPPCPAPEAAG |
| | | PA) | GPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALAAPIEKTISKAK |
| | | | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVMHEALHNHYTQKSLSLS |
| | | | PGK |
| Ab061 | 330 | FL VH- | QVQLVESGGGSVQSGGSLTLSCAASGSTYSSHCMGWFRQAPGKEREGVALMT |
| | | Ig Fc | RSRGTSYADSVKGRFTISQDNTKNILYLQMNSLKPEDTAMYYCAAVPRAGIE |
| | | (LALA | SGAYCKWNMKDSGSWGQGTLVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab062 | 331 | FL VH- | QVQLVESGGGSVQSGGSLTLSCAASGSTYASHCMGWFRQAPGKEREGVALMT |
| | | Ig Fc | RSRGTSYADSVKGRFTISQDNIKNILYLQMNSLKPEDTAMYYCAAVPRAGIE |
| | | (LALA | SGAYCKWNMKDSGSWGQGTLVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab063 | 332 | FL VH- | QVQLVESGGGSVQSGGSLTLSCAASGSTYSSHCMGWFRQAPGKEREGVALMA |
| | | Ig Fc | RSRGTSYADSVKGRFTISQDNTKNILYLQMNSLKPEDTAMYYCAAVPRAGIE |
| | | (LALA | SGAYCKWNMKDSGSWGQGTLVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab064 | 333 | FL VH- | QVQLVESGGGSVQSGGSLTLSCAASGSTYSSHCMGWFRQAPGKEREGVALMT |
| | | Ig Fc | RSRGTSYADSVKGRFTISQDNTKNILYLQMNSLKPEDTAMYYCAAVPRAGIE |
| | | (LALA | SGAYCKANMKDSGSWGQGTLVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab065 | 334 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | YSGAYCKWNMKDSGSWGQGTQVTVSSGGGGSEPKSSDKTHTCPPCPAPEAAG |
| | | PA) | GPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALAAPIEKTISKAK |
| | | | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVMHEALHNHYTQKSLSLS |
| | | | PGK |
| Ab066 | 335 | FL VH- | EVQLVESGGGLVQPGGSLRLSCAASGSTYASHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab067 | 336 | FL VH- | EVQLVESGGGLVQPGGSLRLSCAASGSTYSSHCMGWYRQAPGKGREFVALMA |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab068 | 337 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKANMKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab069 | 338 | FL VH- | EVQLVESGGGLVQPGGSLRLSCAASGSTYSSHCMGWYRQAPGKGREFVALMT |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab070 | 339 | FL VH- | EVQLVESGGGLVQPGGSLRLSCAASGSTYSSHCMGWYRQAPGKGREFVALMT |
| | | Ig Fc | HSRGTSYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PA) | GPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALAAPIEKTISKAK |
| | | | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVMHEALHNHYTQKSLSLS |
| | | | PGK |
| Ab071 | 340 | FL VH- | EVQLVESGGGLVQPGGSLRLSCAASGLTFSSHCMGWYRQAPGKGREFVAAIQ |
| | | Ig Fc | GSRGSTYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PA) | GPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALAAPIEKTISKAK |
| | | | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVMHEALHNHYTQKSLSLS |
| | | | PGK |
| Ab072 | 341 | FL VH- | EVQLVESGGGLVQPGGSLRLSCAASGLTFSSHCMGWYRQAPGKGREFVAAIQ |
| | | Ig Fc | HSRGSTYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PA) | GPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALAAPIEKTISKAK |
| | | | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVMHEALHNHYTQKSLSLS |
| | | | PGK |
| Ab073 | 342 | FL VH- | EVQLVESGGGLVQPGGSLRLSCAASGLTFSSHCMGWYRQAPGKGREFVAAIQ |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab074 | 343 | FL VH- | EVQLVESGGGLVQPGGSLRLSCAASGLTFSSHCMGWYRQAPGKGREFVAAIQ |
| | | Ig Fc | RSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab075 | 344 | FL VH- | EVQLVESGGGLVQPGGSLRLSCAASGLTFSSHCMGWYRQAPGKGREFVAAIS |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHICPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab076 | 345 | FL VH- | EVQLVESGGGLVQPGGSLRLSCAASGLTFSSHCMGWYRQAPGKGREFVAAIS |
| | | Ig Fc | GSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab077 | 346 | FL VH- | EVQLVESGGGLVQPGGSLRLSCAASGLTFSSHCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab078 | 347 | FL VH- | EVQLVESGGGLVQPGGSLRLSCAASGLTFSSHCMGWYRQAPGKGREFVAAIQ |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab079 | 348 | FL VH- | EVQLVESGGGLVQPGGSLRLSCAASGLTFSSHCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab080 | 349 | FL VH- | EVQLVESGGGLVQPGGSLRLSCAASGFTFSSHCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab081 | 350 | FL VH- | EVQLVESGGGLVQPGGSLRLSCAASGFTFSSHCMGWYRQAPGKGREFVAAIQ |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab082 | 351 | FL VH- | EVQLVESGGGLVQPGGSLRLSCAASGFTFSSHCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab083 | 352 | FL VH- | EVQLVESGGGLVQPGGSLRLSCAASGFTFSSYCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab084 | 353 | FL VH- | EVQLVESGGGLVQPGGSLRLSCAASGFTFSSYCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PA) | PSVFLFPPKPKDTLMISRTPEVTCVVVDVSHEDPEVKFNWYVDGVEVHNAKT |
| | | | KPREEQYNSTYRVVSVLTVLHQDWLNGKEYKCKVSNKALAAPIEKTISKAKG |
| | | | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMHEALHNHYTQKSLSLSP |
| | | | GK |
| Ab001 | 464 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab002 | 465 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIIKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab003 | 466 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab004 | 467 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRYQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab005 | 468 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWRYQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab006 | 469 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIIQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab007 | 470 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWIIQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab008 | 471 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPRKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab009 | 472 | FL VH- | EVQLVESGGGLVQPGGSLRLSCAASGSTYSSHCMGWYRQAPGKGREFVALMT |
| | | Ig Fc | RSYGTSYADSVKGRFTISRDNSKNTVYLQMNSLRAEDTAVYYCAAVPRAGIE |
| | | (LALA | SGAYCKWPRKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab010 | 473 | FL VH- | EVQLVESGGGLVQPGGSLRLSCAASGSTYSSHCMGWYRQAPGKGREFVALMT |
| | | Ig Fc | RSYGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab011 | 474 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab012 | 475 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab013 | 476 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab014 | 477 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab015 | 478 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPHKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab016 | 479 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPHQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab017 | 480 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPHQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab018 | 481 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab019 | 482 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab020 | 483 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab021 | 484 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab022 | 485 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWKNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab023 | 486 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWKYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab024 | 487 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab025 | 488 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab026 | 489 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab027 | 490 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWQYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab028 | 491 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab029 | 492 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab030 | 493 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab031 | 494 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIHKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab032 | 495 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWILKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab033 | 496 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWINKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab034 | 497 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTHKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab035 | 498 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTIKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab036 | 499 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTLKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab037 | 500 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWINKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab038 | 501 | FL VH- | EVQLVESGGGLVQPGGSLRLSCAASGYNYVFHCMGWYRQAPGKGREFVALMS |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab039 | 502 | FL VH- | EVQLVESGGGLVQPGGSLRLSCAASGITYVSHCMGWYRQAPGKGREFVAAMS |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab040 | 503 | FL VH- | EVQLVESGGGLVQPGGSLRLSCAASGITYSSHCMGWYRQAPGKGREFVAAMQ |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab041 | 504 | FL VH- | EVQLVESGGGLVQPGGSLRLSCAASGITYQSHCMGWYRQAPGKGREFVALIQ |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab042 | 505 | FL VH- | EVQLVESGGGLVQPGGSLRLSCAASGLHYHSHCMGWYRQAPGKGREFVAAMS |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab043 | 506 | FL VH- | EVQLVESGGGLVQPGGSLRLSCAASGIHYSSHCMGWYRQAPGKGREFVALMS |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab044 | 507 | FL VH- | EVQLVESGGGLVQPGGSLRLSCAASGFTYQSHCMGWYRQAPGKGREFVALMQ |
| | | Ig Fc | HSRGTYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab045 | 508 | FL VH- | EVQLVESGGGLVQPGGSLRLSCAASGFLYSFHCMGWYRQAPGKGREFVALIT |
| | | Ig Fc | HSRGYSYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab046 | 509 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab047 | 510 | FL VH- | EVQLVESGGGLVQPGGSLRLSCAASGSTYSSHCMGWYRQAPGKGREFVALMT |
| | | Ig Fc | HSRGIYYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab048 | 511 | FL VH- | EVQLVESGGGLVQPGGSLRLSCAASGSTYSSHCMGWYRQAPGKGREFVALMT |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab049 | 512 | FL VH- | EVQLVESGGGLVQPGGSLRLSCAASGSTYSSHCMGWYRQAPGKGREFVALMT |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab050 | 513 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab051 | 514 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab052 | 515 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab053 | 516 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab054 | 517 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMLHEALHSHYTQKSLSLS |
| | | | PGK |
| Ab055 | 518 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab056 | 519 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab057 | 520 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab058 | 521 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab059 | 522 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab065 | 528 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | YSGAYCKWNMKDSGSWGQGTQVTVSSGGGGSEPKSSDKTHTCPPCPAPEAAG |
| | | PALS) + | GPSVFLFPPKPKDTLMISRTPEVTCVVVDVSHEDPEVKFNWYVDGVEVHNAK |
| | | c term | TKPREEQYNSTYRVVSVLTVLHQDWLNGKEYKCKVSNKALAAPIEKTISKAK |
| | | K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PGK |
| Ab066 | 529 | FL VH- | EVQLVESGGGLVQPGGSLRLSCAASGSTYASHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab067 | 530 | FL VH- | EVQLVESGGGLVQPGGSLRLSCAASGSTYSSHCMGWYRQAPGKGREFVALMA |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab068 | 531 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKANMKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab069 | 532 | FL VH- | EVQLVESGGGLVQPGGSLRLSCAASGSTYSSHCMGWYRQAPGKGREFVALMT |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab070 | 533 | FL VH- | EVQLVESGGGLVQPGGSLRLSCAASGSTYSSHCMGWYRQAPGKGREFVALMT |
| | | Ig Fc | HSRGTSYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PALS) + | GPSVFLFPPKPKDTLMISRTPEVTCVVVDVSHEDPEVKFNWYVDGVEVHNAK |
| | | c term | TKPREEQYNSTYRVVSVLTVLHQDWLNGKEYKCKVSNKALAAPIEKTISKAK |
| | | K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PGK |
| Ab071 | 534 | FL VH- | EVQLVESGGGLVQPGGSLRLSCAASGLTFSSHCMGWYRQAPGKGREFVAAIQ |
| | | Ig Fc | GSRGSTYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PALS) + | GPSVFLFPPKPKDTLMISRTPEVTCVVVDVSHEDPEVKFNWYVDGVEVHNAK |
| | | c term | TKPREEQYNSTYRVVSVLTVLHQDWLNGKEYKCKVSNKALAAPIEKTISKAK |
| | | K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PGK |
| Ab072 | 535 | FL VH- | EVQLVESGGGLVQPGGSLRLSCAASGLTFSSHCMGWYRQAPGKGREFVAAIQ |
| | | Ig Fc | HSRGSTYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PALS) + | GPSVFLFPPKPKDTLMISRTPEVTCVVVDVSHEDPEVKFNWYVDGVEVHNAK |
| | | c term | TKPREEQYNSTYRVVSVLTVLHQDWLNGKEYKCKVSNKALAAPIEKTISKAK |
| | | K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PGK |
| Ab073 | 536 | FL VH- | EVQLVESGGGLVQPGGSLRLSCAASGLTFSSHCMGWYRQAPGKGREFVAAIQ |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab074 | 537 | FL VH- | EVQLVESGGGLVQPGGSLRLSCAASGLTFSSHCMGWYRQAPGKGREFVAAIQ |
| | | Ig Fc | RSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab075 | 538 | FL VH- | EVQLVESGGGLVQPGGSLRLSCAASGLTFSSHCMGWYRQAPGKGREFVAAIS |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab076 | 539 | FL VH- | EVQLVESGGGLVQPGGSLRLSCAASGLTFSSHCMGWYRQAPGKGREFVAAIS |
| | | Ig Fc | GSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab077 | 540 | FL VH- | EVQLVESGGGLVQPGGSLRLSCAASGLTFSSHCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab078 | 541 | FL VH- | EVQLVESGGGLVQPGGSLRLSCAASGLTFSSHCMGWYRQAPGKGREFVAAIQ |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab079 | 542 | FL VH- | EVQLVESGGGLVQPGGSLRLSCAASGLTFSSHCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab080 | 543 | FL VH- | EVQLVESGGGLVQPGGSLRLSCAASGFTFSSHCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab081 | 544 | FL VH- | EVQLVESGGGLVQPGGSLRLSCAASGFTFSSHCMGWYRQAPGKGREFVAAIQ |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab082 | 545 | FL VH- | EVQLVESGGGLVQPGGSLRLSCAASGFTFSSHCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab083 | 546 | FL VH- | EVQLVESGGGLVQPGGSLRLSCAASGFTFSSYCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab084 | 547 | FL VH- | EVQLVESGGGLVQPGGSLRLSCAASGFTFSSYCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) + | PSVFLFPPKPKDTLMISRTPEVTCVVVDVSHEDPEVKFNWYVDGVEVHNAKT |
| | | c term | KPREEQYNSTYRVVSVLTVLHQDWLNGKEYKCKVSNKALAAPIEKTISKAKG |
| | | K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | GK |
| Ab001 | 548 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab002 | 549 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIIKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab003 | 550 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab004 | 551 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRYQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab005 | 552 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWRYQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab006 | 553 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIIQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab007 | 554 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWIIQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab008 | 555 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPRKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab009 | 556 | FL VH- | EVQLVESGGGLVQPGGSLRLSCAASGSTYSSHCMGWYRQAPGKGREFVALMT |
| | | Ig Fc | RSYGTSYADSVKGRFTISRDNSKNTVYLQMNSLRAEDTAVYYCAAVPRAGIE |
| | | (LALA | SGAYCKWPRKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab010 | 557 | FL VH- | EVQLVESGGGLVQPGGSLRLSCAASGSTYSSHCMGWYRQAPGKGREFVALMT |
| | | Ig Fc | RSYGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab011 | 558 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab012 | 559 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPRQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab013 | 560 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab014 | 561 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab015 | 562 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPHKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab016 | 563 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWPHQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab017 | 564 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWPHQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab018 | 565 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab019 | 566 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab020 | 567 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab021 | 568 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWHYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab022 | 569 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWKNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab023 | 570 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWKYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab024 | 571 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab025 | 572 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab026 | 573 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab027 | 574 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWQYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab028 | 575 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWRNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab029 | 576 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab030 | 577 | FL VH- | EVQLVESGGGLVQPGGSLRLSCAASGSTYSSHCMGWYRQAPGKGREFVALMT |
| | | Ig Fc | HSRGTSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab031 | 578 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWIHKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab032 | 579 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWILKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab033 | 580 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWINKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab034 | 581 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTHKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab035 | 582 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTIKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab036 | 583 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWILKDSGSWGQGTQVTVSSGGGGSEPKSSDKTHTCPPCPAPEAAGG |
| | | PALS) | PSVFLFPPKPKDTIMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab037 | 584 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWTNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab038 | 585 | FL VH- | EVQLVESGGGLVQPGGSLRLSCAASGYNYVFHCMGWYRQAPGKGREFVALMS |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab039 | 586 | FL VH- | EVQLVESGGGLVQPGGSLRLSCAASGITYVSHCMGWYRQAPGKGREFVAAMS |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab040 | 587 | FL VH- | EVQLVESGGGLVQPGGSLRLSCAASGITYSSHCMGWYRQAPGKGREFVAAMQ |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab041 | 588 | FL VH- | EVQLVESGGGLVQPGGSLRLSCAASGITYQSHCMGWYRQAPGKGREFVALIQ |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab042 | 589 | FL VH- | EVQLVESGGGLVQPGGSLRLSCAASGLHYHSHCMGWYRQAPGKGREFVAAMS |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab043 | 590 | FL VH- | EVQLVESGGGLVQPGGSLRLSCAASGIHYSSHCMGWYRQAPGKGREFVALMS |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab044 | 591 | FL VH- | EVQLVESGGGLVQPGGSLRLSCAASGFTYQSHCMGWYRQAPGKGREFVALMQ |
| | | Ig Fc | HSRGTYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab045 | 592 | FL VH- | EVQLVESGGGLVQPGGSLRLSCAASGFLYSFHCMGWYRQAPGKGREFVALIT |
| | | Ig Fc | HSRGYSYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab046 | 593 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab047 | 594 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWRLQDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab048 | 595 | FL VH- | EVQLVESGGGLVQPGGSLRLSCAASGSTYSSHCMGWYRQAPGKGREFVALMT |
| | | Ig Fc | RSRGYYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab049 | 596 | FL VH- | EVQLVESGGGLVQPGGSLRLSCAASGSTYSSHCMGWYRQAPGKGREFVALMT |
| | | Ig Fc | RSRGTYYADSVKGRFTISRDNSKNTVYLQMNSLRAEDTAVYYCAAVPRAGIE |
| | | (LALA | SGAYCKWHGKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab050 | 597 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab051 | 598 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWHNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab052 | 599 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab053 | 600 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWNNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab054 | 601 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVMLHEALHSHYTQKSLSLS |
| | | | PG |
| Ab055 | 602 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab056 | 603 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab057 | 604 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWQNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab058 | 605 | FL VH- | EVQLVESGGGLVQPGGSLRLSCAASGSTYSSHCMGWYRQAPGKGREFVALMT |
| | | Ig Fc | HSRGYYYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab059 | 606 | FL VH- | EVQLVESGGGLVQPGGSLRLSCAASGSTYSSHCMGWYRQAPGKGREFVALMT |
| | | Ig Fc | HSRGTYYADSVKGRFTISRDNSKNTVYLQMNSLRAEDTAVYYCAAVPRAGIE |
| | | (LALA | SGAYCKWSNKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab065 | 612 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | YSGAYCKWNMKDSGSWGQGTQVTVSSGGGGSEPKSSDKTHTCPPCPAPEAAG |
| | | PALS) | GPSVFLFPPKPKDTLMISRTPEVTCVVVDVSHEDPEVKFNWYVDGVEVHNAK |
| | | no c | TKPREEQYNSTYRVVSVLTVLHQDWLNGKEYKCKVSNKALAAPIEKTISKAK |
| | | term K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PG |
| Ab066 | 613 | FL VH- | EVQLVESGGGLVQPGGSLRLSCAASGSTYASHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab067 | 614 | FL VH- | EVQLVESGGGLVQPGGSLRLSCAASGSTYSSHCMGWYRQAPGKGREFVALMA |
| | | Ig Fc | RSRGTSYADSVKGRFTISRDNSKNTVYLQMNSLRAEDTAVYYCAAVPRAGIE |
| | | (LALA | SGAYCKWNMKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab068 | 615 | FL VH- | EVQLVESGGGLVQPGGSLRLSCAASGSTYSSHCMGWYRQAPGKGREFVALMT |
| | | Ig Fc | RSRGTSYADSVKGRFTISRDNSKNTVYLQMNSLRAEDTAVYYCAAVPRAGIE |
| | | (LALA | SGAYCKANMKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab069 | 616 | FL VH- | EVQLVESGGGLVQPGGSLRLSCAASGSTYSSHCMGWYRQAPGKGREFVALMT |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab070 | 617 | FL VH- | EVQLVESGGGLVQPGGSLRLSCAASGSTYSSHCMGWYRQAPGKGREFVALMT |
| | | Ig Fc | HSRGTSYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PALS) | GPSVFLFPPKPKDTLMISRTPEVTCVVVDVSHEDPEVKFNWYVDGVEVHNAK |
| | | no c | TKPREEQYNSTYRVVSVLTVLHQDWLNGKEYKCKVSNKALAAPIEKTISKAK |
| | | term K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PG |
| Ab071 | 618 | FL VH- | EVQLVESGGGLVQPGGSLRLSCAASGLTFSSHCMGWYRQAPGKGREFVAAIQ |
| | | Ig Fc | GSRGSTYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PALS) | GPSVFLFPPKPKDTLMISRTPEVTCVVVDVSHEDPEVKFNWYVDGVEVHNAK |
| | | no c | TKPREEQYNSTYRVVSVLTVLHQDWLNGKEYKCKVSNKALAAPIEKTISKAK |
| | | term K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PG |
| Ab072 | 619 | FL VH- | EVQLVESGGGLVQPGGSLRLSCAASGLTFSSHCMGWYRQAPGKGREFVAAIQ |
| | | Ig Fc | HSRGSTYYADSVKGRFTISRDNSKNTVYLQMNSLRAEDTAVYYCAAVPRAGI |
| | | (LALA | ESGAYCKWSYKDSGSWGQGTQVTVSSGGGGSEPKSSDKTHTCPPCPAPEAAG |
| | | PALS) | GPSVFLFPPKPKDTLMISRTPEVTCVVVDVSHEDPEVKFNWYVDGVEVHNAK |
| | | no c | TKPREEQYNSTYRVVSVLTVLHQDWLNGKEYKCKVSNKALAAPIEKTISKAK |
| | | term K | GQPREPQVYTLPPSRDELTKNQVSLTCLVKGFYPSDIAVEWESNGQPENNYK |
| | | | TTPPVLDSDGSFFLYSKLTVDKSRWQQGNVFSCSVLHEALHSHYTQKSLSLS |
| | | | PG |
| Ab073 | 620 | FL VH- | EVQLVESGGGLVQPGGSLRLSCAASGLTFSSHCMGWYRQAPGKGREFVAAIQ |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab074 | 621 | FL VH- | EVQLVESGGGLVQPGGSLRLSCAASGLTFSSHCMGWYRQAPGKGREFVAAIQ |
| | | Ig Fc | RSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab075 | 622 | FL VH- | EVQLVESGGGLVQPGGSLRLSCAASGLTFSSHCMGWYRQAPGKGREFVAAIS |
| | | Ig Fc | HSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab76 | 623 | FL VH- | EVQLVESGGGLVQPGGSLRLSCAASGLTFSSHCMGWYRQAPGKGREFVAAIS |
| | | Ig Fc | GSRGYSYADSVKGRFTISRDNSKNTVYLQMNSLRAEDTAVYYCAAVPRAGIE |
| | | (LALA | SGAYCKWSYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab077 | 624 | FL VH- | EVQLVESGGGLVQPGGSLRLSCAASGLTFSSHCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab078 | 625 | FL VH- | EVQLVESGGGLVQPGGSLRLSCAASGLTFSSHCMGWYRQAPGKGREFVAAIQ |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab079 | 626 | FL VH- | EVQLVESGGGLVQPGGSLRLSCAASGLTFSSHCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab080 | 627 | FL VH- | EVQLVESGGGLVQPGGSLRLSCAASGFTFSSHCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab081 | 628 | FL VH- | EVQLVESGGGLVQPGGSLRLSCAASGFTFSSHCMGWYRQAPGKGREFVAAIQ |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab082 | 629 | FL VH- | EVQLVESGGGLVQPGGSLRLSCAASGFTFSSHCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab083 | 630 | FL VH- | EVQLVESGGGLVQPGGSLRLSCAASGFTFSSYCMGWYRQAPGKGREFVAAIS |
| | | Ig Fc | HSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab084 | 631 | FL VH- | EVQLVESGGGLVQPGGSLRLSCAASGFTFSSYCMGWYRQAPGKGREFVAAIQ |
| | | Ig Fc | GSRGSSYADSVKGRFTISRDNSKNTVYLQMNSLRAEDTAVYYCAAVPRAGIE |
| | | (LALA | SGAYCKWNYKDSGSWGQGTQVTVSSGGGGSEPKSSDKTHTCPPCPAPEAAGG |
| | | PALS) | PSVFLFPPKPKDTLMISRTPEVTCVVVDVSHEDPEVKFNWYVDGVEVHNAKT |
| | | no c | KPREEQYNSTYRVVSVLTVLHQDWLNGKEYKCKVSNKALAAPIEKTISKAKG |
| | | term K | QPREPQVYTLPPSRDELTKNQVSLTCLVKGFYPSDIAVEWESNGQPENNYKT |
| | | | TPPVLDSDGSFFLYSKLTVDKSRWQQGNVFSCSVLHEALHSHYTQKSLSLSP |
| | | | G |
| Ab121 | 751 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSDKTHTCPPCPAPEAAGG |
| | | (LALA | PSVFLFPPKPKDTLMISRTPEVTCVVVDVSHEDPEVKFNWYVDGVEVHNAKT |
| | | PALS) + | KPREEQYNSTYRVVSVLTVLHQDWLNGKEYKCKVSNKALAAPIEKTISKAKG |
| | | c term | QPREPQVYTLPPSRDELTKNQVSLTCLVKGFYPSDIAVEWESNGQPENNYKT |
| | | K | TPPVLDSDGSFFLYSKLTVDKSRWQQGNVFSCSVLHEALHSHYTQKSLSLSP |
| | | | GK |
| Ab122 | 752 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSDKTHTCPPCPAPEAAGG |
| | | (LALA | PSVFLFPPKPKDTLMISRTPEVTCVVVDVSHEDPEVKFNWYVDGVEVHNAKT |
| | | PALS) + | KPREEQYNSTYRVVSVLTVLHQDWLNGKEYKCKVSNKALAAPIEKTISKAKG |
| | | c term | QPREPQVYTLPPSRDELTKNQVSLTCLVKGFYPSDIAVEWESNGQPENNYKT |
| | | K | TPPVLDSDGSFFLYSKLTVDKSRWQQGNVFSCSVLHEALHSHYTQKSLSLSP |
| | | | GK |
| Ab123 | 753 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSTCPPCPAPEAAGGPSVFLFPPK |
| | | (LALA | PKDTLMISRTPEVTCVVVDVSHEDPEVKFNWYVDGVEVHNAKIKPREEQYNS |
| | | PALS; | TYRVVSVLTVLHQDWLNGKEYKCKVSNKALAAPIEKTISKAKGQPREPQVYT |
| | | Del1) + | LPPSRDELTKNQVSLTCLVKGFYPSDIAVEWESNGQPENNYKTTPPVLDSDG |
| | | c term | SFFLYSKLTVDKSRWQQGNVFSCSVLHEALHSHYTQKSLSLSPGK |
| | | K | |
| Ab124 | 754 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSTCPPCPAPEAAGGPSVF |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del2) + | PQVYTLPPSRDELTKNQVSLTCLVKGFYPSDIAVEWESNGQPENNYKTTPPV |
| | | c term | LDSDGSFFLYSKLTVDKSRWQQGNVFSCSVLHEALHSHYTQKSLSLSPGK |
| | | K | |
| Ab125 | 755 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSAPEAAGGPSVFLFPPKPKDTLM |
| | | (LALA | ISRTPEVTCVVVDVSHEDPEVKFNWYVDGVEVHNAKTKPREEQYNSTYRVVS |
| | | PALS; | VLIVLHQDWLNGKEYKCKVSNKALAAPIEKTISKAKGQPREPQVYTLPPSRD |
| | | Del3) + | ELTKNQVSLTCLVKGFYPSDIAVEWESNGQPENNYKITPPVLDSDGSFFLYS |
| | | c term | KLTVDKSRWQQGNVFSCSVLHEALHSHYTQKSLSLSPGK |
| | | K | |
| Ab126 | 756 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSTHTCPPCPAPEAAGGPSVFLFP |
| | | (LALA | PKPKDTLMISRTPEVTCVVVDVSHEDPEVKFNWYVDGVEVHNAKTKPREEQY |
| | | PALS; | NSTYRVVSVLTVLHQDWLNGKEYKCKVSNKALAAPIEKTISKAKGQPREPQV |
| | | Del4) + | YTLPPSRDELTKNQVSLTCLVKGFYPSDIAVEWESNGQPENNYKITPPVLDS |
| | | c term | DGSFFLYSKLTVDKSRWQQGNVFSCSVLHEALHSHYTQKSLSLSPGK |
| | | K | |
| Ab127 | 757 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSTCPPCPAPEAAGGPSVFLFPPK |
| | | (LALA | PKDTLMISRTPEVTCVVVDVSHEDPEVKFNWYVDGVEVHNAKIKPREEQYNS |
| | | PALS; | TYRVVSVLTVLHQDWLNGKEYKCKVSNKALAAPIEKTISKAKGQPREPQVYT |
| | | Del1) + | LPPSRDELTKNQVSLTCLVKGFYPSDIAVEWESNGQPENNYKTTPPVLDSDG |
| | | c term | SFFLYSKLTVDKSRWQQGNVFSCSVLHEALHSHYTQKSLSLSPGK |
| | | K | |
| Ab128 | 758 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSTCPPCPAPEAAGGPSVF |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del2) + | PQVYTLPPSRDELTKNQVSLTCLVKGFYPSDIAVEWESNGQPENNYKTTPPV |
| | | c term | LDSDGSFFLYSKLTVDKSRWQQGNVFSCSVLHEALHSHYTQKSLSLSPGK |
| | | K | |
| Ab129 | 759 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSAPEAAGGPSVFLFPPKPKDTLM |
| | | (LALA | ISRTPEVTCVVVDVSHEDPEVKFNWYVDGVEVHNAKTKPREEQYNSTYRVVS |
| | | PALS; | VLTVLHQDWLNGKEYKCKVSNKALAAPIEKTISKAKGQPREPQVYTLPPSRD |
| | | Del3) + | ELIKNQVSLTCLVKGFYPSDIAVEWESNGQPENNYKTTPPVLDSDGSFFLYS |
| | | c term | KLTVDKSRWQQGNVFSCSVLHEALHSHYTQKSLSLSPGK |
| | | K | |
| Ab130 | 760 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSTHTCPPCPAPEAAGGPSVFLFP |
| | | (LALA | PKPKDTLMISRTPEVTCVVVDVSHEDPEVKFNWYVDGVEVHNAKTKPREEQY |
| | | PALS; | NSTYRVVSVLTVLHQDWLNGKEYKCKVSNKALAAPIEKTISKAKGQPREPQV |
| | | Del4) + | YTLPPSRDELTKNQVSLTCLVKGFYPSDIAVEWESNGQPENNYKITPPVLDS |
| | | c term | DGSFFLYSKLTVDKSRWQQGNVFSCSVLHEALHSHYTQKSLSLSPGK |
| | | K | |
| Ab131 | 761 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSTCPPCPAPEAAGGPSVF |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del5) + | PQVYTLPPSRDELTKNQVSLTCLVKGFYPSDIAVEWESNGQPENNYKITPPV |
| | | c term | LDSDGSFFLYSKLTVDKSRWQQGNVFSCSVLHEALHSHYTQKSLSLSPGK |
| | | K | |
| Ab132 | 762 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSGGGGTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del6) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab133 | 763 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGGGGSTCPPCPAPEAAGGPSVF |
| | | (LALA | LFPPKPKDTLMISRTPEVTCVVVDVSHEDPEVKFNWYVDGVEVHNAKIKPRE |
| | | PALS; | EQYNSTYRVVSVLTVLHQDWLNGKEYKCKVSNKALAAPIEKTISKAKGQPRE |
| | | Del5) + | PQVYTLPPSRDELTKNQVSLICLVKGFYPSDIAVEWESNGQPENNYKITPPV |
| | | c term | LDSDGSFFLYSKLTVDKSRWQQGNVFSCSVLHEALHSHYTQKSLSLSPGK |
| | | K | |
| Ab134 | 764 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGGGGSGGGGTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del6) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab135 | 765 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSAHHPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del7) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab136 | 766 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSAQQPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del8) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab137 | 767 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGTNEVCKCPKCPAPEAAGGPSV |
| | | (LALA | FLFPPKPKDTLMISRTPEVTCVVVDVSHEDPEVKFNWYVDGVEVHNAKTKPR |
| | | PALS; | EEQYNSTYRVVSVLTVLHQDWLNGKEYKCKVSNKALAAPIEKTISKAKGQPR |
| | | Del9) + | EPQVYTLPPSRDELTKNQVSLTCLVKGFYPSDIAVEWESNGQPENNYKTTPP |
| | | c term | VLDSDGSFFLYSKLTVDKSRWQQGNVFSCSVLHEALHSHYTQKSLSLSPGK |
| | | K | |
| Ab138 | 768 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSAHHPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del7) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab139 | 769 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSAQQPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del8) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab140 | 770 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGTNEVCKCPKCPAPEAAGGPSV |
| | | (LALA | FLFPPKPKDTLMISRTPEVTCVVVDVSHEDPEVKFNWYVDGVEVHNAKTKPR |
| | | PALS; | EEQYNSTYRVVSVLTVLHQDWLNGKEYKCKVSNKALAAPIEKTISKAKGQPR |
| | | Del9) + | EPQVYTLPPSRDELTKNQVSLTCLVKGFYPSDIAVEWESNGQPENNYKTTPP |
| | | c term | VLDSDGSFFLYSKLTVDKSRWQQGNVFSCSVLHEALHSHYTQKSLSLSPGK |
| | | K | |
| Ab141 | 771 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSKTHTCPPCPAPEAAGGP |
| | | (LALA | SVFLFPPKPKDTLMISRTPEVTCVVVDVSHEDPEVKFNWYVDGVEVHNAKTK |
| | | PALS; | PREEQYNSTYRVVSVLTVLHQDWLNGKEYKCKVSNKALAAPIEKTISKAKGQ |
| | | Del10) | PREPQVYTLPPSRDELTKNQVSLTCLVKGFYPSDIAVEWESNGQPENNYKTT |
| | | c term | PPVLDSDGSFFLYSKLTVDKSRWQQGNVFSCSVLHEALHSHYTQKSLSLSPG |
| | | K | K |
| Ab142 | 772 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSKTHTCPPCPAPEAAGGPS |
| | | (LALA | VFLFPPKPKDTLMISRTPEVTCVVVDVSHEDPEVKFNWYVDGVEVHNAKTKP |
| | | PALS; | REEQYNSTYRVVSVLTVLHQDWLNGKEYKCKVSNKALAAPIEKTISKAKGQP |
| | | Del11) + | REPQVYTLPPSRDELTKNQVSLTCLVKGFYPSDIAVEWESNGQPENNYKTTP |
| | | c term | PVLDSDGSFFLYSKLTVDKSRWQQGNVFSCSVLHEALHSHYTQKSLSLSPGK |
| | | K | |
| Ab143 | 773 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del12) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab144 | 774 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSAQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del13) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab145 | 775 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSATCPPCPAPEAAGGPSVF |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del14) + | PQVYTLPPSRDELTKNQVSLTCLVKGFYPSDIAVEWESNGQPENNYKITPPV |
| | | c term | LDSDGSFFLYSKLTVDKSRWQQGNVFSCSVLHEALHSHYTQKSLSLSPGK |
| | | K | |
| Ab146 | 776 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSGGGGQCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del15) + | QPREPQVYTLPPSRDELTKNQVSLICLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab147 | 777 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSGGGGACPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del16) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab148 | 778 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSKTHTCPPCPAPEAAGGP |
| | | (LALA | SVFLFPPKPKDTLMISRTPEVTCVVVDVSHEDPEVKFNWYVDGVEVHNAKTK |
| | | PALS; | PREEQYNSTYRVVSVLTVLHQDWLNGKEYKCKVSNKALAAPIEKTISKAKGQ |
| | | Del10) + | PREPQVYTLPPSRDELTKNQVSLTCLVKGFYPSDIAVEWESNGQPENNYKTT |
| | | c term | PPVLDSDGSFFLYSKLTVDKSRWQQGNVFSCSVLHEALHSHYTQKSLSLSPG |
| | | K | K |
| Ab149 | 779 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSKTHTCPPCPAPEAAGGPS |
| | | (LALA | VFLFPPKPKDTLMISRTPEVTCVVVDVSHEDPEVKFNWYVDGVEVHNAKTKP |
| | | PALS; | REEQYNSTYRVVSVLTVLHQDWLNGKEYKCKVSNKALAAPIEKTISKAKGQP |
| | | Del11) + | REPQVYTLPPSRDELTKNQVSLTCLVKGFYPSDIAVEWESNGQPENNYKTTP |
| | | c term | PVLDSDGSFFLYSKLTVDKSRWQQGNVFSCSVLHEALHSHYTQKSLSLSPGK |
| | | K | |
| Ab150 | 780 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del12) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab151 | 781 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSAQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del13) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab152 | 782 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSATCPPCPAPEAAGGPSVE |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del14) + | PQVYTLPPSRDELTKNQVSLTCLVKGFYPSDIAVEWESNGQPENNYKTTPPV |
| | | c term | LDSDGSFFLYSKLTVDKSRWQQGNVFSCSVLHEALHSHYTQKSLSLSPGK |
| | | K | |
| Ab153 | 783 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGGGGGSGGGGQCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del15) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab154 | 784 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGGGGSGGGGACPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del16) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVLHEALHSHYTQKSLSLSP |
| | | K | GK |
| Ab121 | 785 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSDKTHTCPPCPAPEAAGG |
| | | (LALA | PSVFLFPPKPKDTLMISRTPEVTCVVVDVSHEDPEVKFNWYVDGVEVHNAKT |
| | | PALS) | KPREEQYNSTYRVVSVLTVLHQDWLNGKEYKCKVSNKALAAPIEKTISKAKG |
| | | no c | QPREPQVYTLPPSRDELTKNQVSLTCLVKGFYPSDIAVEWESNGQPENNYKT |
| | | term K | TPPVLDSDGSFFLYSKLTVDKSRWQQGNVFSCSVLHEALHSHYTQKSLSLSP |
| | | | G |
| Ab122 | 786 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSDKTHTCPPCPAPEAAGG |
| | | (LALA | PSVFLFPPKPKDTLMISRTPEVTCVVVDVSHEDPEVKFNWYVDGVEVHNAKT |
| | | PALS) | KPREEQYNSTYRVVSVLTVLHQDWLNGKEYKCKVSNKALAAPIEKTISKAKG |
| | | no c | QPREPQVYTLPPSRDELTKNQVSLTCLVKGFYPSDIAVEWESNGQPENNYKT |
| | | term K | TPPVLDSDGSFFLYSKLTVDKSRWQQGNVFSCSVLHEALHSHYTQKSLSLSP |
| | | | G |
| Ab123 | 787 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSTCPPCPAPEAAGGPSVFLFPPK |
| | | (LALA | PKDTLMISRTPEVTCVVVDVSHEDPEVKFNWYVDGVEVHNAKTKPREEQYNS |
| | | PALS; | TYRVVSVLTVLHQDWLNGKEYKCKVSNKALAAPIEKTISKAKGQPREPQVYT |
| | | Del1) | LPPSRDELTKNQVSLTCLVKGFYPSDIAVEWESNGQPENNYKTTPPVLDSDG |
| | | no c | SFFLYSKLTVDKSRWQQGNVFSCSVLHEALHSHYTQKSLSLSPG |
| | | term K | |
| Ab124 | 788 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSTCPPCPAPEAAGGPSVF |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del2) | PQVYTLPPSRDELTKNQVSLTCLVKGFYPSDIAVEWESNGQPENNYKTTPPV |
| | | no c | LDSDGSFFLYSKLTVDKSRWQQGNVFSCSVLHEALHSHYTQKSLSLSPG |
| | | term K | |
| Ab125 | 789 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSAPEAAGGPSVFLFPPKPKDTLM |
| | | (LALA | ISRTPEVTCVVVDVSHEDPEVKFNWYVDGVEVHNAKTKPREEQYNSTYRVVS |
| | | PALS; | VLIVLHQDWLNGKEYKCKVSNKALAAPIEKTISKAKGQPREPQVYTLPPSRD |
| | | Del3) | ELTKNQVSLTCLVKGFYPSDIAVEWESNGQPENNYKTTPPVLDSDGSFFLYS |
| | | no c | KLTVDKSRWQQGNVFSCSVLHEALHSHYTQKSLSLSPG |
| | | term K | |
| Ab126 | 790 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSTHTCPPCPAPEAAGGPSVFLFP |
| | | (LALA | PKPKDTLMISRTPEVTCVVVDVSHEDPEVKFNWYVDGVEVHNAKTKPREEQY |
| | | PALS; | NSTYRVVSVLTVLHQDWLNGKEYKCKVSNKALAAPIEKTISKAKGQPREPQV |
| | | Del4) | YTLPPSRDELTKNQVSLTCLVKGFYPSDIAVEWESNGQPENNYKTTPPVLDS |
| | | no c | DGSFFLYSKLTVDKSRWQQGNVFSCSVLHEALHSHYTQKSLSLSPG |
| | | term K | |
| Ab127 | 791 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSTCPPCPAPEAAGGPSVFLFPPK |
| | | (LALA | PKDTLMISRTPEVTCVVVDVSHEDPEVKFNWYVDGVEVHNAKTKPREEQYNS |
| | | PALS; | TYRVVSVLTVLHQDWLNGKEYKCKVSNKALAAPIEKTISKAKGQPREPQVYT |
| | | Del1) | LPPSRDELTKNQVSLTCLVKGFYPSDIAVEWESNGQPENNYKTTPPVLDSDG |
| | | no c | SFFLYSKLTVDKSRWQQGNVFSCSVLHEALHSHYTQKSLSLSPG |
| | | term K | |
| Ab128 | 792 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSTCPPCPAPEAAGGPSVF |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del2) | PQVYTLPPSRDELTKNQVSLTCLVKGFYPSDIAVEWESNGQPENNYKITPPV |
| | | no c | LDSDGSFFLYSKLTVDKSRWQQGNVFSCSVLHEALHSHYTQKSLSLSPG |
| | | term K | |
| Ab129 | 793 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSAPEAAGGPSVFLFPPKPKDTLM |
| | | (LALA | ISRTPEVTCVVVDVSHEDPEVKFNWYVDGVEVHNAKTKPREEQYNSTYRVVS |
| | | PALS; | VLIVLHQDWLNGKEYKCKVSNKALAAPIEKTISKAKGQPREPQVYTLPPSRD |
| | | Del3) | ELTKNQVSLTCLVKGFYPSDIAVEWESNGQPENNYKTTPPVLDSDGSFFLYS |
| | | no c | KLTVDKSRWQQGNVFSCSVLHEALHSHYTQKSLSLSPG |
| | | term K | |
| Ab130 | 794 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSTHTCPPCPAPEAAGGPSVFLFP |
| | | (LALA | PKPKDTLMISRTPEVTCVVVDVSHEDPEVKFNWYVDGVEVHNAKIKPREEQY |
| | | PALS; | NSTYRVVSVLTVLHQDWLNGKEYKCKVSNKALAAPIEKTISKAKGQPREPQV |
| | | Del4) | YILPPSRDELTKNQVSLTCLVKGFYPSDIAVEWESNGQPENNYKITPPVLDS |
| | | no c | DGSFFLYSKLTVDKSRWQQGNVFSCSVLHEALHSHYTQKSLSLSPG |
| | | term K | |
| Ab131 | 795 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSTCPPCPAPEAAGGPSVF |
| | | (LALA | LFPPKPKDTLMISRTPEVTCVVVDVSHEDPEVKFNWYVDGVEVHNAKTKPRE |
| | | PALS; | EQYNSTYRVVSVLTVLHQDWLNGKEYKCKVSNKALAAPIEKTISKAKGQPRE |
| | | Del5) | PQVYTLPPSRDELTKNQVSLTCLVKGFYPSDIAVEWESNGQPENNYKITPPV |
| | | no c | LDSDGSFFLYSKLTVDKSRWQQGNVFSCSVLHEALHSHYTQKSLSLSPG |
| | | term K | |
| Ab132 | 796 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSGGGGTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del6) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab133 | 797 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGGGGSTCPPCPAPEAAGGPSVF |
| | | (LALA | LFPPKPKDTLMISRTPEVTCVVVDVSHEDPEVKFNWYVDGVEVHNAKIKPRE |
| | | PALS; | EQYNSTYRVVSVLTVLHQDWLNGKEYKCKVSNKALAAPIEKTISKAKGQPRE |
| | | Del5) | PQVYTLPPSRDELTKNQVSLTCLVKGFYPSDIAVEWESNGQPENNYKITPPV |
| | | no c | LDSDGSFFLYSKLTVDKSRWQQGNVFSCSVLHEALHSHYTQKSLSLSPG |
| | | term K | |
| Ab134 | 798 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGGGGSGGGGTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del6) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab135 | 799 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSAHHPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del7) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab136 | 800 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSAQQPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del8) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab137 | 801 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGTNEVCKCPKCPAPEAAGGPSV |
| | | (LALA | FLFPPKPKDTLMISRTPEVTCVVVDVSHEDPEVKFNWYVDGVEVHNAKTKPR |
| | | PALS; | EEQYNSTYRVVSVLTVLHQDWLNGKEYKCKVSNKALAAPIEKTISKAKGQPR |
| | | Del9) | EPQVYTLPPSRDELTKNQVSLTCLVKGFYPSDIAVEWESNGQPENNYKITPP |
| | | no c | VLDSDGSFFLYSKLTVDKSRWQQGNVFSCSVLHEALHSHYTQKSLSLSPG |
| | | term K | |
| Ab138 | 802 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSAHHPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del7) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab139 | 803 | Ab078 | EVQLVESGGGLVQPGGSLRLSCAASGLTESSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSAQQPEEPSSQCPKCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del8) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab140 | 804 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGTNEVCKCPKCPAPEAAGGPSV |
| | | (LALA | FLFPPKPKDTLMISRTPEVTCVVVDVSHEDPEVKFNWYVDGVEVHNAKTKPR |
| | | PALS; | EEQYNSTYRVVSVLTVLHQDWLNGKEYKCKVSNKALAAPIEKTISKAKGQPR |
| | | Del9) | EPQVYTLPPSRDELTKNQVSLTCLVKGFYPSDIAVEWESNGQPENNYKTTPP |
| | | no c | VLDSDGSFFLYSKLTVDKSRWQQGNVFSCSVLHEALHSHYTQKSLSLSPG |
| | | term K | |
| Ab141 | 805 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSKTHTCPPCPAPEAAGGP |
| | | (LALA | SVFLFPPKPKDTLMISRTPEVTCVVVDVSHEDPEVKFNWYVDGVEVHNAKTK |
| | | PALS; | PREEQYNSTYRVVSVLTVLHQDWLNGKEYKCKVSNKALAAPIEKTISKAKGQ |
| | | Del10) | PREPQVYTLPPSRDELTKNQVSLTCLVKGFYPSDIAVEWESNGQPENNYKTT |
| | | no c | PPVLDSDGSFFLYSKLTVDKSRWQQGNVFSCSVLHEALHSHYTQKSLSLSPG |
| | | term K | |
| Ab142 | 806 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSKTHTCPPCPAPEAAGGPS |
| | | (LALA | VFLFPPKPKDTLMISRTPEVTCVVVDVSHEDPEVKFNWYVDGVEVHNAKTKP |
| | | PALS; | REEQYNSTYRVVSVLTVLHQDWLNGKEYKCKVSNKALAAPIEKTISKAKGQP |
| | | Del11) | REPQVYTLPPSRDELTKNQVSLTCLVKGFYPSDIAVEWESNGQPENNYKTTP |
| | | no c | PVLDSDGSFFLYSKLTVDKSRWQQGNVFSCSVLHEALHSHYTQKSLSLSPG |
| | | term K | |
| Ab143 | 807 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSSQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del12) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab144 | 808 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSAQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del13) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab145 | 809 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSEPKSATCPPCPAPEAAGGPSVF |
| | | (LALA | LFPPKPKDTLMISRTPEVTCVVVDVSHEDPEVKFNWYVDGVEVHNAKIKPRE |
| | | PALS; | EQYNSTYRVVSVLTVLHQDWLNGKEYKCKVSNKALAAPIEKTISKAKGQPRE |
| | | Del14) | PQVYTLPPSRDELTKNQVSLTCLVKGFYPSDIAVEWESNGQPENNYKITPPV |
| | | no c | LDSDGSFFLYSKLTVDKSRWQQGNVFSCSVLHEALHSHYTQKSLSLSPG |
| | | term K | |
| Ab146 | 810 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSGGGGQCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del15) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab147 | 811 | Ab076 | EVQLVESGGGLVQPGGSLRLSCAASGLTFSSHCMGWYRQAPGKGREFVAAIS |
| | | VH-Ig | GSRGYSYADSVKGRFTISRDNSKNTVYLQMNSLRAEDTAVYYCAAVPRAGIE |
| | | Fc | SGAYCKWSYKDSGSWGQGTQVTVSSGGGGSGGGGSGGGGACPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del16) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab148 | 812 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSKTHTCPPCPAPEAAGGP |
| | | (LALA | SVFLFPPKPKDTLMISRTPEVTCVVVDVSHEDPEVKFNWYVDGVEVHNAKTK |
| | | PALS; | PREEQYNSTYRVVSVLTVLHQDWLNGKEYKCKVSNKALAAPIEKTISKAKGQ |
| | | Del10) | PREPQVYTLPPSRDELTKNQVSLTCLVKGFYPSDIAVEWESNGQPENNYKIT |
| | | no c | PPVLDSDGSFFLYSKLTVDKSRWQQGNVFSCSVLHEALHSHYTQKSLSLSPG |
| | | term K | |
| Ab149 | 813 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSKTHTCPPCPAPEAAGGPS |
| | | (LALA | VFLFPPKPKDTLMISRTPEVTCVVVDVSHEDPEVKFNWYVDGVEVHNAKTKP |
| | | PALS; | REEQYNSTYRVVSVLTVLHQDWLNGKEYKCKVSNKALAAPIEKTISKAKGQP |
| | | Del11) | REPQVYTLPPSRDELTKNQVSLTCLVKGFYPSDIAVEWESNGQPENNYKTTP |
| | | no c | PVLDSDGSFFLYSKLTVDKSRWQQGNVFSCSVLHEALHSHYTQKSLSLSPG |
| | | term K | |
| Ab150 | 814 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSSQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del12) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab151 | 815 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSAQKTHTCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del13) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab152 | 816 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSEPKSATCPPCPAPEAAGGPSVF |
| | | (LALA | LFPPKPKDTLMISRTPEVTCVVVDVSHEDPEVKFNWYVDGVEVHNAKIKPRE |
| | | PALS; | EQYNSTYRVVSVLTVLHQDWLNGKEYKCKVSNKALAAPIEKTISKAKGQPRE |
| | | Del14) | PQVYTLPPSRDELTKNQVSLTCLVKGFYPSDIAVEWESNGQPENNYKTTPPV |
| | | no c | LDSDGSFFLYSKLTVDKSRWQQGNVFSCSVLHEALHSHYTQKSLSLSPG |
| | | term K | |
| Ab153 | 817 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGGGGSGGGGQCPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del15) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab154 | 818 | Ab078 | EVQLVESGGGLVQPGGSLRLSCAASGLTFSSHCMGWYRQAPGKGREFVAAIQ |
| | | VH-Ig | HSRGSSYADSVKGRFTISRDNSKNTVYLQMNSLRAEDTAVYYCAAVPRAGIE |
| | | Fc | SGAYCKWNYKDSGSWGQGTQVTVSSGGGGSGGGGSGGGGACPPCPAPEAAGG |
| | | (LALA | PSVFLFPPKPKDTLMISRTPEVTCVVVDVSHEDPEVKFNWYVDGVEVHNAKT |
| | | PALS; | KPREEQYNSTYRVVSVLTVLHQDWLNGKEYKCKVSNKALAAPIEKTISKAKG |
| | | Del16) | QPREPQVYTLPPSRDELTKNQVSLTCLVKGFYPSDIAVEWESNGQPENNYKT |
| | | no c | TPPVLDSDGSFFLYSKLTVDKSRWQQGNVFSCSVLHEALHSHYTQKSLSLSP |
| | | term K | G |
| Ab155 | 853 | Ab065- | EVQLVESGGGLVQPGGSLRLSCAASGSTYSSHCMGWYRQAPGKGREFVALMT |
| | | Y105P | RSRGTSYADSVKGRFTISRDNSKNTVYLQMNSLRAEDTAVYYCAAVPRAGIE |
| | | VH-Ig | PSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) + | GQPREPQVYTLPPSRDELTKNQVSLTCLVKGFYPSDIAVEWESNGQPENNYK |
| | | c term | TTPPVLDSDGSFFLYSKLTVDKSRWQQGNVFSCSVMHEALHNHYTQKSLSLS |
| | | K | PGK |
| Ab156 | 854 | Ab065- | EVQLVESGGGLVQPGGSLRLSCAASGSTYSSHCMGWYRQAPGKGREFVALMT |
| | | Y105R | RSRGTSYADSVKGRFTISRDNSKNTVYLQMNSLRAEDTAVYYCAAVPRAGIE |
| | | VH-Ig | RSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) + | GQPREPQVYTLPPSRDELTKNQVSLTCLVKGFYPSDIAVEWESNGQPENNYK |
| | | c term | TTPPVLDSDGSFFLYSKLTVDKSRWQQGNVFSCSVMHEALHNHYTQKSLSLS |
| | | K | PGK |
| Ab157 | 855 | Ab065- | EVQLVESGGGLVQPGGSLRLSCAASGSTYSSHCMGWYRQAPGKGREFVALMT |
| | | Y105W | RSRGTSYADSVKGRFTISRDNSKNTVYLQMNSLRAEDTAVYYCAAVPRAGIE |
| | | VH-Ig | WSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) + | GQPREPQVYTLPPSRDELTKNQVSLTCLVKGFYPSDIAVEWESNGQPENNYK |
| | | c term | TTPPVLDSDGSFFLYSKLTVDKSRWQQGNVFSCSVMHEALHNHYTQKSLSLS |
| | | K | PGK |
| Ab158 | 856 | Ab065- | EVQLVESGGGLVQPGGSLRLSCAASGSTYSSHCMGWYRQAPGKGREFVALMT |
| | | Y105K | RSRGTSYADSVKGRFTISRDNSKNTVYLQMNSLRAEDTAVYYCAAVPRAGIE |
| | | VH-Ig | KSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) + | GQPREPQVYTLPPSRDELTKNQVSLTCLVKGFYPSDIAVEWESNGQPENNYK |
| | | c term | TTPPVLDSDGSFFLYSKLTVDKSRWQQGNVFSCSVMHEALHNHYTQKSLSLS |
| | | K | PGK |
| Ab159 | 857 | Ab001- | EVQLVESGGGLVQPGGSLRLSCAASGSTYSSHCMGWYRQAPGKGREFVALMT |
| | | W111F | RSRGTSYADSVKGRFTISRDNSKNTVYLQMNSLRAEDTAVYYCAAVPRAGIE |
| | | VH-Ig | SGAYCKENMKDSGSWGQGTQVTVSSGGGGSEPKSSDKTHTCPPCPAPEAAGG |
| | | Fc | PSVFLFPPKPKDTLMISRTPEVTCVVVDVSHEDPEVKFNWYVDGVEVHNAKT |
| | | (LALA | KPREEQYNSTYRVVSVLTVLHQDWLNGKEYKCKVSNKALAAPIEKTISKAKG |
| | | PALS) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVMHEALHNHYTQKSLSLSP |
| | | K | GK |
| Ab160 | 858 | Ab001- | EVQLVESGGGLVQPGGSLRLSCAASGSTYSSHCMGWYRQAPGKGREFVALMT |
| | | W111Y | RSRGTSYADSVKGRFTISRDNSKNTVYLQMNSLRAEDTAVYYCAAVPRAGIE |
| | | VH-Ig | SGAYCKYNMKDSGSWGQGTQVTVSSGGGGSEPKSSDKTHTCPPCPAPEAAGG |
| | | Fc | PSVFLFPPKPKDTLMISRTPEVTCVVVDVSHEDPEVKFNWYVDGVEVHNAKT |
| | | (LALA | KPREEQYNSTYRVVSVLTVLHQDWLNGKEYKCKVSNKALAAPIEKTISKAKG |
| | | PALS) + | QPREPQVYTLPPSRDELTKNQVSLTCLVKGFYPSDIAVEWESNGQPENNYKT |
| | | c term | TPPVLDSDGSFFLYSKLTVDKSRWQQGNVFSCSVMHEALHNHYTQKSLSLSP |
| | | K | GK |
| Ab155 | 859 | Ab065- | EVQLVESGGGLVQPGGSLRLSCAASGSTYSSHCMGWYRQAPGKGREFVALMT |
| | | Y105P | RSRGTSYADSVKGRFTISRDNSKNTVYLQMNSLRAEDTAVYYCAAVPRAGIE |
| | | VH-Ig | PSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) | GQPREPQVYTLPPSRDELTKNQVSLTCLVKGFYPSDIAVEWESNGQPENNYK |
| | | no term | TTPPVLDSDGSFFLYSKLTVDKSRWQQGNVFSCSVMHEALHNHYTQKSLSLS |
| | | K | PG |
| Ab156 | 860 | Ab065- | EVQLVESGGGLVQPGGSLRLSCAASGSTYSSHCMGWYRQAPGKGREFVALMT |
| | | Y105R | RSRGTSYADSVKGRFTISRDNSKNTVYLQMNSLRAEDTAVYYCAAVPRAGIE |
| | | VH-Ig | RSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) | GQPREPQVYTLPPSRDELTKNQVSLTCLVKGFYPSDIAVEWESNGQPENNYK |
| | | no term | TTPPVLDSDGSFFLYSKLTVDKSRWQQGNVFSCSVMHEALHNHYTQKSLSLS |
| | | K | PG |
| Ab157 | 861 | Ab065- | EVQLVESGGGLVQPGGSLRLSCAASGSTYSSHCMGWYRQAPGKGREFVALMT |
| | | Y105W | RSRGTSYADSVKGRFTISRDNSKNTVYLQMNSLRAEDTAVYYCAAVPRAGIE |
| | | VH-Ig | WSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) | GQPREPQVYTLPPSRDELTKNQVSLTCLVKGFYPSDIAVEWESNGQPENNYK |
| | | term K | TTPPVLDSDGSFFLYSKLTVDKSRWQQGNVFSCSVMHEALHNHYTQKSLSLS |
| | | | PG |
| Ab158 | 862 | Ab065- | EVQLVESGGGLVQPGGSLRLSCAASGSTYSSHCMGWYRQAPGKGREFVALMT |
| | | Y105K | RSRGTSYADSVKGRFTISRDNSKNTVYLQMNSLRAEDTAVYYCAAVPRAGIE |
| | | VH-Ig | KSGAYCKWNMKDSGSWGQGTQVTVSSGGGGSEPKSSDKTHTCPPCPAPEAAG |
| | | Fc | GPSVFLFPPKPKDTLMISRTPEVTCVVVDVSHEDPEVKFNWYVDGVEVHNAK |
| | | (LALA | TKPREEQYNSTYRVVSVLTVLHQDWLNGKEYKCKVSNKALAAPIEKTISKAK |
| | | PALS) | GQPREPQVYTLPPSRDELTKNQVSLTCLVKGFYPSDIAVEWESNGQPENNYK |
| | | no term | TTPPVLDSDGSFFLYSKLTVDKSRWQQGNVFSCSVMHEALHNHYTQKSLSLS |
| | | K | PG |
| Ab159 | 863 | Ab001- | EVQLVESGGGLVQPGGSLRLSCAASGSTYSSHCMGWYRQAPGKGREFVALMT |
| | | W111F | RSRGTSYADSVKGRFTISRDNSKNTVYLQMNSLRAEDTAVYYCAAVPRAGIE |
| | | VH-Ig | SGAYCKFNMKDSGSWGQGTQVTVSSGGGGSEPKSSDKTHTCPPCPAPEAAGG |
| | | Fc | PSVFLFPPKPKDTLMISRTPEVTCVVVDVSHEDPEVKFNWYVDGVEVHNAKT |
| | | (LALA | KPREEQYNSTYRVVSVLTVLHQDWLNGKEYKCKVSNKALAAPIEKTISKAKG |
| | | PALS) | QPREPQVYTLPPSRDELTKNQVSLTCLVKGFYPSDIAVEWESNGQPENNYKT |
| | | no term | TPPVLDSDGSFFLYSKLTVDKSRWQQGNVFSCSVMHEALHNHYTQKSLSLSP |
| | | K | G |
| Ab160 | 864 | Ab001- | EVQLVESGGGLVQPGGSLRLSCAASGSTYSSHCMGWYRQAPGKGREFVALMT |
| | | W111Y | RSRGTSYADSVKGRFTISRDNSKNTVYLQMNSLRAEDTAVYYCAAVPRAGIE |
| | | VH-Ig | SGAYCKYNMKDSGSWGQGTQVTVSSGGGGSEPKSSDKTHTCPPCPAPEAAGG |
| | | Fc | PSVFLFPPKPKDTLMISRTPEVTCVVVDVSHEDPEVKFNWYVDGVEVHNAKT |
| | | (LALA | KPREEQYNSTYRVVSVLTVLHQDWLNGKEYKCKVSNKALAAPIEKTISKAKG |
| | | PALS) | QPREPQVYTLPPSRDELTKNQVSLTCLVKGFYPSDIAVEWESNGQPENNYKT |
| | | no term | TPPVLDSDGSFFLYSKLTVDKSRWQQGNVFSCSVMHEALHNHYTQKSLSLSP |
| | | K | G |

In certain embodiments, the instant disclosure provides an antibody that specifically binds to APJ (e.g., human APJ), the antibody comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to any one of the amino acid sequences shown in Table 13. In certain embodiments, the antibody comprises or consists of any one of the amino acid sequences shown in Table 13.

TABLE 13
Anti-APJ antibody amino acid sequences.

| | SEQ ID | | |
|---|---|---|---|
| Ab | NO | Description | Sequence |
| Ab085 | 632 | FL VH- | EVQLVESGGGLVQPGGSLRLSCAASGFTFSSYAMGWYRQAPGKGREFVAAIS |
| | | Ig Fc | GSGGSTYYADSVKGRFTISRDNSKNTVYLQMNSLRAEDTAVYYCARTVQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab086 | 633 | FL VH- | EVQLVESGGGLVQPGGSLRLSCAASGFTFSSYAMGWYRQAPGKGREFVAAIS |
| | | Ig Fc | GSGGSTYYADSVKGRFTISRDNSKNTVYLQMNSLRAEDTAVYYCRSRKQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab087 | 634 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab088 | 635 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGFRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab089 | 636 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGYRTVYDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab090 | 637 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGFRTVYDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab091 | 638 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab092 | 639 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGFRTVYDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab093 | 640 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab094 | 641 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGFRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab095 | 642 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTVYDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab096 | 643 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGFRTVYDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab097 | 644 | FL VH- | EVQLVESGGGLVQPGGSLRLSCAASQQTFSSYAMGWYRQAPGKGREFVASTS |
| | | Ig Fc | GSGGSTYYADSVKGRFTISRDNSKNTVYLQMNSLRAEDTAVYYCRAMRQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab098 | 645 | FL VH- | EVQLVESGGGLVQPGGSLRLSCAASQVTFSSYAMGWYRQAPGKGREFVASIS |
| | | Ig Fc | HYDGSTYYADSVKGRFTISRDNSKNTVYLQMNSLRAEDTAVYYCRAGRQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab099 | 646 | FL VH- | EVQLVESGGGLVQPGGSLRLSCAASGFTFPPYAMGWYRQAPGKGREFVASIS |
| | | Ig Fc | GSGGSTYYADSVKGRFTISRDNSKNTVYLQMNSLRAEDTAVYYCRGMKQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab100 | 647 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab101 | 648 | FL VH- | EVQLVESGGGLVQPGGSLRLSCAASGFAISSYAMGWYRQAPGKGREFVAAIS |
| | | Ig Fc | FIAGSTYYADSVKGRFTISRDNSKNTVYLQMNSLRAEDTAVYYCCAVKQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab102 | 649 | FL VH- | EVQLVESGGGLVQPGGSLRLSCAASGFAISSYAMGWYRQAPGKGREFVAAIS |
| | | Ig Fc | FIAGSTYYADSVKGRFTISRDNSKNTVYLQMNSLRAEDTAVYYCSAVKQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab103 | 650 | FL VH- | EVQLVESGGGLVQPGGSLRLSCAASGFPFSSYAMGWYRQAPGKGREFVAAIS |
| | | Ig Fc | FLEGSTYYADSVKGRFTISRDNSKNTVYLQMNSLRAEDTAVYYCRAGKQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab104 | 651 | FL VH- | EVQLVESGGGLVQPGGSLRLSCAASGFDISSYAMGWYRQAPGKGREFVAAIS |
| | | Ig Fc | FVAGSTYYADSVKGRFTISRDNSKNTVYLQMNSLRAEDTAVYYCEARRQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab105 | 652 | FL VH- | EVQLVESGGGLVQPGGSLRLSCAASGFVGSSYAMGWYRQAPGKGREFVASIG |
| | | Ig Fc | GSGGSTYYADSVKGRFTISRDNSKNTVYLQMNSLRAEDTAVYYCRVARQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab106 | 653 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab107 | 654 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQHRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab108 | 655 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRVSLQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab109 | 656 | FL VH- | EVQLVESGGGLVQPGGSLRLSCAASGFPHPSYPMGWYRQAPGKGREFVAGPG |
| | | Ig Fc | GSGGSTYYADSVKGRFTISRDNSKNTVYLQMNSLRAEDTAVYYCRGARQRRT |
| | | (LALA | LDGYRSSFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab110 | 657 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab111 | 658 | FL VH- | EVQLVESGGGLVQPGGSLRLSCAASGFTFSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PA | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab112 | 659 | FL VH- | EVQLVESGGGLVQPGGSLRLSCAASGFTFSSYAMGWYRQAPGKGREFVASIS |
| | | Ig Fc | FSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab113 | 660 | FL VH- | EVQLVESGGGLVQPGGSLRLSCAASGFTFSSYAMGWYRQAPGKGREFVASIS |
| | | Ig Fc | GSGGSTQYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab114 | 661 | FL VH- | EVQLVESGGGLVQPGGSLRLSCAASGFTFSSYAMGWYRQAPGKGREFVAAIS |
| | | Ig Fc | FSGGSTQYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab115 | 662 | FL VH- | EVQLVESGGGLVQPGGSLRLSCAASGFTFSSYAMGWYRQAPGKGREFVAAIS |
| | | Ig Fc | FSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab116 | 663 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab117 | 664 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMVSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab118 | 665 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab119 | 666 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRMVSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab120 | 667 | FL VH- | EVQLVESGGGLVQPGGSLRLSCAASGFTHSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PA) | LFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALAAPIEKTISKAKGQPRE |
| | | | PQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| Ab085 | 668 | FL VH- | EVQLVESGGGLVQPGGSLRLSCAASGFTFSSYAMGWYRQAPGKGREFVAAIS |
| | | Ig Fc | GSGGSTYYADSVKGRFTISRDNSKNTVYLQMNSLRAEDTAVYYCARTVQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab086 | 669 | FL VH- | EVQLVESGGGLVQPGGSLRLSCAASGFTFSSYAMGWYRQAPGKGREFVAAIS |
| | | Ig Fc | GSGGSTYYADSVKGRFTISRDNSKNTVYLQMNSLRAEDTAVYYCRSRKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab087 | 670 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab088 | 671 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGFRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab089 | 672 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGYRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab090 | 673 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGFRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab091 | 674 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab092 | 675 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGFRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab093 | 676 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab094 | 677 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGFRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab095 | 678 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab096 | 679 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGFRTVYDYWGQGTQVTVSSGGGGSEPKSSDKTHTCPPCPAPEAAGGPSVE |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab097 | 680 | FL VH- | EVQLVESGGGLVQPGGSLRLSCAASQQTFSSYAMGWYRQAPGKGREFVASTS |
| | | Ig Fc | GSGGSTYYADSVKGRFTISRDNSKNIVYLQMNSLRAEDTAVYYCRAMRQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab098 | 681 | FL VH- | EVQLVESGGGLVQPGGSLRLSCAASQVTFSSYAMGWYRQAPGKGREFVASIS |
| | | Ig Fc | HYDGSTYYADSVKGRFTISRDNSKNTVYLQMNSLRAEDTAVYYCRAGRQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab099 | 682 | FL VH- | EVQLVESGGGLVQPGGSLRLSCAASGFTFPPYAMGWYRQAPGKGREFVASIS |
| | | Ig Fc | GSGGSTYYADSVKGRFTISRDNSKNTVYLQMNSLRAEDTAVYYCRGMKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab100 | 683 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab101 | 684 | FL VH- | EVQLVESGGGLVQPGGSLRLSCAASGFAISSYAMGWYRQAPGKGREFVAAIS |
| | | Ig Fc | FIAGSTYYADSVKGRFTISRDNSKNTVYLQMNSLRAEDTAVYYCCAVKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab102 | 685 | FL VH- | EVQLVESGGGLVQPGGSLRLSCAASGFAISSYAMGWYRQAPGKGREFVAAIS |
| | | Ig Fc | FIAGSTYYADSVKGRFTISRDNSKNTVYLQMNSLRAEDTAVYYCSAVKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab103 | 686 | FL VH- | EVQLVESGGGLVQPGGSLRLSCAASGFPFSSYAMGWYRQAPGKGREFVAAIS |
| | | Ig Fc | FLEGSTYYADSVKGRFTISRDNSKNTVYLQMNSLRAEDTAVYYCRAGKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab104 | 687 | FL VH- | EVQLVESGGGLVQPGGSLRLSCAASGFDISSYAMGWYRQAPGKGREFVAAIS |
| | | Ig Fc | FVAGSTYYADSVKGRFTISRDNSKNTVYLQMNSLRAEDTAVYYCEARRQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab105 | 688 | FL VH- | EVQLVESGGGLVQPGGSLRLSCAASGFVGSSYAMGWYRQAPGKGREFVASIG |
| | | Ig Fc | GSGGSTYYADSVKGRFTISRDNSKNTVYLQMNSLRAEDTAVYYCRVARQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab106 | 689 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab107 | 690 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQHRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab108 | 691 | FL VH- | EVQLVESGGGLVQPGGSLRLSCAASGFTFSSPHMGWYRQAPGKGREFVAAIS |
| | | Ig Fc | GSGIAGYYADSVKGRFTISRDNSKNTVYLQMNSLRAEDTAVYYCRVSLQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab109 | 692 | FL VH- | EVQLVESGGGLVQPGGSLRLSCAASGFPHPSYPMGWYRQAPGKGREFVAGPG |
| | | Ig Fc | GSGGSTYYADSVKGRFTISRDNSKNTVYLQMNSLRAEDTAVYYCRGARQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab110 | 693 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab111 | 694 | FL VH- | EVQLVESGGGLVQPGGSLRLSCAASGFTFSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab112 | 695 | FL VH- | EVQLVESGGGLVQPGGSLRLSCAASGFTFSSYAMGWYRQAPGKGREFVASIS |
| | | Ig Fc | FSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab113 | 696 | FL VH- | EVQLVESGGGLVQPGGSLRLSCAASGFTFSSYAMGWYRQAPGKGREFVASIS |
| | | Ig Fc | GSGGSTQYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab114 | 697 | FL VH- | EVQLVESGGGLVQPGGSLRLSCAASGFTFSSYAMGWYRQAPGKGREFVAAIS |
| | | Ig Fc | FSGGSTQYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab115 | 698 | FL VH- | EVQLVESGGGLVQPGGSLRLSCAASGFTFSSYAMGWYRQAPGKGREFVAAIS |
| | | Ig Fc | FSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab116 | 699 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab117 | 700 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMVSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab118 | 701 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab119 | 702 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRMVSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab120 | 703 | FL VH- | EVQLVESGGGLVQPGGSLRLSCAASGFTHSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | + c term | EQYNSTYRVVSVLTVLHQDWLNGKEYKCKVSNKALAAPIEKTISKAKGQPRE |
| | | K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPGK |
| Ab085 | 391 | FL VH- | EVQLVESGGGLVQPGGSLRLSCAASGFTFSSYAMGWYRQAPGKGREFVAAIS |
| | | Ig Fc | GSGGSTYYADSVKGRFTISRDNSKNTVYLQMNSLRAEDTAVYYCARTVQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab086 | 392 | FL VH- | EVQLVESGGGLVQPGGSLRLSCAASGFTFSSYAMGWYRQAPGKGREFVAAIS |
| | | Ig Fc | GSGGSTYYADSVKGRFTISRDNSKNTVYLQMNSLRAEDTAVYYCRSRKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab087 | 393 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab088 | 394 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGFRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab089 | 395 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGYRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab090 | 396 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRSVLQRRT |
| | | (LALA | LDGFRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab091 | 397 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab092 | 398 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGFRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab093 | 399 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab094 | 400 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGFRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab095 | 401 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab096 | 402 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGFRTVYDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab097 | 403 | FL VH- | EVQLVESGGGLVQPGGSLRLSCAASQQTFSSYAMGWYRQAPGKGREFVASTS |
| | | Ig Fc | GSGGSTYYADSVKGRFTISRDNSKNTVYLQMNSLRAEDTAVYYCRAMRQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab098 | 404 | FL VH- | EVQLVESGGGLVQPGGSLRLSCAASQVTFSSYAMGWYRQAPGKGREFVASIS |
| | | Ig Fc | HYDGSTYYADSVKGRFTISRDNSKNTVYLQMNSLRAEDTAVYYCRAGRQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab099 | 405 | FL VH- | EVQLVESGGGLVQPGGSLRLSCAASGFTFPPYAMGWYRQAPGKGREFVASIS |
| | | Ig Fc | GSGGSTYYADSVKGRFTISRDNSKNTVYLQMNSLRAEDTAVYYCRGMKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab100 | 406 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab101 | 407 | FL VH- | EVQLVESGGGLVQPGGSLRLSCAASGFAISSYAMGWYRQAPGKGREFVAAIS |
| | | Ig Fc | FIAGSTYYADSVKGRFTISRDNSKNTVYLQMNSLRAEDTAVYYCCAVKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab102 | 408 | FL VH- | EVQLVESGGGLVQPGGSLRLSCAASGFAISSYAMGWYRQAPGKGREFVAAIS |
| | | Ig Fc | FIAGSTYYADSVKGRFTISRDNSKNTVYLQMNSLRAEDTAVYYCSAVKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab103 | 409 | FL VH- | EVQLVESGGGLVQPGGSLRLSCAASGFPFSSYAMGWYRQAPGKGREFVAAIS |
| | | Ig Fc | FLEGSTYYADSVKGRFTISRDNSKNTVYLQMNSLRAEDTAVYYCRAGKQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab104 | 410 | FL VH- | EVQLVESGGGLVQPGGSLRLSCAASGFDISSYAMGWYRQAPGKGREFVAAIS |
| | | Ig Fc | FVAGSTYYADSVKGRFTISRDNSKNTVYLQMNSLRAEDTAVYYCEARRQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab105 | 411 | FL VH- | EVQLVESGGGLVQPGGSLRLSCAASGFVGSSYAMGWYRQAPGKGREFVASIG |
| | | Ig Fc | GSGGSTYYADSVKGRFTISRDNSKNTVYLQMNSLRAEDTAVYYCRVARQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab106 | 412 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab107 | 413 | FL VH- | EVQLVESGGGLVQPGGSLRLSCAASGFTHSSYAMGWYRQAPGKGREFVAVIS |
| | | Ig Fc | GSGGSTQLLDSVKGRFTISRDNSKNTVYLQMNSLRAEDTAVYYCRAERQHRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab108 | 414 | FL VH- | EVQLVESGGGLVQPGGSLRLSCAASGFTFSSPHMGWYRQAPGKGREFVAAIS |
| | | Ig Fc | GSGTAGYYADSVKGRFTISRDNSKNTVYLQMNSLRAEDTAVYYCRVSLQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab109 | 415 | FL VH- | EVQLVESGGGLVQPGGSLRLSCAASGFPHPSYPMGWYRQAPGKGREFVAGPG |
| | | Ig Fc | GSGGSTYYADSVKGRFTISRDNSKNTVYLQMNSLRAEDTAVYYCRGARQRRT |
| | | (LALA | LDGYRSSFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab110 | 416 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab111 | 417 | FL VH- | EVQLVESGGGLVQPGGSLRLSCAASGFTFSSYAMGWYRQAPGKGREFVASIS |
| | | Ig Fc | GSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab112 | 418 | FL VH- | EVQLVESGGGLVQPGGSLRLSCAASGFTFSSYAMGWYRQAPGKGREFVASIS |
| | | Ig Fc | FSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab113 | 419 | FL VH- | EVQLVESGGGLVQPGGSLRLSCAASGFTFSSYAMGWYRQAPGKGREFVASIS |
| | | Ig Fc | GSGGSTQYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab114 | 420 | FL VH- | EVQLVESGGGLVQPGGSLRLSCAASGFTFSSYAMGWYRQAPGKGREFVAAIS |
| | | Ig Fc | FSGGSTQYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab115 | 421 | FL VH- | EVQLVESGGGLVQPGGSLRLSCAASGFTFSSYAMGWYRQAPGKGREFVAAIS |
| | | Ig Fc | FSGGSTYYADSVKGRFTISRDNSKNTVYLQMNSLRAEDTAVYYCRMMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab116 | 422 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab117 | 423 | FL VH- | EVQLVESGGGLVQPGGSLRLSCAASGFHHSSYAMGWYRQAPGKGREFVASIS |
| | | Ig Fc | GSGGSTYYADSVKGRFTISRDNSKNTVYLQMNSLRAEDTAVYYCRMVSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab118 | 424 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab119 | 425 | FL VH- | EVQLVESGGGLVQPGGSLRLSCAASGFHFSSYAMGWYRQAPGKGREFVASIS |
| | | Ig Fc | GSGGSTYYADSVKGRFTISRDNSKNTVYLQMNSLRAEDTAVYYCRMVSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |
| Ab120 | 426 | FL VH- | EVQLVESGGGLVQPGGSLRLSCAASGFTHSSYAMGWYRQAPGKGREFVASIS |
| | | Ig Fc | GSGGSTYYADSVKGRFTISRDNSKNTVYLQMNSLRAEDTAVYYCRSMSQRRT |
| | | (LALA | LDGYRTIFDYWGQGTQVTVSSGGGGSEPKSSDKTHTCPPCPAPEAAGGPSVF |
| | | PALS) | LFPPKPKDTLMISRTPEVTCVVVDVSHEDPEVKFNWYVDGVEVHNAKTKPRE |
| | | no c | EQYNSTYRVVSVLTVLHQDWLNGKEYKCKVSNKALAAPIEKTISKAKGQPRE |
| | | term K | PQVYTLPPSRDELTKNQVSLTCLVKGFYPSDIAVEWESNGQPENNYKTTPPV |
| | | | LDSDGSFFLYSKLTVDKSRWQQGNVFSCSVLHEALHSHYTQKSLSLSPG |

In certain embodiments, the instant disclosure provides an antibody that specifically binds to APJ (e.g., human APJ), the antibody comprising any one of the VH and Ig Fc combinations shown in Table 14.

TABLE 14
VH-Ig Fc combinations.

| VH-Ig Fc | | VH-Ig Fc | | VH-Ig Fc | | VH-Ig Fc | | VH-Ig Fc | | VH-Ig Fc | |
|---|---|---|---|---|---|---|---|---|---|---|---|
| VH | Ig Fc | VH | Ig Fc | VH | Ig Fc | VH | Ig Fc | VH | Ig Fc | VH | Ig Fc |
| SEQ ID NO | | SEQ ID NO | | SEQ ID NO | | SEQ ID NO | | SEQ ID NO | | SEQ ID NO | |
| 1 | 253 | 1 | 254 | 1 | 255 | 1 | 256 | 1 | 257 | 1 | 258 |
| 1 | 259 | 1 | 260 | 1 | 450 | 1 | 451 | 1 | 452 | 1 | 453 |
| 1 | 454 | 1 | 455 | 1 | 456 | 1 | 261 | 1 | 262 | 1 | 263 |
| 1 | 264 | 1 | 265 | 1 | 266 | 1 | 267 | 1 | 268 | 1 | 463 |
| 1 | 457 | 1 | 458 | 1 | 459 | 1 | 460 | 1 | 461 | 1 | 462 |
| 2 | 253 | 2 | 254 | 2 | 255 | 2 | 256 | 2 | 257 | 2 | 258 |
| 2 | 259 | 2 | 260 | 2 | 450 | 2 | 451 | 2 | 452 | 2 | 453 |
| 2 | 454 | 2 | 455 | 2 | 456 | 2 | 261 | 2 | 262 | 2 | 263 |
| 2 | 264 | 2 | 265 | 2 | 266 | 2 | 267 | 2 | 268 | 2 | 463 |
| 2 | 457 | 2 | 458 | 2 | 459 | 2 | 460 | 2 | 461 | 2 | 462 |
| 3 | 253 | 3 | 254 | 3 | 255 | 3 | 256 | 3 | 257 | 3 | 258 |
| 3 | 259 | 3 | 260 | 3 | 450 | 3 | 451 | 3 | 452 | 3 | 453 |
| 3 | 454 | 3 | 455 | 3 | 456 | 3 | 261 | 3 | 262 | 3 | 263 |
| 3 | 264 | 3 | 265 | 3 | 266 | 3 | 267 | 3 | 268 | 3 | 463 |
| 3 | 457 | 3 | 458 | 3 | 459 | 3 | 460 | 3 | 461 | 3 | 462 |
| 4 | 253 | 4 | 254 | 4 | 255 | 4 | 256 | 4 | 257 | 4 | 258 |
| 4 | 259 | 4 | 260 | 4 | 450 | 4 | 451 | 4 | 452 | 4 | 453 |
| 4 | 454 | 4 | 455 | 4 | 456 | 4 | 261 | 4 | 262 | 4 | 263 |
| 4 | 264 | 4 | 265 | 4 | 266 | 4 | 267 | 4 | 268 | 4 | 463 |
| 4 | 457 | 4 | 458 | 4 | 459 | 4 | 460 | 4 | 461 | 4 | 462 |
| 5 | 253 | 5 | 254 | 5 | 255 | 5 | 256 | 5 | 257 | 5 | 258 |
| 5 | 259 | 5 | 260 | 5 | 450 | 5 | 451 | 5 | 452 | 5 | 453 |
| 5 | 454 | 5 | 455 | 5 | 456 | 5 | 261 | 5 | 262 | 5 | 263 |
| 5 | 264 | 5 | 265 | 5 | 266 | 5 | 267 | 5 | 268 | 5 | 463 |
| 5 | 457 | 5 | 458 | 5 | 459 | 5 | 460 | 5 | 461 | 5 | 462 |
| 6 | 253 | 6 | 254 | 6 | 255 | 6 | 256 | 6 | 257 | 6 | 258 |
| 6 | 259 | 6 | 260 | 6 | 450 | 6 | 451 | 6 | 452 | 6 | 453 |
| 6 | 454 | 6 | 455 | 6 | 456 | 6 | 261 | 6 | 262 | 6 | 263 |
| 6 | 264 | 6 | 265 | 6 | 266 | 6 | 267 | 6 | 268 | 6 | 463 |
| 6 | 457 | 6 | 458 | 6 | 459 | 6 | 460 | 6 | 461 | 6 | 462 |
| 7 | 253 | 7 | 254 | 7 | 255 | 7 | 256 | 7 | 257 | 7 | 258 |
| 7 | 259 | 7 | 260 | 7 | 450 | 7 | 451 | 7 | 452 | 7 | 453 |
| 7 | 454 | 7 | 455 | 7 | 456 | 7 | 261 | 7 | 262 | 7 | 263 |
| 7 | 264 | 7 | 265 | 7 | 266 | 7 | 267 | 7 | 268 | 7 | 463 |
| 7 | 457 | 7 | 458 | 7 | 459 | 7 | 460 | 7 | 461 | 7 | 462 |
| 8 | 253 | 8 | 254 | 8 | 255 | 8 | 256 | 8 | 257 | 8 | 258 |
| 8 | 259 | 8 | 260 | 8 | 450 | 8 | 451 | 8 | 452 | 8 | 453 |
| 8 | 454 | 8 | 455 | 8 | 456 | 8 | 261 | 8 | 262 | 8 | 263 |
| 8 | 264 | 8 | 265 | 8 | 266 | 8 | 267 | 8 | 268 | 8 | 463 |
| 8 | 457 | 8 | 458 | 8 | 459 | 8 | 460 | 8 | 461 | 8 | 462 |
| 9 | 253 | 9 | 254 | 9 | 255 | 9 | 256 | 9 | 257 | 9 | 258 |
| 9 | 259 | 9 | 260 | 9 | 450 | 9 | 451 | 9 | 452 | 9 | 453 |
| 9 | 454 | 9 | 455 | 9 | 456 | 9 | 261 | 9 | 262 | 9 | 263 |
| 9 | 264 | 9 | 265 | 9 | 266 | 9 | 267 | 9 | 268 | 9 | 463 |
| 9 | 457 | 9 | 458 | 9 | 459 | 9 | 460 | 9 | 461 | 9 | 462 |
| 10 | 253 | 10 | 254 | 10 | 255 | 10 | 256 | 10 | 257 | 10 | 258 |
| 10 | 259 | 10 | 260 | 10 | 450 | 10 | 451 | 10 | 452 | 10 | 453 |
| 10 | 454 | 10 | 455 | 10 | 456 | 10 | 261 | 10 | 262 | 10 | 263 |
| 10 | 264 | 10 | 265 | 10 | 266 | 10 | 267 | 10 | 268 | 10 | 463 |
| 10 | 457 | 10 | 458 | 10 | 459 | 10 | 460 | 10 | 461 | 10 | 462 |
| 11 | 253 | 11 | 254 | 11 | 255 | 11 | 256 | 11 | 257 | 11 | 258 |
| 11 | 259 | 11 | 260 | 11 | 450 | 11 | 451 | 11 | 452 | 11 | 453 |
| 11 | 454 | 11 | 455 | 11 | 456 | 11 | 261 | 11 | 262 | 11 | 263 |
| 11 | 264 | 11 | 265 | 11 | 266 | 11 | 267 | 11 | 268 | 11 | 463 |
| 11 | 457 | 11 | 458 | 11 | 459 | 11 | 460 | 11 | 461 | 11 | 462 |
| 12 | 253 | 12 | 254 | 12 | 255 | 12 | 256 | 12 | 257 | 12 | 258 |
| 12 | 259 | 12 | 260 | 12 | 450 | 12 | 451 | 12 | 452 | 12 | 453 |
| 12 | 454 | 12 | 455 | 12 | 456 | 12 | 261 | 12 | 262 | 12 | 263 |
| 12 | 264 | 12 | 265 | 12 | 266 | 12 | 267 | 12 | 268 | 12 | 463 |
| 12 | 457 | 12 | 458 | 12 | 459 | 12 | 460 | 12 | 461 | 12 | 462 |
| 13 | 253 | 13 | 254 | 13 | 255 | 13 | 256 | 13 | 257 | 13 | 258 |
| 13 | 259 | 13 | 260 | 13 | 450 | 13 | 451 | 13 | 452 | 13 | 453 |
| 13 | 454 | 13 | 455 | 13 | 456 | 13 | 261 | 13 | 262 | 13 | 263 |
| 13 | 264 | 13 | 265 | 13 | 266 | 13 | 267 | 13 | 268 | 13 | 463 |
| 13 | 457 | 13 | 458 | 13 | 459 | 13 | 460 | 13 | 461 | 13 | 462 |
| 14 | 253 | 14 | 254 | 14 | 255 | 14 | 256 | 14 | 257 | 14 | 258 |
| 14 | 259 | 14 | 260 | 14 | 450 | 14 | 451 | 14 | 452 | 14 | 453 |
| 14 | 454 | 14 | 455 | 14 | 456 | 14 | 261 | 14 | 262 | 14 | 263 |
| 14 | 264 | 14 | 265 | 14 | 266 | 14 | 267 | 14 | 268 | 14 | 463 |
| 14 | 457 | 14 | 458 | 14 | 459 | 14 | 460 | 14 | 461 | 14 | 462 |
| 15 | 253 | 15 | 254 | 15 | 255 | 15 | 256 | 15 | 257 | 15 | 258 |
| 15 | 259 | 15 | 260 | 15 | 450 | 15 | 451 | 15 | 452 | 15 | 453 |
| 15 | 454 | 15 | 455 | 15 | 456 | 15 | 261 | 15 | 262 | 15 | 263 |
| 15 | 264 | 15 | 265 | 15 | 266 | 15 | 267 | 15 | 268 | 15 | 463 |
| 15 | 457 | 15 | 458 | 15 | 459 | 15 | 460 | 15 | 461 | 15 | 462 |
| 16 | 253 | 16 | 254 | 16 | 255 | 16 | 256 | 16 | 257 | 16 | 258 |
| 16 | 259 | 16 | 260 | 16 | 450 | 16 | 451 | 16 | 452 | 16 | 453 |
| 16 | 454 | 16 | 455 | 16 | 456 | 16 | 261 | 16 | 262 | 16 | 263 |
| 16 | 264 | 16 | 265 | 16 | 266 | 16 | 267 | 16 | 268 | 16 | 463 |
| 16 | 457 | 16 | 458 | 16 | 459 | 16 | 460 | 16 | 461 | 16 | 462 |
| 17 | 253 | 17 | 254 | 17 | 255 | 17 | 256 | 17 | 257 | 17 | 258 |
| 17 | 259 | 17 | 260 | 17 | 450 | 17 | 451 | 17 | 452 | 17 | 453 |
| 17 | 454 | 17 | 455 | 17 | 456 | 17 | 261 | 17 | 262 | 17 | 263 |
| 17 | 264 | 17 | 265 | 17 | 266 | 17 | 267 | 17 | 268 | 17 | 463 |
| 17 | 457 | 17 | 458 | 17 | 459 | 17 | 460 | 17 | 461 | 17 | 462 |
| 18 | 253 | 18 | 254 | 18 | 255 | 18 | 256 | 18 | 257 | 18 | 258 |
| 18 | 259 | 18 | 260 | 18 | 450 | 18 | 451 | 18 | 452 | 18 | 453 |
| 18 | 454 | 18 | 455 | 18 | 456 | 18 | 261 | 18 | 262 | 18 | 263 |
| 18 | 264 | 18 | 265 | 18 | 266 | 18 | 267 | 18 | 268 | 18 | 463 |
| 18 | 457 | 18 | 458 | 18 | 459 | 18 | 460 | 18 | 461 | 18 | 462 |
| 19 | 253 | 19 | 254 | 19 | 255 | 19 | 256 | 19 | 257 | 19 | 258 |
| 19 | 259 | 19 | 260 | 19 | 450 | 19 | 451 | 19 | 452 | 19 | 453 |
| 19 | 454 | 19 | 455 | 19 | 456 | 19 | 261 | 19 | 262 | 19 | 263 |
| 19 | 264 | 19 | 265 | 19 | 266 | 19 | 267 | 19 | 268 | 19 | 463 |
| 19 | 457 | 19 | 458 | 19 | 459 | 19 | 460 | 19 | 461 | 19 | 462 |
| 20 | 253 | 20 | 254 | 20 | 255 | 20 | 256 | 20 | 257 | 20 | 258 |
| 20 | 259 | 20 | 260 | 20 | 450 | 20 | 451 | 20 | 452 | 20 | 453 |
| 20 | 454 | 20 | 455 | 20 | 456 | 20 | 261 | 20 | 262 | 20 | 263 |
| 20 | 264 | 20 | 265 | 20 | 266 | 20 | 267 | 20 | 268 | 20 | 463 |
| 20 | 457 | 20 | 458 | 20 | 459 | 20 | 460 | 20 | 461 | 20 | 462 |
| 21 | 253 | 21 | 254 | 21 | 255 | 21 | 256 | 21 | 257 | 21 | 258 |
| 21 | 259 | 21 | 260 | 21 | 450 | 21 | 451 | 21 | 452 | 21 | 453 |
| 21 | 454 | 21 | 455 | 21 | 456 | 21 | 261 | 21 | 262 | 21 | 263 |
| 21 | 264 | 21 | 265 | 21 | 266 | 21 | 267 | 21 | 268 | 21 | 463 |
| 21 | 457 | 21 | 458 | 21 | 459 | 21 | 460 | 21 | 461 | 21 | 462 |
| 22 | 253 | 22 | 254 | 22 | 255 | 22 | 256 | 22 | 257 | 22 | 258 |
| 22 | 259 | 22 | 260 | 22 | 450 | 22 | 451 | 22 | 452 | 22 | 453 |
| 22 | 454 | 22 | 455 | 22 | 456 | 22 | 261 | 22 | 262 | 22 | 263 |
| 22 | 264 | 22 | 265 | 22 | 266 | 22 | 267 | 22 | 268 | 22 | 463 |
| 22 | 457 | 22 | 458 | 22 | 459 | 22 | 460 | 22 | 461 | 22 | 462 |
| 23 | 253 | 23 | 254 | 23 | 255 | 23 | 256 | 23 | 257 | 23 | 258 |
| 23 | 259 | 23 | 260 | 23 | 450 | 23 | 451 | 23 | 452 | 23 | 453 |
| 23 | 454 | 23 | 455 | 23 | 456 | 23 | 261 | 23 | 262 | 23 | 263 |
| 23 | 264 | 23 | 265 | 23 | 266 | 23 | 267 | 23 | 268 | 23 | 463 |
| 23 | 457 | 23 | 458 | 23 | 459 | 23 | 460 | 23 | 461 | 23 | 462 |
| 24 | 253 | 24 | 254 | 24 | 255 | 24 | 256 | 24 | 257 | 24 | 258 |
| 24 | 259 | 24 | 260 | 24 | 450 | 24 | 451 | 24 | 452 | 24 | 453 |
| 24 | 454 | 24 | 455 | 24 | 456 | 24 | 261 | 24 | 262 | 24 | 263 |
| 24 | 264 | 24 | 265 | 24 | 266 | 24 | 267 | 24 | 268 | 24 | 463 |
| 24 | 457 | 24 | 458 | 24 | 459 | 24 | 460 | 24 | 461 | 24 | 462 |
| 25 | 253 | 25 | 254 | 25 | 255 | 25 | 256 | 25 | 257 | 25 | 258 |
| 25 | 259 | 25 | 260 | 25 | 450 | 25 | 451 | 25 | 452 | 25 | 453 |
| 25 | 454 | 25 | 455 | 25 | 456 | 25 | 261 | 25 | 262 | 25 | 263 |
| 25 | 264 | 25 | 265 | 25 | 266 | 25 | 267 | 25 | 268 | 25 | 463 |
| 25 | 457 | 25 | 458 | 25 | 459 | 25 | 460 | 25 | 461 | 25 | 462 |
| 26 | 253 | 26 | 254 | 26 | 255 | 26 | 256 | 26 | 257 | 26 | 258 |
| 26 | 259 | 26 | 260 | 26 | 450 | 26 | 451 | 26 | 452 | 26 | 453 |
| 26 | 454 | 26 | 455 | 26 | 456 | 26 | 261 | 26 | 262 | 26 | 263 |
| 26 | 264 | 26 | 265 | 26 | 266 | 26 | 267 | 26 | 268 | 26 | 463 |
| 26 | 457 | 26 | 458 | 26 | 459 | 26 | 460 | 26 | 461 | 26 | 462 |
| 27 | 253 | 27 | 254 | 27 | 255 | 27 | 256 | 27 | 257 | 27 | 258 |
| 27 | 259 | 27 | 260 | 27 | 450 | 27 | 451 | 27 | 452 | 27 | 453 |
| 27 | 454 | 27 | 455 | 27 | 456 | 27 | 261 | 27 | 262 | 27 | 263 |
| 27 | 264 | 27 | 265 | 27 | 266 | 27 | 267 | 27 | 268 | 27 | 463 |
| 27 | 457 | 27 | 458 | 27 | 459 | 27 | 460 | 27 | 461 | 27 | 462 |
| 28 | 253 | 28 | 254 | 28 | 255 | 28 | 256 | 28 | 257 | 28 | 258 |
| 28 | 259 | 28 | 260 | 28 | 450 | 28 | 451 | 28 | 452 | 28 | 453 |
| 28 | 454 | 28 | 455 | 28 | 456 | 28 | 261 | 28 | 262 | 28 | 263 |
| 28 | 264 | 28 | 265 | 28 | 266 | 28 | 267 | 28 | 268 | 28 | 463 |
| 28 | 457 | 28 | 458 | 28 | 459 | 28 | 460 | 28 | 461 | 28 | 462 |
| 29 | 253 | 29 | 254 | 29 | 255 | 29 | 256 | 29 | 257 | 29 | 258 |
| 29 | 259 | 29 | 260 | 29 | 450 | 29 | 451 | 29 | 452 | 29 | 453 |
| 29 | 454 | 29 | 455 | 29 | 456 | 29 | 261 | 29 | 262 | 29 | 263 |
| 29 | 264 | 29 | 265 | 29 | 266 | 29 | 267 | 29 | 268 | 29 | 463 |
| 29 | 457 | 29 | 458 | 29 | 459 | 29 | 460 | 29 | 461 | 29 | 462 |
| 30 | 253 | 30 | 254 | 30 | 255 | 30 | 256 | 30 | 257 | 30 | 258 |
| 30 | 259 | 30 | 260 | 30 | 450 | 30 | 451 | 30 | 452 | 30 | 453 |
| 30 | 454 | 30 | 455 | 30 | 456 | 30 | 261 | 30 | 262 | 30 | 263 |
| 30 | 264 | 30 | 265 | 30 | 266 | 30 | 267 | 30 | 268 | 30 | 463 |
| 30 | 457 | 30 | 458 | 30 | 459 | 30 | 460 | 30 | 461 | 30 | 462 |
| 31 | 253 | 31 | 254 | 31 | 255 | 31 | 256 | 31 | 257 | 31 | 258 |
| 31 | 259 | 31 | 260 | 31 | 450 | 31 | 451 | 31 | 452 | 31 | 453 |
| 31 | 454 | 31 | 455 | 31 | 456 | 31 | 261 | 31 | 262 | 31 | 263 |
| 31 | 264 | 31 | 265 | 31 | 266 | 31 | 267 | 31 | 268 | 31 | 463 |
| 31 | 457 | 31 | 458 | 31 | 459 | 31 | 460 | 31 | 461 | 31 | 462 |
| 32 | 253 | 32 | 254 | 32 | 255 | 32 | 256 | 32 | 257 | 32 | 258 |
| 32 | 259 | 32 | 260 | 32 | 450 | 32 | 451 | 32 | 452 | 32 | 453 |
| 32 | 454 | 32 | 455 | 32 | 456 | 32 | 261 | 32 | 262 | 32 | 263 |
| 32 | 264 | 32 | 265 | 32 | 266 | 32 | 267 | 32 | 268 | 32 | 463 |
| 32 | 457 | 32 | 458 | 32 | 459 | 32 | 460 | 32 | 461 | 32 | 462 |
| 33 | 253 | 33 | 254 | 33 | 255 | 33 | 256 | 33 | 257 | 33 | 258 |
| 33 | 259 | 33 | 260 | 33 | 450 | 33 | 451 | 33 | 452 | 33 | 453 |
| 33 | 454 | 33 | 455 | 33 | 456 | 33 | 261 | 33 | 262 | 33 | 263 |
| 33 | 264 | 33 | 265 | 33 | 266 | 33 | 267 | 33 | 268 | 33 | 463 |
| 33 | 457 | 33 | 458 | 33 | 459 | 33 | 460 | 33 | 461 | 33 | 462 |
| 34 | 253 | 34 | 254 | 34 | 255 | 34 | 256 | 34 | 257 | 34 | 258 |
| 34 | 259 | 34 | 260 | 34 | 450 | 34 | 451 | 34 | 452 | 34 | 453 |
| 34 | 454 | 34 | 455 | 34 | 456 | 34 | 261 | 34 | 262 | 34 | 263 |
| 34 | 264 | 34 | 265 | 34 | 266 | 34 | 267 | 34 | 268 | 34 | 463 |
| 34 | 457 | 34 | 458 | 34 | 459 | 34 | 460 | 34 | 461 | 34 | 462 |
| 35 | 253 | 35 | 254 | 35 | 255 | 35 | 256 | 35 | 257 | 35 | 258 |
| 35 | 259 | 35 | 260 | 35 | 450 | 35 | 451 | 35 | 452 | 35 | 453 |
| 35 | 454 | 35 | 455 | 35 | 456 | 35 | 261 | 35 | 262 | 35 | 263 |
| 35 | 264 | 35 | 265 | 35 | 266 | 35 | 267 | 35 | 268 | 35 | 463 |
| 35 | 457 | 35 | 458 | 35 | 459 | 35 | 460 | 35 | 461 | 35 | 462 |
| 36 | 253 | 36 | 254 | 36 | 255 | 36 | 256 | 36 | 257 | 36 | 258 |
| 36 | 259 | 36 | 260 | 36 | 450 | 36 | 451 | 36 | 452 | 36 | 453 |
| 36 | 454 | 36 | 455 | 36 | 456 | 36 | 261 | 36 | 262 | 36 | 263 |
| 36 | 264 | 36 | 265 | 36 | 266 | 36 | 267 | 36 | 268 | 36 | 463 |
| 36 | 457 | 36 | 458 | 36 | 459 | 36 | 460 | 36 | 461 | 36 | 462 |
| 37 | 253 | 37 | 254 | 37 | 255 | 37 | 256 | 37 | 257 | 37 | 258 |
| 37 | 259 | 37 | 260 | 37 | 450 | 37 | 451 | 37 | 452 | 37 | 453 |
| 37 | 454 | 37 | 455 | 37 | 456 | 37 | 261 | 37 | 262 | 37 | 263 |
| 37 | 264 | 37 | 265 | 37 | 266 | 37 | 267 | 37 | 268 | 37 | 463 |
| 37 | 457 | 37 | 458 | 37 | 459 | 37 | 460 | 37 | 461 | 37 | 462 |
| 38 | 253 | 38 | 254 | 38 | 255 | 38 | 256 | 38 | 257 | 38 | 258 |
| 38 | 259 | 38 | 260 | 38 | 450 | 38 | 451 | 38 | 452 | 38 | 453 |
| 38 | 454 | 38 | 455 | 38 | 456 | 38 | 261 | 38 | 262 | 38 | 263 |
| 38 | 264 | 38 | 265 | 38 | 266 | 38 | 267 | 38 | 268 | 38 | 463 |
| 38 | 457 | 38 | 458 | 38 | 459 | 38 | 460 | 38 | 461 | 38 | 462 |
| 39 | 253 | 39 | 254 | 39 | 255 | 39 | 256 | 39 | 257 | 39 | 258 |
| 39 | 259 | 39 | 260 | 39 | 450 | 39 | 451 | 39 | 452 | 39 | 453 |
| 39 | 454 | 39 | 455 | 39 | 456 | 39 | 261 | 39 | 262 | 39 | 263 |
| 39 | 264 | 39 | 265 | 39 | 266 | 39 | 267 | 39 | 268 | 39 | 463 |
| 39 | 457 | 39 | 458 | 39 | 459 | 39 | 460 | 39 | 461 | 39 | 462 |
| 40 | 253 | 40 | 254 | 40 | 255 | 40 | 256 | 40 | 257 | 40 | 258 |
| 40 | 259 | 40 | 260 | 40 | 450 | 40 | 451 | 40 | 452 | 40 | 453 |
| 40 | 454 | 40 | 455 | 40 | 456 | 40 | 261 | 40 | 262 | 40 | 263 |
| 40 | 264 | 40 | 265 | 40 | 266 | 40 | 267 | 40 | 268 | 40 | 463 |
| 40 | 457 | 40 | 458 | 40 | 459 | 40 | 460 | 40 | 461 | 40 | 462 |
| 41 | 253 | 41 | 254 | 41 | 255 | 41 | 256 | 41 | 257 | 41 | 258 |
| 41 | 259 | 41 | 260 | 41 | 450 | 41 | 451 | 41 | 452 | 41 | 453 |
| 41 | 454 | 41 | 455 | 41 | 456 | 41 | 261 | 41 | 262 | 41 | 263 |
| 41 | 264 | 41 | 265 | 41 | 266 | 41 | 267 | 41 | 268 | 41 | 463 |
| 41 | 457 | 41 | 458 | 41 | 459 | 41 | 460 | 41 | 461 | 41 | 462 |
| 42 | 253 | 42 | 254 | 42 | 255 | 42 | 256 | 42 | 257 | 42 | 258 |
| 42 | 259 | 42 | 260 | 42 | 450 | 42 | 451 | 42 | 452 | 42 | 453 |
| 42 | 454 | 42 | 455 | 42 | 456 | 42 | 261 | 42 | 262 | 42 | 263 |
| 42 | 264 | 42 | 265 | 42 | 266 | 42 | 267 | 42 | 268 | 42 | 463 |
| 42 | 457 | 42 | 458 | 42 | 459 | 42 | 460 | 42 | 461 | 42 | 462 |
| 43 | 253 | 43 | 254 | 43 | 255 | 43 | 256 | 43 | 257 | 43 | 258 |
| 43 | 259 | 43 | 260 | 43 | 450 | 43 | 451 | 43 | 452 | 43 | 453 |
| 43 | 454 | 43 | 455 | 43 | 456 | 43 | 261 | 43 | 262 | 43 | 263 |
| 43 | 264 | 43 | 265 | 43 | 266 | 43 | 267 | 43 | 268 | 43 | 463 |
| 43 | 457 | 43 | 458 | 43 | 459 | 43 | 460 | 43 | 461 | 43 | 462 |
| 44 | 253 | 44 | 254 | 44 | 255 | 44 | 256 | 44 | 257 | 44 | 258 |
| 44 | 259 | 44 | 260 | 44 | 450 | 44 | 451 | 44 | 452 | 44 | 453 |
| 44 | 454 | 44 | 455 | 44 | 456 | 44 | 261 | 44 | 262 | 44 | 263 |
| 44 | 264 | 44 | 265 | 44 | 266 | 44 | 267 | 44 | 268 | 44 | 463 |
| 44 | 457 | 44 | 458 | 44 | 459 | 44 | 460 | 44 | 461 | 44 | 462 |
| 45 | 253 | 45 | 254 | 45 | 255 | 45 | 256 | 45 | 257 | 45 | 258 |
| 45 | 259 | 45 | 260 | 45 | 450 | 45 | 451 | 45 | 452 | 45 | 453 |
| 45 | 454 | 45 | 455 | 45 | 456 | 45 | 261 | 45 | 262 | 45 | 263 |
| 45 | 264 | 45 | 265 | 45 | 266 | 45 | 267 | 45 | 268 | 45 | 463 |
| 45 | 457 | 45 | 458 | 45 | 459 | 45 | 460 | 45 | 461 | 45 | 462 |
| 46 | 253 | 46 | 254 | 46 | 255 | 46 | 256 | 46 | 257 | 46 | 258 |
| 46 | 259 | 46 | 260 | 46 | 450 | 46 | 451 | 46 | 452 | 46 | 453 |
| 46 | 454 | 46 | 455 | 46 | 456 | 46 | 261 | 46 | 262 | 46 | 263 |
| 46 | 264 | 46 | 265 | 46 | 266 | 46 | 267 | 46 | 268 | 46 | 463 |
| 46 | 457 | 46 | 458 | 46 | 459 | 46 | 460 | 46 | 461 | 46 | 462 |
| 47 | 253 | 47 | 254 | 47 | 255 | 47 | 256 | 47 | 257 | 47 | 258 |
| 47 | 259 | 47 | 260 | 47 | 450 | 47 | 451 | 47 | 452 | 47 | 453 |
| 47 | 454 | 47 | 455 | 47 | 456 | 47 | 261 | 47 | 262 | 47 | 263 |
| 47 | 264 | 47 | 265 | 47 | 266 | 47 | 267 | 47 | 268 | 47 | 463 |
| 47 | 457 | 47 | 458 | 47 | 459 | 47 | 460 | 47 | 461 | 47 | 462 |
| 48 | 253 | 48 | 254 | 48 | 255 | 48 | 256 | 48 | 257 | 48 | 258 |
| 48 | 259 | 48 | 260 | 48 | 450 | 48 | 451 | 48 | 452 | 48 | 453 |
| 48 | 454 | 48 | 455 | 48 | 456 | 48 | 261 | 48 | 262 | 48 | 263 |
| 48 | 264 | 48 | 265 | 48 | 266 | 48 | 267 | 48 | 268 | 48 | 463 |
| 48 | 457 | 48 | 458 | 48 | 459 | 48 | 460 | 48 | 461 | 48 | 462 |
| 49 | 253 | 49 | 254 | 49 | 255 | 49 | 256 | 49 | 257 | 49 | 258 |
| 49 | 259 | 49 | 260 | 49 | 450 | 49 | 451 | 49 | 452 | 49 | 453 |
| 49 | 454 | 49 | 455 | 49 | 456 | 49 | 261 | 49 | 262 | 49 | 263 |
| 49 | 264 | 49 | 265 | 49 | 266 | 49 | 267 | 49 | 268 | 49 | 463 |
| 49 | 457 | 49 | 458 | 49 | 459 | 49 | 460 | 49 | 461 | 49 | 462 |
| 50 | 253 | 50 | 254 | 50 | 255 | 50 | 256 | 50 | 257 | 50 | 258 |
| 50 | 259 | 50 | 260 | 50 | 450 | 50 | 451 | 50 | 452 | 50 | 453 |
| 50 | 454 | 50 | 455 | 50 | 456 | 50 | 261 | 50 | 262 | 50 | 263 |
| 50 | 264 | 50 | 265 | 50 | 266 | 50 | 267 | 50 | 268 | 50 | 463 |
| 50 | 457 | 50 | 458 | 50 | 459 | 50 | 460 | 50 | 461 | 50 | 462 |
| 51 | 253 | 51 | 254 | 51 | 255 | 51 | 256 | 51 | 257 | 51 | 258 |
| 51 | 259 | 51 | 260 | 51 | 450 | 51 | 451 | 51 | 452 | 51 | 453 |
| 51 | 454 | 51 | 455 | 51 | 456 | 51 | 261 | 51 | 262 | 51 | 263 |
| 51 | 264 | 51 | 265 | 51 | 266 | 51 | 267 | 51 | 268 | 51 | 463 |
| 51 | 457 | 51 | 458 | 51 | 459 | 51 | 460 | 51 | 461 | 51 | 462 |
| 52 | 253 | 52 | 254 | 52 | 255 | 52 | 256 | 52 | 257 | 52 | 258 |
| 52 | 259 | 52 | 260 | 52 | 450 | 52 | 451 | 52 | 452 | 52 | 453 |
| 52 | 454 | 52 | 455 | 52 | 456 | 52 | 261 | 52 | 262 | 52 | 263 |
| 52 | 264 | 52 | 265 | 52 | 266 | 52 | 267 | 52 | 268 | 52 | 463 |
| 52 | 457 | 52 | 458 | 52 | 459 | 52 | 460 | 52 | 461 | 52 | 462 |
| 53 | 253 | 53 | 254 | 53 | 255 | 53 | 256 | 53 | 257 | 53 | 258 |
| 53 | 259 | 53 | 260 | 53 | 450 | 53 | 451 | 53 | 452 | 53 | 453 |
| 53 | 454 | 53 | 455 | 53 | 456 | 53 | 261 | 53 | 262 | 53 | 263 |
| 53 | 264 | 53 | 265 | 53 | 266 | 53 | 267 | 53 | 268 | 53 | 463 |
| 53 | 457 | 53 | 458 | 53 | 459 | 53 | 460 | 53 | 461 | 53 | 462 |
| 54 | 253 | 54 | 254 | 54 | 255 | 54 | 256 | 54 | 257 | 54 | 258 |
| 54 | 259 | 54 | 260 | 54 | 450 | 54 | 451 | 54 | 452 | 54 | 453 |
| 54 | 454 | 54 | 455 | 54 | 456 | 54 | 261 | 54 | 262 | 54 | 263 |
| 54 | 264 | 54 | 265 | 54 | 266 | 54 | 267 | 54 | 268 | 54 | 463 |
| 54 | 457 | 54 | 458 | 54 | 459 | 54 | 460 | 54 | 461 | 54 | 462 |
| 55 | 253 | 55 | 254 | 55 | 255 | 55 | 256 | 55 | 257 | 55 | 258 |
| 55 | 259 | 55 | 260 | 55 | 450 | 55 | 451 | 55 | 452 | 55 | 453 |
| 55 | 454 | 55 | 455 | 55 | 456 | 55 | 261 | 55 | 262 | 55 | 263 |
| 55 | 264 | 55 | 265 | 55 | 266 | 55 | 267 | 55 | 268 | 55 | 463 |
| 55 | 457 | 55 | 458 | 55 | 459 | 55 | 460 | 55 | 461 | 55 | 462 |
| 56 | 253 | 56 | 254 | 56 | 255 | 56 | 256 | 56 | 257 | 56 | 258 |
| 56 | 259 | 56 | 260 | 56 | 450 | 56 | 451 | 56 | 452 | 56 | 453 |
| 56 | 454 | 56 | 455 | 56 | 456 | 56 | 261 | 56 | 262 | 56 | 263 |
| 56 | 264 | 56 | 265 | 56 | 266 | 56 | 267 | 56 | 268 | 56 | 463 |
| 56 | 457 | 56 | 458 | 56 | 459 | 56 | 460 | 56 | 461 | 56 | 462 |
| 57 | 253 | 57 | 254 | 57 | 255 | 57 | 256 | 57 | 257 | 57 | 258 |
| 57 | 259 | 57 | 260 | 57 | 450 | 57 | 451 | 57 | 452 | 57 | 453 |
| 57 | 454 | 57 | 455 | 57 | 456 | 57 | 261 | 57 | 262 | 57 | 263 |
| 57 | 264 | 57 | 265 | 57 | 266 | 57 | 267 | 57 | 268 | 57 | 463 |
| 57 | 457 | 57 | 458 | 57 | 459 | 57 | 460 | 57 | 461 | 57 | 462 |
| 58 | 253 | 58 | 254 | 58 | 255 | 58 | 256 | 58 | 257 | 58 | 258 |
| 58 | 259 | 58 | 260 | 58 | 450 | 58 | 451 | 58 | 452 | 58 | 453 |
| 58 | 454 | 58 | 455 | 58 | 456 | 58 | 261 | 58 | 262 | 58 | 263 |
| 58 | 264 | 58 | 265 | 58 | 266 | 58 | 267 | 58 | 268 | 58 | 463 |
| 58 | 457 | 58 | 458 | 58 | 459 | 58 | 460 | 58 | 461 | 58 | 462 |
| 59 | 253 | 59 | 254 | 59 | 255 | 59 | 256 | 59 | 257 | 59 | 258 |
| 59 | 259 | 59 | 260 | 59 | 450 | 59 | 451 | 59 | 452 | 59 | 453 |
| 59 | 454 | 59 | 455 | 59 | 456 | 59 | 261 | 59 | 262 | 59 | 263 |
| 59 | 264 | 59 | 265 | 59 | 266 | 59 | 267 | 59 | 268 | 59 | 463 |
| 59 | 457 | 59 | 458 | 59 | 459 | 59 | 460 | 59 | 461 | 59 | 462 |
| 62 | 253 | 62 | 254 | 62 | 255 | 62 | 256 | 62 | 257 | 62 | 258 |
| 62 | 259 | 62 | 260 | 62 | 450 | 62 | 451 | 62 | 452 | 62 | 453 |
| 62 | 454 | 62 | 455 | 62 | 456 | 62 | 261 | 62 | 262 | 62 | 263 |
| 62 | 264 | 62 | 265 | 62 | 266 | 62 | 267 | 62 | 268 | 62 | 463 |
| 62 | 457 | 62 | 458 | 62 | 459 | 62 | 460 | 62 | 461 | 62 | 462 |
| 63 | 253 | 63 | 254 | 63 | 255 | 63 | 256 | 63 | 257 | 63 | 258 |
| 63 | 259 | 63 | 260 | 63 | 450 | 63 | 451 | 63 | 452 | 63 | 453 |
| 63 | 454 | 63 | 455 | 63 | 456 | 63 | 261 | 63 | 262 | 63 | 263 |
| 63 | 264 | 63 | 265 | 63 | 266 | 63 | 267 | 63 | 268 | 63 | 463 |
| 63 | 457 | 63 | 458 | 63 | 459 | 63 | 460 | 63 | 461 | 63 | 462 |
| 64 | 253 | 64 | 254 | 64 | 255 | 64 | 256 | 64 | 257 | 64 | 258 |
| 64 | 259 | 64 | 260 | 64 | 450 | 64 | 451 | 64 | 452 | 64 | 453 |
| 64 | 454 | 64 | 455 | 64 | 456 | 64 | 261 | 64 | 262 | 64 | 263 |
| 64 | 264 | 64 | 265 | 64 | 266 | 64 | 267 | 64 | 268 | 64 | 463 |
| 64 | 457 | 64 | 458 | 64 | 459 | 64 | 460 | 64 | 461 | 64 | 462 |
| 65 | 253 | 65 | 254 | 65 | 255 | 65 | 256 | 65 | 257 | 65 | 258 |
| 65 | 259 | 65 | 260 | 65 | 450 | 65 | 451 | 65 | 452 | 65 | 453 |
| 65 | 454 | 65 | 455 | 65 | 456 | 65 | 261 | 65 | 262 | 65 | 263 |
| 65 | 264 | 65 | 265 | 65 | 266 | 65 | 267 | 65 | 268 | 65 | 463 |
| 65 | 457 | 65 | 458 | 65 | 459 | 65 | 460 | 65 | 461 | 65 | 462 |
| 66 | 253 | 66 | 254 | 66 | 255 | 66 | 256 | 66 | 257 | 66 | 258 |
| 66 | 259 | 66 | 260 | 66 | 450 | 66 | 451 | 66 | 452 | 66 | 453 |
| 66 | 454 | 66 | 455 | 66 | 456 | 66 | 261 | 66 | 262 | 66 | 263 |
| 66 | 264 | 66 | 265 | 66 | 266 | 66 | 267 | 66 | 268 | 66 | 463 |
| 66 | 457 | 66 | 458 | 66 | 459 | 66 | 460 | 66 | 461 | 66 | 462 |
| 67 | 253 | 67 | 254 | 67 | 255 | 67 | 256 | 67 | 257 | 67 | 258 |
| 67 | 259 | 67 | 260 | 67 | 450 | 67 | 451 | 67 | 452 | 67 | 453 |
| 67 | 454 | 67 | 455 | 67 | 456 | 67 | 261 | 67 | 262 | 67 | 263 |
| 67 | 264 | 67 | 265 | 67 | 266 | 67 | 267 | 67 | 268 | 67 | 463 |
| 67 | 457 | 67 | 458 | 67 | 459 | 67 | 460 | 67 | 461 | 67 | 462 |
| 68 | 253 | 68 | 254 | 68 | 255 | 68 | 256 | 68 | 257 | 68 | 258 |
| 68 | 259 | 68 | 260 | 68 | 450 | 68 | 451 | 68 | 452 | 68 | 453 |
| 68 | 454 | 68 | 455 | 68 | 456 | 68 | 261 | 68 | 262 | 68 | 263 |
| 68 | 264 | 68 | 265 | 68 | 266 | 68 | 267 | 68 | 268 | 68 | 463 |
| 68 | 457 | 68 | 458 | 68 | 459 | 68 | 460 | 68 | 461 | 68 | 462 |
| 69 | 253 | 69 | 254 | 69 | 255 | 69 | 256 | 69 | 257 | 69 | 258 |
| 69 | 259 | 69 | 260 | 69 | 450 | 69 | 451 | 69 | 452 | 69 | 453 |
| 69 | 454 | 69 | 455 | 69 | 456 | 69 | 261 | 69 | 262 | 69 | 263 |
| 69 | 264 | 69 | 265 | 69 | 266 | 69 | 267 | 69 | 268 | 69 | 463 |
| 69 | 457 | 69 | 458 | 69 | 459 | 69 | 460 | 69 | 461 | 69 | 462 |
| 70 | 253 | 70 | 254 | 70 | 255 | 70 | 256 | 70 | 257 | 70 | 258 |
| 70 | 259 | 70 | 260 | 70 | 450 | 70 | 451 | 70 | 452 | 70 | 453 |
| 70 | 454 | 70 | 455 | 70 | 456 | 70 | 261 | 70 | 262 | 70 | 263 |
| 70 | 264 | 70 | 265 | 70 | 266 | 70 | 267 | 70 | 268 | 70 | 463 |
| 70 | 457 | 70 | 458 | 70 | 459 | 70 | 460 | 70 | 461 | 70 | 462 |
| 71 | 253 | 71 | 254 | 71 | 255 | 71 | 256 | 71 | 257 | 71 | 258 |
| 71 | 259 | 71 | 260 | 71 | 450 | 71 | 451 | 71 | 452 | 71 | 453 |
| 71 | 454 | 71 | 455 | 71 | 456 | 71 | 261 | 71 | 262 | 71 | 263 |
| 71 | 264 | 71 | 265 | 71 | 266 | 71 | 267 | 71 | 268 | 71 | 463 |
| 71 | 457 | 71 | 458 | 71 | 459 | 71 | 460 | 71 | 461 | 71 | 462 |
| 72 | 253 | 72 | 254 | 72 | 255 | 72 | 256 | 72 | 257 | 72 | 258 |
| 72 | 259 | 72 | 260 | 72 | 450 | 72 | 451 | 72 | 452 | 72 | 453 |
| 72 | 454 | 72 | 455 | 72 | 456 | 72 | 261 | 72 | 262 | 72 | 263 |
| 72 | 264 | 72 | 265 | 72 | 266 | 72 | 267 | 72 | 268 | 72 | 463 |
| 72 | 457 | 72 | 458 | 72 | 459 | 72 | 460 | 72 | 461 | 72 | 462 |
| 73 | 253 | 73 | 254 | 73 | 255 | 73 | 256 | 73 | 257 | 73 | 258 |
| 73 | 259 | 73 | 260 | 73 | 450 | 73 | 451 | 73 | 452 | 73 | 453 |
| 73 | 454 | 73 | 455 | 73 | 456 | 73 | 261 | 73 | 262 | 73 | 263 |
| 73 | 264 | 73 | 265 | 73 | 266 | 73 | 267 | 73 | 268 | 73 | 463 |
| 73 | 457 | 73 | 458 | 73 | 459 | 73 | 460 | 73 | 461 | 73 | 462 |
| 74 | 253 | 74 | 254 | 74 | 255 | 74 | 256 | 74 | 257 | 74 | 258 |
| 74 | 259 | 74 | 260 | 74 | 450 | 74 | 451 | 74 | 452 | 74 | 453 |
| 74 | 454 | 74 | 455 | 74 | 456 | 74 | 261 | 74 | 262 | 74 | 263 |
| 74 | 264 | 74 | 265 | 74 | 266 | 74 | 267 | 74 | 268 | 74 | 463 |
| 74 | 457 | 74 | 458 | 74 | 459 | 74 | 460 | 74 | 461 | 74 | 462 |
| 75 | 253 | 75 | 254 | 75 | 255 | 75 | 256 | 75 | 257 | 75 | 258 |
| 75 | 259 | 75 | 260 | 75 | 450 | 75 | 451 | 75 | 452 | 75 | 453 |
| 75 | 454 | 75 | 455 | 75 | 456 | 75 | 261 | 75 | 262 | 75 | 263 |
| 75 | 264 | 75 | 265 | 75 | 266 | 75 | 267 | 75 | 268 | 75 | 463 |
| 75 | 457 | 75 | 458 | 75 | 459 | 75 | 460 | 75 | 461 | 75 | 462 |
| 76 | 253 | 76 | 254 | 76 | 255 | 76 | 256 | 76 | 257 | 76 | 258 |
| 76 | 259 | 76 | 260 | 76 | 450 | 76 | 451 | 76 | 452 | 76 | 453 |
| 76 | 454 | 76 | 455 | 76 | 456 | 76 | 261 | 76 | 262 | 76 | 263 |
| 76 | 264 | 76 | 265 | 76 | 266 | 76 | 267 | 76 | 268 | 76 | 463 |
| 76 | 457 | 76 | 458 | 76 | 459 | 76 | 460 | 76 | 461 | 76 | 462 |
| 77 | 253 | 77 | 254 | 77 | 255 | 77 | 256 | 77 | 257 | 77 | 258 |
| 77 | 259 | 77 | 260 | 77 | 450 | 77 | 451 | 77 | 452 | 77 | 453 |
| 77 | 454 | 77 | 455 | 77 | 456 | 77 | 261 | 77 | 262 | 77 | 263 |
| 77 | 264 | 77 | 265 | 77 | 266 | 77 | 267 | 77 | 268 | 77 | 463 |
| 77 | 457 | 77 | 458 | 77 | 459 | 77 | 460 | 77 | 461 | 77 | 462 |
| 78 | 253 | 78 | 254 | 78 | 255 | 78 | 256 | 78 | 257 | 78 | 258 |
| 78 | 259 | 78 | 260 | 78 | 450 | 78 | 451 | 78 | 452 | 78 | 453 |
| 78 | 454 | 78 | 455 | 78 | 456 | 78 | 261 | 78 | 262 | 78 | 263 |
| 78 | 264 | 78 | 265 | 78 | 266 | 78 | 267 | 78 | 268 | 78 | 463 |
| 78 | 457 | 78 | 458 | 78 | 459 | 78 | 460 | 78 | 461 | 78 | 462 |
| 79 | 253 | 79 | 254 | 79 | 255 | 79 | 256 | 79 | 257 | 79 | 258 |
| 79 | 259 | 79 | 260 | 79 | 450 | 79 | 451 | 79 | 452 | 79 | 453 |
| 79 | 454 | 79 | 455 | 79 | 456 | 79 | 261 | 79 | 262 | 79 | 263 |
| 79 | 264 | 79 | 265 | 79 | 266 | 79 | 267 | 79 | 268 | 79 | 463 |
| 79 | 457 | 79 | 458 | 79 | 459 | 79 | 460 | 79 | 461 | 79 | 462 |
| 80 | 253 | 80 | 254 | 80 | 255 | 80 | 256 | 80 | 257 | 80 | 258 |
| 80 | 259 | 80 | 260 | 80 | 450 | 80 | 451 | 80 | 452 | 80 | 453 |
| 80 | 454 | 80 | 455 | 80 | 456 | 80 | 261 | 80 | 262 | 80 | 263 |
| 80 | 264 | 80 | 265 | 80 | 266 | 80 | 267 | 80 | 268 | 80 | 463 |
| 80 | 457 | 80 | 458 | 80 | 459 | 80 | 460 | 80 | 461 | 80 | 462 |
| 81 | 253 | 81 | 254 | 81 | 255 | 81 | 256 | 81 | 257 | 81 | 258 |
| 81 | 259 | 81 | 260 | 81 | 450 | 81 | 451 | 81 | 452 | 81 | 453 |
| 81 | 454 | 81 | 455 | 81 | 456 | 81 | 261 | 81 | 262 | 81 | 263 |
| 81 | 264 | 81 | 265 | 81 | 266 | 81 | 267 | 81 | 268 | 81 | 463 |
| 81 | 457 | 81 | 458 | 81 | 459 | 81 | 460 | 81 | 461 | 81 | 462 |
| 82 | 253 | 82 | 254 | 82 | 255 | 82 | 256 | 82 | 257 | 82 | 258 |
| 82 | 259 | 82 | 260 | 82 | 450 | 82 | 451 | 82 | 452 | 82 | 453 |
| 82 | 454 | 82 | 455 | 82 | 456 | 82 | 261 | 82 | 262 | 82 | 263 |
| 82 | 264 | 82 | 265 | 82 | 266 | 82 | 267 | 82 | 268 | 82 | 463 |
| 82 | 457 | 82 | 458 | 82 | 459 | 82 | 460 | 82 | 461 | 82 | 462 |
| 83 | 253 | 83 | 254 | 83 | 255 | 83 | 256 | 83 | 257 | 83 | 258 |
| 83 | 259 | 83 | 260 | 83 | 450 | 83 | 451 | 83 | 452 | 83 | 453 |
| 83 | 454 | 83 | 455 | 83 | 456 | 83 | 261 | 83 | 262 | 83 | 263 |
| 83 | 264 | 83 | 265 | 83 | 266 | 83 | 267 | 83 | 268 | 83 | 463 |
| 83 | 457 | 83 | 458 | 83 | 459 | 83 | 460 | 83 | 461 | 83 | 462 |
| 84 | 253 | 84 | 254 | 84 | 255 | 84 | 256 | 84 | 257 | 84 | 258 |
| 84 | 259 | 84 | 260 | 84 | 450 | 84 | 451 | 84 | 452 | 84 | 453 |
| 84 | 454 | 84 | 455 | 84 | 456 | 84 | 261 | 84 | 262 | 84 | 263 |
| 84 | 264 | 84 | 265 | 84 | 266 | 84 | 267 | 84 | 268 | 84 | 463 |
| 84 | 457 | 84 | 458 | 84 | 459 | 84 | 460 | 84 | 461 | 84 | 462 |
| 156 | 253 | 156 | 254 | 156 | 255 | 156 | 256 | 156 | 257 | 156 | 258 |
| 156 | 259 | 156 | 260 | 156 | 450 | 156 | 451 | 156 | 452 | 156 | 453 |
| 156 | 454 | 156 | 455 | 156 | 456 | 156 | 261 | 156 | 262 | 156 | 263 |
| 156 | 264 | 156 | 265 | 156 | 266 | 156 | 267 | 156 | 268 | 156 | 463 |
| 156 | 457 | 156 | 458 | 156 | 459 | 156 | 460 | 156 | 461 | 156 | 462 |
| 157 | 253 | 157 | 254 | 157 | 255 | 157 | 256 | 157 | 257 | 157 | 258 |
| 157 | 259 | 157 | 260 | 157 | 450 | 157 | 451 | 157 | 452 | 157 | 453 |
| 157 | 454 | 157 | 455 | 157 | 456 | 157 | 261 | 157 | 262 | 157 | 263 |
| 157 | 264 | 157 | 265 | 157 | 266 | 157 | 267 | 157 | 268 | 157 | 463 |
| 157 | 457 | 157 | 458 | 157 | 459 | 157 | 460 | 157 | 461 | 157 | 462 |
| 158 | 253 | 158 | 254 | 158 | 255 | 158 | 256 | 158 | 257 | 158 | 258 |
| 158 | 259 | 158 | 260 | 158 | 450 | 158 | 451 | 158 | 452 | 158 | 453 |
| 158 | 454 | 158 | 455 | 158 | 456 | 158 | 261 | 158 | 262 | 158 | 263 |
| 158 | 264 | 158 | 265 | 158 | 266 | 158 | 267 | 158 | 268 | 158 | 463 |
| 158 | 457 | 158 | 458 | 158 | 459 | 158 | 460 | 158 | 461 | 158 | 462 |
| 159 | 253 | 159 | 254 | 159 | 255 | 159 | 256 | 159 | 257 | 159 | 258 |
| 159 | 259 | 159 | 260 | 159 | 450 | 159 | 451 | 159 | 452 | 159 | 453 |
| 159 | 454 | 159 | 455 | 159 | 456 | 159 | 261 | 159 | 262 | 159 | 263 |
| 159 | 264 | 159 | 265 | 159 | 266 | 159 | 267 | 159 | 268 | 159 | 463 |
| 159 | 457 | 159 | 458 | 159 | 459 | 159 | 460 | 159 | 461 | 159 | 462 |
| 160 | 253 | 160 | 254 | 160 | 255 | 160 | 256 | 160 | 257 | 160 | 258 |
| 160 | 259 | 160 | 260 | 160 | 450 | 160 | 451 | 160 | 452 | 160 | 453 |
| 160 | 454 | 160 | 455 | 160 | 456 | 160 | 261 | 160 | 262 | 160 | 263 |
| 160 | 264 | 160 | 265 | 160 | 266 | 160 | 267 | 160 | 268 | 160 | 463 |
| 160 | 457 | 160 | 458 | 160 | 459 | 160 | 460 | 160 | 461 | 160 | 462 |
| 161 | 253 | 161 | 254 | 161 | 255 | 161 | 256 | 161 | 257 | 161 | 258 |
| 161 | 259 | 161 | 260 | 161 | 450 | 161 | 451 | 161 | 452 | 161 | 453 |
| 161 | 454 | 161 | 455 | 161 | 456 | 161 | 261 | 161 | 262 | 161 | 263 |
| 161 | 264 | 161 | 265 | 161 | 266 | 161 | 267 | 161 | 268 | 161 | 463 |
| 161 | 457 | 161 | 458 | 161 | 459 | 161 | 460 | 161 | 461 | 161 | 462 |
| 162 | 253 | 162 | 254 | 162 | 255 | 162 | 256 | 162 | 257 | 162 | 258 |
| 162 | 259 | 162 | 260 | 162 | 450 | 162 | 451 | 162 | 452 | 162 | 453 |
| 162 | 454 | 162 | 455 | 162 | 456 | 162 | 261 | 162 | 262 | 162 | 263 |
| 162 | 264 | 162 | 265 | 162 | 266 | 162 | 267 | 162 | 268 | 162 | 463 |
| 162 | 457 | 162 | 458 | 162 | 459 | 162 | 460 | 162 | 461 | 162 | 462 |
| 163 | 253 | 163 | 254 | 163 | 255 | 163 | 256 | 163 | 257 | 163 | 258 |
| 163 | 259 | 163 | 260 | 163 | 450 | 163 | 451 | 163 | 452 | 163 | 453 |
| 163 | 454 | 163 | 455 | 163 | 456 | 163 | 261 | 163 | 262 | 163 | 263 |
| 163 | 264 | 163 | 265 | 163 | 266 | 163 | 267 | 163 | 268 | 163 | 463 |
| 163 | 457 | 163 | 458 | 163 | 459 | 163 | 460 | 163 | 461 | 163 | 462 |
| 164 | 253 | 164 | 254 | 164 | 255 | 164 | 256 | 164 | 257 | 164 | 258 |
| 164 | 259 | 164 | 260 | 164 | 450 | 164 | 451 | 164 | 452 | 164 | 453 |
| 164 | 454 | 164 | 455 | 164 | 456 | 164 | 261 | 164 | 262 | 164 | 263 |
| 164 | 264 | 164 | 265 | 164 | 266 | 164 | 267 | 164 | 268 | 164 | 463 |
| 164 | 457 | 164 | 458 | 164 | 459 | 164 | 460 | 164 | 461 | 164 | 462 |
| 165 | 253 | 165 | 254 | 165 | 255 | 165 | 256 | 165 | 257 | 165 | 258 |
| 165 | 259 | 165 | 260 | 165 | 450 | 165 | 451 | 165 | 452 | 165 | 453 |
| 165 | 454 | 165 | 453 | 165 | 456 | 165 | 261 | 165 | 262 | 165 | 263 |
| 165 | 264 | 165 | 265 | 165 | 266 | 165 | 267 | 165 | 268 | 165 | 463 |
| 165 | 457 | 165 | 458 | 165 | 459 | 165 | 460 | 165 | 461 | 165 | 462 |
| 166 | 253 | 166 | 254 | 166 | 255 | 166 | 256 | 166 | 257 | 166 | 258 |
| 166 | 259 | 166 | 260 | 166 | 450 | 166 | 451 | 166 | 452 | 166 | 453 |
| 166 | 454 | 166 | 455 | 166 | 456 | 166 | 261 | 166 | 262 | 166 | 263 |
| 166 | 264 | 166 | 265 | 166 | 266 | 166 | 267 | 166 | 268 | 166 | 463 |
| 166 | 457 | 166 | 458 | 166 | 459 | 166 | 460 | 166 | 461 | 166 | 462 |
| 167 | 253 | 167 | 254 | 167 | 255 | 167 | 256 | 167 | 257 | 167 | 258 |
| 167 | 259 | 167 | 260 | 167 | 450 | 167 | 451 | 167 | 452 | 167 | 453 |
| 167 | 454 | 167 | 455 | 167 | 456 | 167 | 261 | 167 | 262 | 167 | 263 |
| 167 | 264 | 167 | 265 | 167 | 266 | 167 | 267 | 167 | 268 | 167 | 463 |
| 167 | 457 | 167 | 458 | 167 | 459 | 167 | 460 | 167 | 461 | 167 | 462 |
| 168 | 253 | 168 | 254 | 168 | 255 | 168 | 256 | 168 | 257 | 168 | 258 |
| 168 | 259 | 168 | 260 | 168 | 450 | 168 | 451 | 168 | 452 | 168 | 453 |
| 168 | 454 | 168 | 455 | 168 | 456 | 168 | 261 | 168 | 262 | 168 | 263 |
| 168 | 264 | 168 | 265 | 168 | 266 | 168 | 267 | 168 | 268 | 168 | 463 |
| 168 | 457 | 168 | 458 | 168 | 459 | 168 | 460 | 168 | 461 | 168 | 462 |
| 169 | 253 | 169 | 254 | 169 | 255 | 169 | 256 | 169 | 257 | 169 | 258 |
| 169 | 259 | 169 | 260 | 169 | 450 | 169 | 451 | 169 | 452 | 169 | 453 |
| 169 | 454 | 169 | 455 | 169 | 456 | 169 | 261 | 169 | 262 | 169 | 263 |
| 169 | 264 | 169 | 265 | 169 | 266 | 169 | 267 | 169 | 268 | 169 | 463 |
| 169 | 457 | 169 | 458 | 169 | 459 | 169 | 460 | 169 | 461 | 169 | 462 |
| 170 | 253 | 170 | 254 | 170 | 255 | 170 | 256 | 170 | 257 | 170 | 258 |
| 170 | 259 | 170 | 260 | 170 | 450 | 170 | 451 | 170 | 452 | 170 | 453 |
| 170 | 454 | 170 | 455 | 170 | 456 | 170 | 261 | 170 | 262 | 170 | 263 |
| 170 | 264 | 170 | 265 | 170 | 266 | 170 | 267 | 170 | 268 | 170 | 463 |
| 170 | 457 | 170 | 458 | 170 | 459 | 170 | 460 | 170 | 461 | 170 | 462 |
| 171 | 253 | 171 | 254 | 171 | 255 | 171 | 256 | 171 | 257 | 171 | 258 |
| 171 | 259 | 171 | 260 | 171 | 450 | 171 | 451 | 171 | 452 | 171 | 453 |
| 171 | 454 | 171 | 455 | 171 | 456 | 171 | 261 | 171 | 262 | 171 | 263 |
| 171 | 264 | 171 | 265 | 171 | 266 | 171 | 267 | 171 | 268 | 171 | 463 |
| 171 | 457 | 171 | 458 | 171 | 459 | 171 | 460 | 171 | 461 | 171 | 462 |
| 172 | 253 | 172 | 254 | 172 | 255 | 172 | 256 | 172 | 257 | 172 | 258 |
| 172 | 259 | 172 | 260 | 172 | 450 | 172 | 451 | 172 | 452 | 172 | 453 |
| 172 | 454 | 172 | 455 | 172 | 456 | 172 | 261 | 172 | 262 | 172 | 263 |
| 172 | 264 | 172 | 265 | 172 | 266 | 172 | 267 | 172 | 268 | 172 | 463 |
| 172 | 457 | 172 | 458 | 172 | 459 | 172 | 460 | 172 | 461 | 172 | 462 |
| 173 | 253 | 173 | 254 | 173 | 255 | 173 | 256 | 173 | 257 | 173 | 258 |
| 173 | 259 | 173 | 260 | 173 | 450 | 173 | 451 | 173 | 452 | 173 | 453 |
| 173 | 454 | 173 | 455 | 173 | 456 | 173 | 261 | 173 | 262 | 173 | 263 |
| 173 | 264 | 173 | 265 | 173 | 266 | 173 | 267 | 173 | 268 | 173 | 463 |
| 173 | 457 | 173 | 458 | 173 | 459 | 173 | 460 | 173 | 461 | 173 | 462 |
| 174 | 253 | 174 | 254 | 174 | 255 | 174 | 256 | 174 | 257 | 174 | 258 |
| 174 | 259 | 174 | 260 | 174 | 450 | 174 | 451 | 174 | 452 | 174 | 453 |
| 174 | 454 | 174 | 455 | 174 | 456 | 174 | 261 | 174 | 262 | 174 | 263 |
| 174 | 264 | 174 | 265 | 174 | 266 | 174 | 267 | 174 | 268 | 174 | 463 |
| 174 | 457 | 174 | 458 | 174 | 459 | 174 | 460 | 174 | 461 | 174 | 462 |
| 175 | 253 | 175 | 254 | 175 | 255 | 175 | 256 | 175 | 257 | 175 | 258 |
| 175 | 259 | 175 | 260 | 175 | 450 | 175 | 451 | 175 | 452 | 175 | 453 |
| 175 | 454 | 175 | 455 | 175 | 456 | 175 | 261 | 175 | 262 | 175 | 263 |
| 175 | 264 | 175 | 265 | 175 | 266 | 175 | 267 | 175 | 268 | 175 | 463 |
| 175 | 457 | 175 | 458 | 175 | 459 | 175 | 460 | 175 | 461 | 175 | 462 |
| 176 | 253 | 176 | 254 | 176 | 255 | 176 | 256 | 176 | 257 | 176 | 258 |
| 176 | 259 | 176 | 260 | 176 | 450 | 176 | 451 | 176 | 452 | 176 | 453 |
| 176 | 454 | 176 | 455 | 176 | 456 | 176 | 261 | 176 | 262 | 176 | 263 |
| 176 | 264 | 176 | 265 | 176 | 266 | 176 | 267 | 176 | 268 | 176 | 463 |
| 176 | 457 | 176 | 458 | 176 | 459 | 176 | 460 | 176 | 461 | 176 | 462 |
| 177 | 253 | 177 | 254 | 177 | 255 | 177 | 256 | 177 | 257 | 177 | 258 |
| 177 | 259 | 177 | 260 | 177 | 450 | 177 | 451 | 177 | 452 | 177 | 453 |
| 177 | 454 | 177 | 455 | 177 | 456 | 177 | 261 | 177 | 262 | 177 | 263 |
| 177 | 264 | 177 | 265 | 177 | 266 | 177 | 267 | 177 | 268 | 177 | 463 |
| 177 | 457 | 177 | 458 | 177 | 459 | 177 | 460 | 177 | 461 | 177 | 462 |
| 178 | 253 | 178 | 254 | 178 | 255 | 178 | 256 | 178 | 257 | 178 | 258 |
| 178 | 259 | 178 | 260 | 178 | 450 | 178 | 451 | 178 | 452 | 178 | 453 |
| 178 | 454 | 178 | 455 | 178 | 456 | 178 | 261 | 178 | 262 | 178 | 263 |
| 178 | 264 | 178 | 265 | 178 | 266 | 178 | 267 | 178 | 268 | 178 | 463 |
| 178 | 457 | 178 | 458 | 178 | 459 | 178 | 460 | 178 | 461 | 178 | 462 |
| 179 | 253 | 179 | 254 | 179 | 255 | 179 | 256 | 179 | 257 | 179 | 258 |
| 179 | 259 | 179 | 260 | 179 | 450 | 179 | 451 | 179 | 452 | 179 | 453 |
| 179 | 454 | 179 | 455 | 179 | 456 | 179 | 261 | 179 | 262 | 179 | 263 |
| 179 | 264 | 179 | 265 | 179 | 266 | 179 | 267 | 179 | 268 | 179 | 463 |
| 179 | 457 | 179 | 458 | 179 | 459 | 179 | 460 | 179 | 461 | 179 | 462 |
| 180 | 253 | 180 | 254 | 180 | 255 | 180 | 256 | 180 | 257 | 180 | 258 |
| 180 | 259 | 180 | 260 | 180 | 450 | 180 | 451 | 180 | 452 | 180 | 453 |
| 180 | 454 | 180 | 455 | 180 | 456 | 180 | 261 | 180 | 262 | 180 | 263 |
| 180 | 264 | 180 | 265 | 180 | 266 | 180 | 267 | 180 | 268 | 180 | 463 |
| 180 | 457 | 180 | 458 | 180 | 459 | 180 | 460 | 180 | 461 | 180 | 462 |
| 181 | 253 | 181 | 254 | 181 | 255 | 181 | 256 | 181 | 257 | 181 | 258 |
| 181 | 259 | 181 | 260 | 181 | 450 | 181 | 451 | 181 | 452 | 181 | 453 |
| 181 | 454 | 181 | 455 | 181 | 456 | 181 | 261 | 181 | 262 | 181 | 263 |
| 181 | 264 | 181 | 265 | 181 | 266 | 181 | 267 | 181 | 268 | 181 | 463 |
| 181 | 457 | 181 | 458 | 181 | 459 | 181 | 460 | 181 | 461 | 181 | 462 |
| 182 | 253 | 182 | 254 | 182 | 255 | 182 | 256 | 182 | 257 | 182 | 258 |
| 182 | 259 | 182 | 260 | 182 | 450 | 182 | 451 | 182 | 452 | 182 | 453 |
| 182 | 454 | 182 | 455 | 182 | 456 | 182 | 261 | 182 | 262 | 182 | 263 |
| 182 | 264 | 182 | 265 | 182 | 266 | 182 | 267 | 182 | 268 | 182 | 463 |
| 182 | 457 | 182 | 458 | 182 | 459 | 182 | 460 | 182 | 461 | 182 | 462 |
| 183 | 253 | 183 | 254 | 183 | 255 | 183 | 256 | 183 | 257 | 183 | 258 |
| 183 | 259 | 183 | 260 | 183 | 450 | 183 | 451 | 183 | 452 | 183 | 453 |
| 183 | 454 | 183 | 455 | 183 | 456 | 183 | 261 | 183 | 262 | 183 | 263 |
| 183 | 264 | 183 | 265 | 183 | 266 | 183 | 267 | 183 | 268 | 183 | 463 |
| 183 | 457 | 183 | 458 | 183 | 459 | 183 | 460 | 183 | 461 | 183 | 462 |
| 184 | 253 | 184 | 254 | 184 | 255 | 184 | 256 | 184 | 257 | 184 | 258 |
| 184 | 259 | 184 | 260 | 184 | 450 | 184 | 451 | 184 | 452 | 184 | 453 |
| 184 | 454 | 184 | 455 | 184 | 456 | 184 | 261 | 184 | 262 | 184 | 263 |
| 184 | 264 | 184 | 265 | 184 | 266 | 184 | 267 | 184 | 268 | 184 | 463 |
| 184 | 457 | 184 | 458 | 184 | 459 | 184 | 460 | 184 | 461 | 184 | 462 |
| 185 | 253 | 185 | 254 | 185 | 255 | 185 | 256 | 185 | 257 | 185 | 258 |
| 185 | 259 | 185 | 260 | 185 | 450 | 185 | 451 | 185 | 452 | 185 | 453 |
| 185 | 454 | 185 | 455 | 185 | 456 | 185 | 261 | 185 | 262 | 185 | 263 |
| 185 | 264 | 185 | 265 | 185 | 266 | 185 | 267 | 185 | 268 | 185 | 463 |
| 185 | 457 | 185 | 458 | 185 | 459 | 185 | 460 | 185 | 461 | 185 | 462 |
| 186 | 253 | 186 | 254 | 186 | 255 | 186 | 256 | 186 | 257 | 186 | 258 |
| 186 | 259 | 186 | 260 | 186 | 450 | 186 | 451 | 186 | 452 | 186 | 453 |
| 186 | 454 | 186 | 455 | 186 | 456 | 186 | 261 | 186 | 262 | 186 | 263 |
| 186 | 264 | 186 | 265 | 186 | 266 | 186 | 267 | 186 | 268 | 186 | 463 |
| 186 | 457 | 186 | 458 | 186 | 459 | 186 | 460 | 186 | 461 | 186 | 462 |
| 187 | 253 | 187 | 254 | 187 | 255 | 187 | 256 | 187 | 257 | 187 | 258 |
| 187 | 259 | 187 | 260 | 187 | 450 | 187 | 451 | 187 | 452 | 187 | 453 |
| 187 | 454 | 187 | 455 | 187 | 456 | 187 | 261 | 187 | 262 | 187 | 263 |
| 187 | 264 | 187 | 265 | 187 | 266 | 187 | 267 | 187 | 268 | 187 | 463 |
| 187 | 457 | 187 | 458 | 187 | 459 | 187 | 460 | 187 | 461 | 187 | 462 |
| 188 | 253 | 188 | 254 | 188 | 255 | 188 | 256 | 188 | 257 | 188 | 258 |
| 188 | 259 | 188 | 260 | 188 | 450 | 188 | 451 | 188 | 452 | 188 | 453 |
| 188 | 454 | 188 | 455 | 188 | 456 | 188 | 261 | 188 | 262 | 188 | 263 |
| 188 | 264 | 188 | 265 | 188 | 266 | 188 | 267 | 188 | 268 | 188 | 463 |
| 188 | 457 | 188 | 458 | 188 | 459 | 188 | 460 | 188 | 461 | 188 | 462 |
| 189 | 253 | 189 | 254 | 189 | 255 | 189 | 256 | 189 | 257 | 189 | 258 |
| 189 | 259 | 189 | 260 | 189 | 450 | 189 | 451 | 189 | 452 | 189 | 453 |
| 189 | 454 | 189 | 455 | 189 | 456 | 189 | 261 | 189 | 262 | 189 | 263 |
| 189 | 264 | 189 | 265 | 189 | 266 | 189 | 267 | 189 | 268 | 189 | 463 |
| 189 | 457 | 189 | 458 | 189 | 459 | 189 | 460 | 189 | 461 | 189 | 462 |
| 190 | 253 | 190 | 254 | 190 | 255 | 190 | 256 | 190 | 257 | 190 | 258 |
| 190 | 259 | 190 | 260 | 190 | 450 | 190 | 451 | 190 | 452 | 190 | 453 |
| 190 | 454 | 190 | 455 | 190 | 456 | 190 | 261 | 190 | 262 | 190 | 263 |
| 190 | 264 | 190 | 265 | 190 | 266 | 190 | 267 | 190 | 268 | 190 | 463 |
| 190 | 457 | 190 | 458 | 190 | 459 | 190 | 460 | 190 | 461 | 190 | 462 |
| 191 | 253 | 191 | 254 | 191 | 255 | 191 | 256 | 191 | 257 | 191 | 258 |
| 191 | 259 | 191 | 260 | 191 | 450 | 191 | 451 | 191 | 452 | 191 | 453 |
| 191 | 454 | 191 | 455 | 191 | 456 | 191 | 261 | 191 | 262 | 191 | 263 |
| 191 | 264 | 191 | 265 | 191 | 266 | 191 | 267 | 191 | 268 | 191 | 463 |
| 191 | 457 | 191 | 458 | 191 | 459 | 191 | 460 | 191 | 461 | 191 | 462 |

In certain embodiments, the instant disclosure provides an antibody that specifically binds to APJ (e.g., human APJ), the antibody comprising any one of the VH-Ig Fc amino acid sequences shown in Table 15, wherein the Ig Fc has a modified hinge region comprising the paired modified hinge amino acid sequence shown in Table 15. In the embodiments shown in Table 15. the indicated modified hinge amino acid sequences are inserted at the region spanning EU positions 216-230 in the paired VH-Ig Fc amino acid sequence.

TABLE 15
VH-Ig Fc hinge modification combinations.

| VH-Ig Fc with | | VH-Ig Fc with | | VH-Ig Fc with | | VH-Ig Fc with | |
|---|---|---|---|---|---|---|---|
| modified hinge region | | modified hinge region | | modified hinge region | | modified hinge region | |
| VH-Ig | Modified | VH-Ig | Modified | VH-Ig | Modified | VH-Ig | Modified |
| Fc | hinge | Fc | hinge | Fc | hinge | Fc | hinge |
| SEQ ID NO | | SEQ ID NO | | SEQ ID NO | | SEQ ID NO | |
| 270 | 704 | 270 | 705 | 270 | Del3* | 270 | 706 |
| 270 | 707 | 270 | 708 | 270 | 709 | 270 | 710 |
| 270 | 711 | 270 | 712 | 270 | 713 | 270 | 714 |
| 270 | 715 | 270 | 716 | 270 | 717 | 270 | 718 |
| 271 | 704 | 271 | 705 | 271 | Del3* | 271 | 706 |
| 271 | 707 | 271 | 708 | 271 | 709 | 271 | 710 |
| 271 | 711 | 271 | 712 | 271 | 713 | 271 | 714 |
| 271 | 715 | 271 | 716 | 271 | 717 | 271 | 718 |
| 272 | 704 | 272 | 705 | 272 | Del3* | 272 | 706 |
| 272 | 707 | 272 | 708 | 272 | 709 | 272 | 710 |
| 272 | 711 | 272 | 712 | 272 | 713 | 272 | 714 |
| 272 | 715 | 272 | 716 | 272 | 717 | 272 | 718 |
| 273 | 704 | 273 | 705 | 273 | Del3* | 273 | 706 |
| 273 | 707 | 273 | 708 | 273 | 709 | 273 | 710 |
| 273 | 711 | 273 | 712 | 273 | 713 | 273 | 714 |
| 273 | 715 | 273 | 716 | 273 | 717 | 273 | 718 |
| 274 | 704 | 274 | 705 | 274 | Del3* | 274 | 706 |
| 274 | 707 | 274 | 708 | 274 | 709 | 274 | 710 |
| 274 | 711 | 274 | 712 | 274 | 713 | 274 | 714 |
| 274 | 715 | 274 | 716 | 274 | 717 | 274 | 718 |
| 275 | 704 | 275 | 705 | 275 | Del3* | 275 | 706 |
| 275 | 707 | 275 | 708 | 275 | 709 | 275 | 710 |
| 275 | 711 | 275 | 712 | 275 | 713 | 275 | 714 |
| 275 | 715 | 275 | 716 | 275 | 717 | 275 | 718 |
| 276 | 704 | 276 | 705 | 276 | Del3* | 276 | 706 |
| 276 | 707 | 276 | 708 | 276 | 709 | 276 | 710 |
| 276 | 711 | 276 | 712 | 276 | 713 | 276 | 714 |
| 276 | 715 | 276 | 716 | 276 | 717 | 276 | 718 |
| 277 | 704 | 277 | 705 | 277 | Del3* | 277 | 706 |
| 277 | 707 | 277 | 708 | 277 | 709 | 277 | 710 |
| 277 | 711 | 277 | 712 | 277 | 713 | 277 | 714 |
| 277 | 715 | 277 | 716 | 277 | 717 | 277 | 718 |
| 278 | 704 | 278 | 705 | 278 | Del3* | 278 | 706 |
| 278 | 707 | 278 | 708 | 278 | 709 | 278 | 710 |
| 278 | 711 | 278 | 712 | 278 | 713 | 278 | 714 |
| 278 | 715 | 278 | 716 | 278 | 717 | 278 | 718 |
| 279 | 704 | 279 | 705 | 279 | Del3* | 279 | 706 |
| 279 | 707 | 279 | 708 | 279 | 709 | 279 | 710 |
| 279 | 711 | 279 | 712 | 279 | 713 | 279 | 714 |
| 279 | 715 | 279 | 716 | 279 | 717 | 279 | 718 |
| 280 | 704 | 280 | 705 | 280 | Del3* | 280 | 706 |
| 280 | 707 | 280 | 708 | 280 | 709 | 280 | 710 |
| 280 | 711 | 280 | 712 | 280 | 713 | 280 | 714 |
| 280 | 715 | 280 | 716 | 280 | 717 | 280 | 718 |
| 281 | 704 | 281 | 705 | 281 | Del3* | 281 | 706 |
| 281 | 707 | 281 | 708 | 281 | 709 | 281 | 710 |
| 281 | 711 | 281 | 712 | 281 | 713 | 281 | 714 |
| 281 | 715 | 281 | 716 | 281 | 717 | 281 | 718 |
| 282 | 704 | 282 | 705 | 282 | Del3* | 282 | 706 |
| 282 | 707 | 282 | 708 | 282 | 709 | 282 | 710 |
| 282 | 711 | 282 | 712 | 282 | 713 | 282 | 714 |
| 282 | 715 | 282 | 716 | 282 | 717 | 282 | 718 |
| 283 | 704 | 283 | 705 | 283 | Del3* | 283 | 706 |
| 283 | 707 | 283 | 708 | 283 | 709 | 283 | 710 |
| 283 | 711 | 283 | 712 | 283 | 713 | 283 | 714 |
| 283 | 715 | 283 | 716 | 283 | 717 | 283 | 718 |
| 284 | 704 | 284 | 705 | 284 | Del3* | 284 | 706 |
| 284 | 707 | 284 | 708 | 284 | 709 | 284 | 710 |
| 284 | 711 | 284 | 712 | 284 | 713 | 284 | 714 |
| 284 | 715 | 284 | 716 | 284 | 717 | 284 | 718 |
| 285 | 704 | 285 | 705 | 285 | Del3* | 285 | 706 |
| 285 | 707 | 285 | 708 | 285 | 709 | 285 | 710 |
| 285 | 711 | 285 | 712 | 285 | 713 | 285 | 714 |
| 285 | 715 | 285 | 716 | 285 | 717 | 285 | 718 |
| 286 | 704 | 286 | 705 | 286 | Del3* | 286 | 706 |
| 286 | 707 | 286 | 708 | 286 | 709 | 286 | 710 |
| 286 | 711 | 286 | 712 | 286 | 713 | 286 | 714 |
| 286 | 715 | 286 | 716 | 286 | 717 | 286 | 718 |
| 287 | 704 | 287 | 705 | 287 | Del3* | 287 | 706 |
| 287 | 707 | 287 | 708 | 287 | 709 | 287 | 710 |
| 287 | 711 | 287 | 712 | 287 | 713 | 287 | 714 |
| 287 | 715 | 287 | 716 | 287 | 717 | 287 | 718 |
| 288 | 704 | 288 | 705 | 288 | Del3* | 288 | 706 |
| 288 | 707 | 288 | 708 | 288 | 709 | 288 | 710 |
| 288 | 711 | 288 | 712 | 288 | 713 | 288 | 714 |
| 288 | 715 | 288 | 716 | 288 | 717 | 288 | 718 |
| 289 | 704 | 289 | 705 | 289 | Del3* | 289 | 706 |
| 289 | 707 | 289 | 708 | 289 | 709 | 289 | 710 |
| 289 | 711 | 289 | 712 | 289 | 713 | 289 | 714 |
| 289 | 715 | 289 | 716 | 289 | 717 | 289 | 718 |
| 290 | 704 | 290 | 705 | 290 | Del3* | 290 | 706 |
| 290 | 707 | 290 | 708 | 290 | 709 | 290 | 710 |
| 290 | 711 | 290 | 712 | 290 | 713 | 290 | 714 |
| 290 | 715 | 290 | 716 | 290 | 717 | 290 | 718 |
| 291 | 704 | 291 | 705 | 291 | Del3* | 291 | 706 |
| 291 | 707 | 291 | 708 | 291 | 709 | 291 | 710 |
| 291 | 711 | 291 | 712 | 291 | 713 | 291 | 714 |
| 291 | 715 | 291 | 716 | 291 | 717 | 291 | 718 |
| 292 | 704 | 292 | 705 | 292 | Del3* | 292 | 706 |
| 292 | 707 | 292 | 708 | 292 | 709 | 292 | 710 |
| 292 | 711 | 292 | 712 | 292 | 713 | 292 | 714 |
| 292 | 715 | 292 | 716 | 292 | 717 | 292 | 718 |
| 293 | 704 | 293 | 705 | 293 | Del3* | 293 | 706 |
| 293 | 707 | 293 | 708 | 293 | 709 | 293 | 710 |
| 293 | 711 | 293 | 712 | 293 | 713 | 293 | 714 |
| 293 | 715 | 293 | 716 | 293 | 717 | 293 | 718 |
| 294 | 704 | 294 | 705 | 294 | Del3* | 294 | 706 |
| 294 | 707 | 294 | 708 | 294 | 709 | 294 | 710 |
| 294 | 711 | 294 | 712 | 294 | 713 | 294 | 714 |
| 294 | 715 | 294 | 716 | 294 | 717 | 294 | 718 |
| 295 | 704 | 295 | 705 | 295 | Del3* | 295 | 706 |
| 295 | 707 | 295 | 708 | 29 | 709 | 295 | 710 |
| 295 | 711 | 295 | 712 | 295 | 713 | 295 | 714 |
| 295 | 715 | 295 | 716 | 295 | 717 | 295 | 718 |
| 296 | 704 | 296 | 705 | 296 | Del3* | 296 | 706 |
| 296 | 707 | 296 | 708 | 296 | 709 | 296 | 710 |
| 296 | 711 | 296 | 712 | 296 | 713 | 296 | 714 |
| 296 | 715 | 296 | 716 | 296 | 717 | 296 | 718 |
| 297 | 704 | 297 | 705 | 297 | Del3* | 297 | 706 |
| 297 | 707 | 297 | 708 | 297 | 709 | 297 | 710 |
| 297 | 711 | 297 | 712 | 297 | 713 | 297 | 714 |
| 297 | 715 | 297 | 716 | 297 | 717 | 297 | 718 |
| 298 | 704 | 298 | 705 | 298 | Del3* | 298 | 706 |
| 298 | 707 | 298 | 708 | 298 | 709 | 298 | 710 |
| 298 | 711 | 298 | 712 | 298 | 713 | 29 | 714 |
| 298 | 715 | 298 | 716 | 298 | 717 | 298 | 718 |
| 299 | 704 | 299 | 705 | 299 | Del3* | 299 | 706 |
| 299 | 707 | 299 | 708 | 299 | 709 | 299 | 710 |
| 299 | 711 | 299 | 712 | 299 | 713 | 299 | 714 |
| 299 | 715 | 299 | 716 | 299 | 717 | 299 | 718 |
| 300 | 704 | 300 | 705 | 300 | Del3* | 300 | 706 |
| 300 | 707 | 300 | 708 | 300 | 709 | 300 | 710 |
| 300 | 711 | 300 | 712 | 300 | 713 | 300 | 714 |
| 300 | 715 | 300 | 716 | 300 | 717 | 300 | 718 |
| 301 | 704 | 301 | 705 | 301 | Del3* | 301 | 706 |
| 301 | 707 | 301 | 708 | 301 | 709 | 301 | 710 |
| 301 | 711 | 301 | 712 | 301 | 713 | 301 | 714 |
| 301 | 715 | 301 | 716 | 301 | 717 | 301 | 718 |
| 302 | 704 | 302 | 705 | 302 | Del3* | 302 | 706 |
| 302 | 707 | 302 | 708 | 302 | 709 | 302 | 710 |
| 302 | 711 | 302 | 712 | 302 | 713 | 302 | 714 |
| 302 | 715 | 302 | 716 | 302 | 717 | 302 | 718 |
| 303 | 704 | 303 | 705 | 303 | Del3* | 303 | 706 |
| 303 | 707 | 303 | 708 | 303 | 709 | 303 | 710 |
| 303 | 711 | 303 | 712 | 303 | 713 | 303 | 714 |
| 303 | 715 | 303 | 716 | 303 | 717 | 303 | 718 |
| 304 | 704 | 304 | 705 | 304 | Del3* | 304 | 706 |
| 304 | 707 | 304 | 708 | 304 | 709 | 304 | 710 |
| 304 | 711 | 304 | 712 | 304 | 713 | 304 | 714 |
| 304 | 715 | 304 | 716 | 304 | 717 | 304 | 718 |
| 305 | 704 | 305 | 705 | 305 | Del3* | 305 | 706 |
| 305 | 707 | 305 | 708 | 305 | 709 | 305 | 710 |
| 305 | 711 | 305 | 712 | 305 | 713 | 305 | 714 |
| 305 | 715 | 305 | 716 | 305 | 717 | 305 | 718 |
| 306 | 704 | 306 | 705 | 306 | Del3* | 306 | 706 |
| 306 | 707 | 306 | 708 | 306 | 709 | 306 | 710 |
| 306 | 711 | 306 | 712 | 306 | 713 | 306 | 714 |
| 306 | 715 | 306 | 716 | 306 | 717 | 306 | 718 |
| 307 | 704 | 307 | 705 | 307 | Del3* | 307 | 706 |
| 307 | 707 | 307 | 708 | 307 | 709 | 307 | 710 |
| 307 | 711 | 307 | 712 | 307 | 713 | 307 | 714 |
| 307 | 715 | 307 | 716 | 307 | 717 | 307 | 718 |
| 308 | 704 | 308 | 705 | 308 | Del3* | 308 | 706 |
| 308 | 707 | 308 | 708 | 308 | 709 | 308 | 710 |
| 308 | 711 | 308 | 712 | 308 | 713 | 308 | 714 |
| 308 | 715 | 308 | 716 | 308 | 717 | 308 | 718 |
| 309 | 704 | 309 | 705 | 309 | Del3* | 309 | 706 |
| 309 | 707 | 309 | 708 | 309 | 709 | 309 | 710 |
| 309 | 711 | 309 | 712 | 309 | 713 | 309 | 714 |
| 309 | 715 | 309 | 716 | 309 | 717 | 309 | 718 |
| 310 | 704 | 310 | 705 | 310 | Del3* | 310 | 706 |
| 310 | 707 | 310 | 708 | 310 | 709 | 310 | 710 |
| 310 | 711 | 310 | 712 | 310 | 713 | 310 | 714 |
| 310 | 715 | 310 | 716 | 310 | 717 | 310 | 718 |
| 311 | 704 | 311 | 705 | 311 | Del3* | 311 | 706 |
| 311 | 707 | 311 | 708 | 311 | 709 | 311 | 710 |
| 311 | 711 | 311 | 712 | 311 | 713 | 311 | 714 |
| 311 | 715 | 311 | 716 | 311 | 717 | 311 | 718 |
| 312 | 704 | 312 | 705 | 312 | Del3* | 312 | 706 |
| 312 | 707 | 312 | 708 | 312 | 709 | 312 | 710 |
| 312 | 711 | 312 | 712 | 312 | 713 | 312 | 714 |
| 312 | 715 | 312 | 716 | 312 | 717 | 312 | 718 |
| 313 | 704 | 313 | 705 | 313 | Del3* | 313 | 706 |
| 313 | 707 | 313 | 708 | 313 | 709 | 313 | 710 |
| 313 | 711 | 313 | 712 | 313 | 713 | 313 | 714 |
| 313 | 715 | 313 | 716 | 313 | 717 | 313 | 718 |
| 314 | 704 | 314 | 705 | 314 | Del3* | 314 | 706 |
| 314 | 707 | 314 | 708 | 314 | 709 | 314 | 710 |
| 314 | 711 | 314 | 712 | 314 | 713 | 314 | 714 |
| 314 | 715 | 314 | 716 | 314 | 717 | 314 | 718 |
| 315 | 704 | 315 | 705 | 315 | Del3* | 315 | 706 |
| 315 | 707 | 315 | 708 | 315 | 709 | 315 | 710 |
| 315 | 711 | 315 | 712 | 315 | 713 | 315 | 714 |
| 315 | 715 | 315 | 716 | 315 | 717 | 315 | 718 |
| 316 | 704 | 316 | 705 | 316 | Del3* | 316 | 706 |
| 316 | 707 | 316 | 708 | 316 | 709 | 316 | 710 |
| 316 | 711 | 316 | 712 | 316 | 713 | 316 | 714 |
| 316 | 715 | 316 | 716 | 316 | 717 | 316 | 718 |
| 317 | 704 | 317 | 705 | 317 | Del3* | 317 | 706 |
| 317 | 707 | 317 | 708 | 317 | 709 | 317 | 710 |
| 317 | 711 | 317 | 712 | 317 | 713 | 317 | 714 |
| 317 | 715 | 317 | 716 | 317 | 717 | 317 | 718 |
| 318 | 704 | 318 | 705 | 318 | Del3* | 318 | 706 |
| 318 | 707 | 318 | 708 | 318 | 709 | 318 | 710 |
| 318 | 711 | 318 | 712 | 318 | 713 | 318 | 714 |
| 318 | 715 | 318 | 716 | 318 | 717 | 318 | 718 |
| 319 | 704 | 319 | 705 | 319 | Del3* | 319 | 706 |
| 319 | 707 | 319 | 708 | 319 | 709 | 319 | 710 |
| 319 | 711 | 319 | 712 | 319 | 713 | 319 | 714 |
| 319 | 715 | 319 | 716 | 319 | 717 | 319 | 718 |
| 320 | 704 | 320 | 705 | 320 | Del3* | 320 | 706 |
| 320 | 707 | 320 | 708 | 320 | 709 | 320 | 710 |
| 320 | 711 | 320 | 712 | 320 | 713 | 320 | 714 |
| 320 | 715 | 320 | 716 | 320 | 717 | 320 | 718 |
| 321 | 704 | 321 | 705 | 321 | Del3* | 321 | 706 |
| 321 | 707 | 321 | 708 | 321 | 709 | 321 | 710 |
| 321 | 711 | 321 | 712 | 321 | 713 | 321 | 714 |
| 321 | 715 | 321 | 716 | 321 | 717 | 321 | 718 |
| 322 | 704 | 322 | 705 | 322 | Del3* | 322 | 706 |
| 322 | 707 | 322 | 708 | 322 | 709 | 322 | 710 |
| 322 | 711 | 322 | 712 | 322 | 713 | 322 | 714 |
| 322 | 715 | 322 | 716 | 322 | 717 | 322 | 718 |
| 323 | 704 | 323 | 705 | 323 | Del3* | 323 | 706 |
| 323 | 707 | 323 | 708 | 323 | 709 | 323 | 710 |
| 323 | 711 | 323 | 712 | 323 | 713 | 323 | 714 |
| 323 | 715 | 323 | 716 | 323 | 717 | 323 | 718 |
| 324 | 704 | 324 | 705 | 324 | Del3* | 324 | 706 |
| 324 | 707 | 324 | 708 | 324 | 709 | 324 | 710 |
| 324 | 711 | 324 | 712 | 324 | 713 | 324 | 714 |
| 324 | 715 | 324 | 716 | 324 | 717 | 324 | 718 |
| 325 | 704 | 325 | 705 | 325 | Del3* | 325 | 706 |
| 325 | 707 | 325 | 708 | 325 | 709 | 325 | 710 |
| 325 | 711 | 325 | 712 | 325 | 713 | 325 | 714 |
| 325 | 715 | 325 | 716 | 325 | 717 | 325 | 718 |
| 326 | 704 | 326 | 705 | 326 | Del3* | 326 | 706 |
| 326 | 707 | 326 | 708 | 326 | 709 | 326 | 710 |
| 326 | 711 | 326 | 712 | 326 | 713 | 326 | 714 |
| 326 | 715 | 326 | 716 | 326 | 717 | 326 | 718 |
| 327 | 704 | 327 | 705 | 327 | Del3* | 327 | 706 |
| 327 | 707 | 327 | 708 | 327 | 709 | 327 | 710 |
| 327 | 711 | 327 | 712 | 327 | 713 | 327 | 714 |
| 327 | 715 | 327 | 716 | 327 | 717 | 327 | 718 |
| 328 | 704 | 328 | 705 | 328 | Del3* | 328 | 706 |
| 328 | 707 | 328 | 708 | 328 | 709 | 328 | 710 |
| 328 | 711 | 328 | 712 | 328 | 713 | 328 | 714 |
| 328 | 715 | 328 | 716 | 328 | 717 | 328 | 718 |
| 329 | 704 | 329 | 705 | 329 | Del3* | 329 | 706 |
| 329 | 707 | 329 | 708 | 329 | 709 | 329 | 710 |
| 329 | 711 | 329 | 712 | 329 | 713 | 329 | 714 |
| 329 | 715 | 329 | 716 | 329 | 717 | 329 | 718 |
| 330 | 704 | 330 | 705 | 330 | Del3* | 330 | 706 |
| 330 | 707 | 330 | 708 | 330 | 709 | 330 | 710 |
| 330 | 711 | 330 | 712 | 330 | 713 | 330 | 714 |
| 330 | 715 | 330 | 716 | 330 | 717 | 330 | 718 |
| 331 | 704 | 331 | 705 | 331 | Del3* | 331 | 706 |
| 331 | 707 | 331 | 708 | 331 | 709 | 331 | 710 |
| 331 | 711 | 331 | 712 | 331 | 713 | 331 | 714 |
| 331 | 715 | 331 | 716 | 331 | 717 | 331 | 718 |
| 332 | 704 | 332 | 705 | 332 | Del3* | 332 | 706 |
| 332 | 707 | 332 | 708 | 332 | 709 | 332 | 710 |
| 332 | 711 | 332 | 712 | 332 | 713 | 332 | 714 |
| 332 | 715 | 332 | 716 | 332 | 717 | 332 | 718 |
| 333 | 704 | 333 | 705 | 333 | Del3* | 333 | 706 |
| 333 | 707 | 333 | 708 | 333 | 709 | 333 | 710 |
| 333 | 711 | 333 | 712 | 333 | 713 | 333 | 714 |
| 333 | 715 | 333 | 716 | 333 | 717 | 333 | 718 |
| 334 | 704 | 334 | 705 | 334 | Del3* | 334 | 706 |
| 334 | 707 | 334 | 708 | 334 | 709 | 334 | 710 |
| 334 | 711 | 334 | 712 | 334 | 713 | 334 | 714 |
| 334 | 715 | 334 | 716 | 334 | 717 | 334 | 718 |
| 335 | 704 | 335 | 705 | 335 | Del3* | 335 | 706 |
| 335 | 707 | 335 | 708 | 335 | 709 | 335 | 710 |
| 335 | 711 | 335 | 712 | 335 | 713 | 335 | 714 |
| 335 | 715 | 335 | 716 | 335 | 717 | 335 | 718 |
| 336 | 704 | 336 | 705 | 336 | Del3* | 336 | 706 |
| 336 | 707 | 336 | 708 | 336 | 709 | 336 | 710 |
| 336 | 711 | 336 | 712 | 336 | 713 | 336 | 714 |
| 336 | 715 | 336 | 716 | 336 | 717 | 336 | 718 |
| 337 | 704 | 337 | 705 | 337 | Del3* | 337 | 706 |
| 337 | 707 | 337 | 708 | 337 | 709 | 337 | 710 |
| 337 | 711 | 337 | 712 | 337 | 713 | 337 | 714 |
| 337 | 715 | 337 | 716 | 337 | 717 | 337 | 718 |
| 338 | 704 | 338 | 705 | 338 | Del3* | 338 | 706 |
| 338 | 707 | 338 | 708 | 338 | 709 | 338 | 710 |
| 338 | 711 | 338 | 712 | 338 | 713 | 338 | 714 |
| 338 | 715 | 338 | 716 | 338 | 717 | 338 | 718 |
| 339 | 704 | 339 | 705 | 339 | Del3* | 339 | 706 |
| 339 | 707 | 339 | 708 | 339 | 709 | 339 | 710 |
| 339 | 711 | 339 | 712 | 339 | 713 | 339 | 714 |
| 339 | 715 | 339 | 716 | 339 | 717 | 339 | 718 |
| 340 | 704 | 340 | 705 | 340 | Del3* | 340 | 706 |
| 340 | 707 | 340 | 708 | 340 | 709 | 340 | 710 |
| 340 | 711 | 340 | 712 | 340 | 713 | 340 | 714 |
| 340 | 715 | 340 | 716 | 340 | 717 | 340 | 718 |
| 341 | 704 | 341 | 705 | 341 | Del3* | 341 | 706 |
| 341 | 707 | 341 | 708 | 341 | 709 | 341 | 710 |
| 341 | 711 | 341 | 712 | 341 | 713 | 341 | 714 |
| 341 | 715 | 341 | 716 | 341 | 717 | 341 | 718 |
| 342 | 704 | 342 | 705 | 342 | Del3* | 342 | 706 |
| 342 | 707 | 342 | 708 | 342 | 709 | 342 | 710 |
| 342 | 711 | 342 | 712 | 342 | 713 | 342 | 714 |
| 342 | 715 | 342 | 716 | 342 | 717 | 342 | 718 |
| 343 | 704 | 343 | 705 | 343 | Del3* | 343 | 706 |
| 343 | 707 | 343 | 708 | 343 | 709 | 343 | 710 |
| 343 | 711 | 343 | 712 | 343 | 713 | 343 | 714 |
| 343 | 715 | 343 | 716 | 343 | 717 | 343 | 718 |
| 344 | 704 | 344 | 705 | 344 | Del3* | 344 | 706 |
| 344 | 707 | 344 | 708 | 344 | 709 | 344 | 710 |
| 344 | 711 | 344 | 712 | 344 | 713 | 344 | 714 |
| 344 | 715 | 344 | 716 | 344 | 717 | 344 | 718 |
| 345 | 704 | 345 | 705 | 345 | Del3* | 345 | 706 |
| 345 | 707 | 345 | 708 | 345 | 709 | 345 | 710 |
| 345 | 711 | 345 | 712 | 345 | 713 | 345 | 714 |
| 345 | 715 | 345 | 716 | 345 | 717 | 345 | 718 |
| 346 | 704 | 346 | 705 | 346 | Del3* | 346 | 706 |
| 346 | 707 | 346 | 708 | 346 | 709 | 346 | 710 |
| 346 | 711 | 346 | 712 | 346 | 713 | 346 | 714 |
| 346 | 715 | 346 | 716 | 346 | 717 | 346 | 718 |
| 347 | 704 | 347 | 705 | 347 | Del3* | 347 | 706 |
| 347 | 707 | 347 | 708 | 347 | 709 | 347 | 710 |
| 347 | 711 | 347 | 712 | 347 | 713 | 347 | 714 |
| 347 | 715 | 347 | 716 | 347 | 717 | 347 | 718 |
| 348 | 704 | 348 | 705 | 348 | Del3* | 348 | 706 |
| 348 | 707 | 348 | 708 | 348 | 709 | 348 | 710 |
| 348 | 711 | 348 | 712 | 348 | 713 | 348 | 714 |
| 348 | 715 | 348 | 716 | 348 | 717 | 348 | 718 |
| 349 | 704 | 349 | 705 | 349 | Del3* | 349 | 706 |
| 349 | 707 | 349 | 708 | 349 | 709 | 349 | 710 |
| 349 | 711 | 34 | 712 | 34 | 713 | 34 | 714 |
| 349 | 715 | 349 | 716 | 349 | 717 | 349 | 718 |
| 350 | 704 | 350 | 705 | 350 | Del3* | 350 | 706 |
| 350 | 707 | 350 | 708 | 350 | 709 | 350 | 710 |
| 350 | 711 | 350 | 712 | 350 | 713 | 350 | 714 |
| 350 | 715 | 350 | 716 | 350 | 717 | 350 | 718 |
| 351 | 704 | 351 | 705 | 351 | Del3* | 351 | 706 |
| 351 | 707 | 351 | 708 | 351 | 709 | 351 | 710 |
| 351 | 711 | 351 | 712 | 351 | 713 | 351 | 714 |
| 351 | 715 | 351 | 716 | 351 | 717 | 351 | 718 |
| 352 | 704 | 352 | 705 | 352 | Del3* | 352 | 706 |
| 352 | 707 | 352 | 708 | 352 | 709 | 352 | 710 |
| 352 | 711 | 352 | 712 | 352 | 713 | 352 | 714 |
| 352 | 715 | 352 | 716 | 352 | 717 | 352 | 718 |
| 353 | 704 | 353 | 705 | 353 | Del3* | 353 | 706 |
| 353 | 707 | 353 | 708 | 353 | 709 | 353 | 710 |
| 353 | 711 | 353 | 712 | 353 | 713 | 353 | 714 |
| 353 | 715 | 353 | 716 | 353 | 717 | 353 | 718 |
| 632 | 704 | 632 | 705 | 632 | Del3* | 632 | 706 |
| 632 | 707 | 632 | 708 | 632 | 709 | 632 | 710 |
| 632 | 711 | 632 | 712 | 632 | 713 | 632 | 714 |
| 632 | 715 | 632 | 716 | 632 | 717 | 632 | 718 |
| 633 | 704 | 633 | 705 | 633 | Del3* | 633 | 706 |
| 633 | 707 | 633 | 708 | 633 | 709 | 633 | 710 |
| 633 | 711 | 633 | 712 | 633 | 713 | 633 | 714 |
| 633 | 715 | 633 | 716 | 633 | 717 | 633 | 718 |
| 634 | 704 | 634 | 705 | 634 | Del3* | 634 | 706 |
| 634 | 707 | 634 | 708 | 634 | 709 | 634 | 710 |
| 634 | 711 | 634 | 712 | 634 | 713 | 634 | 714 |
| 634 | 715 | 634 | 716 | 634 | 717 | 634 | 718 |
| 635 | 704 | 635 | 705 | 635 | Del3* | 635 | 706 |
| 635 | 707 | 635 | 708 | 635 | 709 | 635 | 710 |
| 635 | 711 | 635 | 712 | 635 | 713 | 635 | 714 |
| 635 | 715 | 635 | 716 | 635 | 717 | 635 | 718 |
| 636 | 704 | 636 | 705 | 636 | Del3* | 636 | 706 |
| 636 | 707 | 636 | 708 | 636 | 709 | 636 | 710 |
| 636 | 711 | 636 | 712 | 636 | 713 | 636 | 714 |
| 636 | 715 | 636 | 716 | 636 | 717 | 636 | 718 |
| 637 | 704 | 637 | 705 | 637 | Del3* | 637 | 706 |
| 637 | 707 | 637 | 708 | 637 | 709 | 637 | 710 |
| 637 | 711 | 637 | 712 | 637 | 713 | 637 | 714 |
| 637 | 715 | 637 | 716 | 637 | 717 | 637 | 718 |
| 638 | 704 | 638 | 705 | 638 | Del3* | 638 | 706 |
| 638 | 707 | 638 | 708 | 638 | 709 | 638 | 710 |
| 638 | 711 | 638 | 712 | 638 | 713 | 638 | 714 |
| 638 | 715 | 638 | 716 | 638 | 717 | 638 | 718 |
| 639 | 704 | 639 | 705 | 639 | Del3* | 639 | 706 |
| 639 | 707 | 639 | 708 | 639 | 709 | 639 | 710 |
| 639 | 711 | 639 | 712 | 639 | 713 | 639 | 714 |
| 639 | 715 | 639 | 716 | 639 | 717 | 639 | 718 |
| 640 | 704 | 640 | 705 | 640 | Del3* | 640 | 706 |
| 640 | 707 | 640 | 708 | 640 | 709 | 640 | 710 |
| 640 | 711 | 640 | 712 | 640 | 713 | 640 | 714 |
| 640 | 715 | 640 | 716 | 640 | 717 | 640 | 718 |
| 641 | 704 | 641 | 705 | 641 | Del3* | 641 | 706 |
| 641 | 707 | 641 | 708 | 641 | 709 | 641 | 710 |
| 641 | 711 | 641 | 712 | 641 | 713 | 641 | 714 |
| 641 | 715 | 641 | 716 | 641 | 717 | 641 | 718 |
| 642 | 704 | 642 | 705 | 642 | Del3* | 642 | 706 |
| 642 | 707 | 642 | 708 | 642 | 709 | 642 | 710 |
| 642 | 711 | 642 | 712 | 642 | 713 | 642 | 714 |
| 642 | 715 | 642 | 716 | 642 | 717 | 642 | 718 |
| 643 | 704 | 643 | 705 | 643 | Del3* | 643 | 706 |
| 643 | 707 | 643 | 708 | 643 | 709 | 643 | 710 |
| 643 | 711 | 643 | 712 | 643 | 713 | 643 | 714 |
| 643 | 715 | 643 | 716 | 643 | 717 | 643 | 718 |
| 644 | 704 | 644 | 705 | 644 | Del3* | 644 | 706 |
| 644 | 707 | 644 | 708 | 644 | 709 | 644 | 710 |
| 644 | 711 | 644 | 712 | 644 | 713 | 644 | 714 |
| 644 | 715 | 644 | 716 | 644 | 717 | 644 | 718 |
| 645 | 704 | 645 | 705 | 645 | Del3* | 645 | 706 |
| 645 | 707 | 645 | 708 | 645 | 709 | 645 | 710 |
| 645 | 711 | 645 | 712 | 645 | 713 | 645 | 714 |
| 645 | 715 | 645 | 716 | 645 | 717 | 645 | 718 |
| 646 | 704 | 646 | 705 | 646 | Del3* | 646 | 706 |
| 646 | 707 | 646 | 708 | 646 | 709 | 646 | 710 |
| 646 | 711 | 646 | 712 | 646 | 713 | 646 | 714 |
| 646 | 715 | 646 | 716 | 646 | 717 | 646 | 718 |
| 647 | 704 | 647 | 705 | 647 | Del3* | 647 | 706 |
| 647 | 707 | 647 | 708 | 647 | 709 | 647 | 710 |
| 647 | 711 | 647 | 712 | 647 | 713 | 647 | 714 |
| 647 | 715 | 647 | 716 | 647 | 717 | 647 | 718 |
| 648 | 704 | 648 | 705 | 648 | Del3* | 648 | 706 |
| 648 | 707 | 648 | 708 | 648 | 709 | 648 | 710 |
| 648 | 711 | 648 | 712 | 648 | 713 | 648 | 714 |
| 648 | 715 | 648 | 716 | 648 | 717 | 648 | 718 |
| 649 | 704 | 649 | 705 | 649 | Del3* | 649 | 706 |
| 649 | 707 | 649 | 708 | 649 | 709 | 649 | 710 |
| 649 | 711 | 649 | 712 | 649 | 713 | 649 | 714 |
| 649 | 715 | 649 | 716 | 649 | 717 | 649 | 718 |
| 650 | 704 | 650 | 705 | 650 | Del3* | 650 | 706 |
| 650 | 707 | 650 | 708 | 650 | 709 | 650 | 710 |
| 650 | 711 | 650 | 712 | 650 | 713 | 650 | 714 |
| 650 | 715 | 650 | 716 | 650 | 717 | 650 | 718 |
| 651 | 704 | 651 | 705 | 651 | Del3* | 651 | 706 |
| 651 | 707 | 651 | 708 | 651 | 709 | 651 | 710 |
| 651 | 711 | 651 | 712 | 651 | 713 | 651 | 714 |
| 651 | 715 | 651 | 716 | 651 | 717 | 651 | 718 |
| 652 | 704 | 652 | 705 | 652 | Del3* | 652 | 706 |
| 652 | 707 | 652 | 708 | 652 | 709 | 652 | 710 |
| 652 | 711 | 652 | 712 | 652 | 713 | 652 | 714 |
| 652 | 715 | 652 | 716 | 652 | 717 | 652 | 718 |
| 653 | 704 | 653 | 705 | 653 | Del3* | 653 | 706 |
| 653 | 707 | 653 | 708 | 653 | 709 | 653 | 710 |
| 653 | 711 | 653 | 712 | 653 | 713 | 653 | 714 |
| 653 | 715 | 653 | 716 | 653 | 717 | 653 | 718 |
| 654 | 704 | 654 | 705 | 654 | Del3* | 654 | 706 |
| 654 | 707 | 654 | 708 | 654 | 709 | 654 | 710 |
| 654 | 711 | 654 | 712 | 654 | 713 | 654 | 714 |
| 654 | 715 | 654 | 716 | 654 | 717 | 654 | 718 |
| 655 | 704 | 655 | 705 | 655 | Del3* | 655 | 706 |
| 655 | 707 | 655 | 708 | 655 | 709 | 655 | 710 |
| 655 | 711 | 655 | 712 | 655 | 713 | 655 | 714 |
| 655 | 715 | 655 | 716 | 655 | 717 | 655 | 718 |
| 656 | 704 | 656 | 705 | 656 | Del3* | 656 | 706 |
| 656 | 707 | 656 | 708 | 656 | 709 | 656 | 710 |
| 656 | 711 | 656 | 712 | 656 | 713 | 656 | 714 |
| 656 | 715 | 656 | 716 | 656 | 717 | 656 | 718 |
| 657 | 704 | 657 | 705 | 657 | Del3* | 657 | 706 |
| 657 | 707 | 657 | 708 | 657 | 709 | 657 | 710 |
| 657 | 711 | 657 | 712 | 657 | 713 | 657 | 714 |
| 657 | 715 | 657 | 716 | 657 | 717 | 657 | 718 |
| 658 | 704 | 658 | 705 | 658 | Del3* | 658 | 706 |
| 658 | 707 | 658 | 708 | 658 | 709 | 658 | 710 |
| 658 | 711 | 658 | 712 | 658 | 713 | 658 | 714 |
| 658 | 715 | 658 | 716 | 658 | 717 | 658 | 718 |
| 659 | 704 | 659 | 705 | 659 | Del3* | 659 | 706 |
| 659 | 707 | 659 | 708 | 659 | 709 | 659 | 710 |
| 659 | 711 | 659 | 712 | 659 | 713 | 659 | 714 |
| 659 | 715 | 659 | 716 | 659 | 717 | 659 | 718 |
| 660 | 704 | 660 | 705 | 660 | Del3* | 660 | 706 |
| 660 | 707 | 660 | 708 | 660 | 709 | 660 | 710 |
| 660 | 711 | 660 | 712 | 660 | 713 | 660 | 714 |
| 660 | 715 | 660 | 716 | 660 | 717 | 660 | 718 |
| 661 | 704 | 661 | 705 | 661 | Del3* | 661 | 706 |
| 661 | 707 | 661 | 708 | 661 | 709 | 661 | 710 |
| 661 | 711 | 661 | 712 | 661 | 713 | 661 | 714 |
| 661 | 715 | 661 | 716 | 661 | 717 | 661 | 718 |
| 662 | 704 | 662 | 705 | 662 | Del3* | 662 | 706 |
| 662 | 707 | 662 | 708 | 662 | 709 | 662 | 710 |
| 662 | 711 | 662 | 712 | 662 | 713 | 662 | 714 |
| 662 | 715 | 662 | 716 | 662 | 717 | 662 | 718 |
| 663 | 704 | 663 | 705 | 663 | Del3* | 663 | 706 |
| 663 | 707 | 663 | 708 | 663 | 709 | 663 | 710 |
| 663 | 711 | 663 | 712 | 663 | 713 | 663 | 714 |
| 663 | 715 | 663 | 716 | 663 | 717 | 663 | 718 |
| 664 | 704 | 664 | 705 | 664 | Del3* | 664 | 706 |
| 664 | 707 | 664 | 708 | 664 | 709 | 664 | 710 |
| 664 | 711 | 664 | 712 | 664 | 713 | 664 | 714 |
| 664 | 715 | 664 | 716 | 664 | 717 | 664 | 718 |
| 665 | 704 | 665 | 705 | 665 | Del3* | 665 | 706 |
| 665 | 707 | 665 | 708 | 665 | 709 | 665 | 710 |
| 665 | 711 | 665 | 712 | 665 | 713 | 665 | 714 |
| 665 | 715 | 665 | 716 | 665 | 717 | 665 | 718 |
| 666 | 704 | 666 | 705 | 666 | Del3* | 666 | 706 |
| 666 | 707 | 666 | 708 | 666 | 709 | 666 | 710 |
| 666 | 711 | 666 | 712 | 666 | 713 | 666 | 714 |
| 666 | 715 | 666 | 716 | 666 | 717 | 666 | 718 |
| 667 | 704 | 667 | 705 | 667 | Del3* | 667 | 706 |
| 667 | 707 | 667 | 708 | 667 | 709 | 667 | 710 |
| 667 | 711 | 667 | 712 | 667 | 713 | 667 | 714 |
| 667 | 715 | 667 | 716 | 667 | 717 | 667 | 718 |
| 464 | 704 | 464 | 705 | 464 | Del3* | 464 | 706 |
| 464 | 707 | 464 | 708 | 464 | 709 | 464 | 710 |
| 464 | 711 | 464 | 712 | 464 | 713 | 464 | 714 |
| 464 | 715 | 464 | 716 | 464 | 717 | 464 | 718 |
| 465 | 704 | 465 | 705 | 465 | Del3* | 465 | 706 |
| 465 | 707 | 465 | 708 | 465 | 709 | 465 | 710 |
| 465 | 711 | 465 | 712 | 465 | 713 | 465 | 714 |
| 465 | 715 | 465 | 716 | 465 | 717 | 465 | 718 |
| 466 | 704 | 466 | 705 | 466 | Del3* | 466 | 706 |
| 466 | 707 | 466 | 708 | 466 | 709 | 466 | 710 |
| 466 | 711 | 466 | 712 | 466 | 713 | 466 | 714 |
| 466 | 715 | 466 | 716 | 466 | 717 | 466 | 718 |
| 467 | 704 | 467 | 705 | 467 | Del3* | 467 | 706 |
| 467 | 707 | 467 | 708 | 467 | 709 | 467 | 710 |
| 467 | 711 | 467 | 712 | 467 | 713 | 467 | 714 |
| 467 | 715 | 467 | 716 | 467 | 717 | 467 | 718 |
| 468 | 704 | 468 | 705 | 468 | Del3* | 468 | 706 |
| 468 | 707 | 468 | 708 | 468 | 709 | 468 | 710 |
| 468 | 711 | 468 | 712 | 468 | 713 | 468 | 714 |
| 468 | 715 | 468 | 716 | 468 | 717 | 468 | 718 |
| 469 | 704 | 469 | 705 | 469 | Del3* | 469 | 706 |
| 469 | 707 | 469 | 708 | 469 | 709 | 469 | 710 |
| 469 | 711 | 469 | 712 | 469 | 713 | 469 | 714 |
| 469 | 715 | 469 | 716 | 469 | 717 | 469 | 718 |
| 470 | 704 | 470 | 705 | 470 | Del3* | 470 | 706 |
| 470 | 707 | 470 | 708 | 470 | 709 | 470 | 710 |
| 470 | 711 | 470 | 712 | 470 | 713 | 470 | 714 |
| 470 | 715 | 470 | 716 | 470 | 717 | 470 | 718 |
| 471 | 704 | 471 | 705 | 471 | Del3* | 471 | 706 |
| 471 | 707 | 471 | 708 | 471 | 709 | 471 | 710 |
| 471 | 711 | 471 | 712 | 471 | 713 | 471 | 714 |
| 471 | 715 | 471 | 716 | 471 | 717 | 471 | 718 |
| 472 | 704 | 472 | 705 | 472 | Del3* | 472 | 706 |
| 472 | 707 | 472 | 708 | 472 | 709 | 472 | 710 |
| 472 | 711 | 472 | 712 | 472 | 713 | 472 | 714 |
| 472 | 715 | 472 | 716 | 472 | 717 | 472 | 718 |
| 473 | 704 | 473 | 705 | 473 | Del3* | 473 | 706 |
| 473 | 707 | 473 | 708 | 473 | 709 | 473 | 710 |
| 473 | 711 | 473 | 712 | 473 | 713 | 473 | 714 |
| 473 | 715 | 473 | 716 | 473 | 717 | 473 | 718 |
| 474 | 704 | 474 | 705 | 474 | Del3* | 474 | 706 |
| 474 | 707 | 474 | 708 | 474 | 709 | 474 | 710 |
| 474 | 711 | 474 | 712 | 474 | 713 | 474 | 714 |
| 474 | 715 | 474 | 716 | 474 | 717 | 474 | 718 |
| 475 | 704 | 475 | 705 | 475 | Del3* | 475 | 706 |
| 475 | 707 | 475 | 708 | 475 | 709 | 475 | 710 |
| 475 | 711 | 475 | 712 | 475 | 713 | 475 | 714 |
| 475 | 715 | 475 | 716 | 475 | 717 | 475 | 718 |
| 476 | 704 | 476 | 705 | 476 | Del3* | 476 | 706 |
| 476 | 707 | 476 | 708 | 476 | 709 | 476 | 710 |
| 476 | 711 | 476 | 712 | 476 | 713 | 476 | 714 |
| 476 | 715 | 476 | 716 | 476 | 717 | 476 | 718 |
| 477 | 704 | 477 | 705 | 477 | Del3* | 477 | 706 |
| 477 | 707 | 477 | 708 | 477 | 709 | 477 | 710 |
| 477 | 711 | 477 | 712 | 477 | 713 | 477 | 714 |
| 477 | 715 | 477 | 716 | 477 | 717 | 477 | 718 |
| 478 | 704 | 478 | 705 | 478 | Del3* | 478 | 706 |
| 478 | 707 | 478 | 708 | 478 | 709 | 478 | 710 |
| 478 | 711 | 478 | 712 | 478 | 713 | 478 | 714 |
| 478 | 715 | 478 | 716 | 478 | 717 | 478 | 718 |
| 479 | 704 | 479 | 705 | 479 | Del3* | 479 | 706 |
| 479 | 707 | 479 | 708 | 479 | 709 | 479 | 710 |
| 479 | 711 | 479 | 712 | 479 | 713 | 479 | 714 |
| 479 | 715 | 479 | 716 | 479 | 717 | 479 | 718 |
| 480 | 704 | 480 | 705 | 480 | Del3* | 480 | 706 |
| 480 | 707 | 480 | 708 | 480 | 709 | 480 | 710 |
| 480 | 711 | 480 | 712 | 480 | 713 | 480 | 714 |
| 480 | 715 | 480 | 716 | 480 | 717 | 480 | 718 |
| 481 | 704 | 481 | 705 | 481 | Del3* | 481 | 706 |
| 481 | 707 | 481 | 708 | 481 | 709 | 481 | 710 |
| 481 | 711 | 481 | 712 | 481 | 713 | 481 | 714 |
| 481 | 715 | 481 | 716 | 481 | 717 | 481 | 718 |
| 482 | 704 | 482 | 705 | 482 | Del3* | 482 | 706 |
| 482 | 707 | 482 | 708 | 482 | 709 | 482 | 710 |
| 482 | 711 | 482 | 712 | 482 | 713 | 482 | 714 |
| 482 | 715 | 482 | 716 | 482 | 717 | 482 | 718 |
| 483 | 704 | 483 | 705 | 483 | Del3* | 483 | 706 |
| 483 | 707 | 483 | 708 | 483 | 709 | 483 | 710 |
| 483 | 711 | 483 | 712 | 483 | 713 | 483 | 714 |
| 483 | 715 | 483 | 716 | 483 | 717 | 483 | 718 |
| 484 | 704 | 484 | 705 | 484 | Del3* | 484 | 706 |
| 484 | 707 | 484 | 708 | 484 | 709 | 484 | 710 |
| 484 | 711 | 484 | 712 | 484 | 713 | 484 | 714 |
| 484 | 715 | 484 | 716 | 484 | 717 | 484 | 718 |
| 485 | 704 | 485 | 705 | 485 | Del3* | 485 | 706 |
| 485 | 707 | 485 | 708 | 485 | 709 | 485 | 710 |
| 485 | 711 | 485 | 712 | 485 | 713 | 485 | 714 |
| 485 | 715 | 485 | 716 | 485 | 717 | 485 | 718 |
| 486 | 704 | 486 | 705 | 486 | Del3* | 486 | 706 |
| 486 | 707 | 486 | 708 | 486 | 709 | 486 | 710 |
| 486 | 711 | 486 | 712 | 486 | 713 | 486 | 714 |
| 486 | 715 | 486 | 716 | 486 | 717 | 486 | 718 |
| 487 | 704 | 487 | 705 | 487 | Del3* | 487 | 706 |
| 487 | 707 | 487 | 708 | 487 | 709 | 487 | 710 |
| 487 | 711 | 487 | 712 | 487 | 713 | 487 | 714 |
| 487 | 715 | 487 | 716 | 487 | 717 | 487 | 718 |
| 488 | 704 | 488 | 705 | 488 | Del3* | 488 | 706 |
| 488 | 707 | 488 | 708 | 488 | 709 | 488 | 710 |
| 488 | 711 | 488 | 712 | 488 | 713 | 488 | 714 |
| 488 | 715 | 488 | 716 | 488 | 717 | 488 | 718 |
| 489 | 704 | 489 | 705 | 489 | Del3* | 489 | 706 |
| 489 | 707 | 489 | 708 | 489 | 709 | 489 | 710 |
| 489 | 711 | 489 | 712 | 489 | 713 | 489 | 714 |
| 489 | 715 | 489 | 716 | 489 | 717 | 489 | 718 |
| 490 | 704 | 490 | 705 | 490 | Del3* | 490 | 706 |
| 490 | 707 | 490 | 708 | 490 | 709 | 490 | 710 |
| 490 | 711 | 490 | 712 | 490 | 713 | 490 | 714 |
| 490 | 715 | 490 | 716 | 490 | 717 | 490 | 718 |
| 491 | 704 | 491 | 705 | 491 | Del3* | 491 | 706 |
| 491 | 707 | 491 | 708 | 491 | 709 | 491 | 710 |
| 491 | 711 | 491 | 712 | 491 | 713 | 491 | 714 |
| 491 | 715 | 491 | 716 | 491 | 717 | 491 | 718 |
| 492 | 704 | 492 | 705 | 492 | Del3* | 492 | 706 |
| 492 | 707 | 492 | 708 | 492 | 709 | 492 | 710 |
| 492 | 711 | 492 | 712 | 492 | 713 | 492 | 714 |
| 492 | 715 | 492 | 716 | 492 | 717 | 492 | 718 |
| 493 | 704 | 493 | 705 | 493 | Del3* | 493 | 706 |
| 493 | 707 | 493 | 708 | 493 | 709 | 493 | 710 |
| 493 | 711 | 493 | 712 | 493 | 713 | 493 | 714 |
| 493 | 715 | 493 | 716 | 493 | 717 | 493 | 718 |
| 494 | 704 | 494 | 705 | 494 | Del3* | 494 | 706 |
| 494 | 707 | 494 | 708 | 494 | 709 | 494 | 710 |
| 494 | 711 | 494 | 712 | 494 | 713 | 494 | 714 |
| 494 | 715 | 494 | 716 | 494 | 717 | 494 | 718 |
| 495 | 704 | 495 | 705 | 495 | Del3* | 495 | 706 |
| 495 | 707 | 495 | 708 | 495 | 709 | 495 | 710 |
| 495 | 711 | 495 | 712 | 495 | 713 | 495 | 714 |
| 495 | 715 | 495 | 716 | 495 | 717 | 495 | 718 |
| 496 | 704 | 496 | 705 | 496 | Del3* | 496 | 706 |
| 496 | 707 | 496 | 708 | 496 | 709 | 496 | 710 |
| 496 | 711 | 496 | 712 | 496 | 713 | 496 | 714 |
| 496 | 715 | 496 | 716 | 496 | 717 | 496 | 718 |
| 497 | 704 | 497 | 705 | 497 | Del3* | 497 | 706 |
| 497 | 707 | 497 | 708 | 497 | 709 | 497 | 710 |
| 497 | 711 | 497 | 712 | 497 | 713 | 497 | 714 |
| 497 | 715 | 497 | 716 | 497 | 717 | 497 | 718 |
| 498 | 704 | 498 | 705 | 498 | Del3* | 498 | 706 |
| 498 | 707 | 498 | 708 | 498 | 709 | 498 | 710 |
| 498 | 711 | 498 | 712 | 498 | 713 | 498 | 714 |
| 498 | 715 | 498 | 716 | 498 | 717 | 498 | 718 |
| 499 | 704 | 499 | 705 | 499 | Del3* | 499 | 706 |
| 499 | 707 | 499 | 708 | 499 | 709 | 499 | 710 |
| 499 | 711 | 499 | 712 | 499 | 713 | 499 | 714 |
| 499 | 715 | 499 | 716 | 499 | 717 | 499 | 718 |
| 500 | 704 | 500 | 705 | 500 | Del3* | 500 | 706 |
| 500 | 707 | 500 | 708 | 500 | 709 | 500 | 710 |
| 500 | 711 | 500 | 712 | 500 | 713 | 500 | 714 |
| 500 | 715 | 500 | 716 | 500 | 717 | 500 | 718 |
| 501 | 704 | 501 | 705 | 501 | Del3* | 501 | 706 |
| 501 | 707 | 501 | 708 | 501 | 709 | 501 | 710 |
| 501 | 711 | 501 | 712 | 501 | 713 | 501 | 714 |
| 501 | 715 | 501 | 716 | 501 | 717 | 501 | 718 |
| 502 | 704 | 502 | 705 | 502 | Del3* | 502 | 706 |
| 502 | 707 | 502 | 708 | 502 | 709 | 502 | 710 |
| 502 | 711 | 502 | 712 | 502 | 713 | 502 | 714 |
| 502 | 715 | 502 | 716 | 502 | 717 | 502 | 718 |
| 503 | 704 | 503 | 705 | 503 | Del3* | 503 | 706 |
| 503 | 707 | 503 | 708 | 503 | 709 | 503 | 710 |
| 503 | 711 | 503 | 712 | 503 | 713 | 503 | 714 |
| 503 | 715 | 503 | 716 | 503 | 717 | 503 | 718 |
| 504 | 704 | 504 | 705 | 504 | Del3* | 504 | 706 |
| 504 | 707 | 504 | 708 | 504 | 709 | 504 | 710 |
| 504 | 711 | 504 | 712 | 504 | 713 | 504 | 714 |
| 504 | 715 | 504 | 716 | 504 | 717 | 504 | 718 |
| 505 | 704 | 505 | 705 | 505 | Del3* | 505 | 706 |
| 505 | 707 | 505 | 708 | 505 | 709 | 505 | 710 |
| 505 | 711 | 505 | 712 | 505 | 713 | 505 | 714 |
| 505 | 715 | 505 | 716 | 505 | 717 | 505 | 718 |
| 506 | 704 | 506 | 705 | 506 | Del3* | 506 | 706 |
| 506 | 707 | 506 | 708 | 506 | 709 | 506 | 710 |
| 506 | 711 | 506 | 712 | 506 | 713 | 506 | 714 |
| 506 | 715 | 506 | 716 | 506 | 717 | 506 | 718 |
| 507 | 704 | 507 | 705 | 507 | Del3* | 507 | 706 |
| 507 | 707 | 507 | 708 | 507 | 709 | 507 | 710 |
| 507 | 711 | 507 | 712 | 507 | 713 | 507 | 714 |
| 507 | 715 | 507 | 716 | 507 | 717 | 507 | 718 |
| 508 | 704 | 508 | 705 | 508 | Del3* | 508 | 706 |
| 508 | 707 | 508 | 708 | 508 | 709 | 508 | 710 |
| 508 | 711 | 508 | 712 | 508 | 713 | 508 | 714 |
| 508 | 715 | 508 | 716 | 508 | 717 | 508 | 718 |
| 509 | 704 | 509 | 705 | 509 | Del3* | 509 | 706 |
| 509 | 707 | 509 | 708 | 509 | 709 | 509 | 710 |
| 509 | 711 | 509 | 712 | 509 | 713 | 509 | 714 |
| 509 | 715 | 509 | 716 | 509 | 717 | 509 | 718 |
| 510 | 704 | 510 | 705 | 510 | Del3* | 510 | 706 |
| 510 | 707 | 510 | 708 | 510 | 709 | 510 | 710 |
| 510 | 711 | 510 | 712 | 510 | 713 | 510 | 714 |
| 510 | 715 | 510 | 716 | 510 | 717 | 510 | 718 |
| 511 | 704 | 511 | 705 | 511 | Del3* | 511 | 706 |
| 511 | 707 | 511 | 708 | 511 | 709 | 511 | 710 |
| 511 | 711 | 511 | 712 | 511 | 713 | 511 | 714 |
| 511 | 715 | 511 | 716 | 511 | 717 | 511 | 718 |
| 512 | 704 | 512 | 705 | 512 | Del3* | 512 | 706 |
| 512 | 707 | 512 | 708 | 512 | 709 | 512 | 710 |
| 512 | 711 | 512 | 712 | 512 | 713 | 512 | 714 |
| 512 | 715 | 512 | 716 | 512 | 717 | 512 | 718 |
| 513 | 704 | 513 | 705 | 513 | Del3* | 513 | 706 |
| 513 | 707 | 513 | 708 | 513 | 709 | 513 | 710 |
| 513 | 711 | 513 | 712 | 513 | 713 | 513 | 714 |
| 513 | 715 | 513 | 716 | 513 | 717 | 513 | 718 |
| 514 | 704 | 514 | 705 | 514 | Del3* | 514 | 706 |
| 514 | 707 | 514 | 708 | 514 | 709 | 514 | 710 |
| 514 | 711 | 514 | 712 | 514 | 713 | 514 | 714 |
| 514 | 715 | 514 | 716 | 514 | 717 | 514 | 718 |
| 515 | 704 | 515 | 705 | 515 | Del3* | 515 | 706 |
| 515 | 707 | 515 | 708 | 515 | 709 | 515 | 710 |
| 515 | 711 | 515 | 712 | 515 | 713 | 515 | 714 |
| 515 | 715 | 515 | 716 | 515 | 717 | 515 | 718 |
| 516 | 704 | 516 | 705 | 516 | Del3* | 516 | 706 |
| 516 | 707 | 516 | 708 | 516 | 709 | 516 | 710 |
| 516 | 711 | 516 | 712 | 516 | 713 | 516 | 714 |
| 516 | 715 | 516 | 716 | 516 | 717 | 516 | 718 |
| 517 | 704 | 517 | 705 | 517 | Del3* | 517 | 706 |
| 517 | 707 | 517 | 708 | 517 | 709 | 517 | 710 |
| 517 | 711 | 517 | 712 | 517 | 713 | 517 | 714 |
| 517 | 715 | 517 | 716 | 517 | 717 | 517 | 718 |
| 518 | 704 | 518 | 705 | 518 | Del3* | 518 | 706 |
| 518 | 707 | 518 | 708 | 518 | 709 | 518 | 710 |
| 518 | 711 | 518 | 712 | 518 | 713 | 518 | 714 |
| 518 | 715 | 518 | 716 | 518 | 717 | 518 | 718 |
| 519 | 704 | 519 | 705 | 519 | Del3* | 519 | 706 |
| 519 | 707 | 519 | 708 | 519 | 709 | 519 | 710 |
| 519 | 711 | 519 | 712 | 519 | 713 | 519 | 714 |
| 519 | 715 | 519 | 716 | 519 | 717 | 519 | 718 |
| 520 | 704 | 520 | 705 | 520 | Del3* | 520 | 706 |
| 520 | 707 | 520 | 708 | 520 | 709 | 520 | 710 |
| 520 | 711 | 520 | 712 | 520 | 713 | 520 | 714 |
| 520 | 715 | 520 | 716 | 520 | 717 | 520 | 718 |
| 521 | 704 | 521 | 705 | 521 | Del3* | 521 | 706 |
| 521 | 707 | 521 | 708 | 521 | 709 | 521 | 710 |
| 521 | 711 | 521 | 712 | 521 | 713 | 521 | 714 |
| 521 | 715 | 521 | 716 | 521 | 717 | 521 | 718 |
| 522 | 704 | 522 | 705 | 522 | Del3* | 522 | 706 |
| 522 | 707 | 522 | 708 | 522 | 709 | 522 | 710 |
| 522 | 711 | 522 | 712 | 522 | 713 | 522 | 714 |
| 522 | 715 | 522 | 716 | 522 | 717 | 522 | 718 |
| 528 | 704 | 528 | 705 | 528 | Del3* | 528 | 706 |
| 528 | 707 | 528 | 708 | 528 | 709 | 528 | 710 |
| 528 | 711 | 528 | 712 | 528 | 713 | 528 | 714 |
| 528 | 715 | 528 | 716 | 528 | 717 | 528 | 718 |
| 529 | 704 | 529 | 705 | 529 | Del3* | 529 | 706 |
| 529 | 707 | 529 | 708 | 529 | 709 | 529 | 710 |
| 529 | 711 | 529 | 712 | 529 | 713 | 529 | 714 |
| 529 | 715 | 529 | 716 | 529 | 717 | 529 | 718 |
| 530 | 704 | 530 | 705 | 530 | Del3* | 530 | 706 |
| 530 | 707 | 530 | 708 | 530 | 709 | 530 | 710 |
| 530 | 711 | 530 | 712 | 530 | 713 | 530 | 714 |
| 530 | 715 | 530 | 716 | 530 | 717 | 530 | 718 |
| 531 | 704 | 531 | 705 | 531 | Del3* | 531 | 706 |
| 531 | 70 | 531 | 708 | 531 | 709 | 531 | 710 |
| 531 | 711 | 531 | 712 | 531 | 713 | 531 | 714 |
| 531 | 715 | 531 | 716 | 531 | 717 | 531 | 718 |
| 532 | 704 | 532 | 705 | 532 | Del3* | 532 | 706 |
| 532 | 707 | 532 | 708 | 532 | 709 | 532 | 710 |
| 532 | 71 | 532 | 712 | 532 | 713 | 532 | 714 |
| 532 | 715 | 532 | 716 | 532 | 717 | 532 | 718 |
| 533 | 704 | 533 | 705 | 533 | Del3* | 533 | 706 |
| 533 | 707 | 533 | 708 | 533 | 709 | 533 | 710 |
| 533 | 711 | 533 | 712 | 533 | 713 | 533 | 714 |
| 533 | 715 | 533 | 716 | 533 | 717 | 533 | 718 |
| 534 | 704 | 534 | 705 | 534 | Del3* | 534 | 706 |
| 534 | 707 | 534 | 708 | 534 | 709 | 534 | 710 |
| 534 | 711 | 534 | 712 | 534 | 713 | 534 | 714 |
| 534 | 715 | 534 | 716 | 534 | 717 | 534 | 718 |
| 535 | 704 | 535 | 705 | 535 | Del3* | 535 | 706 |
| 535 | 707 | 535 | 708 | 535 | 709 | 535 | 710 |
| 535 | 711 | 535 | 712 | 535 | 713 | 535 | 714 |
| 535 | 715 | 535 | 716 | 535 | 717 | 535 | 718 |
| 536 | 704 | 536 | 705 | 536 | Del3* | 536 | 706 |
| 536 | 707 | 536 | 708 | 536 | 709 | 536 | 710 |
| 536 | 711 | 536 | 712 | 536 | 713 | 536 | 714 |
| 536 | 715 | 536 | 716 | 536 | 717 | 536 | 718 |
| 537 | 704 | 537 | 705 | 537 | Del3* | 537 | 706 |
| 537 | 707 | 537 | 708 | 537 | 709 | 537 | 710 |
| 537 | 711 | 537 | 712 | 537 | 713 | 537 | 714 |
| 537 | 715 | 537 | 716 | 537 | 717 | 537 | 718 |
| 538 | 704 | 538 | 705 | 538 | Del3* | 538 | 706 |
| 538 | 707 | 538 | 708 | 538 | 709 | 538 | 710 |
| 538 | 711 | 538 | 712 | 538 | 713 | 538 | 714 |
| 538 | 715 | 538 | 716 | 538 | 717 | 538 | 718 |
| 539 | 704 | 539 | 705 | 539 | Del3* | 539 | 706 |
| 539 | 707 | 539 | 708 | 539 | 709 | 539 | 710 |
| 539 | 711 | 539 | 712 | 539 | 713 | 539 | 714 |
| 539 | 715 | 539 | 716 | 539 | 717 | 539 | 718 |
| 540 | 704 | 540 | 705 | 540 | Del3* | 540 | 706 |
| 540 | 707 | 540 | 708 | 540 | 709 | 540 | 710 |
| 540 | 711 | 540 | 712 | 540 | 713 | 540 | 714 |
| 540 | 715 | 540 | 716 | 540 | 717 | 540 | 718 |
| 541 | 704 | 541 | 705 | 541 | Del3* | 541 | 706 |
| 541 | 707 | 541 | 708 | 541 | 709 | 541 | 710 |
| 541 | 711 | 541 | 712 | 541 | 713 | 541 | 714 |
| 541 | 715 | 541 | 716 | 541 | 717 | 541 | 718 |
| 542 | 704 | 542 | 705 | 542 | Del3* | 542 | 706 |
| 542 | 707 | 542 | 708 | 542 | 709 | 542 | 710 |
| 542 | 711 | 542 | 712 | 542 | 713 | 542 | 714 |
| 542 | 715 | 542 | 716 | 542 | 717 | 542 | 718 |
| 543 | 704 | 543 | 705 | 543 | Del3* | 543 | 706 |
| 543 | 707 | 543 | 708 | 543 | 709 | 543 | 710 |
| 543 | 711 | 543 | 712 | 543 | 713 | 543 | 714 |
| 543 | 715 | 543 | 716 | 543 | 717 | 543 | 718 |
| 544 | 704 | 544 | 705 | 544 | Del3* | 544 | 706 |
| 544 | 707 | 544 | 708 | 544 | 709 | 544 | 710 |
| 544 | 711 | 544 | 712 | 544 | 713 | 544 | 714 |
| 544 | 715 | 544 | 716 | 544 | 717 | 544 | 718 |
| 545 | 704 | 545 | 705 | 545 | Del3* | 545 | 706 |
| 545 | 707 | 545 | 708 | 545 | 709 | 545 | 710 |
| 545 | 711 | 545 | 712 | 545 | 713 | 545 | 714 |
| 545 | 715 | 545 | 716 | 545 | 717 | 545 | 718 |
| 546 | 704 | 546 | 705 | 546 | Del3* | 546 | 706 |
| 546 | 707 | 546 | 708 | 546 | 709 | 546 | 710 |
| 546 | 711 | 546 | 712 | 546 | 713 | 546 | 714 |
| 546 | 715 | 546 | 716 | 546 | 717 | 546 | 718 |
| 547 | 704 | 547 | 705 | 547 | Del3* | 547 | 706 |
| 547 | 707 | 547 | 708 | 547 | 709 | 547 | 710 |
| 547 | 711 | 547 | 712 | 547 | 713 | 547 | 714 |
| 547 | 715 | 547 | 716 | 547 | 717 | 547 | 718 |
| 668 | 704 | 668 | 705 | 668 | Del3* | 668 | 706 |
| 668 | 707 | 668 | 708 | 668 | 709 | 668 | 710 |
| 668 | 711 | 668 | 712 | 668 | 713 | 668 | 714 |
| 668 | 715 | 668 | 716 | 668 | 717 | 668 | 718 |
| 669 | 704 | 669 | 705 | 669 | Del3* | 669 | 706 |
| 669 | 707 | 669 | 708 | 669 | 709 | 669 | 710 |
| 669 | 711 | 669 | 712 | 669 | 713 | 669 | 714 |
| 669 | 715 | 669 | 716 | 669 | 717 | 669 | 718 |
| 670 | 704 | 670 | 705 | 670 | Del3* | 670 | 706 |
| 670 | 707 | 670 | 708 | 670 | 709 | 670 | 710 |
| 670 | 711 | 670 | 712 | 670 | 713 | 670 | 714 |
| 670 | 715 | 670 | 716 | 670 | 717 | 670 | 718 |
| 671 | 704 | 671 | 705 | 671 | Del3* | 671 | 706 |
| 671 | 707 | 671 | 708 | 671 | 709 | 671 | 710 |
| 671 | 711 | 671 | 712 | 671 | 713 | 671 | 714 |
| 671 | 715 | 671 | 716 | 671 | 717 | 671 | 718 |
| 672 | 704 | 672 | 705 | 672 | Del3* | 672 | 706 |
| 672 | 707 | 672 | 708 | 672 | 709 | 672 | 710 |
| 672 | 711 | 672 | 712 | 672 | 713 | 672 | 714 |
| 672 | 715 | 672 | 716 | 672 | 717 | 672 | 718 |
| 673 | 704 | 673 | 705 | 673 | Del3* | 673 | 706 |
| 673 | 707 | 673 | 708 | 673 | 709 | 673 | 710 |
| 673 | 711 | 673 | 712 | 673 | 713 | 673 | 714 |
| 673 | 715 | 673 | 716 | 673 | 717 | 673 | 718 |
| 674 | 704 | 674 | 705 | 674 | Del3* | 674 | 706 |
| 674 | 707 | 674 | 708 | 674 | 709 | 674 | 710 |
| 674 | 711 | 674 | 712 | 674 | 713 | 674 | 714 |
| 674 | 715 | 674 | 716 | 674 | 717 | 674 | 718 |
| 675 | 704 | 675 | 705 | 675 | Del3* | 675 | 706 |
| 675 | 707 | 675 | 708 | 675 | 709 | 675 | 710 |
| 675 | 711 | 675 | 712 | 675 | 713 | 675 | 714 |
| 675 | 715 | 675 | 716 | 675 | 717 | 675 | 718 |
| 676 | 704 | 676 | 705 | 676 | Del3* | 676 | 706 |
| 676 | 707 | 676 | 708 | 676 | 709 | 676 | 710 |
| 676 | 711 | 676 | 712 | 676 | 713 | 676 | 714 |
| 676 | 715 | 676 | 716 | 676 | 717 | 676 | 718 |
| 677 | 704 | 677 | 705 | 677 | Del3* | 677 | 706 |
| 677 | 707 | 677 | 708 | 677 | 709 | 677 | 710 |
| 677 | 711 | 677 | 712 | 677 | 713 | 677 | 714 |
| 677 | 715 | 677 | 716 | 677 | 717 | 677 | 718 |
| 678 | 704 | 678 | 705 | 678 | Del3* | 678 | 706 |
| 678 | 707 | 678 | 708 | 678 | 709 | 678 | 710 |
| 678 | 711 | 678 | 712 | 678 | 713 | 678 | 714 |
| 678 | 715 | 678 | 716 | 678 | 717 | 678 | 718 |
| 679 | 704 | 679 | 705 | 679 | Del3* | 679 | 706 |
| 679 | 707 | 679 | 708 | 679 | 709 | 679 | 710 |
| 679 | 711 | 679 | 712 | 679 | 713 | 679 | 714 |
| 679 | 715 | 679 | 716 | 679 | 717 | 679 | 718 |
| 680 | 704 | 680 | 705 | 680 | Del3* | 680 | 706 |
| 680 | 707 | 680 | 708 | 680 | 709 | 680 | 710 |
| 680 | 711 | 680 | 712 | 680 | 713 | 680 | 714 |
| 680 | 715 | 680 | 716 | 680 | 717 | 680 | 718 |
| 681 | 704 | 681 | 705 | 681 | Del3* | 681 | 706 |
| 681 | 707 | 681 | 708 | 681 | 709 | 681 | 710 |
| 681 | 711 | 681 | 712 | 681 | 713 | 681 | 714 |
| 681 | 715 | 681 | 716 | 681 | 71 | 681 | 718 |
| 682 | 704 | 682 | 705 | 682 | Del3* | 682 | 706 |
| 682 | 707 | 682 | 708 | 682 | 709 | 682 | 710 |
| 682 | 711 | 682 | 712 | 682 | 713 | 682 | 714 |
| 682 | 715 | 682 | 716 | 682 | 717 | 682 | 718 |
| 683 | 704 | 683 | 705 | 683 | Del3* | 683 | 706 |
| 683 | 707 | 683 | 708 | 683 | 709 | 683 | 710 |
| 683 | 711 | 683 | 712 | 683 | 713 | 683 | 714 |
| 683 | 715 | 683 | 716 | 683 | 717 | 683 | 718 |
| 684 | 704 | 684 | 705 | 684 | Del3* | 684 | 706 |
| 684 | 707 | 684 | 708 | 684 | 709 | 684 | 710 |
| 684 | 711 | 684 | 712 | 684 | 713 | 684 | 714 |
| 684 | 715 | 684 | 716 | 684 | 717 | 684 | 718 |
| 685 | 704 | 685 | 705 | 685 | Del3* | 685 | 706 |
| 685 | 707 | 685 | 708 | 685 | 709 | 685 | 710 |
| 685 | 711 | 685 | 712 | 685 | 713 | 685 | 714 |
| 685 | 715 | 685 | 716 | 685 | 717 | 685 | 718 |
| 686 | 704 | 686 | 705 | 686 | Del3* | 686 | 706 |
| 686 | 707 | 686 | 708 | 686 | 709 | 686 | 710 |
| 686 | 711 | 686 | 712 | 686 | 713 | 686 | 714 |
| 686 | 715 | 686 | 716 | 686 | 717 | 686 | 718 |
| 687 | 704 | 687 | 705 | 687 | Del3* | 687 | 706 |
| 687 | 707 | 687 | 708 | 687 | 709 | 687 | 710 |
| 687 | 711 | 687 | 712 | 687 | 713 | 687 | 714 |
| 687 | 715 | 687 | 716 | 687 | 717 | 687 | 718 |
| 688 | 704 | 688 | 705 | 688 | Del3* | 688 | 706 |
| 688 | 707 | 688 | 708 | 688 | 709 | 688 | 710 |
| 688 | 711 | 688 | 712 | 688 | 713 | 688 | 714 |
| 688 | 715 | 688 | 716 | 688 | 717 | 688 | 718 |
| 689 | 704 | 689 | 705 | 689 | Del3* | 689 | 706 |
| 689 | 707 | 689 | 708 | 689 | 709 | 689 | 710 |
| 689 | 711 | 689 | 712 | 689 | 713 | 689 | 714 |
| 689 | 715 | 689 | 716 | 689 | 717 | 689 | 718 |
| 690 | 704 | 690 | 705 | 690 | Del3* | 690 | 706 |
| 690 | 707 | 690 | 708 | 690 | 709 | 690 | 710 |
| 690 | 711 | 690 | 712 | 690 | 713 | 690 | 714 |
| 690 | 715 | 690 | 716 | 690 | 717 | 690 | 718 |
| 691 | 704 | 691 | 705 | 691 | Del3* | 691 | 706 |
| 691 | 707 | 691 | 708 | 691 | 709 | 691 | 710 |
| 691 | 711 | 691 | 712 | 691 | 713 | 691 | 714 |
| 691 | 715 | 691 | 716 | 691 | 717 | 691 | 718 |
| 692 | 704 | 692 | 705 | 692 | Del3* | 692 | 706 |
| 692 | 707 | 692 | 708 | 692 | 709 | 692 | 710 |
| 692 | 711 | 692 | 712 | 692 | 713 | 692 | 714 |
| 692 | 715 | 692 | 716 | 692 | 717 | 692 | 718 |
| 693 | 704 | 693 | 705 | 693 | Del3* | 693 | 706 |
| 693 | 707 | 693 | 708 | 693 | 709 | 693 | 710 |
| 693 | 711 | 693 | 712 | 693 | 713 | 693 | 714 |
| 693 | 715 | 693 | 716 | 693 | 717 | 693 | 718 |
| 694 | 704 | 694 | 705 | 694 | Del3* | 694 | 706 |
| 694 | 707 | 694 | 708 | 694 | 709 | 694 | 710 |
| 694 | 711 | 694 | 712 | 694 | 713 | 694 | 714 |
| 694 | 715 | 694 | 716 | 694 | 717 | 694 | 718 |
| 695 | 704 | 695 | 705 | 695 | Del3* | 695 | 706 |
| 695 | 707 | 695 | 708 | 695 | 709 | 695 | 710 |
| 695 | 711 | 695 | 712 | 695 | 713 | 695 | 714 |
| 695 | 715 | 695 | 716 | 695 | 717 | 695 | 718 |
| 696 | 704 | 696 | 705 | 696 | Del3* | 696 | 706 |
| 696 | 707 | 696 | 708 | 696 | 709 | 696 | 710 |
| 696 | 711 | 696 | 712 | 696 | 713 | 696 | 714 |
| 696 | 715 | 696 | 716 | 696 | 717 | 696 | 718 |
| 697 | 704 | 697 | 705 | 697 | Del3* | 697 | 706 |
| 697 | 707 | 697 | 708 | 697 | 709 | 697 | 710 |
| 697 | 711 | 697 | 712 | 697 | 713 | 697 | 714 |
| 697 | 715 | 697 | 716 | 697 | 717 | 697 | 718 |
| 698 | 704 | 698 | 705 | 698 | Del3* | 698 | 706 |
| 698 | 707 | 698 | 708 | 698 | 709 | 698 | 710 |
| 698 | 711 | 698 | 712 | 698 | 713 | 698 | 714 |
| 698 | 715 | 698 | 716 | 698 | 717 | 698 | 718 |
| 699 | 704 | 699 | 705 | 699 | Del3* | 699 | 706 |
| 699 | 707 | 699 | 708 | 699 | 709 | 699 | 710 |
| 699 | 711 | 699 | 712 | 699 | 713 | 699 | 714 |
| 699 | 715 | 699 | 716 | 699 | 717 | 699 | 718 |
| 700 | 704 | 700 | 705 | 700 | Del3* | 700 | 706 |
| 700 | 707 | 700 | 708 | 700 | 709 | 700 | 710 |
| 700 | 711 | 700 | 712 | 700 | 713 | 700 | 714 |
| 700 | 715 | 700 | 716 | 700 | 717 | 700 | 718 |
| 701 | 704 | 701 | 705 | 701 | Del3* | 701 | 706 |
| 701 | 707 | 701 | 708 | 701 | 709 | 701 | 710 |
| 701 | 711 | 701 | 712 | 701 | 713 | 701 | 714 |
| 701 | 715 | 701 | 716 | 701 | 717 | 701 | 718 |
| 702 | 704 | 702 | 705 | 702 | Del3* | 702 | 706 |
| 702 | 707 | 702 | 708 | 702 | 709 | 702 | 710 |
| 702 | 711 | 702 | 712 | 702 | 713 | 702 | 714 |
| 702 | 715 | 702 | 716 | 702 | 717 | 702 | 718 |
| 703 | 704 | 703 | 705 | 703 | Del3* | 703 | 706 |
| 703 | 707 | 703 | 708 | 703 | 709 | 703 | 710 |
| 703 | 711 | 703 | 712 | 703 | 713 | 703 | 714 |
| 703 | 715 | 703 | 716 | 703 | 717 | 703 | 718 |
| 548 | 704 | 548 | 705 | 548 | Del3* | 548 | 706 |
| 548 | 707 | 548 | 708 | 548 | 709 | 548 | 710 |
| 548 | 711 | 548 | 712 | 548 | 713 | 548 | 714 |
| 548 | 715 | 548 | 716 | 548 | 717 | 548 | 718 |
| 549 | 704 | 549 | 705 | 549 | Del3* | 549 | 706 |
| 549 | 707 | 549 | 708 | 549 | 709 | 549 | 710 |
| 549 | 711 | 549 | 712 | 549 | 713 | 549 | 714 |
| 549 | 715 | 549 | 716 | 549 | 717 | 549 | 718 |
| 550 | 704 | 550 | 705 | 550 | Del3* | 550 | 706 |
| 550 | 707 | 550 | 708 | 550 | 709 | 550 | 710 |
| 550 | 711 | 550 | 712 | 550 | 713 | 550 | 714 |
| 550 | 715 | 550 | 716 | 550 | 717 | 550 | 718 |
| 551 | 704 | 551 | 705 | 551 | Del3* | 551 | 706 |
| 551 | 707 | 551 | 708 | 551 | 709 | 551 | 710 |
| 551 | 711 | 551 | 712 | 551 | 713 | 551 | 714 |
| 551 | 715 | 551 | 716 | 551 | 717 | 551 | 718 |
| 552 | 704 | 552 | 705 | 552 | Del3* | 552 | 706 |
| 552 | 707 | 552 | 708 | 552 | 709 | 552 | 710 |
| 552 | 711 | 552 | 712 | 552 | 713 | 552 | 714 |
| 552 | 715 | 552 | 716 | 552 | 717 | 552 | 718 |
| 553 | 704 | 553 | 705 | 553 | Del3* | 553 | 706 |
| 553 | 707 | 553 | 708 | 553 | 709 | 553 | 710 |
| 553 | 711 | 553 | 712 | 553 | 713 | 553 | 714 |
| 553 | 715 | 553 | 716 | 553 | 717 | 553 | 718 |
| 554 | 704 | 554 | 705 | 554 | Del3* | 554 | 706 |
| 554 | 707 | 554 | 708 | 554 | 709 | 554 | 710 |
| 554 | 711 | 554 | 712 | 554 | 713 | 554 | 714 |
| 554 | 715 | 554 | 716 | 554 | 717 | 554 | 718 |
| 555 | 704 | 555 | 705 | 555 | Del3* | 555 | 706 |
| 555 | 707 | 555 | 708 | 555 | 709 | 555 | 710 |
| 555 | 711 | 555 | 712 | 555 | 713 | 555 | 714 |
| 555 | 715 | 555 | 716 | 555 | 717 | 555 | 718 |
| 556 | 704 | 556 | 705 | 556 | Del3* | 556 | 706 |
| 556 | 707 | 556 | 708 | 556 | 709 | 556 | 710 |
| 556 | 711 | 556 | 712 | 556 | 713 | 556 | 714 |
| 556 | 715 | 556 | 716 | 556 | 717 | 556 | 718 |
| 557 | 704 | 557 | 705 | 557 | Del3* | 557 | 706 |
| 557 | 707 | 557 | 708 | 557 | 709 | 557 | 710 |
| 557 | 711 | 557 | 712 | 557 | 713 | 557 | 714 |
| 557 | 715 | 557 | 716 | 557 | 717 | 557 | 718 |
| 558 | 704 | 558 | 705 | 558 | Del3* | 558 | 706 |
| 558 | 707 | 558 | 708 | 558 | 709 | 558 | 710 |
| 558 | 711 | 558 | 712 | 558 | 713 | 558 | 714 |
| 558 | 715 | 558 | 716 | 558 | 717 | 558 | 718 |
| 559 | 704 | 559 | 705 | 559 | Del3* | 559 | 706 |
| 559 | 707 | 559 | 708 | 559 | 709 | 559 | 710 |
| 559 | 711 | 559 | 712 | 559 | 713 | 559 | 714 |
| 559 | 715 | 559 | 716 | 559 | 717 | 559 | 718 |
| 560 | 704 | 560 | 705 | 560 | Del3* | 560 | 706 |
| 560 | 707 | 560 | 708 | 560 | 709 | 560 | 710 |
| 560 | 711 | 560 | 712 | 560 | 713 | 560 | 714 |
| 560 | 715 | 560 | 716 | 560 | 717 | 560 | 718 |
| 561 | 704 | 561 | 705 | 561 | Del3* | 561 | 706 |
| 561 | 707 | 561 | 708 | 561 | 709 | 561 | 710 |
| 561 | 711 | 561 | 712 | 561 | 713 | 561 | 714 |
| 561 | 715 | 561 | 716 | 561 | 717 | 561 | 718 |
| 562 | 704 | 562 | 705 | 562 | Del3* | 562 | 706 |
| 562 | 707 | 562 | 708 | 562 | 709 | 562 | 710 |
| 562 | 711 | 562 | 712 | 562 | 713 | 562 | 714 |
| 562 | 715 | 562 | 716 | 562 | 717 | 562 | 718 |
| 563 | 704 | 563 | 705 | 563 | Del3* | 563 | 706 |
| 563 | 707 | 563 | 708 | 563 | 709 | 563 | 710 |
| 563 | 711 | 563 | 712 | 563 | 713 | 563 | 714 |
| 563 | 715 | 563 | 716 | 563 | 717 | 563 | 718 |
| 564 | 704 | 564 | 705 | 564 | Del3* | 564 | 706 |
| 564 | 707 | 564 | 708 | 564 | 709 | 564 | 710 |
| 564 | 711 | 564 | 712 | 564 | 713 | 564 | 714 |
| 564 | 715 | 564 | 716 | 564 | 717 | 564 | 718 |
| 565 | 704 | 565 | 705 | 565 | Del3* | 565 | 706 |
| 565 | 707 | 565 | 708 | 565 | 709 | 565 | 710 |
| 565 | 711 | 565 | 712 | 565 | 713 | 565 | 714 |
| 565 | 715 | 565 | 716 | 565 | 717 | 565 | 718 |
| 566 | 704 | 566 | 705 | 566 | Del3* | 566 | 706 |
| 566 | 707 | 566 | 708 | 566 | 709 | 566 | 710 |
| 566 | 711 | 566 | 712 | 566 | 713 | 566 | 714 |
| 566 | 715 | 566 | 716 | 566 | 717 | 566 | 718 |
| 567 | 704 | 567 | 705 | 567 | Del3* | 567 | 706 |
| 567 | 707 | 567 | 708 | 567 | 709 | 567 | 710 |
| 567 | 711 | 567 | 712 | 567 | 713 | 567 | 714 |
| 567 | 715 | 567 | 716 | 567 | 717 | 567 | 718 |
| 568 | 704 | 568 | 705 | 568 | Del3* | 568 | 706 |
| 568 | 707 | 568 | 708 | 568 | 709 | 568 | 710 |
| 568 | 711 | 568 | 712 | 568 | 713 | 568 | 714 |
| 568 | 715 | 568 | 716 | 568 | 717 | 568 | 718 |
| 569 | 704 | 569 | 705 | 569 | Del3* | 569 | 706 |
| 569 | 707 | 569 | 708 | 569 | 709 | 569 | 710 |
| 569 | 711 | 569 | 712 | 569 | 713 | 569 | 714 |
| 569 | 715 | 569 | 716 | 569 | 717 | 569 | 718 |
| 570 | 704 | 570 | 705 | 570 | Del3* | 570 | 706 |
| 570 | 707 | 570 | 708 | 570 | 709 | 570 | 710 |
| 570 | 711 | 570 | 712 | 570 | 713 | 570 | 714 |
| 570 | 715 | 570 | 716 | 570 | 717 | 570 | 718 |
| 571 | 704 | 571 | 705 | 571 | Del3* | 571 | 706 |
| 571 | 707 | 571 | 708 | 571 | 709 | 571 | 710 |
| 571 | 711 | 571 | 712 | 571 | 713 | 571 | 714 |
| 571 | 715 | 571 | 716 | 571 | 717 | 571 | 718 |
| 572 | 704 | 572 | 705 | 572 | Del3* | 572 | 706 |
| 572 | 707 | 572 | 708 | 572 | 709 | 572 | 710 |
| 572 | 711 | 572 | 712 | 572 | 713 | 572 | 714 |
| 572 | 715 | 572 | 716 | 572 | 717 | 572 | 718 |
| 573 | 704 | 573 | 705 | 573 | Del3* | 573 | 706 |
| 573 | 707 | 573 | 708 | 573 | 709 | 573 | 710 |
| 573 | 711 | 573 | 712 | 573 | 713 | 573 | 714 |
| 573 | 715 | 573 | 716 | 573 | 717 | 573 | 718 |
| 574 | 704 | 574 | 705 | 574 | Del3* | 574 | 706 |
| 574 | 707 | 574 | 708 | 574 | 709 | 574 | 710 |
| 574 | 711 | 574 | 712 | 574 | 713 | 574 | 714 |
| 574 | 715 | 574 | 716 | 574 | 717 | 574 | 718 |
| 575 | 704 | 575 | 705 | 575 | Del3* | 575 | 706 |
| 575 | 707 | 575 | 708 | 575 | 709 | 575 | 710 |
| 575 | 711 | 575 | 712 | 575 | 713 | 575 | 714 |
| 575 | 715 | 575 | 716 | 575 | 717 | 575 | 718 |
| 576 | 704 | 576 | 705 | 576 | Del3* | 576 | 706 |
| 576 | 707 | 576 | 708 | 576 | 709 | 576 | 710 |
| 576 | 711 | 576 | 712 | 576 | 713 | 576 | 714 |
| 576 | 715 | 576 | 716 | 576 | 717 | 576 | 718 |
| 577 | 704 | 577 | 705 | 577 | Del3* | 577 | 706 |
| 577 | 707 | 577 | 708 | 577 | 709 | 577 | 710 |
| 577 | 711 | 577 | 712 | 577 | 713 | 577 | 714 |
| 577 | 715 | 577 | 716 | 577 | 717 | 577 | 718 |
| 578 | 704 | 578 | 705 | 578 | Del3* | 578 | 706 |
| 578 | 707 | 578 | 708 | 578 | 709 | 578 | 710 |
| 578 | 711 | 578 | 712 | 578 | 713 | 578 | 714 |
| 578 | 715 | 578 | 716 | 578 | 717 | 578 | 718 |
| 579 | 704 | 579 | 705 | 579 | Del3* | 579 | 706 |
| 579 | 707 | 579 | 708 | 579 | 709 | 579 | 710 |
| 579 | 711 | 579 | 712 | 579 | 713 | 579 | 714 |
| 579 | 715 | 579 | 716 | 579 | 717 | 579 | 718 |
| 580 | 704 | 580 | 705 | 580 | Del3* | 580 | 706 |
| 580 | 707 | 580 | 708 | 580 | 709 | 580 | 710 |
| 580 | 711 | 580 | 712 | 580 | 713 | 580 | 714 |
| 580 | 715 | 580 | 716 | 580 | 717 | 580 | 718 |
| 581 | 704 | 581 | 705 | 581 | Del3* | 581 | 706 |
| 581 | 707 | 581 | 708 | 581 | 709 | 581 | 710 |
| 581 | 711 | 581 | 712 | 581 | 713 | 581 | 714 |
| 581 | 715 | 581 | 716 | 581 | 717 | 581 | 718 |
| 582 | 704 | 582 | 705 | 582 | Del3* | 582 | 706 |
| 582 | 707 | 582 | 708 | 582 | 709 | 582 | 710 |
| 582 | 711 | 582 | 712 | 582 | 713 | 582 | 714 |
| 582 | 715 | 582 | 716 | 582 | 717 | 582 | 718 |
| 583 | 704 | 583 | 705 | 583 | Del3* | 583 | 706 |
| 583 | 707 | 583 | 708 | 583 | 709 | 583 | 710 |
| 583 | 711 | 583 | 712 | 583 | 713 | 583 | 714 |
| 583 | 715 | 583 | 716 | 583 | 717 | 583 | 718 |
| 584 | 704 | 584 | 705 | 584 | Del3* | 584 | 706 |
| 584 | 707 | 584 | 708 | 584 | 709 | 584 | 710 |
| 584 | 711 | 584 | 712 | 584 | 713 | 584 | 714 |
| 584 | 715 | 584 | 716 | 584 | 717 | 584 | 718 |
| 585 | 704 | 585 | 705 | 585 | Del3* | 585 | 706 |
| 585 | 707 | 585 | 708 | 585 | 709 | 585 | 710 |
| 585 | 711 | 585 | 712 | 585 | 713 | 585 | 714 |
| 585 | 715 | 585 | 716 | 585 | 717 | 585 | 718 |
| 586 | 704 | 586 | 705 | 586 | Del3* | 586 | 706 |
| 586 | 707 | 586 | 708 | 586 | 709 | 586 | 710 |
| 586 | 711 | 586 | 712 | 586 | 713 | 586 | 714 |
| 586 | 715 | 586 | 716 | 586 | 717 | 586 | 718 |
| 587 | 704 | 587 | 705 | 587 | Del3* | 587 | 706 |
| 587 | 707 | 587 | 708 | 587 | 709 | 587 | 710 |
| 587 | 711 | 587 | 712 | 587 | 713 | 587 | 714 |
| 587 | 715 | 587 | 716 | 587 | 717 | 587 | 718 |
| 588 | 704 | 588 | 705 | 588 | Del3* | 588 | 706 |
| 588 | 707 | 588 | 708 | 588 | 709 | 588 | 710 |
| 588 | 711 | 588 | 712 | 588 | 713 | 588 | 714 |
| 588 | 715 | 588 | 716 | 588 | 717 | 588 | 718 |
| 589 | 704 | 589 | 705 | 589 | Del3* | 589 | 706 |
| 589 | 707 | 589 | 708 | 589 | 709 | 589 | 710 |
| 589 | 711 | 589 | 712 | 589 | 713 | 589 | 714 |
| 589 | 715 | 589 | 716 | 589 | 717 | 589 | 718 |
| 590 | 704 | 590 | 705 | 590 | Del3* | 590 | 706 |
| 590 | 707 | 590 | 708 | 590 | 709 | 590 | 710 |
| 590 | 711 | 590 | 712 | 590 | 713 | 590 | 714 |
| 590 | 715 | 590 | 716 | 590 | 717 | 590 | 718 |
| 591 | 704 | 591 | 705 | 591 | Del3* | 591 | 706 |
| 591 | 707 | 591 | 708 | 591 | 709 | 591 | 710 |
| 591 | 711 | 591 | 712 | 591 | 713 | 591 | 714 |
| 591 | 715 | 591 | 716 | 591 | 717 | 591 | 718 |
| 592 | 704 | 592 | 705 | 592 | Del3* | 592 | 706 |
| 592 | 707 | 592 | 708 | 592 | 709 | 592 | 710 |
| 592 | 711 | 592 | 712 | 592 | 713 | 592 | 714 |
| 592 | 715 | 592 | 716 | 592 | 717 | 592 | 718 |
| 593 | 704 | 593 | 705 | 593 | Del3* | 593 | 706 |
| 593 | 707 | 593 | 708 | 593 | 709 | 593 | 710 |
| 593 | 711 | 593 | 712 | 593 | 713 | 593 | 714 |
| 593 | 715 | 593 | 716 | 593 | 717 | 593 | 718 |
| 594 | 704 | 594 | 705 | 594 | Del3* | 594 | 706 |
| 594 | 707 | 594 | 708 | 594 | 709 | 594 | 710 |
| 594 | 711 | 594 | 712 | 594 | 713 | 594 | 714 |
| 594 | 715 | 594 | 716 | 594 | 717 | 594 | 718 |
| 595 | 704 | 595 | 705 | 595 | Del3* | 595 | 706 |
| 595 | 707 | 595 | 708 | 595 | 709 | 595 | 710 |
| 595 | 711 | 595 | 712 | 595 | 713 | 595 | 714 |
| 595 | 715 | 595 | 716 | 595 | 717 | 595 | 718 |
| 596 | 704 | 596 | 705 | 596 | Del3* | 596 | 706 |
| 596 | 707 | 596 | 708 | 596 | 709 | 596 | 710 |
| 596 | 711 | 596 | 712 | 596 | 713 | 596 | 714 |
| 596 | 715 | 596 | 716 | 596 | 717 | 596 | 718 |
| 597 | 704 | 597 | 705 | 597 | Del3* | 597 | 706 |
| 597 | 707 | 597 | 708 | 597 | 709 | 597 | 710 |
| 597 | 711 | 597 | 712 | 597 | 713 | 597 | 714 |
| 597 | 715 | 597 | 716 | 597 | 717 | 597 | 718 |
| 598 | 704 | 598 | 705 | 598 | Del3* | 598 | 706 |
| 598 | 707 | 598 | 708 | 598 | 709 | 598 | 710 |
| 598 | 711 | 598 | 712 | 598 | 713 | 598 | 714 |
| 598 | 715 | 598 | 716 | 598 | 717 | 598 | 718 |
| 599 | 704 | 599 | 705 | 599 | Del3* | 599 | 706 |
| 599 | 707 | 599 | 708 | 599 | 709 | 599 | 710 |
| 599 | 711 | 599 | 712 | 599 | 713 | 599 | 714 |
| 599 | 715 | 599 | 716 | 599 | 717 | 599 | 718 |
| 600 | 704 | 600 | 705 | 600 | Del3* | 600 | 706 |
| 600 | 707 | 600 | 708 | 600 | 709 | 600 | 710 |
| 600 | 711 | 600 | 712 | 600 | 713 | 600 | 714 |
| 600 | 715 | 600 | 716 | 600 | 717 | 600 | 718 |
| 601 | 704 | 601 | 705 | 601 | Del3* | 601 | 706 |
| 601 | 707 | 601 | 708 | 601 | 709 | 601 | 710 |
| 601 | 711 | 601 | 712 | 601 | 713 | 601 | 714 |
| 601 | 715 | 601 | 716 | 601 | 717 | 601 | 718 |
| 602 | 704 | 602 | 705 | 602 | Del3* | 602 | 706 |
| 602 | 707 | 602 | 708 | 602 | 709 | 602 | 710 |
| 602 | 711 | 602 | 712 | 602 | 713 | 602 | 714 |
| 602 | 715 | 602 | 716 | 602 | 717 | 602 | 718 |
| 603 | 704 | 603 | 705 | 603 | Del3* | 603 | 706 |
| 603 | 707 | 603 | 708 | 603 | 709 | 603 | 710 |
| 603 | 711 | 603 | 712 | 603 | 713 | 603 | 714 |
| 603 | 715 | 603 | 716 | 603 | 717 | 603 | 718 |
| 604 | 704 | 604 | 705 | 604 | Del3* | 604 | 706 |
| 604 | 707 | 604 | 708 | 604 | 709 | 604 | 710 |
| 604 | 711 | 604 | 712 | 604 | 713 | 604 | 714 |
| 604 | 715 | 604 | 716 | 604 | 717 | 604 | 718 |
| 605 | 704 | 605 | 705 | 605 | Del3* | 605 | 706 |
| 605 | 707 | 605 | 708 | 605 | 709 | 605 | 710 |
| 605 | 711 | 605 | 712 | 605 | 713 | 605 | 714 |
| 605 | 715 | 605 | 716 | 605 | 717 | 605 | 718 |
| 606 | 704 | 606 | 705 | 606 | Del3* | 606 | 706 |
| 606 | 707 | 606 | 708 | 606 | 709 | 606 | 710 |
| 606 | 711 | 606 | 712 | 606 | 713 | 606 | 714 |
| 606 | 715 | 606 | 716 | 606 | 717 | 606 | 718 |
| 612 | 704 | 612 | 705 | 612 | Del3* | 612 | 706 |
| 612 | 707 | 612 | 708 | 612 | 709 | 612 | 710 |
| 612 | 711 | 612 | 712 | 612 | 713 | 612 | 714 |
| 612 | 715 | 612 | 716 | 612 | 717 | 612 | 718 |
| 613 | 704 | 613 | 705 | 613 | Del3* | 613 | 706 |
| 613 | 707 | 613 | 708 | 613 | 709 | 613 | 710 |
| 613 | 711 | 613 | 712 | 613 | 713 | 613 | 714 |
| 613 | 715 | 613 | 716 | 613 | 717 | 613 | 718 |
| 614 | 704 | 614 | 705 | 614 | Del3* | 614 | 706 |
| 614 | 707 | 614 | 708 | 614 | 709 | 614 | 710 |
| 614 | 711 | 614 | 712 | 614 | 713 | 614 | 714 |
| 614 | 715 | 614 | 716 | 614 | 717 | 614 | 718 |
| 615 | 704 | 615 | 705 | 615 | Del3* | 615 | 706 |
| 615 | 707 | 615 | 708 | 615 | 709 | 615 | 710 |
| 615 | 711 | 615 | 712 | 615 | 713 | 615 | 714 |
| 615 | 715 | 615 | 716 | 615 | 717 | 615 | 718 |
| 616 | 704 | 616 | 705 | 616 | Del3* | 616 | 706 |
| 616 | 707 | 616 | 708 | 616 | 709 | 616 | 710 |
| 616 | 711 | 616 | 712 | 616 | 713 | 616 | 714 |
| 616 | 715 | 616 | 716 | 616 | 717 | 616 | 718 |
| 617 | 704 | 617 | 705 | 617 | Del3* | 617 | 706 |
| 617 | 707 | 617 | 708 | 617 | 709 | 617 | 710 |
| 617 | 711 | 617 | 712 | 617 | 713 | 617 | 714 |
| 617 | 715 | 617 | 716 | 617 | 717 | 617 | 718 |
| 618 | 704 | 618 | 705 | 618 | Del3* | 618 | 706 |
| 618 | 707 | 618 | 708 | 618 | 709 | 618 | 710 |
| 618 | 711 | 618 | 712 | 618 | 713 | 618 | 714 |
| 618 | 715 | 618 | 716 | 618 | 717 | 618 | 718 |
| 619 | 704 | 619 | 705 | 619 | Del3* | 619 | 706 |
| 619 | 707 | 619 | 708 | 619 | 709 | 619 | 710 |
| 619 | 711 | 619 | 712 | 619 | 713 | 619 | 714 |
| 619 | 715 | 619 | 716 | 619 | 717 | 619 | 718 |
| 620 | 704 | 620 | 705 | 620 | Del3* | 620 | 706 |
| 620 | 707 | 620 | 708 | 620 | 709 | 620 | 710 |
| 620 | 711 | 620 | 712 | 620 | 713 | 620 | 714 |
| 620 | 715 | 620 | 716 | 620 | 717 | 620 | 718 |
| 621 | 704 | 621 | 705 | 621 | Del3* | 621 | 706 |
| 621 | 707 | 621 | 708 | 621 | 709 | 621 | 710 |
| 621 | 711 | 621 | 712 | 621 | 713 | 621 | 714 |
| 621 | 715 | 621 | 716 | 621 | 717 | 621 | 718 |
| 622 | 704 | 622 | 705 | 622 | Del3* | 622 | 706 |
| 622 | 707 | 622 | 708 | 622 | 709 | 622 | 710 |
| 622 | 711 | 622 | 712 | 622 | 713 | 622 | 714 |
| 622 | 715 | 622 | 716 | 622 | 717 | 622 | 718 |
| 623 | 704 | 623 | 705 | 623 | Del3* | 623 | 706 |
| 623 | 707 | 623 | 708 | 623 | 709 | 623 | 710 |
| 623 | 711 | 623 | 712 | 623 | 713 | 623 | 714 |
| 623 | 715 | 623 | 716 | 623 | 717 | 623 | 718 |
| 624 | 704 | 624 | 705 | 624 | Del3* | 624 | 706 |
| 624 | 707 | 624 | 708 | 624 | 709 | 624 | 710 |
| 624 | 711 | 624 | 712 | 624 | 713 | 624 | 714 |
| 624 | 715 | 624 | 716 | 624 | 717 | 624 | 718 |
| 625 | 704 | 625 | 705 | 625 | Del3* | 625 | 706 |
| 625 | 707 | 625 | 708 | 625 | 709 | 625 | 710 |
| 625 | 711 | 625 | 712 | 625 | 713 | 625 | 714 |
| 625 | 715 | 625 | 716 | 625 | 717 | 625 | 718 |
| 626 | 704 | 626 | 705 | 626 | Del3* | 626 | 706 |
| 626 | 707 | 626 | 708 | 626 | 709 | 626 | 710 |
| 626 | 711 | 626 | 712 | 626 | 713 | 626 | 714 |
| 626 | 715 | 626 | 716 | 626 | 717 | 626 | 718 |
| 627 | 704 | 627 | 705 | 627 | Del3* | 627 | 706 |
| 627 | 707 | 627 | 708 | 627 | 709 | 627 | 710 |
| 627 | 711 | 627 | 712 | 627 | 713 | 627 | 714 |
| 627 | 715 | 627 | 716 | 627 | 717 | 627 | 718 |
| 628 | 704 | 628 | 705 | 628 | Del3* | 628 | 706 |
| 628 | 707 | 628 | 708 | 628 | 709 | 628 | 710 |
| 628 | 711 | 628 | 712 | 628 | 713 | 628 | 714 |
| 628 | 715 | 628 | 716 | 628 | 717 | 628 | 718 |
| 629 | 704 | 629 | 705 | 629 | Del3* | 629 | 706 |
| 629 | 707 | 629 | 708 | 629 | 709 | 629 | 710 |
| 629 | 711 | 629 | 712 | 629 | 713 | 629 | 714 |
| 629 | 715 | 629 | 716 | 629 | 717 | 629 | 718 |
| 630 | 704 | 630 | 705 | 630 | Del3* | 630 | 706 |
| 630 | 707 | 630 | 708 | 630 | 709 | 630 | 710 |
| 630 | 711 | 630 | 712 | 630 | 713 | 630 | 714 |
| 630 | 715 | 630 | 716 | 630 | 717 | 630 | 718 |
| 631 | 704 | 631 | 705 | 631 | Del3* | 631 | 706 |
| 631 | 707 | 631 | 708 | 631 | 709 | 631 | 710 |
| 631 | 711 | 631 | 712 | 631 | 713 | 631 | 714 |
| 631 | 715 | 631 | 716 | 631 | 717 | 631 | 718 |
| 391 | 704 | 391 | 705 | 391 | Del3* | 391 | 706 |
| 391 | 707 | 391 | 708 | 391 | 709 | 391 | 710 |
| 391 | 711 | 391 | 712 | 391 | 713 | 391 | 714 |
| 391 | 715 | 391 | 716 | 391 | 717 | 391 | 718 |
| 392 | 704 | 392 | 705 | 392 | Del3* | 392 | 706 |
| 392 | 707 | 392 | 708 | 392 | 709 | 392 | 710 |
| 392 | 711 | 392 | 712 | 392 | 713 | 392 | 714 |
| 392 | 715 | 392 | 716 | 392 | 717 | 392 | 718 |
| 393 | 704 | 393 | 705 | 393 | Del3* | 393 | 706 |
| 393 | 707 | 393 | 708 | 393 | 709 | 393 | 710 |
| 393 | 711 | 393 | 712 | 393 | 713 | 393 | 714 |
| 393 | 715 | 393 | 716 | 393 | 717 | 393 | 718 |
| 394 | 704 | 394 | 705 | 394 | Del3* | 394 | 706 |
| 394 | 707 | 394 | 708 | 394 | 709 | 394 | 710 |
| 394 | 711 | 394 | 712 | 394 | 713 | 394 | 714 |
| 394 | 715 | 394 | 716 | 394 | 717 | 394 | 718 |
| 395 | 704 | 395 | 705 | 395 | Del3* | 395 | 706 |
| 395 | 707 | 395 | 708 | 395 | 709 | 395 | 710 |
| 395 | 711 | 395 | 712 | 395 | 713 | 395 | 714 |
| 395 | 715 | 395 | 716 | 395 | 717 | 395 | 718 |
| 396 | 704 | 396 | 705 | 396 | Del3* | 396 | 706 |
| 396 | 707 | 396 | 708 | 396 | 709 | 396 | 710 |
| 396 | 711 | 396 | 712 | 396 | 713 | 396 | 714 |
| 396 | 715 | 396 | 716 | 396 | 717 | 396 | 718 |
| 397 | 704 | 397 | 705 | 397 | Del3* | 397 | 706 |
| 397 | 707 | 397 | 708 | 397 | 709 | 397 | 710 |
| 397 | 711 | 397 | 712 | 397 | 713 | 397 | 714 |
| 397 | 715 | 397 | 716 | 397 | 717 | 397 | 718 |
| 398 | 704 | 398 | 705 | 398 | Del3* | 398 | 706 |
| 398 | 707 | 398 | 708 | 398 | 709 | 398 | 710 |
| 398 | 711 | 398 | 712 | 398 | 713 | 398 | 714 |
| 398 | 715 | 398 | 716 | 398 | 717 | 398 | 718 |
| 399 | 704 | 399 | 705 | 399 | Del3* | 399 | 706 |
| 399 | 707 | 399 | 708 | 399 | 709 | 399 | 710 |
| 399 | 711 | 399 | 712 | 399 | 713 | 399 | 714 |
| 399 | 715 | 399 | 716 | 399 | 717 | 399 | 718 |
| 400 | 704 | 400 | 705 | 400 | Del3* | 400 | 706 |
| 400 | 707 | 400 | 708 | 400 | 709 | 400 | 710 |
| 400 | 711 | 400 | 712 | 400 | 713 | 400 | 714 |
| 400 | 715 | 400 | 716 | 400 | 717 | 400 | 718 |
| 401 | 704 | 401 | 705 | 401 | Del3* | 401 | 706 |
| 401 | 707 | 401 | 708 | 401 | 709 | 401 | 710 |
| 401 | 711 | 401 | 712 | 401 | 713 | 401 | 714 |
| 401 | 715 | 401 | 716 | 401 | 717 | 401 | 718 |
| 402 | 704 | 402 | 705 | 402 | Del3* | 402 | 706 |
| 402 | 707 | 402 | 708 | 402 | 709 | 402 | 710 |
| 402 | 711 | 402 | 712 | 402 | 713 | 402 | 714 |
| 402 | 715 | 402 | 716 | 402 | 717 | 402 | 718 |
| 403 | 704 | 403 | 705 | 403 | Del3* | 403 | 706 |
| 403 | 707 | 403 | 708 | 403 | 709 | 403 | 710 |
| 403 | 711 | 403 | 712 | 403 | 713 | 403 | 714 |
| 403 | 715 | 403 | 716 | 403 | 717 | 403 | 718 |
| 404 | 704 | 404 | 705 | 404 | Del3* | 404 | 706 |
| 404 | 707 | 404 | 708 | 404 | 709 | 404 | 710 |
| 404 | 711 | 404 | 712 | 404 | 713 | 404 | 714 |
| 404 | 715 | 404 | 716 | 404 | 717 | 404 | 718 |
| 405 | 704 | 405 | 705 | 405 | Del3* | 405 | 706 |
| 405 | 707 | 405 | 708 | 405 | 709 | 405 | 710 |
| 405 | 711 | 405 | 712 | 405 | 713 | 405 | 714 |
| 405 | 715 | 405 | 716 | 405 | 717 | 405 | 718 |
| 406 | 704 | 406 | 705 | 406 | Del3* | 406 | 706 |
| 406 | 707 | 406 | 708 | 406 | 709 | 406 | 710 |
| 406 | 711 | 406 | 712 | 406 | 713 | 406 | 714 |
| 406 | 715 | 406 | 716 | 406 | 717 | 406 | 718 |
| 407 | 704 | 407 | 705 | 407 | Del3* | 407 | 706 |
| 407 | 707 | 407 | 708 | 407 | 709 | 407 | 710 |
| 407 | 711 | 407 | 712 | 407 | 713 | 407 | 714 |
| 407 | 715 | 407 | 716 | 407 | 717 | 407 | 718 |
| 408 | 704 | 408 | 705 | 408 | Del3* | 408 | 706 |
| 408 | 707 | 408 | 708 | 408 | 709 | 408 | 710 |
| 408 | 711 | 408 | 712 | 408 | 713 | 408 | 714 |
| 408 | 715 | 408 | 716 | 408 | 717 | 408 | 718 |
| 409 | 704 | 409 | 705 | 409 | Del3* | 409 | 706 |
| 409 | 707 | 409 | 708 | 409 | 709 | 409 | 710 |
| 409 | 711 | 409 | 712 | 409 | 713 | 409 | 714 |
| 409 | 715 | 409 | 716 | 409 | 717 | 409 | 718 |
| 410 | 704 | 410 | 705 | 410 | Del3* | 410 | 706 |
| 410 | 707 | 410 | 708 | 410 | 709 | 410 | 710 |
| 410 | 711 | 410 | 712 | 410 | 713 | 410 | 714 |
| 410 | 715 | 410 | 716 | 410 | 717 | 410 | 718 |
| 411 | 704 | 411 | 705 | 411 | Del3* | 411 | 706 |
| 411 | 707 | 411 | 708 | 411 | 709 | 411 | 710 |
| 411 | 711 | 411 | 712 | 411 | 713 | 411 | 714 |
| 411 | 715 | 411 | 716 | 411 | 717 | 411 | 718 |
| 412 | 704 | 412 | 705 | 412 | Del3* | 412 | 706 |
| 412 | 707 | 412 | 708 | 412 | 709 | 412 | 710 |
| 412 | 711 | 412 | 712 | 412 | 713 | 412 | 714 |
| 412 | 715 | 412 | 716 | 412 | 717 | 412 | 718 |
| 413 | 704 | 413 | 705 | 413 | Del3* | 413 | 706 |
| 413 | 707 | 413 | 708 | 413 | 709 | 413 | 710 |
| 413 | 711 | 413 | 712 | 413 | 713 | 413 | 714 |
| 413 | 715 | 413 | 716 | 413 | 717 | 413 | 718 |
| 414 | 704 | 414 | 705 | 414 | Del3* | 414 | 706 |
| 414 | 707 | 414 | 708 | 414 | 709 | 414 | 710 |
| 414 | 711 | 414 | 712 | 414 | 713 | 414 | 714 |
| 414 | 715 | 414 | 716 | 414 | 717 | 414 | 718 |
| 415 | 704 | 415 | 705 | 415 | Del3* | 415 | 706 |
| 415 | 707 | 415 | 708 | 415 | 709 | 415 | 710 |
| 415 | 711 | 415 | 712 | 415 | 713 | 415 | 714 |
| 415 | 715 | 415 | 716 | 415 | 717 | 415 | 718 |
| 416 | 704 | 416 | 705 | 416 | Del3* | 416 | 706 |
| 416 | 707 | 416 | 708 | 416 | 709 | 416 | 710 |
| 416 | 711 | 416 | 712 | 416 | 713 | 416 | 714 |
| 416 | 715 | 416 | 716 | 416 | 717 | 416 | 718 |
| 417 | 704 | 417 | 705 | 417 | Del3* | 417 | 706 |
| 417 | 707 | 417 | 708 | 417 | 709 | 417 | 710 |
| 417 | 711 | 417 | 712 | 417 | 713 | 417 | 714 |
| 417 | 715 | 417 | 716 | 417 | 717 | 417 | 718 |
| 418 | 704 | 418 | 705 | 418 | Del3* | 418 | 706 |
| 418 | 707 | 418 | 708 | 418 | 709 | 418 | 710 |
| 418 | 711 | 418 | 712 | 418 | 713 | 418 | 714 |
| 418 | 715 | 418 | 716 | 418 | 717 | 418 | 718 |
| 419 | 704 | 419 | 705 | 419 | Del3* | 419 | 706 |
| 419 | 707 | 419 | 708 | 419 | 709 | 419 | 710 |
| 419 | 711 | 419 | 712 | 419 | 713 | 419 | 714 |
| 419 | 715 | 419 | 716 | 419 | 717 | 419 | 718 |
| 420 | 704 | 420 | 705 | 420 | Del3* | 420 | 706 |
| 420 | 707 | 420 | 708 | 420 | 709 | 420 | 710 |
| 420 | 711 | 420 | 712 | 420 | 713 | 420 | 714 |
| 420 | 715 | 420 | 716 | 420 | 717 | 420 | 718 |
| 421 | 704 | 421 | 705 | 421 | Del3* | 421 | 706 |
| 421 | 707 | 421 | 708 | 421 | 709 | 421 | 710 |
| 421 | 711 | 421 | 712 | 421 | 713 | 421 | 714 |
| 421 | 715 | 421 | 716 | 421 | 717 | 421 | 718 |
| 422 | 704 | 422 | 705 | 422 | Del3* | 422 | 706 |
| 422 | 707 | 422 | 708 | 422 | 709 | 422 | 710 |
| 422 | 711 | 422 | 712 | 422 | 713 | 422 | 714 |
| 422 | 715 | 422 | 716 | 422 | 717 | 422 | 718 |
| 423 | 704 | 423 | 705 | 423 | Del3* | 423 | 706 |
| 423 | 707 | 423 | 708 | 423 | 709 | 423 | 710 |
| 423 | 711 | 423 | 712 | 423 | 713 | 423 | 714 |
| 423 | 715 | 423 | 716 | 423 | 717 | 423 | 718 |
| 424 | 704 | 424 | 705 | 424 | Del3* | 424 | 706 |
| 424 | 707 | 424 | 708 | 424 | 709 | 424 | 710 |
| 424 | 711 | 424 | 712 | 424 | 713 | 424 | 714 |
| 424 | 715 | 424 | 716 | 424 | 717 | 424 | 718 |
| 425 | 704 | 425 | 705 | 425 | Del3* | 425 | 706 |
| 425 | 707 | 425 | 708 | 425 | 709 | 425 | 710 |
| 425 | 711 | 425 | 712 | 425 | 713 | 425 | 714 |
| 425 | 715 | 425 | 716 | 425 | 717 | 425 | 718 |
| 426 | 704 | 426 | 705 | 426 | Del3* | 426 | 706 |
| 426 | 707 | 426 | 708 | 426 | 709 | 426 | 710 |
| 426 | 711 | 426 | 712 | 426 | 713 | 426 | 714 |
| 426 | 715 | 426 | 716 | 426 | 717 | 426 | 718 |

*Del3 indicates a deletion of the region spanning EU positions 216-230 (see Table 10).

### Polypeptides

In another aspect, provided herein are polypeptides comprising one or more sequences set forth in Tables 1-7, 12, or 13, above. In certain embodiments, the polypeptide comprises the CDRH1, CDRH2, and/or CDRH3 of a VH amino acid sequence set forth in any one of SEQ ID NOs: 1-84, as determined by any of the methods discussed above. In certain embodiments, the polypeptide comprises a VH comprising the CDRH1, CDRH2, and CDRH3 amino acid sequences of the VH amino acid sequence set forth in any one of SEQ ID NOs: 1-84. In certain embodiments, the polypeptide comprises the CDRH1, CDRH2, and CDRH3 amino acid sequences set forth in SEQ ID NOs: 374, 384, and 141; 354, 355, and 87; 354, 355, and 90; 354, 357, and 93; 354, 357, and 96; 354, 355, and 96; 354, 355, and 99; 354, 357, and 99; 354, 355, and 102; 354, 359, and 102; 354, 359, and 105; 354, 355, and 105; 354, 357, and 105; 354, 355, and 108; 354, 357, and 108; 354, 357, and 111; 354, 357, and 114; 354, 355, and 114; 354, 357, and 117; 354, 355, and 117; 354, 357, and 120; 354, 357, and 123; 354, 357, and 125; 354, 357, and 127; 354, 357, and 129; 354, 357, and 131; 354, 357, and 133; 354, 357, and 135; 354, 357, and 137; 354, 357, and 139; 354, 357, and 141; 354, 355, and 143; 354, 355, and 145; 354, 355, and 147; 354, 355, and 149; 354, 355, and 151; 354, 355, and 152; 354, 355, and 153; 356, 361, and 117; 358, 361, and 117; 360, 363, and 117; 362, 365, and 117; 364, 367, and 133; 366, 367, and 133; 368, 369, and 133; 370, 371, and 133; 354, 373, and 108; 354, 375, and 117; 354, 373, and 120; 354, 373, and 129; 354, 373, and 131; 354, 373, and 133; 354, 373, and 139; 354, 355, and 154; 372, 355, and 87; 354, 377, and 87; 354, 355, and 155; 354, 373, and 141; 374, 379, and 141; 374, 380, and 141; 374, 381, and 141; 374, 382, and 141; 374, 383, and 141; 374, 385, and 131; 374, 380, and 131; 374, 379, and 131; 376, 385, and 131; 376, 380, and 131; 376, 379, and 131; 378, 385, and 131; or 378, 379, and 131, respectively. In certain embodiments, the polypeptide comprises the CDRH1, CDRH2, and CDRH3 amino acid sequences set forth in SEQ ID NOs: 115, 144, and 141; 85, 86, and 87; 85, 86, and 90; 85, 89, and 93; 85, 89, and 96; 85, 86, and 96; 85, 86, and 99; 85, 89, and 99; 85, 86, and 102; 85, 92, and 102; 85, 92, and 105; 85, 86, and 105; 85, 89, and 105; 85, 86, and 108; 85, 89, and 108; 85, 89, and 111; 85, 89, and 114; 85, 86, and 114; 85, 89, and 117; 85, 86, and 117; 85, 89, and 120; 85, 89, and 123; 85, 89, and 125; 85, 89, and 127; 85, 89, and 129; 85, 89, and 131; 85, 89, and 133; 85, 89, and 135; 85, 89, and 137; 85, 89, and 139; 85, 89, and 141; 85, 86, and 143; 85, 86, and 145; 85, 86, and 147; 85, 86, and 149; 85, 86, and 151; 85, 86, and 152; 85, 86, and 153; 88, 95, and 117; 91, 98, and 117; 94, 101, and 117; 97, 104, and 117; 100, 107, and 133; 103, 110, and 133; 106, 113, and 133; 109, 116, and 133; 85, 119, and 108; 85, 122, and 108; 85, 124, and 117; 85, 126, and 117; 85, 119, and 120; 85, 122, and 120; 85, 119, and 129; 85, 122, and 129; 85, 119, and 131; 85, 122, and 131; 85, 119, and 133; 85, 122, and 133; 85, 119, and 139; 85, 122, and 139; 85, 86, and 154; 112, 86, and 87; 85, 128, and 87; 85, 86, and 155; 85, 130, and 141; 85, 132, and 141; 115, 134, and 141; 115, 136, and 141; 115, 138, and 141; 115, 140, and 141; 115, 142, and 141; 115, 146, and 131; 115, 148, and 131; 115, 150, and 131; 118, 146, and 131; 118, 148, and 131; 118, 150, and 131; 121, 146, and 131; or 121, 150, and 131, respectively. In certain embodiments, the polypeptide does not comprise the amino acid sequence set forth in any one of SEQ ID NOs: 60-64 and 823-830. In certain embodiments, the polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence set forth in Table 12, above.

In certain embodiments, a polypeptide provided herein comprises the CDRH1, CDRH2, and/or CDRH3 of a VH amino acid sequence set forth in any one of SEQ ID NOs: 156-191, as determined by any of the methods discussed above. In certain embodiments, the polypeptide comprises a VH comprising the CDRH1, CDRH2, and CDRH3 amino acid sequences of the VH amino acid sequence set forth in any one of SEQ ID NOs: 156-191. In certain embodiments, the polypeptide comprises the CDRH1, CDRH2, and CDRH3 amino acid sequences set forth in SEQ ID NOs: 429, 430, and 243; 427, 428, and 194; 427, 428, and 197; 429, 430, and 200; 429, 430, and 203; 429, 430, and 206; 429, 430, and 209; 431, 428, and 212; 431, 428, and 215; 433, 428, and 218; 433, 428, and 221; 433, 428, and 224; 433, 428, and 227; 435, 432, and 230; 437, 434, and 232; 439, 428, and 234; 433, 428, and 235; 441, 436, and 236; 441, 436, and 237; 443, 438, and 238; 445, 440, and 239; 447, 442, and 240; 431, 428, and 241; 431, 428, and 242; 448, 444, and 244; 449, 428, and 218; 427, 428, and 218; 427, 446, and 218; 433, 428, and 245; 433, 428, and 246; 449, 428, and 245; 449, 428, and 246; or 431, 428, and 245, respectively. In certain embodiments, the polypeptide comprises the CDRH1, CDRH2, and CDRH3 amino acid sequences set forth in SEQ ID NOs: 195, 834, and 243; 192, 833, and 194; 192, 833, and 197; 195, 834, and 200; 195, 834, and 203; 195, 834, and 206; 195, 834, and 209; 198, 835, and 212; 198, 835, and 215; 201, 836, and 218; 201, 836, and 221; 201, 836, and 224; 201, 836, and 227; 204, 837, and 230; 207, 838, and 232; 210, 836, and 234; 201, 836, and 235; 213, 839, and 236; 213, 839, and 237; 216, 840, and 238; 219, 841, and 239; 222, 842, and 240; 198, 835, and 241; 198, 835, and 242; 225, 843, and 244; 228, 836, and 218; 192, 836, and 218; 192, 844, and 218; 192, 845, and 218; 192, 846, and 218; 192, 847, and 218; 201, 836, and 245; 201, 836, and 246; 228, 836, and 245; 228, 836, and 246; or 198, 836, and 245, respectively. In certain embodiments, the polypeptide comprises the CDRH1, CDRH2, and CDRH3 amino acid sequences set forth in SEQ ID NOs: 195, 196, and 243; 192, 193, and 194; 192, 193, and 197; 195, 196, and 200; 195, 196, and 203; 195, 196, and 206; 195, 196, and 209; 198, 199, and 212; 198, 199, and 215; 201, 202, and 218; 201, 202, and 221; 201, 202, and 224; 201, 202, and 227; 204, 205, and 230; 207, 208, and 232; 202, 210, and 234; 201, 202, and 235; 211, 213, and 236; 211, 213, and 237; 214, 216, and 238; 217, 219, and 239; 220, 222, and 240; 198, 199, and 241; 198, 199, and 242; 223, 225, and 244; 202, 218, and 228; 192, 202, and 218; 192, 218, and 226; 192, 218, and 229; 192, 218, and 231; 192, 218, and 233; 201, 202, and 245; 201, 202, and 246; 202, 228, and 245; 202, 228, and 246; or 198, 202, and 245, respectively. In certain embodiments, the polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence set forth in Table 13, above.

In another aspect, provided herein are polypeptides comprising one or more sequences set forth in Tables 10 or 11, above. In certain embodiments, the polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of the amino acid sequences set forth in SEQ ID NOs: 704-750. In certain embodiments, the polypeptide comprises or consists of any one of the amino acid sequences set forth in SEQ ID NOs: 704-750. In certain embodiments, the polypeptide comprises an IgG Fc comprising any one of the amino acid sequences set forth in SEQ ID NOs: 704-750.

### Pharmaceutical Compositions

The present disclosure provides pharmaceutical compositions comprising the anti-APJ antibodies and/or peptides described herein and/or the nucleic acid molecules and/or expression vectors that encode them. The pharmaceutical compositions described herein are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also, Powell et al., “Compendium of excipients for parenteral formulations” PDA (1998) J Pharm Sci Technol 52:238-311.

The dose of anti-APJ antibodies and/or peptides described herein and/or nucleic acid molecules and/or expression vectors that encode them administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering the anti-APJ antibodies and/or peptides described herein and/or nucleic acid molecules and/or expression vectors that encode them may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition disclosed herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Any pharmaceutical composition described herein can be delivered subcutaneously or intravenously with a standard needle and syringe. In addition, with respect to subcutaneous delivery, a pen delivery device readily has applications in delivering a pharmaceutical composition disclosed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see, Langer, supra; Sefton, 1987, CRC Crit. Ref Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending, or emulsifying any of the antibodies described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

In a specific embodiment, pharmaceutical compositions provided herein comprise anti-APJ antibodies and/or peptides described herein and/or nucleic acid molecules and/or expression vectors that encode them, and optionally one or more additional prophylactic or therapeutic agents, in a pharmaceutically acceptable carrier. In a specific embodiment, pharmaceutical compositions comprise an anti-APJ antibody disclosed herein, and optionally one or more additional prophylactic or therapeutic agents, in a pharmaceutically acceptable carrier. In certain embodiments, the antibody is the only active ingredient included in the pharmaceutical composition. Pharmaceutical compositions described herein can be useful in decreasing or blocking APJ (e.g., human APJ) activity and treating an APJ-associated disease or disorder. In certain embodiments, the present disclosure relates to a pharmaceutical composition of the present disclosure comprising anti-APJ antibodies and/or peptides described herein and/or nucleic acid molecules and/or expression vectors that encode them for use as a medicament. In another embodiment, the present disclosure relates to a pharmaceutical composition of the present disclosure for use in a method for the treatment of an APJ-associated disease or disorder.

### Methods of Use

### Monotherapy

In another aspect, the instant disclosure provides a method of treating a subject using an antibody that specifically binds to human APJ (e.g., an antibody or polypeptide disclosed herein). In certain embodiments, the subject has or is suspected of having an APJ-associated disease or disorder. As used herein, the term “APJ-associated disease or disorder” refers to a disease, disorder, or condition that is caused by, or associated with, APJ protein production and/or APJ protein activity. The term “APJ-associated disease or disorder” includes a disease, disorder or condition that would benefit from an increase in or a decrease in APJ protein activity.

In certain embodiments, the APJ-associated disease or disorder is characterized by overexpression of APJ (e.g., human APJ). In certain embodiments, the APJ-associated disease or disorder is characterized by dysregulation of one or more APJ functions.

In certain embodiments, the APJ-associated disease or disorder is associated with pathological angiogenesis. In certain embodiments, the APJ-associated disease or disorder is associated with angiodysplasia, abnormal vessel formation, hypervascularization, arteriovenous malformation (AVM) formation, and/or excessive bleeding. In certain embodiments, the APJ-associated disease or disorder is associated with insulin regulation.

In certain aspects, provided herein are methods of treating an APJ-associated disease or disorder in a subject, the methods comprising administering to the subject an effective amount of antibody that specifically binds to human APJ, a polynucleotide encoding the antibody, a vector comprising the polynucleotide, a recombinant host cell comprising the polynucleotide or the vector, and/or a composition comprising the antibody, polynucleotide, vector, and/or recombinant host cell and a pharmaceutically acceptable carrier or excipient. In certain aspects, provided herein are methods of treating an APJ-associated disease or disorder in a subject, the methods comprising administering to the subject an effective amount of an anti-APJ antibody, polynucleotide, vector, host cell, or composition described herein. In certain embodiments, the APJ-associated disease or disorder is hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and/or autosomal dominant polycystic kidney disease (ADPKD).

In certain embodiments, the APJ-associated disease or disorder is HHT (e.g., HHT1, HHT2, HHT3, HHT4, HHT5, or JP-HHT). In certain embodiments, the APJ-associated disease or disorder is angiodysplasia, AVM, brain AVM, bleeding, and/or telangiectasia, and the subject has been diagnosed with HHT (e.g., HHT1, HHT2, HHT3, HHT4, HHT5, or JP-HHT). In certain embodiments, the APJ-associated disease or disorder is telangiectasia, and the subject has been diagnosed with pulmonary hypertension. In certain embodiments, the APJ-associated disease or disorder is telangiectasia, the subject has been diagnosed with pulmonary hypertension, and the subject has been treated with and/or is being treated with sotatarcept. In certain embodiments, the APJ-associated disease or disorder is heart failure, acute decompensated heart failure, and/or congestive heart failure, and the subject has been treated with and/or is being treated with a left ventricular assist device (LVAD). In certain embodiments, the LVAD is a continuous-flow LVAD.

In certain embodiments, the APJ-associated disease or disorder is a vascular eye disease or disorder selected from diabetic retinopathy, proliferative diabetic retinopathy, diabetic macular edema, macular degeneration, age-related macular degeneration, wet age-related macular degeneration, geographic atrophy, retinal neovascularization, central retinal vein occlusion, branched retinal vein occlusion, polypoidal choroidal vasculopathy, choroidal neovascularization (CNV), degenerative myopia (myopic CNV), neovascular glaucoma, and retinopathy of prematurity.

In certain embodiments, the APJ-associated disease or disorder is stroke, and administration of the antibody, polynucleotide, vector, host cell, and/or composition prevents or delays the stroke.

In certain embodiments, the APJ-associated disease or disorder is idiopathic PAH, heritable PAH, toxin- or drug-induced PAH, and/or PAH associated with one or more of the following: congenital heart disease, connective tissue disorders (e.g., scleroderma, systemic lupus erythematosus, systemic sclerosis, Hashimoto's thyroiditis, Sjögren's Syndrome, and antiphospholipid antibody syndrome), portal hypertension, a BMPR2 mutation, Schistosomiasis, and HIV infection. In certain embodiments, the APJ-associated disease or disorder is fibrosis associated with an organ or tissue selected from lung, liver, heart, mediastinum, bone marrow, retroperitoneum, skin, intestine, joint, a reproductive organ, and a combination thereof. In certain embodiments, the APJ-associated disease or disorder is a connective tissue disorder selected from scleroderma, systemic lupus erythematosus, systemic sclerosis, Hashimoto's thyroiditis, Sjögren's Syndrome, and antiphospholipid antibody syndrome.

In certain embodiments, the APJ-associated disease or disorder is any one of the diseases or disorders described in International Publication Nos. WO2019169193A1; WO2020073011A1; and WO2024099382A1, which are herein incorporated by reference in their entireties.

In certain embodiments, administration of an anti-APJ antibody described herein to a subject results in a reduction or inhibition of an APJ function in the subject. In such embodiments, the anti-APJ antibody can be considered an antagonist antibody. In certain embodiments, the APJ function is reduced or inhibited by at least 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art, relative to the APJ function in a subject to which the antagonist antibody has not been administered. In certain embodiments, the APJ function is reduced or inhibited by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein and/or known to one of skill in the art, relative to the APJ function in a subject to which the antagonist antibody has not been administered. Non-limiting examples of APJ functions include APJ signaling, APJ binding to ligands (e.g., apelin, elabela), cyclic AMP production, and β-arrestin production. In certain embodiments, reduction or inhibition of a function of APJ is assessed as described in the Examples herein.

In certain aspects, provided herein are methods of treating an APJ-associated disease or disorder in a subject, the methods comprising administering to the subject an effective amount of an antagonist anti-APJ antibody described herein. In certain embodiments, the APJ-associated disease or disorder is any of those listed above. In certain embodiments, the antagonist anti-APJ antibody comprises a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in any one of SEQ ID NOs: 1-59, 66-84, or 156-191. In certain embodiments, the antagonist anti-APJ antibody comprises a VH comprising an amino acid sequence set forth in any one of SEQ ID NOs: 1-59, 66-84, or 156-191. In certain embodiments, the amino acid sequence of the antagonist anti-APJ antibody VH consists of the amino acid sequence set forth in any one of SEQ ID NOs: 1-59, 66-84, or 156-191.

In certain embodiments, provided herein is a method of treating an APJ-associated disease or disorder in a subject, the method comprising administering to the subject an effective amount of antibody that specifically binds to human APJ, a polynucleotide encoding the antibody, a vector comprising the polynucleotide, a recombinant host cell comprising the polynucleotide or the vector, and/or a composition comprising the antibody, polynucleotide, vector, and/or recombinant host cell and a pharmaceutically acceptable carrier or excipient, wherein the antibody is an APJ antagonist antibody, and wherein the APJ-associated disease or disorder is selected from hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and/or autosomal dominant polycystic kidney disease (ADPKD). In certain embodiments, the APJ antagonist antibody comprises a VH comprising an amino acid sequence selected from the group consisting of SEQ ID NOs: 1-59, 66-84, and 156-191.

In certain embodiments, provided herein is a method of treating an APJ-associated disease or disorder in a subject, the method comprising administering to the subject an effective amount of antibody that specifically binds to human APJ, a polynucleotide encoding the antibody, a vector comprising the polynucleotide, a recombinant host cell comprising the polynucleotide or the vector, and/or a composition comprising the antibody, polynucleotide, vector, and/or recombinant host cell and a pharmaceutically acceptable carrier or excipient, wherein the antibody is an APJ antagonist antibody, and wherein the APJ-associated disease or disorder is selected from hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD). In certain embodiments, the APJ antagonist antibody comprises a VH comprising an amino acid sequence selected from the group consisting of SEQ ID NOs: 1-59, 66-84, and 156-191.

In certain embodiments, administration of an anti-APJ antibody described herein to a subject results in an increase of an APJ function in the subject. In such embodiments, the anti-APJ antibody can be considered an agonist antibody. In certain embodiments, the APJ function is increased by at least 5%, 10%, 15%, 20%, 25%, 30%, 35%, 40%, 45%, 50%, 55%, 60%, 65%, 70%, 75%, 80%, 85%, 90%, 95%, 98%, 99%, 100%, or more as assessed by methods described herein and/or known to one of skill in the art, relative to the APJ function in a subject to which the agonist antibody has not been administered. In certain embodiments, the APJ function is increased by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or more as assessed by methods described herein and/or known to one of skill in the art, relative to the APJ function in a subject to which the agonist antibody has not been administered. In certain embodiments, increase of a function of APJ is assessed as described in the Examples herein.

In certain aspects, provided herein are methods of treating an APJ-associated disease or disorder in a subject, the methods comprising administering to the subject an effective amount of an agonist anti-APJ antibody described herein. In certain embodiments, the APJ-associated disease or disorder is any of those listed above. In certain embodiments, the APJ-associated disease or disorder is pulmonary arterial hypertension (PAH), obesity, and/or muscle-sparing obesity. In certain embodiments, the agonist anti-APJ antibody comprises a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 65. In certain embodiments, the agonist anti-APJ antibody comprises a VH comprising an amino acid sequence set forth SEQ ID NO: 65. In certain embodiments, the amino acid sequence of the agonist anti-APJ antibody VH consists of the amino acid sequence set forth in SEQ ID NO: 65.

In certain embodiments, provided herein is a method of treating an APJ-associated disease or disorder in a subject, the method comprising administering to the subject an effective amount of antibody that specifically binds to human APJ, a polynucleotide encoding the antibody, a vector comprising the polynucleotide, a recombinant host cell comprising the polynucleotide or the vector, and/or a composition comprising the antibody, polynucleotide, vector, and/or recombinant host cell and a pharmaceutically acceptable carrier or excipient, wherein the antibody is an APJ agonist antibody, and wherein the APJ-associated disease or disorder is selected from hereditary hemorrhagic telangiectasia (HHT) (e.g., hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), or juvenile polyposis/hereditary hemorrhagic telangiectasia (JP-HHT)), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and/or autosomal dominant polycystic kidney disease (ADPKD). In certain embodiments, the APJ agonist antibody comprises a VH comprising the amino acid sequence set forth in SEQ ID NO: 65.

In certain embodiments, provided herein is a method of treating an APJ-associated disease or disorder in a subject, the method comprising administering to the subject an effective amount of antibody that specifically binds to human APJ, a polynucleotide encoding the antibody, a vector comprising the polynucleotide, a recombinant host cell comprising the polynucleotide or the vector, and/or a composition comprising the antibody, polynucleotide, vector, and/or recombinant host cell and a pharmaceutically acceptable carrier or excipient, wherein the antibody is an APJ agonist antibody, and wherein the APJ-associated disease or disorder is selected from obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD). In certain embodiments, the APJ agonist antibody comprises a VH comprising the amino acid sequence set forth in SEQ ID NO: 65.

Administration of the compositions according to the methods described herein may result in a reduction of the severity, signs, symptoms, or markers of an APJ-associated disease or disorder in a patient with an APJ-associated disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

The present disclosure also provides methods of increasing or decreasing an APJ-related activity in a primary cell, comprising contacting the primary cell with an anti-APJ antibody described herein. In certain embodiments, contacting the primary cell with the anti-APJ antibody results in enhanced APJ activity in the cell, e.g., as described above. In certain embodiments, contacting the primary cell with the anti-APJ antibody results in decreased APJ activity in the cell, e.g., as described above. In certain embodiments, the primary cell is within a subject.

### Combination Therapies and Formulations

The present disclosure also provides compositions and therapeutic formulations comprising the anti-APJ antibodies and/or polypeptides described herein and/or the nucleic acid molecules and/or the expression vectors that encode them in combination with one or more additional therapeutically active components (i.e., therapeutic agents), and methods of treatment comprising administering such combinations to subjects in need thereof.

Exemplary additional therapeutic agents include any therapeutic agents that may be used for the treatment of any APJ-related diseases or disorders described herein. In certain embodiments, the additional therapeutic agents may be used for the treatment of hereditary hemorrhagic telangiectasia. In certain embodiments, the additional therapeutic agent is an antifibrinolytic agent (e.g., tranexamic acid). In certain embodiments, the additional therapeutic agent is an anti-angiogenic agent. In certain embodiments, the anti-angiogenic agent is a VEGF pathway inhibitor (e.g., bevacizumab, nintedanib, pazopanib, thalidomide, or other immunomodulators), an mTOR inhibitor (e.g., sirolimus), a calcineurin inhibitor (e.g., tacrolimus), an anti-Ang2 antibody (e.g., LC10, faricimab), a CDK4/6 inhibitor (e.g., palbociclib, ribociclib), a PI3K inhibitor (e.g., Buparlisib), and/or an AKT inhibitor (e.g., VAD044). In certain embodiments, the additional therapeutic agent is intravenous iron replacement. In certain embodiments, the additional therapeutic agent is desmopressin. In certain embodiments, the additional therapeutic agent is recombinant von Willebrand factor. In certain embodiments, the additional therapeutic agent is an oral anticoagulant (e.g., heparin, warfarin).

The additional therapeutically active component(s) may be administered just prior to, concurrent with, or shortly after the administration of the anti-APJ antibodies and/or polypeptides described herein and/or the nucleic acid molecules and/or the expression vectors that encode them.

The present disclosure provides pharmaceutical compositions in which the anti-APJ antibodies and/or polypeptides described herein and/or the nucleic acid molecules and/or the expression vectors that encode them are co-formulated with one or more of the additional therapeutically active component(s) as described elsewhere herein.

### Administration Regimens

The anti-APJ antibodies and/or polypeptides described herein and/or the nucleic acid molecules and/or the expression vectors that encode them may be delivered to a subject by a variety of routes. These include, but are not limited to, parenteral, intranasal, intratracheal, oral, intradermal, topical, intramuscular, intraperitoneal, transdermal, intravenous, intratumoral, conjunctival, intra-arterial, and subcutaneous routes. Pulmonary administration can also be employed, e.g., by use of an inhaler or nebulizer, and formulation with an aerosolizing agent for use as a spray. In certain embodiments, the anti-APJ antibodies and/or polypeptides described herein and/or the nucleic acid molecules and/or the expression vectors that encode them are delivered subcutaneously or intravenously. In certain embodiments, the anti-APJ antibodies and/or polypeptides described herein and/or the nucleic acid molecules and/or the expression vectors that encode them are delivered intra-arterially. In certain embodiments, the anti-APJ antibodies and/or polypeptides described herein and/or the nucleic acid molecules and/or the expression vectors that encode them are delivered intratumorally. In certain embodiments, the anti-APJ antibodies and/or polypeptides described herein and/or the nucleic acid molecules and/or the expression vectors that encode them are delivered into a tumor draining lymph node.

The amount of anti-APJ antibodies and/or polypeptides described herein and/or the nucleic acid molecules and/or the expression vectors that encode them which will be effective in the treatment and/or prevention of a condition will depend on the nature of the disease and can be determined by standard clinical techniques.

The precise dose to be employed in a composition will also depend on the route of administration, and the seriousness of the infection or disease caused by it and should be decided according to the judgment of the practitioner and each subject's circumstances. For example, effective doses may also vary depending upon means of administration, target site, physiological state of the patient (including age, body weight, and health), whether the patient is human or an animal, other medications administered, or whether treatment is prophylactic or therapeutic. Usually, the patient is a human, but non-human mammals, including transgenic mammals, can also be treated. Treatment dosages are optimally titrated to optimize safety and efficacy.

### Other Uses

An anti-APJ antibody described herein can also be used to assay APJ (e.g., human APJ) protein levels in a biological sample using classical immunohistological methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (¹²⁵I, ¹²¹I) carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labels can be used to label an antibody described herein. Alternatively, a second antibody that recognizes an anti-APJ antibody described herein can be labeled and used in combination with an anti-APJ antibody to detect APJ (e.g., human APJ) protein levels. Therefore, in certain embodiments, the present disclosure relates to the use of an anti-APJ antibody of the present disclosure for in vitro detection of APJ (e.g., human APJ) protein in a biological sample. In a further embodiment, the present disclosure relates to the use of an anti-APJ antibody of the disclosure, for assaying and/or detecting APJ (e.g., human APJ) protein levels in a biological sample in vitro, optionally wherein the anti-APJ antibody is conjugated to a radionuclide or detectable label, and/or carries a label described herein, and/or wherein an immunohistological method is used.

Assaying for the expression level of APJ (e.g., human APJ) protein is intended to include qualitatively or quantitatively measuring or estimating the level of APJ (e.g., human APJ) protein in a first biological sample either directly (e.g., by determining or estimating absolute protein level) or relatively (e.g., by comparing to the disease associated protein level in a second biological sample). APJ (e.g., human APJ) polypeptide expression level in the first biological sample can be measured or estimated and compared to a standard APJ (e.g., human APJ) protein level, the standard being taken, for example, from a second biological sample obtained from an individual not having the disorder or being determined by averaging levels from a population of individuals not having the disorder. As will be appreciated in the art, once the “standard” APJ (e.g., human APJ) polypeptide level is known, it can be used repeatedly as a standard for comparison. Therefore, in a further embodiment, the present disclosure relates to an in vitro method for assaying and/or detecting APJ protein levels, for example human APJ protein levels, in a biological sample, comprising qualitatively or quantitatively measuring or estimating the level of APJ protein, for example of human APJ protein, in a biological sample, by an immunohistological method.

As used herein, the term “biological sample” refers to any biological sample obtained from a subject, cell line, tissue, or other source of cells potentially expressing APJ (e.g., human APJ). Methods for obtaining tissue biopsies and body fluids from animals (e.g., humans or cynomolgus monkeys) are well known in the art. Biological samples include peripheral blood mononuclear cells (PBMCs).

An anti-APJ antibody described herein can be used for prognostic, diagnostic, monitoring, and screening applications, including in vitro and in vivo applications well known and standard to the skilled artisan and based on the present description. Prognostic, diagnostic, monitoring, and screening assays and kits for in vitro assessment and evaluation of APJ-associated diseases or disorders may be utilized to predict, diagnose, and monitor to evaluate patient samples, including those known to have or suspected of having an APJ-associated disease or disorder. Therefore, in certain embodiments, the present disclosure relates to an anti-APJ antibody described herein for use as a diagnostic. In certain embodiments, the present disclosure relates to an anti-APJ antibody described herein for use in a method for the prediction, diagnosis, and/or monitoring of a subject having or suspected to have an APJ-associated disease or disorder.

### Polynucleotides, Vectors, and Methods of Producing Polypeptides

In another aspect, provided herein are polynucleotides encoding an anti-APJ antibody or polypeptide described herein or a fragment thereof and vectors comprising such polynucleotides. In certain embodiments, the polynucleotides and vectors are useful for recombinant expression of the anti-APJ antibody or polypeptide in host cells (e.g., E. coli and mammalian cells). Also provided herein are recombinant host cells comprising a polynucleotide and/or a vector described herein.

In a specific embodiment, a nucleic acid molecule(s) encoding a polypeptide (e.g., antibody) described herein is isolated or purified.

In certain aspects, provided herein are polynucleotides encoding an anti-APJ antibody or polypeptide described herein. The polynucleotides can comprise nucleotide sequences encoding a heavy chain comprising the FRs and CDRs of an anti-APJ antibody or polypeptide described herein (see, e.g., Tables 1-7). In certain embodiments, a polynucleotide provided herein encodes a VH of an anti-APJ antibody described herein. In certain embodiments, a polynucleotide provided herein encodes a heavy chain constant region or a portion thereof (e.g., a portion comprising an Ig Fc) described herein. In certain embodiments, a polynucleotide provided herein encodes an anti-APJ antibody described herein (see, e.g., Tables 12 and 13).

Also provided herein are polynucleotides encoding an anti-APJ antibody or polypeptide described herein that are optimized, e.g., by codon/RNA optimization, replacement with heterologous signal sequences, and elimination of mRNA instability elements. Methods to generate optimized nucleic acids encoding an anti-APJ antibody or polypeptide described herein or a fragment thereof (e.g., a VH or a heavy chain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of an anti-APJ antibody or polypeptide described herein or a fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-APJ antibody or polypeptide described herein or a fragment thereof encoded by polynucleotides that have not been optimized.

In certain embodiments, an optimized polynucleotide sequence encoding an anti-APJ antibody or polypeptide described herein or a fragment thereof (e.g., a VH or a heavy chain) can hybridize to an antisense (e.g., complementary) polynucleotide of an unoptimized polynucleotide sequence encoding an anti-APJ antibody or polypeptide described herein or a fragment thereof (e.g., a VH or a heavy chain). In specific embodiments, an optimized nucleotide sequence encoding an anti-APJ antibody or polypeptide described herein or a fragment thereof (e.g., a VH or a heavy chain) hybridizes under high stringency conditions to antisense polynucleotide of an unoptimized polynucleotide sequence encoding an anti-APJ antibody or polypeptide described herein or a fragment thereof (e.g., a VH or a heavy chain). In a specific embodiment, an optimized nucleotide sequence encoding an anti-APJ antibody or polypeptide described herein or a fragment thereof (e.g., a VH or a heavy chain) hybridizes under high stringency, intermediate or lower stringency hybridization conditions to an antisense polynucleotide of an unoptimized nucleotide sequence encoding an anti-APJ antibody or polypeptide described herein or a fragment thereof (e.g., a VH or a heavy chain). Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding anti-APJ antibodies or polypeptides described herein and modified versions of these polypeptides can be determined using methods well known in the art, i.e., nucleotide codons known to encode particular amino acids are assembled in such a way to generate a nucleic acid that encodes the polypeptide. Such a polynucleotide encoding the polypeptide (e.g., antibody) can be assembled from chemically synthesized oligonucleotides (e.g., as described in Kutmeier G et al., (1994), BioTechniques 17: 242-6, herein incorporated by reference in its entirety), which, briefly, involves the synthesis of overlapping oligonucleotides containing portions of the sequence encoding the polypeptide, annealing, and ligating of those oligonucleotides, and then amplification of the ligated oligonucleotides by PCR.

Alternatively, a polynucleotide encoding an antigen-binding region of anti-APJ antibody or polypeptide described herein can be generated from nucleic acids from a suitable source using methods well known in the art (e.g., PCR and other molecular cloning methods). Such PCR amplification methods can be used to obtain nucleic acids comprising the sequence encoding the anti-APJ antibody or polypeptide described herein or a fragment thereof (e.g., a VH or a heavy chain). The amplified nucleic acids can be cloned into vectors for expression in host cells and for further cloning.

Also provided are polynucleotides that hybridize under high stringency, intermediate or lower stringency hybridization conditions to polynucleotides that encode an anti-APJ antibody or polypeptide described herein. In specific embodiments, polynucleotides described herein hybridize under high stringency, intermediate or lower stringency hybridization conditions to polynucleotides encoding an anti-APJ antibody or polypeptide provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In certain aspects, provided herein are cells (e.g., host cells) expressing (e.g., recombinantly) an anti-APJ antibody or polypeptide described herein or a fragment thereof, and related polynucleotides and expression vectors. In certain embodiments, the host cells are recombinant host cells. In certain embodiments, the recombinant host cells comprise any of the polynucleotides and/or vectors described above. Also provided herein are vectors (e.g., expression vectors) comprising polynucleotides comprising nucleotide sequences encoding an anti-APJ antibody or polypeptide described herein or a fragment thereof (e.g., a VH or a heavy chain) for recombinant expression in host cells, e.g., in mammalian cells. In certain embodiments, the vectors comprise any of the polynucleotides described above. Also provided herein are host cells comprising such vectors for recombinantly expressing an anti-APJ antibody or polypeptide described herein or a fragment thereof (e.g., a VH or a heavy chain). In a particular aspect, provided herein are methods for producing an anti-APJ antibody or polypeptide described herein, comprising culturing a host cell described herein under suitable conditions such that the polynucleotide or vector is expressed and the antibody is produced.

Recombinant expression of an anti-APJ antibody or polypeptide described generally involves construction of an expression vector containing a polynucleotide that encodes the polypeptide. Once a polynucleotide encoding an anti-APJ antibody or polypeptide described herein or a fragment thereof (e.g., a VH or a heavy chain) has been obtained, the vector for the production of the polypeptide can be produced by recombinant DNA technology using techniques well known in the art. Thus, methods for preparing a protein by expressing a polynucleotide containing a nucleotide sequence encoding an anti-APJ antibody or polypeptide described herein or a fragment thereof (e.g., a VH or a heavy chain) are described herein. Methods which are well known to those skilled in the art can be used to construct expression vectors containing a nucleotide sequence encoding an anti-APJ antibody or polypeptide described herein or a fragment thereof (e.g., a VH or a heavy chain) and appropriate transcriptional and translational control signals. These methods include, for example, in vitro recombinant DNA techniques, synthetic techniques, and in vivo genetic recombination. Also provided are replicable vectors comprising a nucleotide sequence encoding a polypeptide (e.g., antibody) described herein, a heavy chain antibody, a VH of an antibody or a fragment thereof, or a heavy chain CDR, operably linked to a promoter. Such vectors can, for example, include the nucleotide sequence encoding the constant region of the anti-APJ antibody or polypeptide (see, e.g., International Publication Nos. WO 86/05807 and WO 89/01036; and U.S. Pat. No. 5,122,464, which are herein incorporated by reference in their entireties) and variable regions of the anti-APJ antibody or polypeptide can be cloned into such a vector for expression of the entire heavy chain.

In certain embodiments, a vector comprises a polynucleotide encoding a VH or a heavy chain of an anti-APJ antibody or polypeptide described herein.

An expression vector can be transferred to a cell (e.g., host cell) by conventional techniques and the resulting cells can then be cultured by conventional techniques to produce an anti-APJ antibody or polypeptide described herein or a fragment thereof (e.g., a VH or a heavy chain). Thus, provided herein are host cells containing a polynucleotide encoding containing an anti-APJ antibody or polypeptide described herein or a fragment thereof (e.g., a VH or a heavy chain), operably linked to a promoter for expression of such sequences in the host cell.

In certain embodiments, a host cell comprises a polynucleotide encoding an anti-APJ antibody or polypeptide described herein or a fragment thereof (e.g., a VH or a heavy chain). In another embodiment, a host cell comprises a vector comprising a polynucleotide encoding an anti-APJ antibody or polypeptide described herein or a fragment thereof (e.g., a VH or a heavy chain).

A variety of host-expression vector systems can be utilized to express polypeptides (e.g., antibodies) described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express a polypeptide (e.g., antibody) described herein in situ. These include but are not limited to microorganisms such as bacteria (e.g., E. coli and B. subtilis) transformed with, e.g., recombinant bacteriophage DNA, plasmid DNA or cosmid DNA expression vectors containing polypeptide (e.g., antibody) coding sequences; yeast (e.g., Saccharomyces and Pichia) transformed with, e.g., recombinant yeast expression vectors containing polypeptide (e.g., antibody) coding sequences; insect cell systems infected with, e.g., recombinant virus expression vectors (e.g., baculovirus) containing polypeptide (e.g., antibody) coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing polypeptide (e.g., antibody) coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, cells for expressing polypeptides (e.g., antibodies) described herein are human cells, e.g., human cell lines. In a specific embodiment, a mammalian expression vector is pOptiVEC™ or pcDNA3.3. In certain embodiments, bacterial cells such as Escherichia coli, or eukaryotic cells (e.g., mammalian cells) are used for the expression of a recombinant polypeptide (e.g., antibody) molecule. For example, mammalian cells such as CHO cells, in conjunction with a vector such as the major intermediate early gene promoter element from human cytomegalovirus, are an effective expression system for polypeptides (e.g., antibodies) (Foecking M K & Hofstetter H (1986) Gene 45: 101-5; and Cockett M I et al., (1990) Biotechnology 8(7): 662-7, each of which is herein incorporated by reference in its entirety). In certain embodiments, polypeptides (e.g., antibodies) described herein are produced by CHO cells or NS0 cells. In a specific embodiment, the expression of nucleotide sequences encoding an anti-APJ antibody or polypeptide described herein or a fragment thereof (e.g., a VH or a heavy chain) is regulated by a constitutive promoter, inducible promoter, or tissue specific promoter.

In bacterial systems, a number of expression vectors can be advantageously selected depending upon the use intended for the polypeptide (e.g., antibody) being expressed. For example, when a large quantity of such a polypeptide (e.g., antibody) is to be produced, for the generation of pharmaceutical compositions of a polypeptide, vectors which direct the expression of high levels of polypeptide products that are readily purified can be desirable. Such vectors include, but are not limited to, the E. coli expression vector pUR278 (Ruether U & Mueller-Hill B (1983) EMBO J 2: 1791-1794), in which the coding sequence can be ligated individually into the vector in frame with the lac Z coding region so that a polypeptide (e.g., antibody) is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (see, e.g., Logan J & Shenk T (1984) PNAS 81(12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, and HsS78Bst cells. In certain embodiments, an anti-APJ antibody or polypeptide described herein or a fragment thereof (e.g., a VH or a heavy chain) described herein is produced in mammalian cells, such as CHO cells.

In a specific embodiment, the polypeptides (e.g., antibodies) described herein have reduced fucose content or no fucose content. Such polypeptides (e.g., antibodies) can be produced using techniques known one skilled in the art. For example, the polypeptides (e.g., antibodies) can be expressed in cells deficient or lacking the ability to fucosylate. In a specific example, cell lines with a knockout of both alleles of al,6-fucosyltransferase can be used to produce polypeptides (e.g., antibodies) with reduced fucose content.

For long-term, high-yield production of recombinant proteins, stable expression cells can be generated. For example, cell lines which stably express an anti-APJ antibody or polypeptide described herein or a fragment thereof (e.g., a VH or a heavy chain) can be engineered. In specific embodiments, a cell provided herein stably expresses an anti-APJ antibody or polypeptide described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-APJ antibody or polypeptide described herein or a fragment thereof (e.g., a VH or a heavy chain). Such engineered cell lines can be particularly useful in screening and evaluation of compositions that interact directly or indirectly with the polypeptide (e.g., antibody).

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034), and adenine phosphoribosyltransferase (Lowy I et al., (1980) Cell 22(3): 817-23) genes in tk-, hgprt-or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbere-Garapin F et al., (1981) J Mol Biol 150: 1-14, all of which are herein incorporated by reference in their entireties.

The expression levels of a polypeptide (e.g., antibody) can be increased by vector amplification (for a review, see Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system is amplifiable, increase in the level of inhibitor present in culture of host cell will increase the number of copies of the marker gene. Since the amplified region is associated with the gene of interest, production of the protein will also increase (Crouse G F et al., (1983) Mol Cell Biol 3: 257-66, which is herein incorporated by reference in its entirety).

Once a polypeptide (e.g., antibody) described herein has been produced by recombinant expression, it can be purified by any method known in the art for purification of a polypeptide (e.g., antibody), for example, by chromatography (e.g., ion exchange, affinity, particularly by affinity for the specific antigen after Protein A, and sizing column chromatography), centrifugation, differential solubility, or by any other standard technique for the purification of proteins. Further, the polypeptides (e.g., antibodies) described herein can be fused to heterologous polypeptide sequences described herein or otherwise known in the art to facilitate purification.

In specific embodiments, a polypeptide (e.g., antibody) described herein is isolated or purified.

An anti-APJ antibody or polypeptide described herein or a fragment thereof (e.g., a VH or a heavy chain) can be produced by any method known in the art for the synthesis of proteins, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In a specific embodiment, a polypeptide (e.g., antibody) described herein is prepared, expressed, created, or isolated by any means that involves creation, e.g., via synthesis, genetic engineering of DNA sequences. In certain embodiments, such a polypeptide (e.g., antibody) comprises sequences (e.g., DNA sequences or amino acid sequences) that do not naturally exist within the antibody germline repertoire of an animal or mammal (e.g., human) in vivo.

In one aspect, provided herein is a method of making an anti-APJ antibody or polypeptide comprising culturing a cell or host cell described herein. In certain embodiments, the method is performed in vitro. In a certain aspect, provided herein is a method of making an anti-APJ antibody or polypeptide comprising expressing (e.g., recombinantly expressing) the polypeptide using a cell or host cell described herein (e.g., a cell or a host cell comprising polynucleotides encoding a polypeptide described herein). In certain embodiments, the cell is an isolated cell. In certain embodiments, the exogenous polynucleotides have been introduced into the cell. In certain embodiments, the method further comprises the step of purifying the polypeptide (e.g., antibody) obtained from the cell or host cell. In certain embodiments, a polypeptide (e.g., antibody) is produced by expressing in a cell a polynucleotide encoding an anti-APJ antibody or polypeptide described herein or a fragment thereof (e.g., a VH or a heavy chain) under suitable conditions so that the polynucleotides are expressed and the polypeptide is produced.

In certain embodiments, an anti-APJ antibody or polypeptide described herein or a fragment thereof (e.g., a VH or a heavy chain) is produced recombinantly from host cells exogenously expressing the polypeptide.

Methods for making multispecific polypeptides (e.g., bispecific antibodies) have been described, see, e.g., U.S. Pat. Nos. 7,951,917; 7,183,076; 8,227,577; 5,837,242; 5,989,830; 5,869,620; 6,132,992; and 8,586,713, all of which are herein incorporated by reference in their entireties.

Bispecific, bivalent polypeptides (e.g., antibodies), and methods of making them, are described, for instance in U.S. Pat. Nos. 5,731,168, 5,807,706, 5,821,333, and U.S. Appl. Publ. Nos. 2003/020734 and 2002/0155537; each of which is herein incorporated by reference in its entirety. Bispecific tetravalent antibodies, and methods of making them are described, for instance, in Int. Appl. Publ. Nos. WO 02/096948 and WO 00/44788, the disclosures of both of which are herein incorporated by reference in its entirety. See, generally, Int. Appl. Publ. Nos. WO 93/17715, WO 92/08802, WO 91/00360, and WO 92/05793; Tutt et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

Human polypeptides (e.g., antibodies) that specifically bind to APJ (e.g., human APJ) can be made by a variety of methods known in the art, including the phage display methods described above using antibody libraries derived from human immunoglobulin sequences. See also, U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741, all of which are herein incorporated by reference in their entireties.

### Kits

Also provided are kits comprising one or more of an anti-APJ antibody or polypeptide described herein or a fragment thereof (e.g., a VH or a heavy chain), or pharmaceutical compositions or conjugates thereof. In a specific embodiment, provided herein is a pharmaceutical pack or kit comprising one or more containers filled with one or more of the ingredients of the pharmaceutical compositions described herein, such as one or more anti-APJ antibodies or polypeptides provided herein. In certain embodiments, the kits contain a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided are kits that can be used in the above methods. In certain embodiments, a kit comprises a polypeptide (e.g., antibody) described herein, preferably purified polypeptide, in one or more containers. In a specific embodiment, kits described herein contain substantially isolated APJ antigen as a control. In another specific embodiment, the kits described herein further comprise a control polypeptide (e.g., antibody) which does not react with APJ antigen. In another specific embodiment, kits described herein contain one or more elements for detecting the binding of a polypeptide (e.g., antibody) to an APJ antigen (e.g., the antibody can be conjugated to a detectable substrate such as a fluorescent compound, an enzymatic substrate, a radioactive compound, or a luminescent compound, or a second antibody which recognizes the first antibody can be conjugated to a detectable substrate). The APJ antigen provided in the kit can also be attached to a solid support. In a more specific embodiment, the detecting means of the above-described kit includes a solid support to which APJ antigen is attached. Such a kit can also include a non-attached reporter-labeled anti-human antibody or anti-mouse/rat antibody. In this embodiment, binding of the polypeptide (e.g., antibody) to the APJ antigen can be detected by binding of the said reporter-labeled antibody. In certain embodiments, the present disclosure relates to the use of a kit of the present disclosure for in vitro assaying and/or detecting APJ antigen in a biological sample.

### EXAMPLES

The examples of the present disclosure are offered by way of illustration and explanation, and are not intended to limit the scope of the present disclosure. The results described are reflective of the particular conditions outlined in each example.

### Example 1. Materials and Methods

The following materials and methods were used to conduct the experiments described in Examples 2-6 below.

### Biophysical Assays

Size exclusion chromatography (SEC): to quantify purity and aggregate content of samples, 10 g of protein was loaded to a Waters BEH200 SEC column pre-equilibrated with aqueous mobile phase and separated by size on an Agilent UPLC. Data was processed to report percent monomer, high molecular weight aggregates (HMW), and low molecular weight aggregates (LMW) for each sample.

Imaged capillary isoelectric focusing (iCIEF): to quantify charge variants of samples, samples were diluted to 1 mg/mL with water and added to a mixture of ampholyte, urea, methylcellulose and pI markers used to calibrate the run. Samples were electrophoretically injected onto an iCIEF cartridge on the Maurice system and separated by applying a voltage to the capillary, with samples migrating either toward the anode or cathode depending on charge. Data was processed to report main peak, acidic and basic variants for each sample.

Capillary electrophoresis sodium dodecyl sulfate (CE-SDS): to quantify purity and fragments of samples, 50 g of protein was added to a mixture of CE-SDS denaturing buffer and TCEP (for reducing) and IAM (for non-reducing) analysis. Samples were denatured at 70° C. for 10 minutes and analyzed on a CE-SDS cartridge on the Maurice system, separated by applying a voltage across the capillary. Data was processed to report percent monomer, HMWs and LMWs for each sample.

Heparin binding: to predict PK properties of samples, heparin chromatography was used. To a pre-equilibrated heparin column, 10 g of protein was injected in a low salt buffer at neutral pH. A gradient from low to high salt concentration in the same buffer was run to elute protein. Normalized heparin scores were determined by multiplying the retention time of the main peak by the salt concentration and gradient length in time. Data was reported as heparin score in mM salt at which it eluted.

Differential scanning fluorimetry (DSF): to evaluate thermal stability of samples, samples were diluted to 1 mg/mL in PBS pH 7.4, loaded in a capillary, and then loaded onto the Prometheus Panta. A thermal ramp from 25° C. to 95° C. was run at a rate of 1° C./minute. Data was processed and reported values include Tₒₙₛₑₜ (° C.), the temperature at which unfolding begins; Tₘ1 (° C.), the first melting transition temperature; and T_{agg} (° C.), the temperature at which aggregation begins.

Baculovirus particle ELISA (BVP): to evaluate polyreactivity of samples, a baculovirus particle ELISA was employed. Briefly, ELISA plates were coated with baculovirus particles at a given concentration. Plates were washed, blocked with protein blocking solution, and washed again. Samples were added at a specific concentration, incubated, washed, incubated with detection antibody, washed, and then developed for 5 minutes. BVP scores were reported as normalized response of the sample divided by the response of the blank wells.

Liquid chromatography-mass spectrometry (LC-MS): For fragmentation analysis, 5 mg of sample was run on a Superdex 200 SEC column to separate fragments by size prior to being subjected to intact mass analysis. 20 g from each pooled fraction was treated with PNGase to remove N-linked glycans. 2 g was loaded onto an LC-MS system. The LC component of the system was equipped with a desalting cartridge to remove salt, followed by MS detection using Intact Protein mode. For O-glycosylation analysis, 20 g of sample was treated with PNGase to remove N-linked glycans. 2 g was loaded onto the LC-MS system. As above, the LC component of the system was equipped with a desalting cartridge to remove salt, followed by MS detection using Intact Protein mode.

### Functional Assays

Cyclic AMP (cAMP) homogeneous time resolved fluorescence (HTRF) assay: to evaluate the potency of samples for APJ antagonism, a highly sensitive HTRF-based competitive immunoassay (Revvity) was used to quantify the amount of second messenger cAMP produced upon inhibition of APJ Gai signaling. The cAMP concentration from lysed cells was determined by interpolating raw HTRF ratios at 665/620 nm from a cAMP standard curve.

CHO-Flp-In cells stably expressing human, mouse, or cynomolgus monkey (“cyno”) APJ were cultured in regular media. On the day of the assay, the cells were detached with cell dissociation media and plated at 2,000 cells per well in 10 μl of stimulation buffer with 0.5 mM IBMX in a 384 well plate. Next, 5 μl of 4× serially diluted antibodies were added to the cells and incubated for 30 minutes at room temperature, followed by sequential addition of 2.5 μl of 8× Ec80 human Apelin-13 (Bachem cat. #4029109) for 15 minutes and 2.5 μl of 8× Ec80 Forskolin (Sigma Cat. #F6886) for 45 minutes at room temperature. Cells were lysed with 10 μl of d2 and 10 μl of Eu reagent made in the lysis and detection buffer. Finally, HTRF was read at 665/620 nm on a Clariostar® plate reader after 1 hr incubation of the assay plate at room temperature.

The cAMP amounts were interpolated from the cAMP standard curve using 4 Parametric non-linear regression analysis with GraphPad Prism software, and IC₅₀ values were deduced from log(inhibitor) vs response—variable slope four parameter non-linear regression equation using GraphPad Prism.

β-arrestin assay: to evaluate the potency of samples for APJ agonism or antagonism, an assay was used to evaluate the activation or inhibition of ligand-activated β-arrestin recruitment by agonists or antagonists. Upon activation of APJ, β-arrestin tagged with enzyme acceptor (EA) is recruited to the PK-tagged APJ, which then complements the two enzyme fragments, resulting in the formation of an active β-galactosidase enzyme. The enzyme reacts with the substrate in detection reagent to generate luminescence.

CHO-K1 PathHunter® human APJ cells (Eurofins DiscoverX cat. #93-0250C2) were cultured in the AssayComplete™ cell culture kit 107 (Eurofins DiscoverX cat. #92-3107G). One day before the assay, the cells were detached, and 5,000 cells were plated into each well of a 384-well plate in 30 μl of cell plating media. The plate was then incubated at 37° C. overnight. On the day of the assay, 5 μl of 8× serially diluted antibodies were added to the cells and incubated at 37° C. for 30 minutes, followed by adding 5 μl of 8× EC80 of human Apelin-13 (Bachem cat. #4029109) for 90 minutes (antagonism assays only). 20 μl of detection reagent was added and incubated for one hour at room temperature. Subsequently, the luminescence readout was measured on a Clariostar® plate reader. The IC₅₀ or EC₅₀ values were determined from dose response curve plotted using log(inhibitor) vs response—variable slope four parameter non-linear regression equation using GraphPad Prism

GloSensor™ cAMP assay: to evaluate the potency of samples for APJ antagonism, an assay using a GloSensor™ biosensor, a mutant luciferase fused to a cAMP binding domain, was used. The fusion protein produces light in the presence of its substrate (luciferin), and the readout of relative luminescent units (RLU) was used as a proxy for cAMP response.

CHO-K1 cells were seeded at 7,000 per well in 40 μl of culture media. The next day, they were co-transfected with mouse, cynomolgus, or rat APJ and a pGloSensor™-22F plasmid (Promega cat. #E2301). After 20 hrs. of transfection, the media was replaced with 30 μl CO2 independent media containing 5% D-luciferin and incubated for 90 min at room temperature, followed by a series of additions: 10 μl of 5× serially diluted antibodies for 30 min at room temperature, 5 μl of 10×Ec80 of human Apelin-13 (Bachem cat. #4029109) for 15 min at room temperature, and 5 μl of 10×Ec80 of Forskolin for 45 min at room temperature. The final luminescence was read on a Clariostar® plate reader. The IC50 values were determined from dose-response curve plotted using log(inhibitor) vs response-variable slope four parameters non-linear regression equation using GraphPad Prism.

### Construct Design and Expression

For APJ structural studies, human APJ (Uniprot ID: P35414) residues 7-350, with a portion of intracellular loop 3 (ICL3) replaced with a BRIL fusion, was generated so that an anti-BRIL Fab (Mukherjee et al., 2020, Nat. Commun. 11:1598) could be utilized as a tool to obtain the cryo-EM structure of APJ bound to the VHH of Ab076. An hAPJ F101A mutation was included in the construct to increase the proportion of monomeric receptor; four additional mutations, V117^{3.40}A and W261^{6.48}K (superscripts indicate Ballesteros-Weinstein numbering) and C325L and C326M, were also introduced based on the crystal structure of an apelin analog bound to modified human APJ (Ma et al., 2017, Structure 25, 858-866). The receptor construct presents an N-terminal haemagglutinin (HA)-membrane targeting signal peptide and C-terminal FLAG (DYKDDDDK (SEQ ID NO: 850)) and human rhinovirus (HRV) 3C protease cleavable His₁₀ (HHHHHHHHHH (SEQ ID NO: 851)) tags for affinity purification. The Ab076 VHH expression construct presents a N-terminal Ig kappa leader sequence for secretion into the media and a C-terminal His₁₀ tag for Ni-NTA affinity purification from supernatant 72 hr after transfection of HEK 293 cells. HEK 293 cells expressing the APJ construct were harvested 30 hr after induction.

### Protein Purification

Cell lysis, solubilization and VHH complexation for APJ-(ICL3)BRIL were done simultaneously by resuspending pellets expressing the receptor in a buffer containing 20 mM HEPES pH 7.5, 10 mM MgCl₂, 150 mM NaCl, benzonase, and protease inhibitor cocktail, homogenizing with Dounce and incubating for 30 mins at room temperature (RT) with 1 μM of purified Ab076 VHH, followed by 90 mins incubation with 0.8% (w/v) lauryl maltose neopentyl glycol (LMNG, Anatrace), 0.2% (w/v) glyco-diosgenin (GDN, Anatrace) and 0.2% (w/v) cholesteryl hemisuccinate (CHS, Anatrace) at 4° C. Insoluble material was removed by ultracentrifugation at 100,000×g for 30 min. Detergent solubilized receptor in complex with Ab076 VHH was purified by two-step affinity chromatography. A first Ni chelate resin was utilized, gradually lowering detergent concentration, in 20 mM HEPES pH 7.5, 150 mM NaCl and 30 mM Imidazole, reaching a final detergent concentration of 0.008% (w/v) LMNG, 0.002% (w/v) GDN and 0.002% (w/v) CHS at elution with 250 mM imidazole. A FLAG (M2) affinity purification in the presence of 0.1 μM of Ab076 VHH was performed in 20 mM HEPES pH 7.5, 150 mM NaCl, 0.008% (w/v) LMNG, 0.002% (w/v) GDN and 0.002% (w/v) CHS. A 1.5 molar excess of anti-BRIL Fab was added to the eluted sample and incubated for 90 mins on ice. After concentration of the sample using a 100 kDa cutoff Amicon concentrator, a final size exclusion chromatography step over a SUPEROSE™ 6 Increase 10/300 GL column (GE Healthcare) was performed for the removal of oligomeric fragments and unbound anti-BRIL Fab with running buffer containing 20 mM HEPES pH 7.5, 150 mM NaCl, 0.1 μM Ab076, 0.004% (w/v) LMNG, 0.001% (w/v) GDN and 0.001% (w/v) CHS. Fractions containing monomeric receptor complex were pooled, concentrated and used for the cryo-EM experiment. The quality and purity of the sample was confirmed by HPLC and SDS-PAGE.

### Cryo-EM Data Acquisition, Processing and Modelling

A volume of 3 μL of purified sample at 4 to 7 mg/mL was applied on glow-discharged UltrAuFoil holey gold grids (Quantifoil, Au300-R1.2/1.3). The grids were blotted using a Vitrobot Mark IV (FEI) with 1 to 2 s blotting time at 4° C. in 100% humidity and plunge-frozen in liquid ethane. A total of 13,449 micrographs were collected on a Titan Krios electron microscope (ThermoFisher Scientific—FEI) operating at 300 kV at a magnification of 105,000 Å, corresponding to a magnified pixel size of 0.838 Å. An energy filter was operated with an energy slit width of 20 eV. Micrographs were recorded using a K3 direct electron camera (Gatan) with defocus values ranging from −1.0 m to −1.8 m. The total exposure time was 1.4 s, and intermediate frames were recorded in 0.04 s intervals resulting in an accumulated dose of about 49.24 e⁻/Å² and a total of 35 frames per micrograph. Around 2 million particles were selected and subjected to 2D and 3D classification rounds using cryoSPARC (Punjani et al., 2017, Nat. Methods 14:290-296). A subset of approximately 203,000 particles contributing to the final reconstruction, went through homogeneous, non-uniform, and local refinement obtaining a 3.6 Å resolution map for the VHH Ab076 bound receptor. The resulting map for the complex was sharpened using DeepEMhancer (Sanchez-Garcia et al., 2021, Commun. Biol. 4:874) and used to build the final model. Coordinates for human APJ bound to JN241 (PDB 6KNM; Ma et al., 2020, Sci. Adv. 6:eaax7379) and AT1R (ICL3) BRIL (PDB 8TH3; Skiba et al., 2024, Nat. Chem. Biol. 20:1577-1585) were used as initial models for docking into the EM density map using Chimera (Pettersen et al., 2004, J. Comput. Chem. 25(13):1605-1612). The model was subjected to iterative rounds of manual refinement in Coot (Emsley et al., 2010, Acta. Crystallogr. D. Biol. Crystallogr. 66(4):486-501) as well as a final validation round using the CCP-EM suite (Wood et al., 2015, Acta. Cryst. D 71:123-126; Burnley et al., 2017, Acta. Cryst. D 73:469-477). The cryoEM map was masked at different levels to avoid noise while model building and for figure preparation. Most residues were unequivocally built in the cryo-EM map.

### Example 2. Generation and Optimization of Anti-APJ Antibodies

### First Screening Round

The CDRs from the camelid-derived anti-APJ single-domain antibody (sdAb) JN241 (Ma et al., 2020, Sci. Adv. 6:eaax7379 (“Ma 2020”), which is incorporated by reference herein in its entirety) were grafted into an engineered human VH3-23-based heavy chain variable domain framework selected for suitability in the single-domain context and fused to a human IgG Fc incorporating the “LALAPA” mutations (L234A/L235A/P329A, numbered according to the EU numbering system; “LALAPA Fc”), resulting in the sdAb Ab001.

To generate a panel of further optimized anti-APJ antibodies, an antibody library was generated based on the sequence of Ab001 and screened for binding to an inactive variant of human APJ. The library was built using a site-directed degenerate codon (NNK) library targeting the codons of two amino acids in CDR2 (R53 and R55) and two amino acids in CDR3 (N112 and M113) in Ab001, with amino acid K114 in CDR3 additionally varied to Q by rational design in some instances. The functional and biophysical characteristics of the resulting clones from this first optimization screen (all incorporating LALAPA Fc) as well as for Ab001 were tested; results are shown in Table 16 and Table 17.

TABLE 16
Functional properties of anti-APJ antibodies
from first optimization screen.

| | Human Beta- | Human cAMP | Cyno GloSensor cAMP |
|---|---|---|---|
| | Arrestin (Mean ± | Stable (Mean ± | Transient CHO (Mean ± |
| Antibody | St. Dev.) (nM) | St. Dev.) (nM) | St. Dev.) (nM) |
| Ab001 | 2.5 ± 2 | 2.9 ± 2 | 3.2 ± 5 |
| Ab002 | 5.0 ± 1 | ND | ND |
| Ab003 | 4.7 ± 0.5 | ND | ND |
| Ab004 | 6.0 ± 2 | ND | ND |
| Ab005 | 1.5 ± 0.5 | ND | ND |
| Ab006 | 1.5 ± 0.5 | ND | ND |
| Ab007 | 3 ± 1 | ND | ND |
| Ab008 | 6.0 ± 2 | ND | ND |
| Ab009 | 11 ± 4 | ND | ND |
| Ab010 | 26 ± 19 | ND | ND |
| Ab011 | 8.3 ± 3 | ND | ND |
| Ab012 | 2 ± 0 | ND | ND |
| Ab013 | 0.8 ± 0.05 | ND | ND |
| Ab014 | 2.5 ± 0.5 | 32 ± 16 | 15 ± 9 |
| Ab015 | 13 ± 0.5 | ND | ND |
| Ab016 | 133 ± 33 | ND | ND |
| Ab017 | 1 ± 0 | ND | ND |
| Ab018 | 2.5 ± 0.5 | ND | ND |
| Ab019 | 4.0 ± 1 | 2.5 ± 3 | 9.1 ± 5 |
| Ab020 | 16 ± 8 | 21 | 7.0 |
| Ab021 | 11 ± 3 | ND | ND |
| Ab022 | 6.7 ± 2 | ND | ND |
| Ab023 | 4.7 ± 1 | ND | ND |
| Ab024 | 4.4 ± 4 | 7.3 | 8 |
| Ab025 | 5.4 ± 4 | 1.7 | 7 |
| Ab026 | 9.1 ± 4 | 17 ± 7 | 18 ± 12 |
| Ab027 | 11 ± 5 | ND | ND |
| Ab028 | 6.0 ± 3 | ND | ND |
| Ab029 | 12 ± 6 | 25 | 4.4 |
| Ab030 | 6.0 ± 2 | 3.8 ± 2 | 20 ± 8 |
| Ab031 | 3.7 ± 1 | ND | ND |
| Ab032 | 4.0 ± 0.8 | ND | ND |
| Ab033 | 4.3 ± 0.9 | ND | ND |
| Ab034 | 4.3 ± 3 | ND | ND |
| Ab035 | 5.0 | ND | ND |
| Ab036 | 3.3 ± 0.9 | ND | ND |
| Ab037 | 3.7 ± 1 | ND | ND |

ND: not determined

TABLE 17
Biophysical properties of anti-APJ antibodies
from first optimization screen.

| | % | Heparin | | | | |
|---|---|---|---|---|---|---|
| Antibody | Monomer | Bind (mM) | BVP | Tₒₙₛₑₜ | Tₘ1 | T_{agg} |
| Ab001 | 96 | 292 | 1 | 60 | 64 | 58 |
| Ab002 | 96 | 408 | 8 | 51 | 56 | 49 |
| Ab003 | 95 | 283 | 8 | 54 | 60 | 56 |
| Ab004 | 92 | 242 | 6 | 55 | 60 | 60 |
| Ab005 | 95 | 317 | 8 | 53 | 59 | 52 |
| Ab006 | 96 | 275 | 6 | 50 | 57 | 51 |
| Ab007 | 94 | 200 | 4 | 52 | 59 | 56 |
| Ab008 | 98 | 458 | 6 | 52 | 59 | 53 |
| Ab009 | 93 | 317 | 5 | 53 | 59 | 54 |
| Ab010 | 91 | 242 | 10 | 56 | 59 | 60 |
| Ab011 | 94 | 308 | 11 | 56 | 58 | 60 |
| Ab012 | 96 | 250 | 5 | 52 | 59 | 53 |
| Ab013 | 96 | 308 | 7 | 53 | 59 | 54 |
| Ab014 | 96 | 242 | 5 | 58 | 63 | 62 |
| Ab015 | 83 | 217 | 3 | 55 | 60 | 66 |
| Ab016 | 75 | 192 | 5 | 56 | 61 | 66 |
| Ab017 | 94 | 292 | 7 | 52 | 59 | 59 |
| Ab018 | 97 | ND | 6 | 52 | 57 | 46 |
| Ab019 | 94 | 292 | 3 | 55 | 59 | 61 |
| Ab020 | 98 | 225 | 4 | 63 | 73 | 66 |
| Ab021 | 98 | 217 | 5 | 60 | 62 | 62 |
| Ab022 | 98 | 292 | 7 | 64 | 74 | 66 |
| Ab023 | 98 | 283 | 9 | 59 | 60 | 61 |
| Ab024 | 98 | 217 | 4 | 63 | 74 | 67 |
| Ab025 | 98 | 208 | 3 | 65 | 73 | 64 |
| Ab026 | 98 | 217 | 5 | 65 | 74 | 66 |
| Ab029 | 98 | 217 | 4 | 65 | 74 | 66 |
| Ab030 | 98 | 217 | 5 | 59 | 61 | 62 |
| Ab031 | 96 | 242 | 12 | 51 | 59 | 54 |
| Ab032 | 96 | 342 | 12 | 54 | 59 | 53 |
| Ab033 | 97 | 292 | 12 | 50 | 59 | 54 |
| Ab034 | 98 | 292 | 12 | 53 | 59 | 61 |
| Ab035 | 97 | 292 | 8 | 54 | 59 | 54 |
| Ab036 | 97 | 292 | 8 | 55 | 60 | 56 |
| Ab037 | 98 | 292 | 8 | 54 | 57 | 64 |

### Second Screening Round

A second screen was performed to further improve the potency of the anti-APJ antibodies identified in the first optimization screen. A library was designed based on an in silico affinity positional scan. Briefly, single point mutations using all 19 amino acid substitutions were made at every position over CDR1 and CDR2 from Ab001. The predicted change in affinity for mouse and human APJ was calculated for every mutation, and mutations that were predicted to improve binding affinity were incorporated into the library. This library was built with the CDR3s from Ab039 and Ab026 (one sub-library for Ab19 CDR3 and one sub-library for Ab026 CDR3).

The library was subjected to three rounds of FACS selection/sorting using decreasing concentrations of an inactive variant of human APJ round over round. The functional and biophysical characteristics of the resulting clones from this second optimization screen (all incorporating LALAPA Fc) were tested; results are shown in Table 18 and Table 19.

TABLE 18
Functional properties of anti-APJ antibodies
from second optimization screen.

| | Human Beta- | Human cAMP | Cyno GloSensor cAMP |
|---|---|---|---|
| | Arrestin (Mean ± | Stable (Mean ± | Transient CHO (Mean ± |
| Antibody | St. Dev.) (nM) | St. Dev.) (nM) | St. Dev.) (nM) |
| Ab038 | 0.055 ± 0.04 | 18 ± 0 | 1 |
| Ab039 | 0.22 ± 0.2 | 53 ± 66 | 0.4 |
| Ab040 | 0.24 ± 0.2 | 5.7 ± 2 | 0.16 ± 0.03 |
| Ab041 | 0.23 ± 0.2 | 8.9 ± 0.2 | 0.9 ± 0.8 |
| Ab042 | 0.59 ± 0.6 | 280 ± 170 | 2.5 ± 0.5 |
| Ab043 | 0.49 ± 0.5 | 11 ± 1 | 1.3 ± 0.4 |
| Ab044 | 0.49 ± 0.02 | 68.5 ± 16 | 5.9 ± 1 |
| Ab045 | 0.54 ± 0.3 | 18 ± 3 | 2.5 ± 2 |
| Ab046 | 3.1 ± 2 | 3.1 ± 2 | 1.9 |
| Ab047 | 2.0 ± 0.4 | 43 ± 4 | 3.6 |
| Ab048 | 1.9 ± 1.0 | 0.74 ± 0.9 | 0.5 |
| Ab049 | 1.3 ± 0.8 | 1.5 ± 2 | 1.4 |
| Ab050 | 2.6 ± 0.8 | 3.6 ± 2 | 1.2 |
| Ab051 | 1.1 ± 0.3 | 9.5 ± 0 | 4.0 |
| Ab052 | 2.7 ± 3 | 6.9 ± 6 | 2.3 |
| Ab053 | 1.8 ± 1 | 8.4 ± 4 | 5.2 |
| Ab054 | 3.3 ± 3 | 2.3 ± 2 | 2.6 |
| Ab055 | 1.4 ± 0.7 | 6.2 ± 0 | 7.2 |
| Ab056 | 2.8 ± 0.4 | 3.6 ± 1 | 2.8 |
| Ab057 | 4.0 ± 3 | 63 ± 53 | 7.6 |
| Ab058 | 2.2 ± 0.4 | 4.9 ± 5 | 23 |
| Ab059 | 1.5 ± 1 | 4 ± 0.7 | 12 |

TABLE 19
Biophysical properties of anti-APJ antibodies
from second optimization screen.

| | % | Heparin | | | | |
|---|---|---|---|---|---|---|
| Antibody | Monomer | Bind (mM) | BVP | Tₒₙₛₑₜ | Tₘ1 | T_{agg} |
| Ab038 | 90 | 308 | 9 | 60 | 73 | 52 |
| Ab039 | 97 | 317 | 4 | 60 | 72 | 57 |
| Ab040 | 97 | 342 | 2 | 61 | 75 | 60 |
| Ab041 | 98 | 333 | 3 | 60 | 72 | 56 |
| Ab042 | 98 | 258 | 3 | 60 | 73 | 62 |
| Ab043 | 96 | 242 | 2 | 60 | 72 | 64 |
| Ab044 | 98 | 233 | 1 | 61 | 73 | 66 |
| Ab045 | 96 | 217 | 5 | 53 | 60 | 54 |
| Ab046 | 93 | 250 | 3 | 61 | 71 | 59 |
| Ab047 | 93 | 250 | 2 | 63 | 66 | 61 |
| Ab048 | 97 | 308 | 2 | 60 | 72 | 57 |
| Ab049 | 96 | 292 | 3 | 61 | 73 | 58 |
| Ab050 | 95 | 233 | 2 | 60 | 72 | 48 |
| Ab051 | 94 | 225 | 1 | 61 | 73 | 67 |
| Ab052 | 96 | 225 | 1 | 60 | 72 | 68 |
| Ab053 | 96 | 217 | 1 | 61 | 73 | 59 |
| Ab054 | 94 | 217 | 2 | 60 | 72 | 64 |
| Ab055 | 94 | 208 | 1 | 61 | 72 | 66 |
| Ab056 | 95 | 225 | 1 | 60 | 72 | 67 |
| Ab057 | 93 | 217 | 1 | 60 | 73 | 68 |
| Ab058 | 96 | 225 | 1 | 61 | 72 | 66 |
| Ab059 | 96 | 217 | 1 | 61 | 73 | 69 |

### Third Screening Round

A third screen was performed to improve the humanness of the antibodies discovered in the first two screens. Amino acids in antibodies of the first two screens that typically are not found in the human VH3-23 germline were allowed to be mutated to the corresponding germline amino acid at that position or to remain the same as the parent molecule. Amino acids that were found to be enriched in the bulk sequence output from the second library screen were also included. This library was built with the CDR3s from clones Ab026 and Ab030. The functional and biophysical characteristics of the resulting clones from this third optimization screen (all incorporating LALAPA Fc) were tested. As shown in Table 20 and Table 21, many of the resulting clones displayed further improved biophysical properties while maintaining potent APJ antagonism. For example, Ab076 and Ab078 displayed single digit average nM potency levels across all in vitro cell assays and favorable biophysical properties.

TABLE 20
Functional properties of anti-APJ antibodies
from third optimization screen.

| | Human Beta- | Human cAMP | Cyno GloSensor cAMP |
|---|---|---|---|
| | Arrestin (Mean ± | Stable (Mean ± | Transient CHO (Mean ± |
| Antibody | St. Dev.) (nM) | St. Dev.) (nM) | St. Dev.) (nM) |
| Ab069 | 2.7 ± 1 | 4.3 ± 4 | 3.4 ± 0.6 |
| Ab070 | ND | 16 ± 21 | 4.3 |
| Ab071 | ND | >3000 | 375 ± 50 |
| Ab072 | 70 ± 35 | 99 ± 112 | 15 ± 9 |
| Ab073 | 2.7 ± 1 | 4.0 ± 1 | 2.4 ± 0.2 |
| Ab074 | ND | 16 ± 12 | 3.8 |
| Ab075 | 2.2 ± 0.4 | 31 ± 40 | 1.7 ± 0.5 |
| Ab076 | 1.0 ± 0.2 | 3.0 ± 0.6 | 0.88 ± 0.5 |
| Ab077 | 3.3 ± 1 | 30 ± 23 | 2.3 ± 0.8 |
| Ab078 | 2.9 ± 0.9 | 7.4 ± 6 | 3.0 ± 0.1 |
| Ab079 | 13 ± 0.4 | 31 ± 1 | 3.0 |
| Ab080 | 26 ± 2 | 128 ± 59 | 1.7 ± 2 |
| Ab081 | 12 ± 1 | 77 ± 78 | 1.9 ± 3 |
| Ab082 | 80 ± 29 | 142 ± 168 | 1.3 ± 2 |
| Ab083 | ND | 370 | ND |
| Ab084 | ND | 470 | ND |

ND: not determined

TABLE 21
Biophysical properties of anti-APJ antibodies
from third optimization screen.

| | % | Heparin | | | | |
|---|---|---|---|---|---|---|
| Antibody | Monomer | Bind (mM) | BVP | Tₒₙₛₑₜ | Tₘ1 | T_{agg} |
| Ab069 | 95 | 233 | 2 | 65 | 73 | 65 |
| Ab070 | 96 | 233 | 5 | 65 | 74 | 65 |
| Ab071 | 93 | 250 | 2 | 63 | 76 | 73 |
| Ab072 | 95 | 250 | 2 | 63 | 75 | 69 |
| Ab073 | 94 | 250 | 2 | 62 | 73 | 67 |
| Ab074 | 96 | 350 | 6 | 62 | 74 | 64 |
| Ab075 | 94 | 250 | 2 | 62 | 72 | 69 |
| Ab076 | 95 | 250 | 1 | 62 | 74 | 70 |
| Ab077 | 98 | 233 | 1 | 61 | 74 | 69 |
| Ab078 | 98 | 242 | 1 | 62 | 74 | 68 |
| Ab079 | 98 | 250 | 1 | 61 | 76 | 73 |
| Ab080 | 97 | 250 | 1 | 61 | 73 | 68 |
| Ab081 | 98 | 250 | 1 | 62 | 74 | 66 |
| Ab082 | 98 | 258 | 1 | 61 | 75 | 69 |
| Ab083 | 99 | 242 | 1 | 61 | 72 | 65 |
| Ab084 | 99 | 258 | 1 | 60 | 74 | 69 |

### Example 3. Stability Optimization of Anti-APJ Antibodies

The stability and manufacturability of Ab076 and Ab078, as well as Fc variants thereof further containing the half-life extending LS mutations (M428L/N434S, numbered according to the EU numbering system; together with the LALAPA Fc mutations, “LALAPALS Fc”) (Abl21 and Ab122, respectively) were assessed. The antibodies were formulated at lower and higher concentrations and stored for 2 weeks or 4 weeks at 25° C. or 40° C., followed by analysis via SEC and CE-SDS (see Example 1).

Fragmentation was observed in each of the tested samples at both concentrations and at both 25° C. and 40° C. In particular, using LC-MS (see Example 1), it was determined that the fragmentation occurred within the hinge region of the anti-APJ antibodies, primarily before or after an Asp (D) residue (EPKSS/D/KTHTCPPCP) (SEQ ID NO: 831). Without being bound by theory, this fragmentation may have been driven by a β-elimination reaction. To mitigate this liability, a set of 16 hinge variants (Del1-Del16, as shown in Table 22) was designed, and corresponding variants of Abl21 incorporating the Del1-Del16 hinge variants were made (with the exception that Abl31, corresponding to Abl21 with the Del5 hinge variant, and Ab135, corresponding to Abl21 with the De17 hinge variant, did not express in the system tested). Similarly, 8 variants of Ab122 incorporating Del1-Del8 were also made. The functional and biophysical characteristics of the hinge variants (all incorporating LALAPALS Fc) were tested and the results are shown in Table 23 and Table 24, respectively.

TABLE 22
Hinge variant sequences.

| Deletion | Hinge Sequence | SEQ ID NO |
|---|---|---|
| Unmodified | EPKSSDKTHTCPPCP | 831 |
| Del1 | ---------TCPPCP | 704 |
| Del2 | EPKSS----TCPPCP | 705 |
| Del3 | --------------- | |
| Del4 | -------THTCPPCP | 706 |
| Del5 | GGGGS----TCPPCP | 707 |
| Del6 | GGGGSGGGGTCPPCP | 708 |
| Del7 | AHHPEEPSSQCPKCP | 709 |
| Del8 | AQQPEEPSSQCPKCP | 710 |
| Del9 | ---GTNEVCKCPKCP | 711 |
| Del10 | EPKSS-KTHTCPPCP | 712 |
| Del11 | EPKS--KTHTCPPCP | 713 |
| Del12 | EPKSSQKTHTCPPCP | 714 |
| Del13 | EPKSAQKTHTCPPCP | 715 |
| Del14 | EPKSA----TCPPCP | 716 |
| Del15 | GGGGSGGGGQCPPCP | 717 |
| Del16 | GGGGSGGGGACPPCP | 718 |

TABLE 23
Functional properties of Ab121 and Ab122 hinge variants.

| | | | | Cyno GloSensor |
|---|---|---|---|---|
| | | Human cAMP | Human beta-arrestin | cAMP |
| | Hinge | (Mean ± St. | (Mean ± St. Dev.) | (Mean ± St. Dev.) |
| Antibody | mutation | Dev.) (nM) | (nM) | (nM) |
| Ab121 | Unmodified | 6.1 ± 5 | 3.6 ± 2 | 6.5 ± 2 |
| Ab123 | Del1 | 19 ± 18 | 68 ± 32 | 72 ± 0.7 |
| Ab124 | Del2 | 4.0 ± 2 | 3.4 ± 1 | 1.3 ± 0.5 |
| Ab125 | Del3 | 15 ± 12 | 6.7 ± 3.3 | 14 ± 0.8 |
| Ab126 | Del4 | 15 ± 10 | 25 ± 19 | 21 ± 1 |
| Ab132 | Del6 | 15 ± 12 | 8.3 | 5.0 ± 3 |
| Ab136 | Del8 | 5.0 ± 2 | 3.9 ± 2 | 2.5 ± 1 |
| Ab137 | Del9 | 7.3 ± 3 | 2.7 | 3.6 ± 1 |
| Ab141 | Del10 | 4.5 ± 2.0 | 4.1 ± 0.1 | 1.2 ± 0.1 |
| Ab142 | Del11 | 3.5 ± 1.0 | 4.1 ± 0 | 1.6 ± 0.2 |
| Ab143 | Del12 | 3.9 ± 1.0 | 4.1 ± 0.6 | 1.6 ± 0.1 |
| Ab144 | Del13 | 8.8 ± 1.1 | 4.4 ± 0.8 | 1.4 ± 0.1 |
| Ab145 | Del14 | 4.9 ± 0.4 | 4.8 ± 0.8 | 2 ± 0.1 |
| Ab146 | Del15 | 4.1 ± 1.6 | 5.4 ± 0.1 | 1.6 ± 0.1 |
| Ab147 | Del16 | 6.1 ± 2.4 | 4.7 ± 0 | 2.1 ± 0.1 |
| Ab122 | Unmodified | 2.6 ± 1 | 3.0 ± 1 | 1.6 ± 1 |
| Ab127 | Del1 | ND | 266 | ND |
| Ab128 | Del2 | 9.2 ± 5 | 6.5 ± 3 | 9.5 ± 2 |
| Ab129 | Del3 | 7.4 ± 5 | 7.9 ± 1 | 13 ± 2 |
| Ab130 | Del4 | 13 ± 5 | 34 ± 23 | 25 ± 8 |
| Ab133 | Del5 | 10 ± 10 | 5.4 | 2.4 ± 1 |
| Ab134 | Del6 | 5.5 ± 4 | 3.4 | 2.0 ± 1 |
| Ab138 | Del7 | 3.5 ± 3 | 4.0 | 2.0 ± 1 |
| Ab139 | Del8 | 3.1 ± 2 | 4.1 ± 1 | 1.9 ± 1 |

ND: not determined

TABLE 24
Functional properties of Ab121 and Ab122 hinge variants.

| | Hinge | % | Heparin | | | |
|---|---|---|---|---|---|---|
| Antibody | mutation | Monomer | Bind (mM) | Tₒₙₛₑₜ | Tₘ1 | T_{agg} |
| Ab121 | Unmodified | 96 | 253 | 60 | 73 | 70 |
| Ab123 | Del1 | 94 | 250 | 59 | 73 | 69 |
| Ab124 | Del2 | 98 | 250 | 60 | 73 | 71 |
| Ab125 | Del3 | 93 | 260 | 52 | 70 | 67 |
| Ab126 | Del4 | 95 | 251 | 60 | 73 | 70 |
| Ab132 | Del6 | 98 | 258 | 60 | 72 | 72 |
| Ab136 | Del8 | 99 | 239 | 61 | 74 | ND |
| Ab137 | Del9 | 99 | 233 | 61 | 74 | 70 |
| Ab141 | Del10 | 99 | 279 | 60 | 73 | 69 |
| Ab142 | Del11 | 99 | 270 | 60 | 73 | 70 |
| Ab143 | Del12 | 99 | 260 | 60 | 74 | 69 |
| Ab144 | Del13 | 99 | 260 | 60 | 74 | 70 |
| Ab145 | Del14 | 99 | 242 | 60 | 74 | 70 |
| Ab146 | Del15 | 99 | 244 | 60 | 73 | 70 |
| Ab147 | Del16 | 99 | 244 | 60 | 73 | 70 |
| Ab122 | Unmodified | 98 | 242 | 62 | 74 | 68 |
| Ab127 | Del1 | ND | ND | ND | ND | ND |
| Ab128 | Del2 | 96 | 243 | 59 | 72 | 67 |
| Ab129 | Del3 | 93 | 254 | 53 | 68 | 65 |
| Ab130 | Del4 | 95 | 245 | 59 | 71 | 65 |
| Ab133 | Del5 | 88 | 239 | 60 | 73 | 78 |
| Ab134 | Del6 | 99 | 238 | 61 | 73 | 78 |
| Ab138 | Del7 | 99 | 238 | 61 | 73 | 77 |
| Ab139 | Del8 | 99 | 233 | 62 | 74 | 79 |

ND: not determined

Further analysis of these hinge variants was carried out to evaluate the presence or absence of fragmentation in the hinge region and O-glycosylation (see Example 1). Results are shown in Table 25 (“x” indicates presence and “-” indicates absence above detection threshold for given assay; “NT” indicates “not tested”).

TABLE 25
Further analysis of Ab121 and Ab122 hinge variants.

| Antibody | Hinge mutation | Fragmentation in Hinge Region | O-Glyc |
|---|---|---|---|
| Ab121 | Unmodified | x | x |
| Ab123 | Del1 | — | x |
| Ab124 | Del2 | — | x |
| Ab125 | Del3 | — | — |
| Ab126 | Del4 | — | — |
| Ab132 | Del6 | — | x |
| Ab136 | Del8 | — | — |
| Ab137 | Del9 | — | NT |
| Ab141 | Del10 | x | — |
| Ab142 | Del11 | x | — |
| Ab143 | Del12 | — | x |
| Ab144 | Del13 | x | — |
| Ab145 | Del14 | — | x |
| Ab146 | Del15 | — | — |
| Ab147 | Del16 | — | — |
| Ab122 | Unmodified | x | — |
| Ab127 | Del1 | — | x |
| Ab128 | Del2 | — | x |
| Ab129 | Del3 | — | — |
| Ab130 | Del4 | — | — |
| Ab133 | Del5 | — | x |
| Ab134 | Del6 | — | x |
| Ab138 | Del7 | — | x |
| Ab139 | Del8 | — | — |

The hinge variants with the largest deletions—Del1, Del3, and Del4 in each of the Abl21 and Abl22 contexts—exhibited reduced potency and/or reduced thermodynamic stability. In particular, Del3 exhibited a substantial reduction in Tₒₙₛₑₜ, indicating reduced molecular stability, likely due to the hinge no longer containing interchain disulfide bonds. Based on these data, there appears to be a dependence for potency on the distance from the C-terminal end of the variable heavy chain domain to the interchain disulfide bond cysteines in the hinge. In addition, numerous hinge variants exhibited fragmentation in the hinge region and/or O-glycosylation. Abl21 variants Abl36 (Del8), Abl37 (Del9), Abl46 (Del15), and Abl47 (Del16), and Abl22 variant Abl39 (Del8), did not exhibit these characteristics under the conditions tested (except that, as noted above, T_{agg} for Abl36 was not determined and O-glycosylation for Abl37 was not tested).

### Example 4. Generation and Characterization of APJ Agonist Antibodies and Additional Antagonist Antibodies

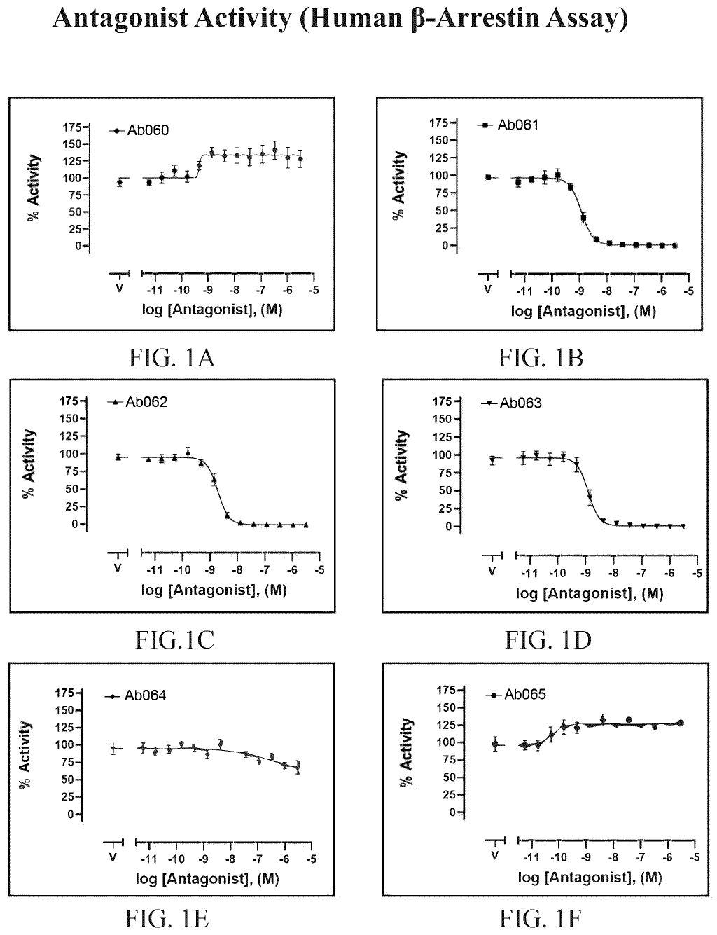

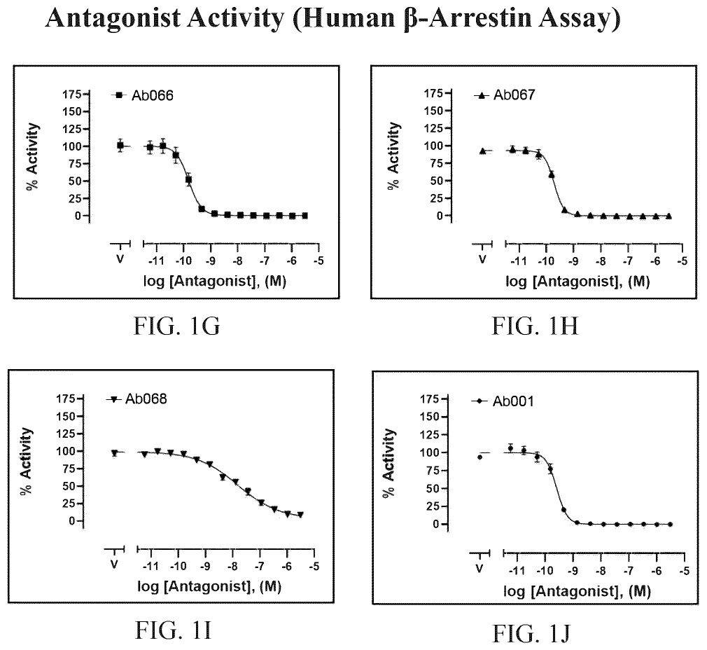

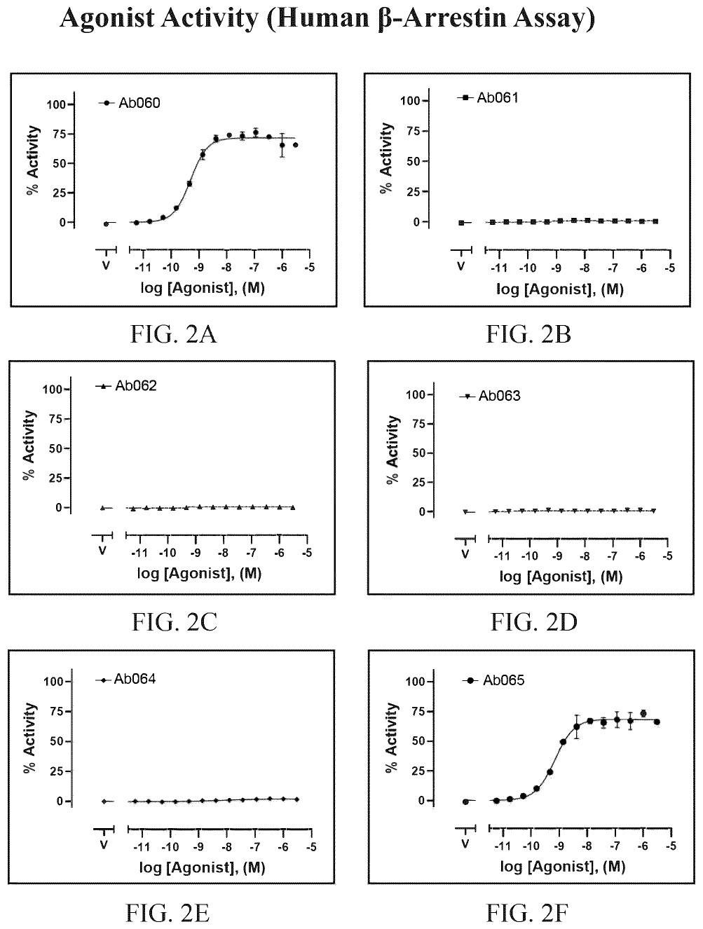

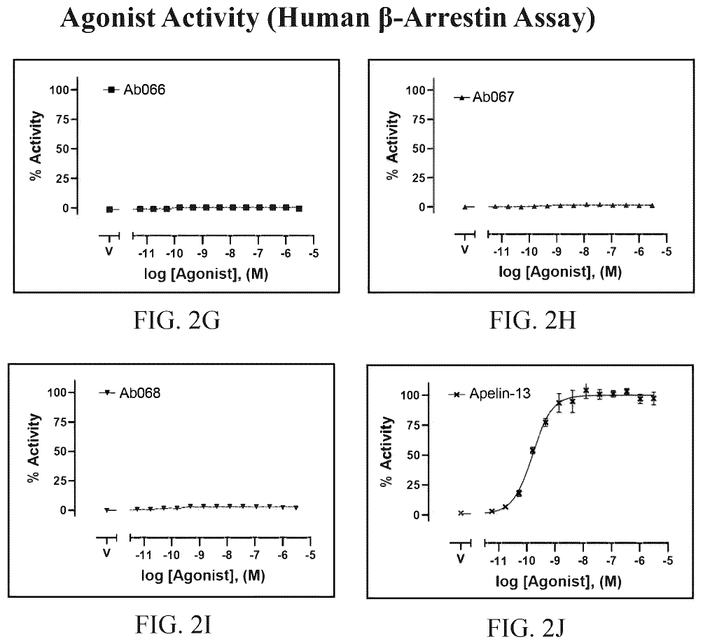

As demonstrated in Ma 2020, insertion of a single tyrosine between E104 and S105 in CDR3 of the JN241 sequence (referred to as JN241-9) resulted in an APJ agonist antibody. A corresponding insertion was made in the Ab001 sequence to generate Ab065. The functional and biophysical characteristics of Ab065 were tested and compared to those of Ab001, JN241, and JN241-9, as well as those of Ab001 and JN241 with one of three alanine substitutions (S30A, T52A, and W111A, in CDR1, CDR2, and CDR3, respectively) described in Ma 2020 (all incorporating LALAPA Fc). The functional characteristics are shown in FIGS. 1A-1J (antagonist assays) and FIGS. 2A-2J (agonist assays) and Table 26, and the biophysical characteristics are shown in Table 27. Ab065 displayed similar potency as an agonist to that of JN241-9. In Ma 2020, all three alanine substitutions were reported to abolish the antagonist function of JN241. In the present experiments, S30A and T52A did not abolish antagonist function of either JN241 or Ab001. Surprisingly, while W111A did abolish the antagonist function of JN241, consistent with Ma 2020, the corresponding substitution only reduced, but did not abolish, antagonist activity of Ab03, highlighting that Ab001 does not interact with APJ in an identical manner to JN241.

TABLE 26
Functional properties of Ab001 and JN241 variants.

| | | Human Beta- | Human Beta- |
|---|---|---|---|
| | | Arrestin | Arrestin |
| Antibody | Description | (nM) | Agonist (nM) |
| Ab060 | JN241-9-Ig Fc | Agonist | 0.50 |
| Ab061 | JN241-Ig Fc | 1.1 | >3000 |
| Ab062 | JN241-Ig Fc | 1.2 | >3000 |
| | S30A | | |
| Ab063 | JN241-Ig Fc | 1.2 | >3000 |
| | T52A | | |
| Ab064 | JN241-Ig Fc | >3000 | >3000 |
| | W111A | | |
| Ab065 | Ab001 Ag-Y | Agonist | 0.68 |
| Ab001 | Ab001 | 0.25 | >3000 |
| Ab066 | Ab001 S30A | 0.15 | >3000 |
| Ab067 | Ab001 T52A | 0.19 | >3000 |
| Ab068 | Ab001 W111A | 16 | >3000 |

TABLE 27
Biophysical properties of Ab001 and JN241 variants.

| | | | Heparin | | | |
|---|---|---|---|---|---|---|
| | | % | Bind | | | |
| Antibody | Description | Monomer | (mM) | Tₒₙₛₑₜ | Tₘ1 | T_{agg} |
| Ab060 | JN241-9-Ig Fc | 98 | 217 | 49 | 61 | 65 |
| Ab061 | JN241-Ig Fc | 99 | 217 | 66 | 69 | 69 |
| Ab062 | JN241-Ig Fc | 98 | 217 | 66 | 69 | ND |
| | S30A | | | | | |
| Ab063 | JN241-Ig Fc | 99 | 217 | 58 | 62 | 56 |
| | T52A | | | | | |
| Ab064 | JN241-Ig Fc | 97 | 208 | 45 | 57 | 68 |
| | W111A | | | | | |
| Ab065 | Ab001 Ag-Y | 97 | 325 | 53 | 60 | 53 |
| Ab001 | Ab001 | 96 | 292 | 60 | 64 | 58 |
| Ab066 | Ab001 S30A | 97 | 308 | 50 | 60 | 58 |
| Ab067 | Ab001 T52A | 98 | 292 | 55 | 61 | 67 |
| Ab068 | Ab001 W111A | 98 | 317 | 48 | 60 | 48 |

ND: not determined

ND: not determined

An additional set of antagonist and agonist assays as described above was performed in duplicate to test several additional amino acid insertions between E104 and S 105 in the Ab001 sequence, as well as two additional mutations of Will in the Ab001 sequence (all incorporating LALAPA Fc). As shown in Table 28, insertion of an arginine, tryptophan, or lysine between E104 and S105 in CDR3 of the Ab001 sequence (Ab156, Ab157, and Ab158, respectively) exhibited similar potencies as agonists to that of Ab065, while insertion of a proline (Ab155) abolished agonist activity. These insertion variants exhibited a range of potencies as antagonists; surprisingly the tryptophan variant (Ab157) maintained similar antagonist activity to that of Ab001, in addition to its agonist activity. Substitution of Will in the Ab001 sequence with tyrosine (Ab160) maintained antagonist potency relative to Ab001, while substitution of Will with phenylalanine (Ab159) slightly reduced antagonist potency.

TABLE 28
Antagonist and agonist properties of additional Ab001 variants.

| | | | | | | Agonist Activity | |
|---|---|---|---|---|---|---|---|
| | | | | | | (% of Apelin | |
| | | | | | | Activity at Max | |
| | | Human Beta-Arrestin | | Human Beta-Arrestin | | Concentration | |
| | | IC₅₀ antagonist (nM) | | EC₅₀ Agonist (nM) | | Tested (0.3 nM)) | |
| | | Expt. 1 | Expt. 2 | Expt. 1 | Expt. 2 | Expt. 1 | Expt. 2 |
| Antibody | Description | (N = 1) | (N = 1) | (N = 1) | (N = 1) | (N = 1) | (N = 1) |
| Apelin-13 | APJ ligand | — | — | 0.89 | 0.43 | 100 | 100 |
| Ab001 | Ab001 | 3.43 | 3.73 | >3000 | >3000 | 0 | 0 |
| Ab065 | Ab001 Ag-Y | >3000 | >3000 | 1.58 | 1.03 | 120 | 81 |
| Ab068 | Ab001 W111A | 278 | 40 | >3000 | >3000 | 0 | 0 |
| Ab155 | Ab065 Y105P | 134 | 74 | >3000 | >3000 | 0 | 0 |
| Ab156 | Ab065 Y105R | ~1000 | ~1000 | 2.37 | 1.35 | 20 | 25 |
| Ab157 | Ab065 Y105W | 4.4 | 3.7 | 1.83 | 1.45 | 7.5 | 10 |
| Ab158 | Ab065 Y105K | ~1000 | >3000 | 6.27 | 1.94 | 35 | 43.6 |
| Ab159 | Ab001 W111F | 10 | 14 | >3000 | >3000 | 0 | 0 |
| Ab160 | Ab001 W111Y | 3.34 | 4.47 | >3000 | >3000 | 0 | 0 |

### Example 5. Structural Differential Analysis of Optimized Anti-APJ Antibody Ab076 VHH and Anti-APJ sdAb JN241 Bound to APJ

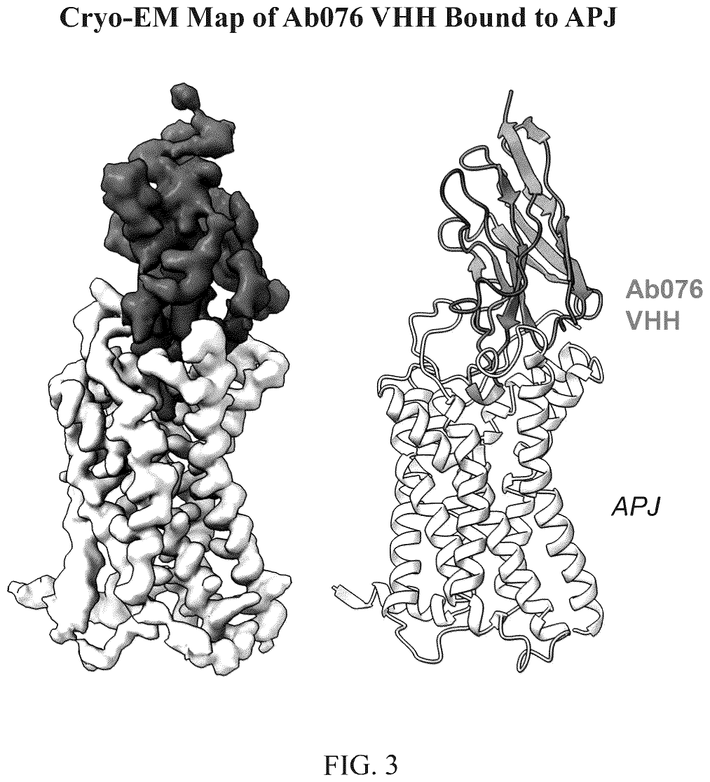

To better understand the structure-activity relationship of the optimized anti-APJ antibody Ab076 VHH (see Example 2), the structure of Ab076 VHH bound to modified human apelin receptor (APJ) was determined by single particle cryo-electron microscopy (cryo-EM) (FIG. 3), as described in Example 1. Table 29 provides the final cryo-EM data collection, model refinement, and validation statistics.

TABLE 29
Cryo-EM data collection, model refinement,
and validation statistics.

| | |
|---|---|
| | Modified Human APJ |
| Structure | (ICL3-BRIL)/Ab076 VHH |

Data collection and processing #

| | |
|---|---|
| Nominal magnification | 105,000 |
| Voltage (kV) | 300 |
| Dose per frame (e−/Å2) | 1.44 |
| Electron exposure (e−/Å2) | 50.33 |
| Defocus Range (μm) | −1.0 to −1.8 |
| Pixel size (Å) | 0.838 |
| Symmetry imposed | C1 |
| Number of Total Micrographs | 13,449 |
| Number of Micrographs selected | 8,328 |
| for processing | |
| Final particle images (no.) | 203,000 |
| Map resolution (Å) | 3.9 |
| FSC threshold | 0.143 |

Refinement Statistics*

| | |
|---|---|
| Model composition | |
| Chains | 3 |
| Total number of atoms | 4063 |
| Number of residues | 532 |
| R.m.s. deviations | |
| Bond lengths (Å) | 0.0137 |
| Bond angles (°) | 1.511 |
| Ramachandran plot | |
| Favored (%) | 82.0 |
| Outlier (%) | 2.9 |

#: Reported by NIS and
*Refmac and Coot in the CCP-EM suite.

An analysis of the cryo-EM structure, specifically of the binding interface between Ab076 VHH and APJ, as well as intramolecular stabilizing interactions for Ab076 VHH, was performed. The structure was also compared with the crystal structure of modified APJ receptor bound to JN241 (PDB 6KNM; Ma 2020). This comparison revealed several inter- and intra-molecular interactions specific for Ab076 VHH that emerged from the affinity maturation and engineering efforts described in Example 2. Described below is an examination of these interactions in combination with experimental data for Ab076, control antibody AbNC, and several illustrative antibodies characterized in Examples 2 and 4. Table 30 lists the illustrative antibodies that are discussed further below, and Tables 31 and 32 summarize potencies for inhibition of cynomolgus monkey APJ (relevant, e.g., for further characterization of molecules prior to administration in human clinical trials) and biophysical characteristics, respectively, obtained for each of the illustrative antibodies. The cynomolgus potency of Ab061 and the biophysical characteristics of AbNC were determined according to the methods of Example 1; the remaining data in Tables 31 and 32 are reproduced from earlier Examples as indicated.

TABLE 30
Description of Ab076, AbNC, and illustrative
antibodies from the optimization process.

| Anti- | |
|---|---|
| body | Description |
| AbNC | Single-domain antibody-Fc negative control incorporating a non- |
| | reactive engineered human VH3-23-based heavy chain variable |
| | domain framework selected for suitability in the single-domain |
| | context and fused to a human IgG Fc incorporating the |
| | “LALAPA” mutations (L234A/L235A/P329A, numbered |
| | according to the EU numbering system) |
| Ab061 | JN241 fused to a human IgG Fc incorporating the “LALAPA” |
| | mutations (described in Example 4 herein) |
| Ab001 | JN241 camelid CDRs grafted into AbNC framework and fused |
| | to a human IgG Fc incorporating the “LALAPA” mutations |
| | (described in Example 2 herein) |
| Ab030 | Ab001 with R53H mutation in CDR2 and N112S and M113Y |
| | mutations in CDR3 (described in Example 2 herein) |
| Ab069 | Ab030 with T57Y mutation in CDR2 (described in Example 2 |
| | herein) |
| Ab076 | Ab069 with S27L and Y29F mutations in CDR1 and L50A, |
| | M51I, T52S, and H53G mutations in CDR2 (described in |
| | Example 2 herein) |

TABLE 31
Cynomolgus potencies of Ab076 and illustrative
antibodies from the optimization process.

| | Cyno GloSensor cAMP Transient |
|---|---|
| Antibody | CHO (Mean ± St. Dev.) (nM) |
| Ab061 | 0.6 ± 0.1 |
| Ab001* | 3.2 ± 5 |
| Ab030* | 20 ± 8 |
| Ab069** | 3.4 ± 0.6 |
| Ab076** | 0.88 ± 0.5 |

*data reproduced from Table 16
**data reproduced from Table 20

TABLE 32
Biophysical properties of Ab076, AbNC, and illustrative
antibodies from the optimization process.

| Antibody | % Monomer | Heparin Bind (mM) | Tₒₙₛₑₜ | Tₘ1 | T_{agg} |
|---|---|---|---|---|---|
| AbNC | 98 | 167 | 60 | 77 | 67 |
| Ab061* | 99 | 217 | 66 | 69 | 69 |
| Ab001** | 96 | 292 | 60 | 64 | 58 |
| Ab030** | 98 | 217 | 59 | 61 | 62 |
| Ab069*** | 95 | 233 | 65 | 73 | 65 |
| Ab076*** | 95 | 250 | 62 | 74 | 70 |

*data reproduced from Table 27
**data reproduced from Table 17
***data reproduced from Table 21

Both Ab061 and AbNC have low heparin binding in vitro (Table 32). However, when the CDRs from Ab061 were grafted onto the AbNC human framework, Ab001 demonstrated substantially increased heparin binding and worse thermostability (Table 32). Without being bound by theory, a potential explanation for this was revealed when comparing the crystal structure of the JN241/APJ complex with the cryo-EM structure of the Ab076 VHH and APJ complex.

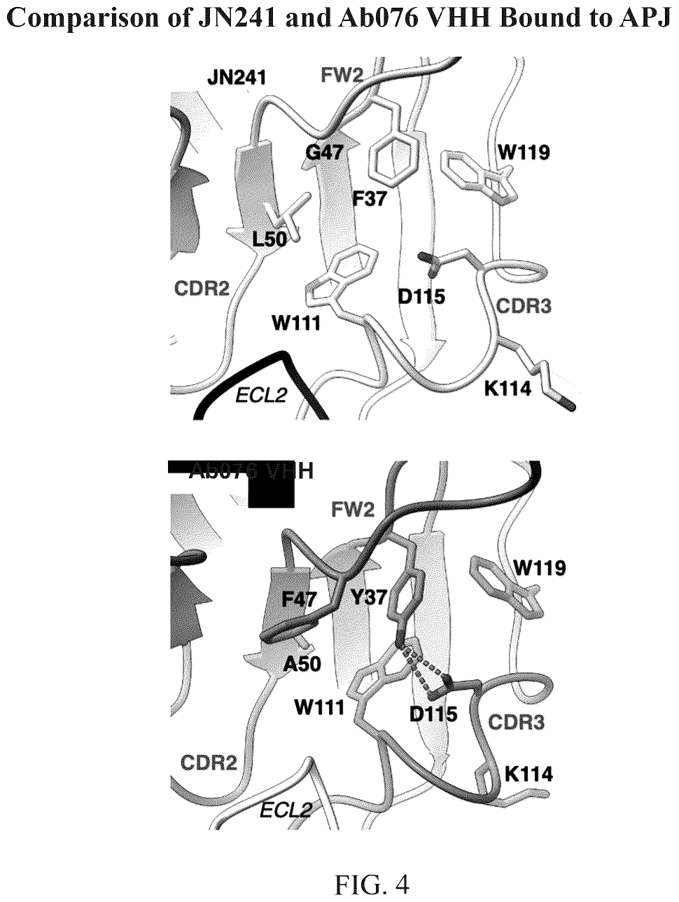

A new intramolecular interaction between FW2 and CDR3 was observed in the Ab076 VHH that was established with the introduction of humanized framework regions in Ab001. In JN241, CDR3 residue D115 did not appear to have significant interactions and was available as a negatively charged counterpart to the neighboring positively charged residue K114 (FIG. 4, top panel). In contrast, in the structure of Ab076, CDR3 residue D115 and FW2 residue Y37 (FIG. 4, bottom panel) engaged in an intramolecular hydrogen bond, apparently leaving K114 positive charge unbalanced. Positive charges tend to increase binding to heparin, which can be a proxy for poor pharmacokinetics (see, e.g., Li et al. (2014), mAbs 6:5, 1255-1264, and Kraft et al. (2020), mAbs 12, 1683432, each of which is herein incorporated by reference in its entirety); accordingly, the engagement of D115 by Y37 may have accounted for the marked increase in heparin binding from 217 mM for Ab061 to 292 mM for Ab001. Notably, the tyrosine at position 37 in the human FW2 region corresponds to F37 in the llama FW2 region, which has no H-bond donors to interact with D115 in CDR3. Residue Y37 in Ab001 is also part of a network of hydrophobic interactions with residues W111, W119 and F47. A similar network is present in JN241 with residues F37, W119, L50 and G47. However, in all the humanized molecules from Ab001 to Ab076, G47 in FW2 was changed to F47, which further stabilizes residue Y37 as it interacts with D115.

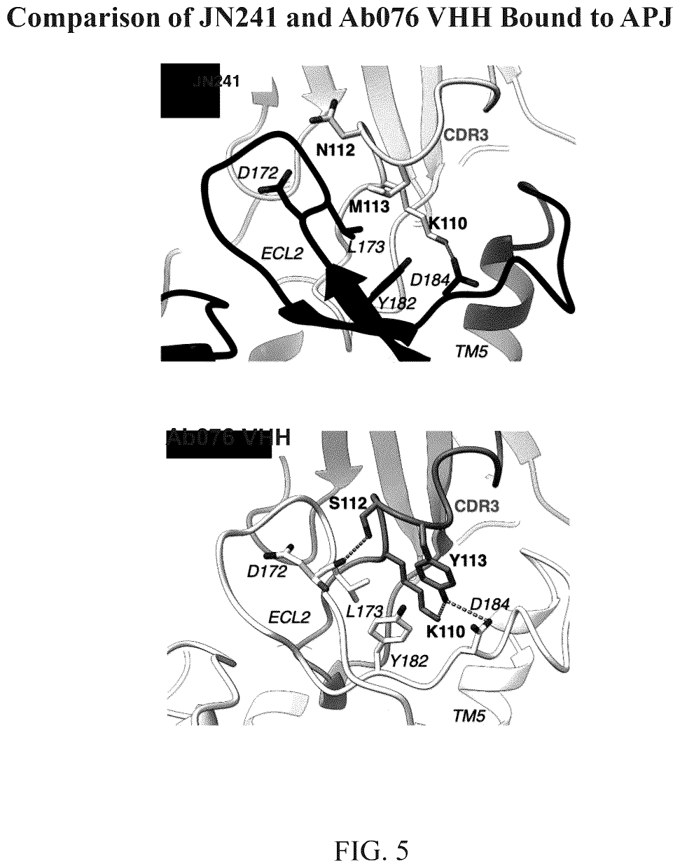

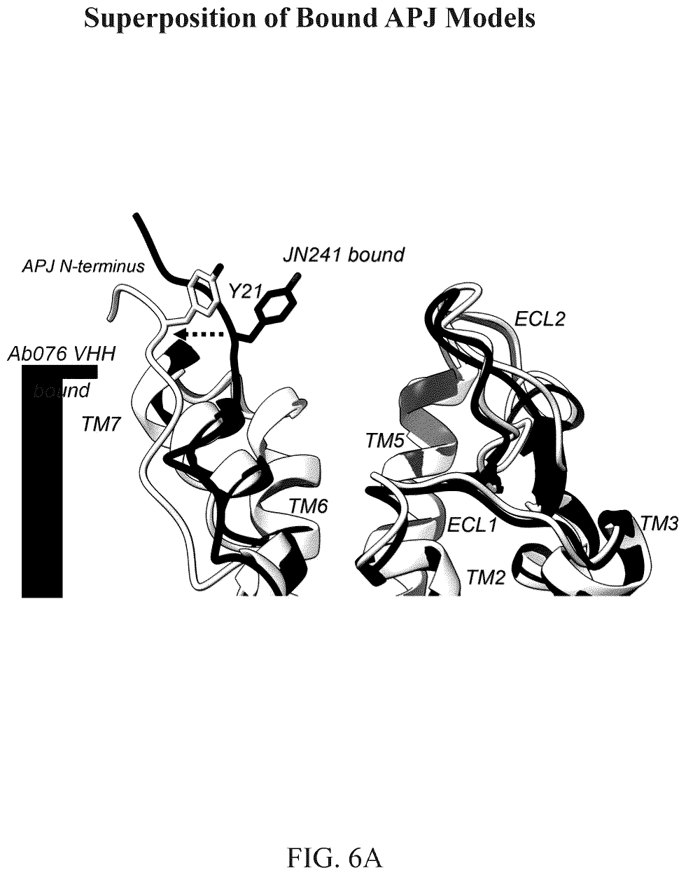

The introduction of CDR3 residues S112 and Y113 in Ab030, Ab069, and Ab076 led to new intermolecular interactions with APJ ECL2 residues, as seen in the Ab076 structure. Specifically, S112 in Ab076 CDR3 interacts via hydrogen bond with the APJ D172 backbone carbonyl oxygen, while Y113 interacts with APJ ECL2 residue D184 and also forms a polar network with K110, sequestering the latter's positive charge (FIG. 5, bottom panel). Interactions involving the corresponding residue N112 in JN241 were not reported in Ma 2020 for JN241, and M113 was described as establishing a hydrophobic interaction with APJ residue L173.

Importantly, incorporation of these changes in Ab030, Ab069, and Ab076 restored binding to heparin from 292 mM in Ab001 back to 217 mM in Ab030. At the same time, as shown in FIG. 5, the stabilizing salt bridge from K110 in JN241 to D184 in APJ was replaced with a weaker H-bond interaction between Y113 in Ab076 and D184 in APJ. Without being bound by theory, this change may have contributed to the loss in cyno potency of Ab030 in comparison to Ab001.

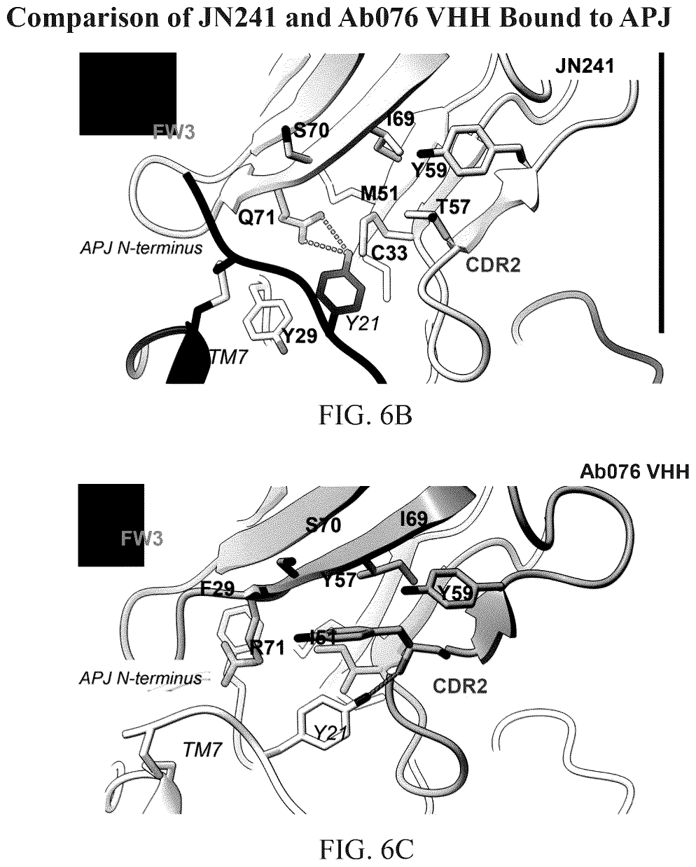

Turning to Ab069, the sole amino acid change relative to Ab030 was the mutation of T57 to Y57 in CDR2. This mutation first arose in the course of affinity maturation in the second screening round described in Example 2. Surprisingly, the single change from T57 to Y57 restored cyno potency and improved thermostability (Tables 31 and 32). In the structure of Ab076, a dramatic change can be seen in the N-terminal conformation of APJ when bound to the two molecules (FIG. 6A). The change in CDR2 from T57 to Y57 established a new interaction with APJ Y21, which moves forward with respect to its position in JN241 (FIGS. 6B and 6C). Y57 also established intra-molecular hydrophobic interactions with other Ab076 residues, which could account for the substantially improved thermostability generated by the T to Y mutation in CDR2 (Table 32).

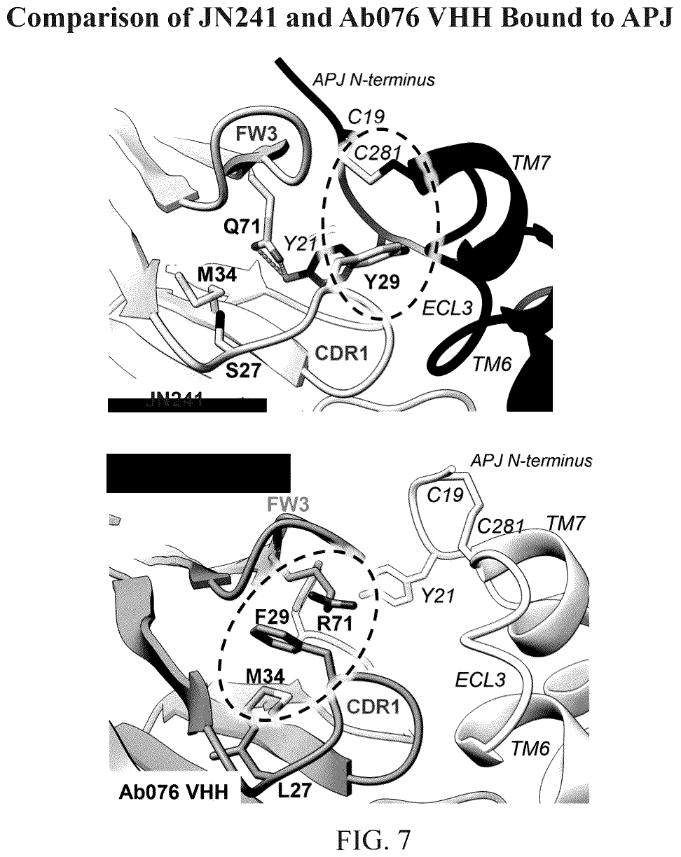

The final VHH modifications incorporated into Ab076 were made in the context of CDR humanization in the third screening round described in Example 2. In CDR1, Ab076 CDR1 residue F29 (changed from Y29) formed a stabilizing intra-molecular network with residue R71 (FW3) and a Pi-sulfur interaction with M34 (CDR1) away from the APJ N-terminus (FIG. 7, bottom panel). The CDR1 S27L change structurally stabilized the stacking interaction, further establishing contacts with M34. Ab076 residues L27, F29, and R71 differ from their corresponding residues in JN241; L27 and F29 originated from CDR1 humanization, and R71 is part of the engineered human VH3-23-based VHH framework.

In turn, residue Y29 in JN241 adopts a different conformation, establishing a Pi-sulfur interaction with APJ residue C281 (FIG. 7, top panel). Intriguingly, since APJ C281 (TM7) forms part of a disulfide bond with residue C19 (TM1), the interaction with Y29 appears to bring the N-terminal region of APJ closer to JN241, resulting in APJ Y21 binding inwards and establishing an H-bond with Q71 (FIG. 6B).

The stacking interaction between R71, F29, and M34 in Ab076 likely increased VHH stability. While the loss of the interaction between the residue in VHH position 29 and APJ C281, on its own, may have reduced potency, the structural analysis suggests that a countervailing effect was created by the introduction of Y57 in CDR2. In particular, with the change of FW3 residue 71 from Q to R, APJ residue Y21 was no longer able to form an H bond with VHH position 71, freeing Y21 to establish a new interaction with VHH Y57 as described above.

Turning to CDR2 of Ab076, the L50A modification, which removed a bulky side chain, elegantly accommodated the introduction of a bulky side chain with the G47F modification in FW2 (FIG. 4). Without being bound by theory, this structural insight may help explain the unexpected observation in Example 4 that while the mutation at residue 111 in Ab064 (JN241-Ig Fc W111A) abolished antagonist function of JN241-Ig Fc in a human beta-arrestin assay, the corresponding mutation in Ab068 (Ab001 W111A) only reduced, but did not abolish, antagonist function (Table 26). In Ab064, residue 47 is glycine and residue 50 is leucine, whereas in Ab068, residue 47 is phenylalanine while residue 50 remains leucine, potentially creating additional hydrophobic interactions in the region corresponding to FIG. 4. The observation that mutation of residue 111 from tryptophan to alanine is substantially better tolerated in the context of Ab068 as compared to Ab064, with respect to inhibition of apelin-dependent APJ activation, may be because the additional hydrophobic interactions contributed by F47 in Ab068 partially offset the loss of the bulky hydrophobic tryptophan side chain at residue 111.

In summary, combining structural, mutational, and functional analysis into the SAR description for VHH humanization and affinity maturation enhanced understanding of the emergence of unexpected favorable properties for anti-APJ antibodies described herein.

### Example 6. Generation of Additional Anti-APJ Antibodies

A naïve yeast display campaign for human APJ binders led to discovery of Ab085. Additional antibodies based on Ab085 were generated via affinity maturation for inactive human and mouse APJ variants and polyreactivity-lowering screens.

The functional and biophysical characteristics of Ab085 and the additional antibodies (all incorporating LALAPA Fc) were tested as described in Example 1, and results are shown in Table 33 and Table 34, respectively. Ab108 was selected for further characterization in in vivo studies in mouse models in the Examples that follow.

TABLE 33
Functional properties of anti-APJ antibodies from Ab085 optimization screen.

| | Human | | Cyno | Mouse | Rat |
|---|---|---|---|---|---|
| | Beta- | Human | GloSensor | GloSensor | GloSensor |
| | Arrestin | cAMP | cAMP | cAMP | cAMP |
| | (Mean ± St. | (Mean ± St. | (Mean ± St. | (Mean ± St. | (Mean ± St. |
| Antibody | Dev.) (nM) | Dev.) (nM) | Dev.) (nM) | Dev.) (nM) | Dev.) (nM) |
| Ab086 | 8.5 ± 6 | 4.8 ± 8 | 90 | 205 ± 100 | >1000 |
| Ab085 | 325 | ND | >3000 | ND | ND |
| Ab087 | >3000 | >3000 | >3000 | 59 ± 8 | >3000 |
| Ab088 | >3000 | >3000 | >3000 | 22 ± 17 | >3000 |
| Ab089 | >3000 | >3000 | >3000 | 35 ± 7 | >3000 |
| Ab090 | >3000 | >3000 | >3000 | 31 ± 21 | >3000 |
| Ab091 | >3000 | >3000 | >3000 | 29 ± 27 | >3000 |
| Ab092 | >3000 | >3000 | >3000 | 109 ± 72 | >3000 |
| Ab093 | >3000 | >3000 | >3000 | 2.4 ± 3 | 87 ± 60 |
| Ab094 | >3000 | >3000 | >3000 | 6.6 | >3000 |
| Ab095 | ~1500 | >3000 | >3000 | 46 | >3000 |
| Ab096 | >3000 | >3000 | >3000 | 62 | >3000 |
| Ab097 | 2.5 ± 0.7 | 5.3 ± 0 | 148 ± 32 | ND | ND |
| Ab098 | 115 ± 5 | 150 ± 71 | >3000 | 120 | >3000 |
| Ab099 | 12 ± 4 | 53 ± 23 | 378 ± 253 | 0.73 | >3000 |
| Ab100 | 91 ± 51 | 118 ± 87 | >3000 | 6.8 | >3000 |
| Ab101 | >3000 | >3000 | ND | 131 | >3000 |
| Ab102 | >3000 | >3000 | ND | 90 | >3000 |
| Ab103 | >3000 | >3000 | ND | 230 | >3000 |
| Ab104 | >3000 | >3000 | ND | 10 | >3000 |
| Ab105 | 13 ± 10 | 23 ± 18 | 593 ± 302 | 4.2 | >3000 |
| Ab106 | >3000 | >3000 | ND | 5.5 | >3000 |
| Ab107 | >3000 | >3000 | ND | 12 | >3000 |
| Ab108 | 260 ± 126 | 122 ± 45 | >3000 | 1.8 ± 2 | >3000 |
| Ab109 | 60 ± 33 | 180 ± 44 | >3000 | ~960 | >3000 |
| Ab110 | 10 | 48 | >3000 | ND | ND |
| Ab111 | 4.5 | 30 | >3000 | ND | ND |
| Ab112 | 72 | 40 | >3000 | ND | ND |
| Ab113 | 163 | 49 | >3000 | ND | ND |
| Ab114 | 51 | 8.0 | >3000 | ND | ND |
| Ab115 | 146 | 5.2 | >3000 | ND | ND |

ND: not determined

TABLE 34
Biophysical properties of anti-APJ antibodies
from Ab085 optimization screen.

| | % | Heparin | | | | |
|---|---|---|---|---|---|---|
| Antibody | Monomer | Bind (mM) | BVP | Tₒₙₛₑₜ | Tₘ1 | T_{agg} |
| Ab086 | 85 | 475 | 12 | 60 | 67 | 58 |
| Ab085 | 92 | 268 | 2 | 49 | 70 | 60 |
| Ab087 | 94 | 292 | 10 | 58 | 66 | 60 |
| Ab088 | 93 | 292 | 11 | 57 | 65 | 59 |
| Ab089 | 94 | 275 | 10 | 58 | 66 | 61 |
| Ab090 | 95 | 283 | 12 | 58 | 66 | 62 |
| Ab091 | 93 | 367 | 8 | 48 | 57 | 51 |
| Ab092 | 93 | 358 | 7 | 48 | 57 | 51 |
| Ab093 | 92 | 308 | 6 | 60 | 74 | 70 |
| Ab094 | 92 | 358 | 9 | 60 | 73 | 69 |
| Ab095 | 94 | 308 | 5 | 59 | 74 | 70 |
| Ab096 | 95 | 383 | 6 | 55 | 73 | 59 |
| Ab097 | 96 | 567 | 10 | 55 | 63 | 56 |
| Ab098 | 97 | 342 | 10 | 58 | 64 | 58 |
| Ab099 | 95 | 358 | 11 | 57 | 66 | 61 |
| Ab100 | 96 | 292 | 10 | 68 | 72 | 66 |
| Ab101 | 92 | 250 | 12 | 57 | 68 | 59 |
| Ab102 | 97 | 258 | 12 | 60 | 64 | 51 |
| Ab103 | 96 | 317 | 14 | 61 | 67 | 61 |
| Ab104 | 97 | 233 | 7 | 60 | 73 | 60 |
| Ab105 | 95 | 358 | 13 | 62 | 69 | 62 |
| Ab106 | 93 | 300 | 18 | 45 | 55 | 50 |
| Ab107 | 93 | 217 | 14 | 49 | 57 | 52 |
| Ab108 | 99 | 250 | 3 | 61 | 66 | 60 |
| Ab109 | 98 | 208 | 23 | 39 | 50 | 46 |
| Ab110 | 94 | 292 | ND | 61 | 73 | 57 |
| Ab111 | 95 | 275 | ND | 60 | 73 | 57 |
| Ab112 | 91 | 267 | ND | 57 | 71 | 64 |
| Ab113 | 95 | 258 | ND | 62 | 74 | 58 |
| Ab114 | 92 | 275 | ND | 61 | 71 | 66 |
| Ab115 | 85 | 283 | ND | 58 | 72 | 68 |

ND: not determined

### Example 7. In Vivo Effect of Anti-APJ Antibody Ab108 in a Mouse HHT Model

Hereditary hemorrhagic telangiectasia (HHT) is a genetic vascular disorder caused, for example, by loss-of-function mutations in the genes encoding endoglin (ENG) (e.g., causing hereditary hemorrhagic telangiectasia type 1 (HHT1)), activin receptor-like kinase 1 (ALK1/ACVRL1) (e.g., causing hereditary hemorrhagic telangiectasia type 2 (HHT2)), Smad4 (e.g., causing juvenile polyposis/hereditary hemorrhagic telangiectasia (JP-HHT)), or bone morphogenetic protein 9 (BMP9) (e.g., causing hereditary hemorrhagic telangiectasia type 5 (HHT5)). In two other less known HHT types, hereditary hemorrhagic telangiectasia type 3 (HHT3) and hereditary hemorrhagic telangiectasia type 4 (HHT4), disease-causing genes have not yet been definitively identified. HHT is characterized by the development of abnormal vascular structure in multiple tissues and is associated with clinical symptoms including recurrent nosebleeds; skin (e.g., fingers, nose, or face), mucous membrane (e.g., lips or tongue), or gastrointestinal tract telangiectasis (abnormal blood vessels and/or hemorrhages); or life-threatening events including internal bleeding and anemia. An underlying cause of HHT is formation of arteriovenous malformations (AVMs), abnormal shunts between arteries and veins without intervening capillaries, in brain, lungs, liver, and/or gastrointestinal tract. Controlling bleeding and diffused AVMs are regarded as critical improvement areas in HHT therapy. The BMP9/10-immunoblocked (BMP9/10ib) neonatal mouse model is reported to be one of the most reliable and robust models of HHT. Neutralization of BMP9 and BMP10 in neonatal mice leads to retinal vascular defects (AVMs and hypervascularization), retinal bleeding, and even anemia, which are also phenotypes observed in ALK1 or ENG conditional KO models (see, e.g., Tual-Chalot et al., 2016, Front. Genet. 18:6-25; Choi et al., 2023, Angiogenesis 26(1):167-186; and Ruiz et al., 2016, Sci. Rep. 5:37366). It has been shown that intraperitoneal (IP) administration of anti-BMP9/10 antibodies at P3 induces increased retinal vascular density and AVM numbers at postnatal day 6 (P6) and increased retinal AVM numbers, retinal bleeding, and decreased hemoglobin level at postnatal day 9 (P9) (see, e.g., Ruiz et al., 2016, Sc. Rep. 5:37366 and Ruiz et al., 2020, J. Clin. Invest. 130(2):942-957). Apelin/APJ has been shown to be involved in pathological angiogenesis, and apelin expression is upregulated in multiple genetic HHT mouse models (see, e.g., Kasai et al., 2010, Areterioscler. Thromb. Vasc. Biol. 30(11):2182-2187 and Zhou et al., 2023, Arterioscler. Thromb. Vasc. Biol. 43(8):1384-1403).

In this Example, the BMP9/10ib neonatal mouse model was validated by demonstrating upregulation of mRNA expression for APLN, the gene encoding apelin, and then was used to demonstrate the efficacy of an anti-APJ antibody antagonist described in Example 6, Ab108.

APLN mRNA Expression Study

An initial study was performed to confirm that mRNA expression of APLN is upregulated in the BMP9/10ib neonatal mouse model used in the rest of this Example. Timed-pregnant C57BL/6J mice (3-4-month-old) were used in this study. The pups from 4 litters were divided into the treatment groups described in Table 35. To induce the HHT-like phenotype, pups at P3 were intraperitoneally injected with anti-BMP9/10 (anti-BMP9, Biotechne cat. #MAB3208-500, anti-BMP10, Biotechne cat. #MAB2926-500) at a dosage of 15 mg/kg body weight per antibody. PBS was used as the vehicle control for the anti-BMP9/anti-BMP10 treatment. At P6, eyeballs were enucleated, retinal tissue was dissected and RNA was extracted using a RNeasy Plus Mini Kit (Qiagen). APLN mRNA levels were determined.

TABLE 35
Group assignments and dosage.

| | | Ab dosage | | Number |
|---|---|---|---|---|
| Group | Ab | (mg/kg) | Ab admin | of pups |
| 1 | None | N/A | IP, P3 | 14 |
| | (PBS)* | | | |
| 2 | Anti- | 15 | IP, P3 | 15 |
| | BMP9/10** | | | |

*PBS dosed at volume of 20 μL/g.
**Anti-BMP9 and Anti-BMP10 prepared separately and mixed 1:1 immediately before the injection and dosed at volume of 20 μL/g.

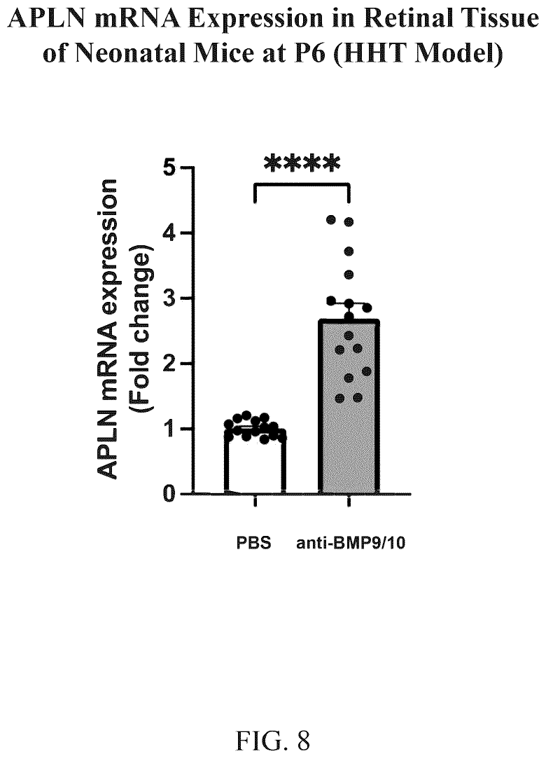

APLN mRNA expression in retinal tissue was significantly upregulated in BMP9/10ib neonatal mice compared to mice receiving vehicle control under the conditions tested (FIG. 8), providing validation of the BMP9/10ib neonatal mouse model used in the following studies.

### P6 Study

Timed-pregnant C57BL/6J mice (3-4-month-old) were used in this study. The pups from 4 litters were divided into the treatment groups described in Table 36. To induce the HHT-like phenotype, pups at P3 were intraperitoneally injected with anti-BMP9/10 (anti-BMP9, Biotechne cat. #MAB3208-500, anti-BMP10, Biotechne cat. #MAB2926-500) at a dosage of 15 mg/kg body weight. As a negative control for the anti-BMP9/10 treatment, a group of pups was treated with mouse IgG2a and IgG2b isotype controls (Biotechne, cat. #MAB003 and MAB004). The anti-BMP9/10-treated pups were then treated daily with AbNC (20 mg/kg), test molecule Ab108 (20 mg/kg), or VEGF neutralizing antibody anti-VEGF G6.31 positive control (10 mg/kg, Absolute Antibody cat. #Ab01022-2.0) from P3 to P5. G6.31 was used as a positive control in this experiment because it has been reported to be effective for epistaxis, a common symptom of HHT disease, and to inhibit the formation of AV shunts in an animal model of HHT2 (Dupuis-Girod et al., 2012, JAMA 307(9):948-955 and Han et al., 2014, Angiogenesis 17(4):823-830). At P6, the eyeballs of neonatal pups were enucleated and fixed in 4% paraformaldehyde for 30 min at room temperature. Retinas were dissected, blocked for 1 hr in blocking buffer, and incubated in blocking buffer at 4° C. overnight with Isolectin B4 (Invitrogen cat. #121411) and anti-α-SMA-Cy3 antibody (Sigma-Aldrich, cat. #C6198). The retinas were washed five times with wash buffer and mounted on microscope glass slides, and images were acquired by confocal microscopy. Retinal plexus area vascular density and AVM numbers were quantified from the images.

TABLE 36
Group assignments and dosage.

| | | Ab dosage | Ab | Test | Test molecule | Test molecule | Number |
|---|---|---|---|---|---|---|---|
| Group | Ab | (mg/kg) | admin | molecule*** | dosage (mg/kg) | admin. | of pups |
| 1 | IgG2a/2b** | 15 | IP, P3 | PBS | — | IP, P3, | 7 |
| | | | | | | P4, P5 | |
| 2 | Anti- | 15 | IP, P3 | AbNC | 20 | IP, P3, | 7 |
| | BMP9/10* | | | | | P4, P5 | |
| 3 | Anti- | 15 | IP, P3 | Ab108 | 20 | IP, P3, | 7 |
| | BMP9/10* | | | | | P4, P5 | |
| 4 | Anti- | 15 | IP, P3 | Anti-VEGF | 10 | IP, P3, | 7 |
| | BMP9/10* | | | (G6.31) | | P4, P5 | |

*Anti-BMP9 and Anti-BMP10 prepared separately and mixed 1:1 immediately before the injection and dosed at volume of 20 μL/g.
**IgG2b served as isotype control for anti-BMP9 and IgG2a served as isotype control for anti-BMP10. They were prepared separately and mixed 1:1 immediately before the injection and dosed at volume of 20 μL/g.
***Test molecules and PBS vehicle groups were dosed at the volume of 10 μL/g.

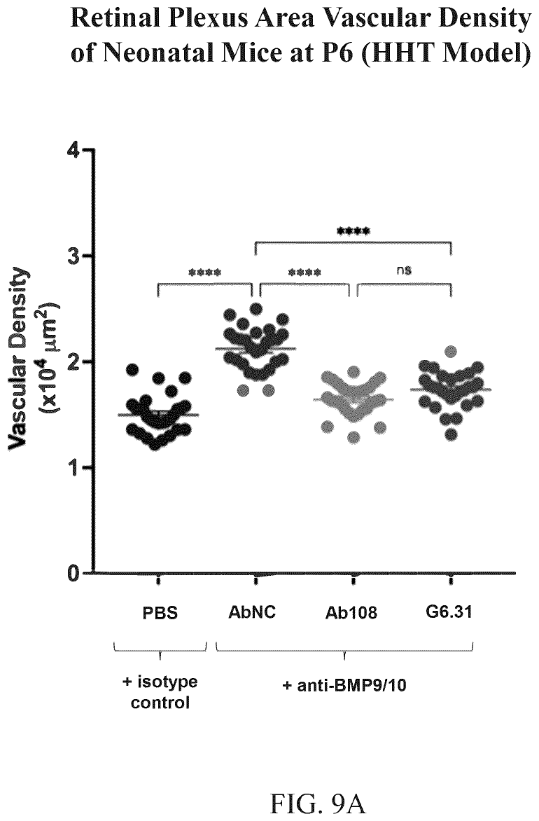

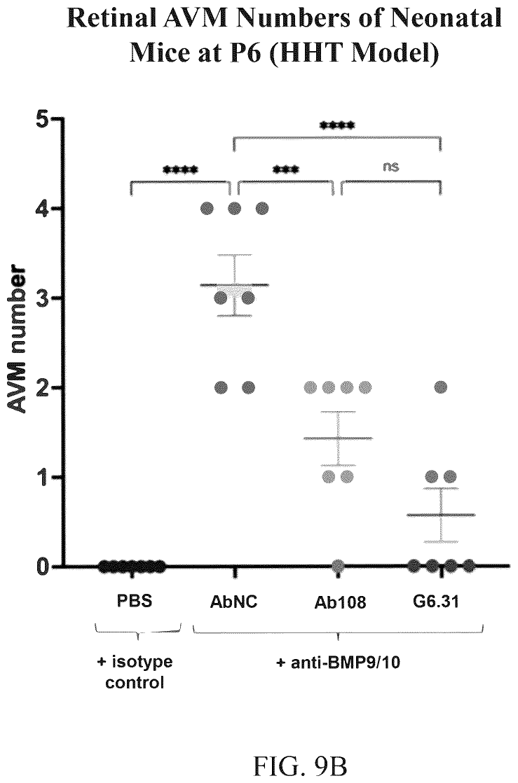

BMP9/10-immunoblocking at P3 increased retinal plexus area vascular density and AVM numbers of neonatal mice at P6 compared to the neonates administered with isotype control IgG2a/2b (FIGS. 9A and 9B). Treatment of the BMP9/10ib neonatal mice with Ab108 significantly reduced retinal vascular density (FIG. 9A) and AVM numbers (FIG. 9B). The result is comparable to treatment with G6.31.

### P9 Study

Timed-pregnant C57BL/6J mice (3-4-month-old) were used in this study. The pups from 8 litters were assigned into the treatment groups described in Table 37. To induce the HHT-like phenotype, pups at P3 were intraperitoneally injected with anti-BMP9/10 (anti-BMP9, Biotechne cat. #MAB3208-500, anti-BMP10, Biotechne cat. #MAB2926-500) at a dosage of 15 mg/kg body weight. As a negative control for the anti-BMP9/10 treatment, a group of pups was treated with mouse IgG2a and IgG2b isotype controls (Biotechne, cat. #MAB003 and MAB004). The anti-BMP9/10-treated pups were then treated daily with AbNC (10 mg/kg) or test molecule Ab108 (10 mg/kg) from P3 to P8. At P9, the pups were euthanized, and blood samples were collected via cardiac puncture. Hemoglobin levels were read using a hemoglobin photometer. Both eyeballs of neonatal pups were enucleated and fixed in 4% paraformaldehyde for 30 min at room temperature. Retinas were dissected, blocked for 1 hr in blocking buffer, and incubated in blocking buffer at 4° C. overnight with Isolectin B4 (Invitrogen cat. #121411) and anti-Ter119 antibody (Invitrogen cat. #MA1-70078). The retinas were then incubated with secondary antibody goat-anti-rat 594 (Invitrogen cat. #A11007) for 1 hour at room temperature and washed five times with wash buffer and mounted on microscope glass slides. Fluorescent images of the whole retinas were acquired using a BioTek Cytation 5 imager. AVM numbers, bleeding area percentage, and vasculature radial length were quantified from the images.

TABLE 37
Group assignments and dosage.

| | | Ab dosage | Ab | Test | Test molecule | Test molecule | Number |
|---|---|---|---|---|---|---|---|
| Group | Ab | (mg/kg) | admin | molecule*** | dosage (mg/kg) | admin. | of pups |
| 1 | IgG2a/2b** | 15 | IP, P3 | PBS | — | IP, P3, P4, P5, | 9 |
| | | | | | | P6, P7, P8 | |
| 2 | Anti- | 15 | IP, P3 | AbNC | 10 | IP, P3, P4, P5, | 18 |
| | BMP9/10* | | | | | P6, P7, P8 | |
| 3 | Anti- | 15 | IP, P3 | Ab108 | 10 | IP, P3, P4, P5, | 14 |
| | BMP9/10* | | | | | P6, P7, P8 | |

*Anti-BMP9 and Anti-BMP10 prepared separately and mixed 1:1 immediately before the injection and dosed at volume of 20 μL/g.
**IgG2b served as isotype control for anti-BMP9 and IgG2a served as isotype control for anti-BMP10. They were prepared separately and mixed 1:1 immediately before the injection and dosed at volume of 20 μL/g.
***Test molecules and PBS vehicle groups were dosed at the volume of 10 μL/g.

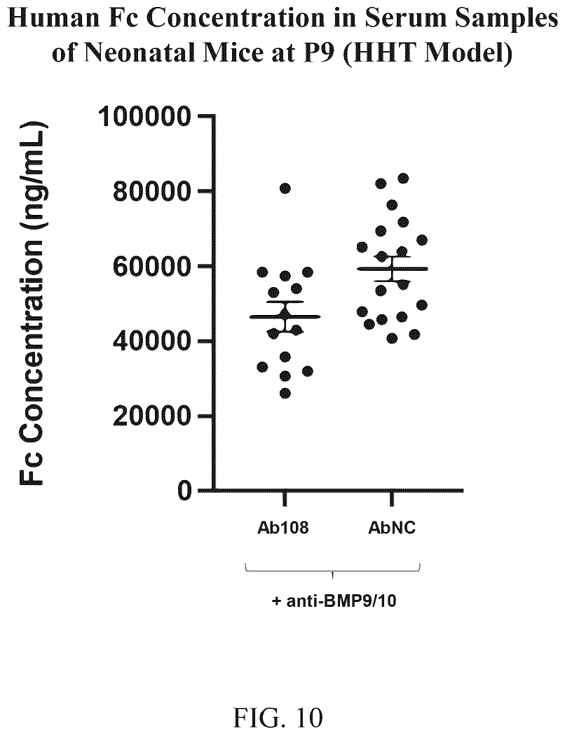

At the endpoint of treatment, human IgG Fc concentration was measured in serum samples from the neonatal mice via ELISA (Syd Labs Human Fc ELISA Reagent Kit, cat. #EK000095-20310). Briefly, ELISA plates were coated with capture antibody. Plates were washed, blocked with protein blocking solution, and washed again. Samples were added at a specific concentration, incubated, washed, incubated with secondary antibody, washed, and then developed for 5 minutes. IgG Fc concentrations were estimated using a standard curve. As shown in FIG. 10, high IgG Fc concentrations were detected in serum samples from the neonatal mice in Ab108 and AbNC treated groups, indicating a high level of test molecule exposure.

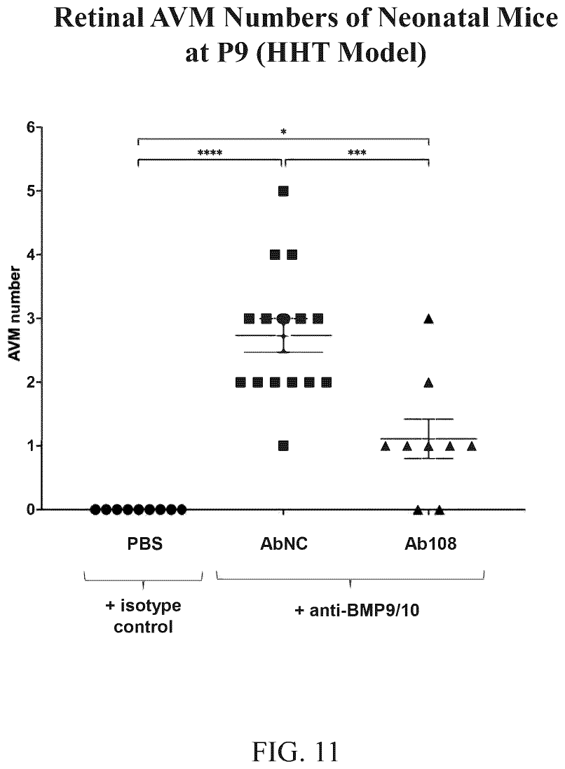

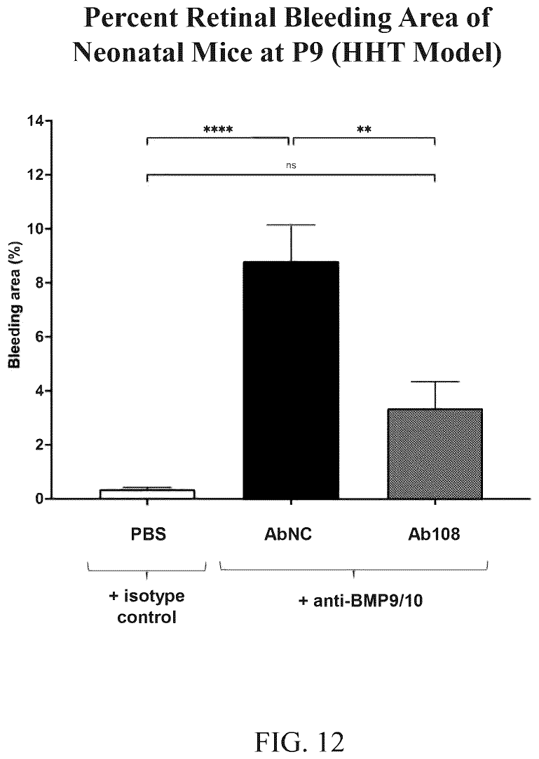

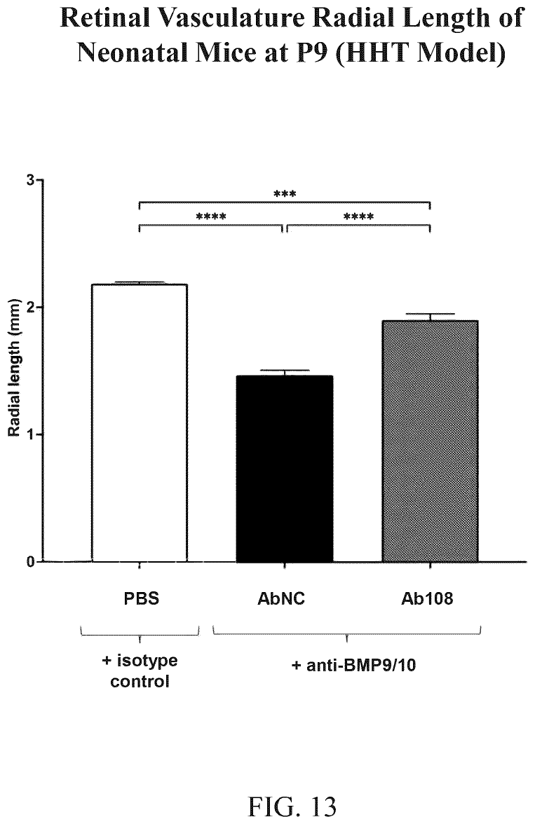

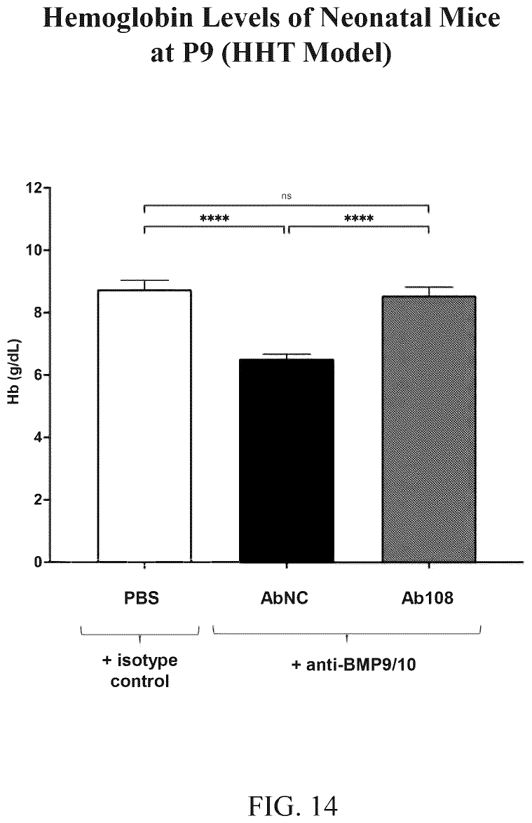

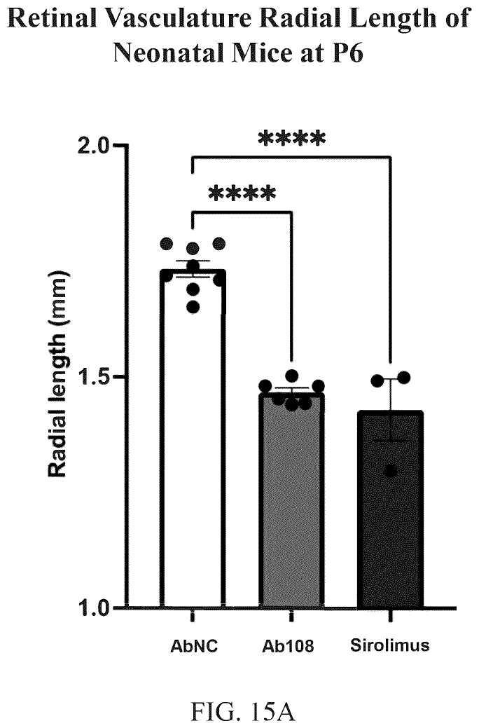

BMP9/10-immunoblocking at P3 increased retinal AVM numbers and bleeding area of neonatal mice at P9 compared to the neonates administered with isotype control IgG2a/2b, and these effects were significantly reduced with treatment of the BMP9/10ib mice with Ab108 (FIG. 11 and FIG. 12). Ab108 treatment also significantly ameliorated vasculature radial length reduction compared to BMP9/10ib neonates treated with AbNC (FIG. 13), and Ab108 treatment further significantly rescued low hemoglobin levels induced by BMP9/10ib in neonates at P9 (FIG. 14).

Taken together, the results described in this Example demonstrate that Ab108, an anti-APJ antagonist antibody, can reduce HHT pathology in a mouse model.

### Example 8. In Vivo Effect of Ab108 in Neonatal Mice

This study was conducted to demonstrate that Ab108 inhibits APJ-mediated vascularization in neonatal mice. Timed-pregnant C57BL/6J mice (3-4-month-old) were used in this study. The pups from 3 litters were assigned into the treatment groups described in Table 38. Ab108 at a 10 mg/kg dose was intraperitoneally injected daily from P1-P5. Sirolimus, an mTOR inhibitor, was used as a positive control and was dosed at 0.5 mg/kg IP daily from P3-P5. At P6, eyeballs of neonatal pups from all groups were enucleated and fixed in 4% paraformaldehyde for 30 min at room temperature. Retinas were dissected, blocked for 1 hr in blocking buffer (0.3% Triton X100, 0.2% BSA in 1×PBS) at room temperature, and incubated in blocking buffer at 4° C. overnight with Isolectin B4 (Invitrogen cat. #I21411). Retinal vasculature radial length and retinal vascularization (percent retinal vascularized area relative to total retinal area) were determined.

TABLE 38
Group assignments and dosage.

| | Test | Test molecule | Test molecule | Number |
|---|---|---|---|---|
| Group | molecule* | dosage (mg/kg) | admin. | of pups |
| 1 | AbNC | 10 | IP, P1, P2, P3, P4, | 7 |
| | | | P5 | |
| 2 | Ab108 | 10 | IP, P1, P2, P3, P4, | 6 |
| | | | P5 | |
| 3 | Sirolimus | 0.5 | IP, P3, P4, P5 | 3 |

*Test molecules were dosed at the volume of 10 μL/g.

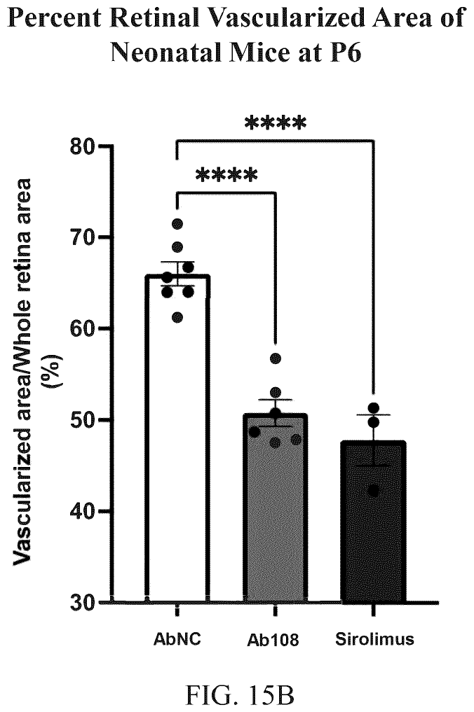

Ab108 significantly reduced the retinal vasculature radial length (FIG. 15A) and the percent retinal vascularized area (FIG. 15B) relative to AbNC in neonatal mice at P6.

### Example 9. Comparison of In Vitro and In Vivo Potencies of Ab108

This experiment was conducted to compare the in vitro and in vivo potencies of Ab108, the former by measuring cAMP levels using a highly sensitive HTRF-based competitive immunoassay (as described in Example 1) in the presence of apelin and increasing amounts of Ab108, and the latter by administering various doses of Ab108 to postnatal pups and assaying the radial length of the vascularized retina (measured as a radial surface distance from the center of the optic disc).

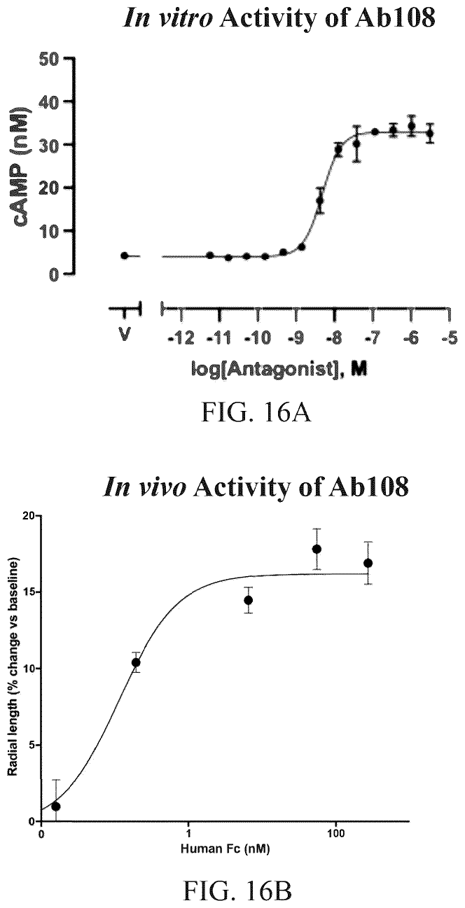

In the in vitro assay, as shown in FIG. 16A, Ab108 exhibited a dose-dependent effect, with an IC₉₀ of 16 nM (1.3 g/mL) and an IC₅₀ of 4.7 nM (0.4 g/mL).

In the in vivo assay, Ab108 was administered once to P1 pups (from timed-pregnant female C57BL/6 females) via intraperitoneal (i.p.) injection at varying doses (0, 1, 3, 10, 30, 60 mg/kg body weight). At P6, eyeballs of neonatal pups from all groups were enucleated and fixed in 4% paraformaldehyde for 30 min at room temperature. Retinas were dissected, blocked for 1 hr at room temperature in blocking buffer, and incubated in blocking buffer at 4° C. overnight with Isolectin B4 (Invitrogen cat. #121411). Fluorescent images of retinas were obtained by BioTek Cytation 5 imager. Radial length was determined.

Blood was collected at P6 via heart puncture and plasma was generated by letting blood clot overnight at 4° C. Plasma concentration of Ab108 was determined using an anti-human Fc ELISA.

As shown in FIG. 16B, Ab108 reduced the retinal vasculature radial length in the postnatal pups in a dose-dependent manner, with an IC₉₀ of 9.2 nM (0.7 μg/mL) and an IC₅₀ of 3.4 nM (0.3 g/mL).

These results confirm that the in vitro-determined and in vivo-determined potencies of Ab108 are comparable under the evaluated conditions.

### Example 10. In Vivo Effect of Anti-APJ Antibody Ab108 in an Adult Mouse HHT Model

As described above, controlling bleeding and diffused arteriovenous malformations (AVMs) are regarded as critical improvement areas in hereditary hemorrhagic telangiectasia (HHT) therapy. Example 7 describes the effect of treatment with an anti-APJ antibody antagonist (Ab108) in a BMP9/10-immunoblocked (BMP9/10ib) neonatal mouse model. Another HHT model, the inducible ALK1 knockout (iALK1 KO) adult model (Park et al., 2009, J. Clin. Invest. 119(11):3487-3496), employs a Cre/Lox system to globally delete the ALK1 (ACVRL1) gene following tamoxifen administration, resulting in biallelic gene deletion. Post-tamoxifen exposure, mice develop severe anemia (hemoglobin <10 g/dL) due to extensive hemorrhage in major organs such as the gastrointestinal tract, brain, and lungs, leading to mortality within 9-12 days. In short, this model recapitulates key features of the most severe forms of human HHT, including extensive systemic vascular malformations, hemorrhage, and anemia.

In this Example, the iALK1 KO model was used to compare the efficacy of an anti-APJ antibody antagonist described in Example 6, Ab108, to that of an anti-VEGFA IgG, G6.31, which has been shown to be protective in the iALK1 KO model. Both Ab108 and the anti-VEGFA comparator were assessed for their ability to reduce hemorrhage and vascular malformations following ALK1 deletion.

Group Assignments and Dosage: The animals used in the study were divided into groups as outlined in Table 39.

TABLE 39
Group assignments and dosage.

| | | | Tamoxifen | | Test | | |
|---|---|---|---|---|---|---|---|
| | | Tamoxifen | Route of | | Molecule | Test Molecule | |
| | Mouse | dosage ** | Admin. and | Test | Dosage | Route of Admin. | Number |
| Group | Genotype | (mg/g) | Time | Molecule *** | (mg/kg) | and Time | of mice |
| 1 | Alk1^{f/f} * | 0.1 | Intraperitoneal, | PBS | — | Intravenous, | 17 |
| | | | once a day at | | | Day 0, 3, 6, | |
| | | | Day 0 and 1 | | | and 9 | |
| 2 | Alk1^{f/f}; | 0.1 | Intraperitoneal, | AbNC | 10 | Intravenous, | 15 |
| | Rosa26^{CreERT2} | | once a day at | | | Day 0, 3, 6, | |
| | | | Day 0 and 1 | | | and 9 | |
| 3 | Alk1^{f/f}; | 0.1 | Intraperitoneal, | Ab108 | 10 | Intravenous, | 15 |
| | Rosa26^{CreERT2} | | once a day at | | | Day 0, 3, 6, | |
| | | | Day 0 and 1 | | | and 9 | |
| 4 | Alk1^{f/f}; | 0.1 | Intraperitoneal, | Anti- | 5 | Intravenous, | 16 |
| | Rosa26^{CreERT2} | | once a day at | VEGF | | Day 0, 3, 6, | |
| | | | Day 0 and 1 | | | and 9 | |

* Alk1^{f/f} mouse without Rosa26^{CreERT2} served as no disease control.
** Tamoxifen injection solution was prepared by dissolving tamoxifen in corn oil at a concentration of 10 mg/mL and dosed at a volume of 10 μL/g.
*** Test molecules and PBS vehicle groups were dosed at a volume of 10 μL/g.

ALK1^{f/f} mice and ALK1^{f/f}; Rosa26^{CreERT2} mice (both males and females, 8-12-month-old; The Jackson Laboratory) were used in this study. The mice were randomly assigned into the treatment groups as outlined in Table 39. To induce ALK1 deletion, all mice at Day 0 and 1 were intraperitoneally injected with tamoxifen at a dosage of 0.1 mg/g body weight. The mice were treated every three days with test molecule Ab108 (10 mg/kg), isotype control AbNC (10 mg/kg), or anti-VEGFA G6.31 (5 mg/kg, Absolute Antibody cat. #Ab01022-2.0) at Day 0, 3, 6, 9. Hemoglobin level was read by a hemoglobin photometer (HemoPoint H2 Photometer, Stanbio Laboratory) at Day 0, 7, 9, 11, and 12. At Day 12, all mice were euthanized, blood samples were collected, and images of cecum were taken. The GI hemorrhage levels were scored by visual inspection of the cecum. The mice were perfused with latex blue dye via left ventricle. The latex blue dye perfused GI tract was then enucleated and fixed in buffered 10% formalin at 4° C. overnight before being cleared with organic solvent (benzyl alcohol/benzyl benzoate, 1:1) after sequential methanol dehydration. The latex blue perfused blood vessels near Peyer's patch were imaged using a camera attached to the dissection microscope. Vascular areas containing latex and diameter of the vein were quantified using ImageJ.

For hemoglobin measurement, blood samples were drawn from mice via submandibular. A 5 mm lancet was used to puncture a small blood vessel near the cheek or jawline of the mouse to collect blood. 1 drop of blood (˜8 μL) was added to a microcuvette and hemoglobin was measured using the Hemopoint H2 Photometer. Values were obtained in duplicate or triplicate and averaged.

To measure hemorrhage index, after anesthesia of the mice with urethane, abdominal and thoracic cavities were opened. Hemorrhages in the gastrointestinal tract were observed and categorized into three levels: weak, moderate, and severe conditions, according to the degree of severity. Hemorrhage levels were graded: 1 for weak, 2 for moderate, and 3 for severe level. The mean values were expressed as severity index.

For human Fc ELISA, plates were coated with 50 μL capture antibody solution (20 μL of detection antibody stock per 5 mL PBS) overnight at 4° C. The plates were washed once with PBS and blocked with 300 μL of blocking reagent (3% BSA on PBS) per well for 2 hours at room temperature before liquid removal. The diluted samples and assay standards were prepared by spinning serum samples at 5000 rpm for 5 minutes at 4° C. Samples were then diluted 1:20000 and 1:40000 in the assay diluent (1% BSA on PBS), and the standard samples were generated by sequential 2-fold dilutions from 50 ng/mL to 0.78125 ng/mL and a blank. 50 μL of sample or standard was added per well, and the plate was incubated for 1 hour at room temperature, then washed 4 times with 300 μL PBS per well. The plates were then incubated with secondary antibody (1:1000 dilution of stock detection antibody in 1% BSA) for 1 hour at room temperature and washed 4 times with 300 μL PBS per well. 50 μL of TMB substrate was added to each well and incubated 3 minutes, until standards could easily be differentiated, and 50 μL of stop solution was then added to each well. The plates were read for absorbance at 450 nm and 570 nm. To eliminate background, the reading for absorbance at 570 nm was subtracted from the reading for absorbance at 450 nm for each well, and the blank well reading was subtracted for each well. The standards were plotted against their concentrations, and unknown values were interpolated using a 4-parameter logistic regression.

For latex blue dye perfusion, 25G infusion needles were carefully pierced into the left ventricle of the mouse heart, and the right atria was cut to release the blood. The mouse was slowly perfused with 10 mL PBS dilator (containing heparin 10 unit/mL, papaverine 0.04 mg/mL, and sodium nitroprusside 100 M) at a rate of 10 mL/min. The mouse was then slowly perfused with another 10 mL 10% formalin at a rate of 10 mL/min. The mouse was then perfused with 1 mL latex blue dye at a rate of 1 mL/min. The perfused animal was briefly washed with PBS, and then the perfused GI tract was enucleated and fixed in 10% Formalin overnight at 4° C. The fixed GI tract was cleared with organic solvent (benzyl alcohol/benzyl benzoate, 1:1) after sequential methanol dehydration.

For image acquisition and analysis, the latex blue perfused blood vessels near Peyer's patch were imaged using a CCD camera attached to the dissection microscope. Vascular density quantification was performed using ImageJ. Quantification was done using the measurement particles tool, working with 8-bit images, adjusting the threshold, and measuring the area percentage occupied by the vasculature in a region of interest of 2×2 mm² located near Peyer's patch. Vein diameter was measured at 2 different locations on the vein near Peyer's patch.

GraphPad Prism software was used for data analysis and plotting. Means and standard error of the mean (SEM) are presented. One-way ANOVA was used to assess statistical significance within multiple comparisons analysis. Tukey's multiple comparison test was performed as post hoc test.

### Results

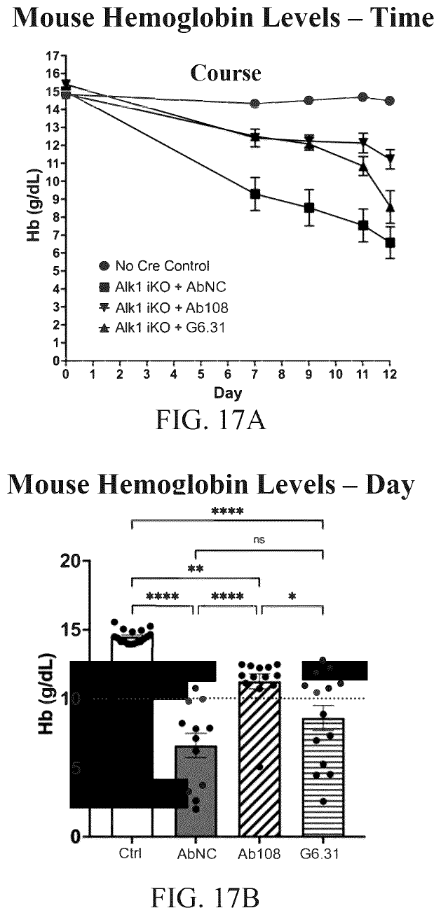

Ab108 treatment significantly mitigated anemia, with over 90% of treated animals maintaining hemoglobin levels above 10 g/dL (FIG. 17A). Even though both Ab108 and anti-VEGFA G6.31 treatment groups maintained their hemoglobin levels above 10 g/dL at day 7, by day 12 only Ab108 continued to prevent severe anemia in most animals, outperforming the anti-VEGFA G6.31 (FIG. 17B).

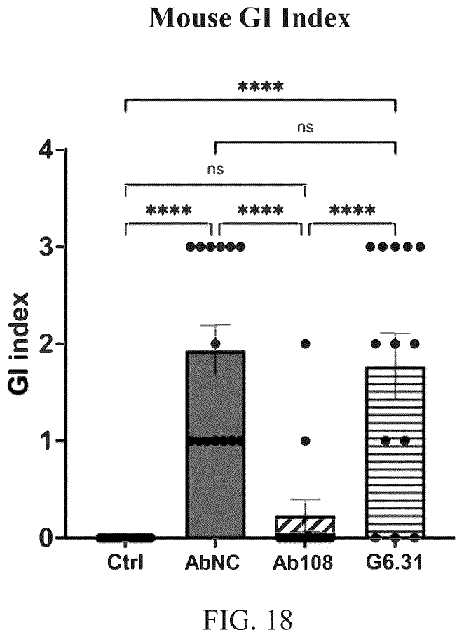

At the end point of treatment, GI tract bleeding was characterized by visual graded cecum score. GI bleeding was markedly reduced in the Ab108 treated group, while anti-VEGFA G6.31 treatment did not show protective effect in reducing GI bleeding relative to negative control antibody AbNC (FIG. 18).

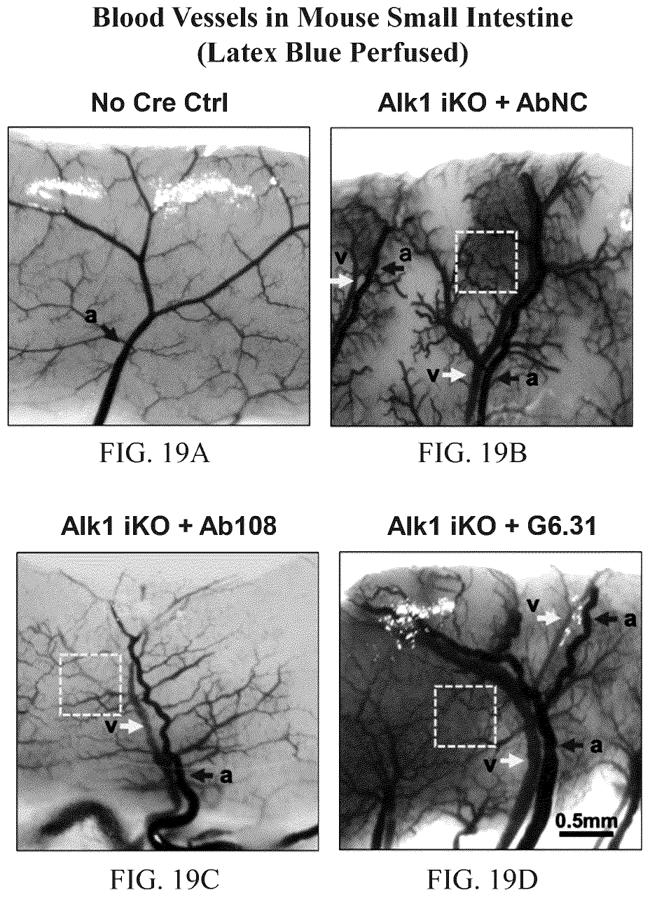

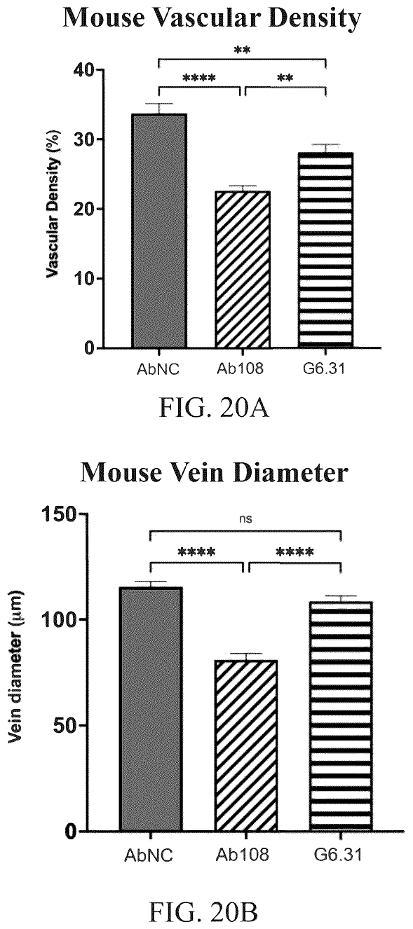

To visualize the vasculature and arteriovenous connections of blood vessels in the small intestine, latex blue dye was injected in the left ventricle. In no-Cre control mice, the latex dye did not cross the capillary beds and was retained within the arterial branches (FIG. 19A). However, in the ALK1 iKO mice, latex dye was found in both the arterial branches and venous branches, indicating the presence of AV shunts (FIGS. 19B-19D). The vascular density and vein diameter were then quantified from the images. Mice that received Ab108 treatment showed significantly less hypervascularization in the capillary bed compared to negative control AbNC or anti-VEGFA treated animals (FIG. 20A). Similarly, Ab108 treated mice showed significantly less hemorrhage (evidenced by diffused blue dye in the capillary bed) and vein dilation, outperforming anti-VEGFA G6.31 treatment (FIG. 20B).

This Example demonstrates that treatment with Ab108 at a dosage of 10 mg/kg significantly reduced GI tract hemorrhage in the ALK1 iKO adult mice model at Day 12. Ab108 treatment also mitigated anemia of ALK1 global deletion in mice by ameliorating the decreased hemoglobin value to normal level.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed:

1. An antibody that specifically binds human apelin receptor (APJ), the antibody comprising a heavy chain variable domain (VH) comprising the CDRH1, CDRH2, and CDRH3 amino acid sequences of the VH amino acid sequence set forth in SEQ ID NO: 76.

2. The antibody of claim 1, wherein the VH comprises the CDRH1, CDRH2, and CDRH3 amino acid sequences, respectively, set forth in SEQ ID NOs: 374, 384, and 141.

3. The antibody of claim 1, wherein the VH comprises the CDRH1, CDRH2, and CDRH3 amino acid sequences, respectively, set forth in SEQ ID NOs: 115, 144, and 141.

4. The antibody of claim 1, wherein the VH comprises the amino acid sequence set forth in SEQ ID NO: 76.

5. The antibody of claim 1, wherein the amino acid sequence of the VH consists of the amino acid sequence set forth in SEQ ID NO: 76.

6. The antibody of claim 1, further comprising an IgG Fc.

7. The antibody of claim 6, wherein the IgG Fc comprises the amino acid sequence of a human IgG1 Fc.

8. The antibody of claim 6, wherein the IgG Fc comprises:
alanine at EU position 329;
alanine at each of EU positions 234 and 235;
alanine at each of EU positions 234, 235, and 329;
leucine and serine at EU positions 428 and 434, respectively; or
alanine, alanine, alanine, leucine, and serine at EU positions 234, 235, 329, 428, and 434, respectively.

9. The antibody of claim 6, wherein the IgG Fc comprises the amino acid sequence set forth in SEQ ID NO: 726 or 742.

10. The antibody of claim 6, wherein the IgG Fc comprises the amino acid sequence set forth in SEQ ID NO: 733 or 749.

11. The antibody of claim 6, wherein the C-terminus of the IgG Fc is linked to the N-terminus of the VH.

12. The antibody of claim 6, wherein the N-terminus of the IgG Fc is linked to the C-terminus of the VH via a linker peptide.

13. The antibody of claim 12, wherein the linker peptide comprises the amino acid sequence set forth in SEQ ID NO:269.

14. The antibody of claim 6, wherein the antibody comprises the amino acid sequence set forth in SEQ ID NO: 766 or 800.

15. The antibody of claim 6, wherein the antibody comprises the amino acid sequence set forth in SEQ ID NO: 776 or 810.

16. The antibody of claim 6, wherein the amino acid sequence of the antibody consists of the amino acid sequence set forth in SEQ ID NO: 766 or 800.

17. The antibody of claim 16, wherein the antibody is dimeric.

18. The antibody of claim 6, wherein the amino acid sequence of the antibody consists of the amino acid sequence set forth in SEQ ID NO: 776 or 810.

19. The antibody of claim 18, wherein the antibody is dimeric.

20. An antibody that specifically binds human apelin receptor (APJ), wherein:
(a) the antibody comprises a heavy chain variable domain (VH) comprising complementarity determining regions CDRH1, CDRH2, and CDRH3, wherein:
the CDRH1 comprises the amino acid sequence of GX₁X₂X₃X₄X₅X₆CX₇X₈ (SEQ ID NO: 247), wherein: X₁ is L, F, I, S, Y, A, H, V, or Q; X₂ is T, H, L, N, Q, or S; X₃ is F, Y, I, L, or V; X₄ is S, A, H, Q, V, I, or T; X₅ is S, F, H, or Y; X₆ is H or Y; X₇ is M or absent; and X₈ is G, S, L, Y, or absent;
the CDRH2 comprises the amino acid sequence of X₉X₁₀X₁₁X₁₂SX₁₃GX₁₄X₁₅X₁₆X₁₇ (SEQ ID NO: 248), wherein: X₉ is A, L, or absent; X₁₀ is I or M; X₁₁ is S, A, Q, or T; X₁₂ is G, H, or R; X₁₃ is R or Y; X₁₄ is Y, S, T, F, or H; X₁₅ is S, T, Y, Q, or absent; X₁₆ is Y or absent; and X₁₇ is absent or Y; and
the CDRH3 comprises the amino acid sequence of AAVPRAGIX₁₈X₁₉X₂₀GAYCKX₂₁X₂₂X₂₃X₂₄DSGS (SEQ ID NO: 249), wherein: X₁₈ is E, F, Y, or W; X₁₉ is absent, Y, F, P, K, R, W, L, or I; X₂₀ is S, F, Y, or W; X₂₁ is W, A, F or Y; X₂₂ is S, H, I, K, N, P, Q, R, or T; X₂₃ is Y, G, H, I, L, M, N, or R; and X₂₄ is K or Q,
wherein the VH does not comprise the amino acid sequence set forth in SEQ ID NO: 60-64 or 823-830;
(b) the antibody comprises a heavy chain variable domain (VH) comprising complementarity determining regions CDRH1, CDRH2, and CDRH3, wherein:
the CDRH1 comprises the amino acid sequence of X₂₅X₂₆X₂₇X₂₈X₂₉X₃₀X₃₁X₃₂X₃₃X₃₄ (SEQ ID NO: 250), wherein: X₂₅ is G or Q; X₂₆ is F, Q, or V; X₂₇ is T, A, D, H, P, V, R, K, or E; X₂₈ is F, G, H, I, or V; X₂₉ is S, P, R, or K; X₃₀ is S or P; X₃₁ is P or Y; X₃₂ is H, A, P, R, or K; X₃₃ is M or absent; and X₃₄ is G, R, K, H, or absent;
the CDRH2 comprises the amino acid sequence of X₃₅X₃₆X₃₇X₃₈X₃₉X₄₀X₄₁X₄₂X₄₃X₄₄X₄₅X₄₆X₄₇X₄₈X₄₉X₅₀ (SEQ ID NO: 251), wherein: X₃₅ is A, G, S, V, R, K, H, or absent; X₃₆ is I, P, or T; X₃₇ is S or G; X₃₈ is G, F, or H; X₃₉ is S, I, L, V, or Y; X₄₀ is G, A, D, or E; X₄₁ is T, G, R, K, or H; X₄₂ is A or S; X₄₃ is G, T, or absent; X₄₄ is Y, Q, R, K, H, or absent; X₄₅ is Y, L, E, D, or absent; X₄₆ is A, L, or absent; X₄₇ is D, H, P, or absent; X₄₈ is S or absent; X₄₉ is V or absent; and X₅₀ is K, Q, or absent; and
the CDRH3 comprises the amino acid sequence of X₅₁X₅₂X₅₃X₅₄X₅₅X₅₆RX₅₇LX₅₈GX₅₉RX₆₀X₆₁X₆₂DY (SEQ ID NO: 252), wherein: X₅₁ is R, A, C, E, or S; X₅₂ is V, A, G, M, R, or S; X₅₃ is S, A, E, G, M, R, T, or V; X₅₄ is L, K, R, S, or V; X₅₅ is Q or G; X₅₆ is R or H; X₅₇ is T, L, or M; X₅₈ is D or E; X₅₉ is Y or F; X₆₀ is S or T; X₆₁ is S, I, V, or L; and X₆₂ is F or Y; or
(c) the APJ comprises the amino acid sequence of SEQ ID NO: 852, wherein:
(i) the antibody specifically interacts with the aspartate residue at position 172 of SEQ ID NO: 852;
(ii) the antibody does not specifically interact with the cysteine residue at position 281 of SEQ ID NO: 852; or
(iii) the antibody comprises a heavy chain variable domain (VH) comprising complementarity determining regions CDRH1, CDRH2, and CDRH3, and wherein:
(a) the antibody comprises a tyrosine residue in the CDRH2 that specifically interacts with the tyrosine residue at position 21 of SEQ ID NO: 852;
(b) the antibody comprises a serine residue in the CDRH3 that specifically interacts with the aspartate residue at position 172 of SEQ ID NO: 852; or
(c) the antibody comprises a tyrosine residue in the CDRH3 that specifically interacts with the aspartate residue at position 184 of SEQ ID NO: 852.

21. A composition comprising the antibody of claim 1 and a pharmaceutically acceptable carrier or excipient.

22. A composition comprising the antibody of claim 20 and a pharmaceutically acceptable carrier or excipient.

23. A polynucleotide encoding the antibody of claim 20.

24. A vector comprising the polynucleotide of claim 23.

25. A recombinant host cell comprising the polynucleotide of claim 23.

26. A method of producing an antibody, the method comprising culturing the recombinant host cell of claim 25 under suitable conditions such that the polynucleotide is expressed, and the antibody is produced.

27. A method of treating an APJ-associated disease or disorder in a subject, the method comprising administering to the subject an effective amount of (a) an antibody that specifically binds human apelin receptor (APJ), (b) a polynucleotide encoding the antibody, (c) a vector comprising the polynucleotide, (d) a recombinant host cell comprising the polynucleotide or the vector, or (e) a composition comprising any of (a)-(d) and a pharmaceutically acceptable carrier or excipient.

28. The method of claim 27, wherein the APJ-associated disease or disorder is selected from the group consisting of hereditary hemorrhagic telangiectasia (HHT), hereditary hemorrhagic telangiectasia type 1 (HHT1), hereditary hemorrhagic telangiectasia type 2 (HHT2), hereditary hemorrhagic telangiectasia type 3 (HHT3), hereditary hemorrhagic telangiectasia type 4 (HHT4), hereditary hemorrhagic telangiectasia type 5 (HHT5), juvenile polyposis/hereditary hemorrhagic telangiectasia (JP-HHT), angiodysplasia, arteriovenous malformation (AVM), brain AVM, bleeding, telangiectasia, von Willebrand Disease (vWD), type 2A vWD, acquired von Willebrand Syndrome (AvWS), pathological angiogenesis, Klippel-Trenaunay syndrome, Parkes-Weber syndrome, CLOVES syndrome, Proteus syndrome, blue rubber bleb nevus syndrome, aortic stenosis, calcific aortic stenosis with bicuspid aortic valve, calcific aortic stenosis without bicuspid aortic valve, Heyde's Syndrome, atherosclerosis, a vascular eye disease or disorder, epilepsy, cancer, glioblastoma, colorectal cancer, metastatic disease, endometriosis, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD).

29. The method of claim 27, wherein the APJ-associated disease or disorder is selected from the group consisting of obesity, muscle-sparing obesity, ischemia, ischemia/reperfusion injury, cerebral ischemia, neuronal injury, syndrome of inappropriate antidiuretic hormone secretion (SIADH), pulmonary arterial hypertension (PAH), cardiovascular disease, myocardial infarction, cardiomyopathy, a connective tissue disorder, fibrosis, idiopathic pulmonary fibrosis (IPF), diabetes, heart failure, acute decompensated heart failure, congestive heart failure, pulmonary hypertension, stroke, neurodegeneration, a fluid homeostasis disorder, and autosomal dominant polycystic kidney disease (ADPKD).
